SEQUENCE LISTING (336 sequences)

SEQ ID NO: 1 - protein, 7 aa, synthetic construct
SGGYYWT

SEQ ID NO: 2 - protein, 16 aa, synthetic construct
SIYYSGSTYYNPALKS

SEQ ID NO: 3 - protein, 10 aa, synthetic construct
ERKNELFFDY

SEQ ID NO: 4 - protein, 13 aa, synthetic construct
TRSSGSIASNYVQ

SEQ ID NO: 5 - protein, 7 aa, synthetic construct
EDKQRPS

SEQ ID NO: 6 - protein, 9 aa, synthetic construct
QSFDSSNRV

SEQ ID NO: 7 - protein, 120 aa, synthetic construct
QVQLQESGPGLVKPSQTLSLTCTVSGGSISSGGYYWTWIRQHPGKCLEWIGSIYYSGSTYYNPALKSRLTISVDTSKNQFSLKLSSVTAADTAVYYCARERKNELFFDYWGQGTLVTVSS

SEQ ID NO: 8 - protein, 110 aa, synthetic construct
NFMLTQPHSVSESPGKTVTISCTRSSGSIASNYVQWYQQRPDSSPTTVIYEDKQRPSGVPDRFSGSIDRSSNSASLTISGLKTEDEADYYCQSFDSSNRVFGCGTKLTVL

SEQ ID NO: 9 - protein, 5 aa, synthetic construct
FYTMN

SEQ ID NO: 10 - protein, 17 aa, synthetic construct
SITTSSRYIYYADSVKG

SEQ ID NO: 11 - protein, 11 aa, synthetic construct
AGTKTHDAFDF

SEQ ID NO: 12 - protein, 13 aa, synthetic construct
SRSSGSIASNYVQ

SEQ ID NO: 13 - protein, 7 aa, synthetic construct
EDNQRPS

SEQ ID NO: 14 - protein, 9 aa, synthetic construct
QSYDNSNRV

SEQ ID NO: 15 - protein, 120 aa, synthetic construct
EVQLVESGGGLVKPGGSLRLSCAASGFTFNFYTMNWVRQAPGKCLEWVSSITTSSRYIYYADSVKGRFTISRDNAKNSLYLQMNSLRAEDTAVYYCARAGTKTHDAFDFWGQGTMVTVSS

SEQ ID NO: 16 - protein, 110 aa, synthetic construct
NFMLTQPHSVSESPGKTVTISCSRSSGSIASNYVQWYQQRPGSSPTTVIYEDNQRPSGVPDRFSGSIDSSSNSASLTISGLKTEDEADYYCQSYDNSNRVFGCGTKLTVL

SEQ ID NO: 17 - protein, 5 aa, synthetic construct
SYSMN

SEQ ID NO: 18 - protein, 17 aa, synthetic construct
TISRSSSYIYYADSVKG

SEQ ID NO: 19 - protein, 10 aa, synthetic construct
EKWNLLYFDS

SEQ ID NO: 20 - protein, 13 aa, synthetic construct
TRSSGSIASNYVQ

SEQ ID NO: 21 - protein, 7 aa, synthetic construct
EDNQRPS

SEQ ID NO: 22 - protein, 9 aa, synthetic construct
QSYDSSNRV

SEQ ID NO: 23 - protein, 119 aa, synthetic construct
EVQLVESGGGLVKPGGSLRLSCAASGFTVSSYSMNWVRQAPGKCLEWVSTISRSSSYIYYADSVKGRFTISRDNAKNSLYLQMNSLRAEDTAVYFCAREKWNLLYFDSWGQGTLVTVSS

SEQ ID NO: 24 - protein, 110 aa, synthetic construct
NFMLTQSHSVSESPGKTVTISCTRSSGSIASNYVQWYQQRPGSSPTTVIYEDNQRPSGVPDRFSGSIDSSSNSASLTISGLKTEDEADYYCQSYDSSNRVFGCGTKLTVL

SEQ ID NO: 25 - protein, 5 aa, synthetic construct
SYSMN

SEQ ID NO: 26 - protein, 17 aa, synthetic construct
AISRSSTYIYYADSVKG

SEQ ID NO: 27 - protein, 10 aa, synthetic construct
ENWDLLYFEF

SEQ ID NO: 28 - protein, 13 aa, synthetic construct
TRSSGSIASNYVQ

SEQ ID NO: 29 - protein, 7 aa, synthetic construct
EDNQRPS

SEQ ID NO: 30 - protein, 9 aa, synthetic construct
QSYDSSNRV

SEQ ID NO: 31 - protein, 119 aa, synthetic construct
EVQLVESGGGLVKPGGSLRLSCAASGFTLSSYSMNWVRQAPGRCLEWVSAISRSSTYIYYADSVKGRFTISRDNAKNSVHLQMSSLRAEDTAVYYCARENWDLLYFEFWGQGTLVTVSS

SEQ ID NO: 32 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNRVFGCGTKLTVL

SEQ ID NO: 33 - protein, 5 aa, synthetic construct
SYSMN

SEQ ID NO: 34 - protein, 17 aa, synthetic construct
YISSSGSYKYYADSVKG

SEQ ID NO: 35 - protein, 9 aa, synthetic construct
DRKQLLLDY

SEQ ID NO: 36 - protein, 11 aa, synthetic construct
SGDALPKKYAY

SEQ ID NO: 37 - protein, 7 aa, synthetic construct
EDSKRPS

SEQ ID NO: 38 - protein, 11 aa, synthetic construct
YSTDSSGNHWV

SEQ ID NO: 39 - protein, 118 aa, synthetic construct
EVQLVESGGGLVKPGGSLRLSCAASGFTFSSYSMNWVRQAPGKGLEWVSYISSSGSYKYYADSVKGRFTISRDNAKNSLYLQMNSLRAEDTAVFYCARDRKQLLLDYWGQGTLVTVSS

SEQ ID NO: 40 - protein, 108 aa, synthetic construct
SYELTQPPSVSVSPGQTARITCSGDALPKKYAYWYQQKSGQAPVLVIYEDSKRPSGIPERFSGSSSGTMATLTISGAQVEDEADYYCYSTDSSGNHWVFACGTKLTVL

SEQ ID NO: 41 - protein, 5 aa, synthetic construct
SYTMN

SEQ ID NO: 42 - protein, 17 aa, synthetic construct
SITRSSRYIYYADSVKG

SEQ ID NO: 43 - protein, 11 aa, synthetic construct
AGAATHDAFDI

SEQ ID NO: 44 - protein, 13 aa, synthetic construct
TRSSGSIASNYVQ

SEQ ID NO: 45 - protein, 7 aa, synthetic construct
EDNQRPS

SEQ ID NO: 46 - protein, 9 aa, synthetic construct
QSYDSNNRV

SEQ ID NO: 47 - protein, 120 aa, synthetic construct
EVQLVESGGGLVTPGGSLRLSCAASGFTFSSYTMNWVRQAPGKCLEWVSSITRSSRYIYYADSVKGRFTISRDNAKNSLYLQMNSLRAEDTAVYYCARAGAATHDAFDIWAQGTMVTVSS

SEQ ID NO: 48 - protein, 110 aa, synthetic construct
NFMLTQPHSVSESPGKTVTISCTRSSGSIASNYVQWYQQRPGSSPATVIYEDNQRPSGVPDRFSGSIDSSSNSASLTISGLKTEDEADYYCQSYDSNNRVFGCGTKLTVL

SEQ ID NO: 49 - protein, 5 aa, synthetic construct
FYTMN

SEQ ID NO: 50 - protein, 17 aa, synthetic construct
SITTSSRYIYYADSVKG

SEQ ID NO: 51 - protein, 11 aa, synthetic construct
AGAKTHDAFDF

SEQ ID NO: 52 - protein, 13 aa, synthetic construct
SRSSGSIASNYVQ

SEQ ID NO: 53 - protein, 7 aa, synthetic construct
EDNQRPS

SEQ ID NO: 54 - protein, 9 aa, synthetic construct
QSYDSSNRV

SEQ ID NO: 55 - protein, 120 aa, synthetic construct
EVQLVESGGGLVKPGGSLRLSCAASGFTFSFYTMNWVRQAPGKCLEWVSSITTSSRYIYYADSVKGRFTISRDNAKNSLYLQMNSLRAEDTAVYYCARAGAKTHDAFDFWGQGTMVTVSS

SEQ ID NO: 56 - protein, 110 aa, synthetic construct
NFMLTQPHSVSESPGKTVTISCSRSSGSIASNYVQWYQQRPGSSPTTVIYEDNQRPSGVPDRFSGSIDSSSNSASLTISGLKTEDEADYYCQSYDSSNRVFGCGTKLTVL

SEQ ID NO: 57 - protein, 5 aa, synthetic construct
SYSMN

SEQ ID NO: 58 - protein, 17 aa, synthetic construct
SISRSTAYIYYADSVKG

SEQ ID NO: 59 - protein, 10 aa, synthetic construct
EKWELLYFDS

SEQ ID NO: 60 - protein, 13 aa, synthetic construct
TRNSGSIASNYVQ

SEQ ID NO: 61 - protein, 7 aa, synthetic construct
EDNQRPS

SEQ ID NO: 62 - protein, 9 aa, synthetic construct
QSYDSSNRV

SEQ ID NO: 63 - protein, 119 aa, synthetic construct
EVQLVESGGGLVKPGGSLRLSCAASGFTLSSYSMNWVRQAPGKCLEWVSSISRSTAYIYYADSVKGRFTISRDNAKNSVHLQMNSLRAEDTAVYFCAREKWELLYFDSWGQGTLVTVSS

SEQ ID NO: 64 - protein, 110 aa, synthetic construct
NFMLTQPHSVSESPGKTVTISCTRNSGSIASNYVQWYQQRPGSSPTTVIYEDNQRPSGVPDRFSGSIDSSSNSASLTISGLKTEDEADYYCQSYDSSNRVFGCGTKLTVL

SEQ ID NO: 65 - protein, 5 aa, synthetic construct
TSPIN

SEQ ID NO: 66 - protein, 17 aa, synthetic construct
SISRISSHKYYADSVKG

SEQ ID NO: 67 - protein, 10 aa, synthetic construct
EKWELLYFDY

SEQ ID NO: 68 - protein, 17 aa, synthetic construct
RSSQSLLDSDDGNTYLD

SEQ ID NO: 69 - protein, 7 aa, synthetic construct
TLSYRAS

SEQ ID NO: 70 - protein, 10 aa, synthetic construct
MQRIEFPSIT

SEQ ID NO: 71 - protein, 119 aa, synthetic construct
EVQVVESGGGLVKPGGSLRLSCAASGFTLSTSPINWVRQAPGKCLEWVSSISRISSHKYYADSVKGRFTISRDNAKNSVYLQMNSLRVEDTAVYYCAREKWELLYFDYWGQGTLVTVSS

SEQ ID NO: 72 - protein, 114 aa, synthetic construct
DIVMTQTPLSLPVTPGEPASISCRSSQSLLDSDDGNTYLDWYLQKPGQSPQLLIYTLSYRASGVPDRFSGSGSGTDFTLKISRVEAEDVGIYYCMQRIEFPSITFGCGTRLVIK

SEQ ID NO: 73 - protein, 5 aa, synthetic construct
TSPIN

SEQ ID NO: 74 - protein, 17 aa, synthetic construct
SISRISSHKYYADSVKG

SEQ ID NO: 75 - protein, 10 aa, synthetic construct
EKWELLYFDY

SEQ ID NO: 76 - protein, 13 aa, synthetic construct
TRSSGSIASNYVQ

SEQ ID NO: 77 - protein, 7 aa, synthetic construct
EDNQRPS

SEQ ID NO: 78 - protein, 9 aa, synthetic construct
QSYDSSNRV

SEQ ID NO: 79 - protein, 119 aa, synthetic construct; identical to SEQ ID NO: 71

SEQ ID NO: 80 - protein, 110 aa, synthetic construct
NFMLTQPHSVSESPGKTVTISCTRSSGSIASNYVQWYQQRPGSSPTAVIYEDNQRPSGVPDRFSGSIDSSSNSASLTISGLKTEDEADYYCQSYDSSNRVFGCGTKLTVL

SEQ ID NO: 81 - protein, 5 aa, synthetic construct
NYILN

SEQ ID NO: 82 - protein, 17 aa, synthetic construct
SISSSSRYIFYADSVKG

SEQ ID NO: 83 - protein, 11 aa, synthetic construct
ESKWELLSFDY

SEQ ID NO: 84 - protein, 13 aa, synthetic construct
TRSSGSIASNYVQ

SEQ ID NO: 85 - protein, 7 aa, synthetic construct
EDNQRPS

SEQ ID NO: 86 - protein, 9 aa, synthetic construct
QSYDRSNRV

SEQ ID NO: 87 - protein, 120 aa, synthetic construct
EVQLVESGGGLVKPGGSLRLSCAASGFTFNNYILNWVRQAPGKCLEWVSSISSSSRYIFYADSVKGRFTISRDNAKNSLYLQMNSLRAEDTGVYYCARESKWELLSFDYWGQGTLVTVSS

SEQ ID NO: 88 - protein, 110 aa, synthetic construct
NFMLTQPHSVSESPGKTVTISCTRSSGSIASNYVQWYQQRPGSSPINVIYEDNQRPSGVPDRFSGSIDSSSNSASLTISGLKTEDEADYYCQSYDRSNRVFGCGTKLTVL

SEQ ID NO: 89 - protein, 7 aa, synthetic construct
SGGYSWS

SEQ ID NO: 90 - protein, 16 aa, synthetic construct
SIYYSGRTYYNPALKS

SEQ ID NO: 91 - protein, 10 aa, synthetic construct
ERKNELFFDY

SEQ ID NO: 92 - protein, 13 aa, synthetic construct
TRSSGSIASNYVQ

SEQ ID NO: 93 - protein, 7 aa, synthetic construct
EDNQRPS

SEQ ID NO: 94 - protein, 9 aa, synthetic construct
QSYDSSNRV

SEQ ID NO: 95 - protein, 120 aa, synthetic construct
QVQLQESGPGLVKPSQTLSLTCTVSGGSISSGGYSWSWIRQHPGKCLEWIGSIYYSGRTYYNPALKSRITISVDTSKNQFSLMLSSVPAADTAVYYCARERKNELFFDYWGQGTLVTVSS

SEQ ID NO: 96 - protein, 110 aa, synthetic construct
NFMLTQPHSVSESPGKTITISCTRSSGSIASNYVQWYQQRPGSSPTAVIYEDNQRPSGVPDRFSGSIDSSSNSASLTISGLKTEDEADYYCQSYDSSNRVFGCGTKLTVL

SEQ ID NO: 97 - protein, 5 aa, synthetic construct
SSLMN

SEQ ID NO: 98 - protein, 17 aa, synthetic construct
SISSSSNYIYYADSVRG

SEQ ID NO: 99 - protein, 10 aa, synthetic construct
ERWVLLYFDY

SEQ ID NO: 100 - protein, 13 aa, synthetic construct
TRSSGSIASNYVQ

SEQ ID NO: 101 - protein, 7 aa, synthetic construct
EDNQRPS

SEQ ID NO: 102 - protein, 9 aa, synthetic construct
QSYDSSIRV

SEQ ID NO: 103 - protein, 119 aa, synthetic construct
EVHLVESGGGLVKPGGSLRLSCAASGFTFSSSLMNWVRQAPGKCLEWVSSISSSSNYIYYADSVRGRFTISRDNAKNSLYLQMHSLRAEDTAVYYCARERWVLLYFDYWGQGTLVTVSS

SEQ ID NO: 104 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSIRVFGCGTKLTVL

SEQ ID NO: 105 - protein, 5 aa, synthetic construct
SSLMN

SEQ ID NO: 106 - protein, 17 aa, synthetic construct
SISSSSNYIYYADSVRG

SEQ ID NO: 107 - protein, 10 aa, synthetic construct
ERWVLLYFDY

SEQ ID NO: 108 - protein, 13 aa, synthetic construct
TRSSGSIASNYVQ

SEQ ID NO: 109 - protein, 7 aa, synthetic construct
EDNQRPS

SEQ ID NO: 110 - protein, 9 aa, synthetic construct
QSYDSSIRV

SEQ ID NO: 111 - protein, 119 aa, synthetic construct; identical to SEQ ID NO: 103

SEQ ID NO: 112 - protein, 110 aa, synthetic construct; identical to SEQ ID NO: 104

SEQ ID NO: 113 - protein, 5 aa, synthetic construct
SYSMN

SEQ ID NO: 114 - protein, 17 aa, synthetic construct
YINSRSSTKYYADSVKG

SEQ ID NO: 115 - protein, 9 aa, synthetic construct
VMRHYGMDV

SEQ ID NO: 116 - protein, 13 aa, synthetic construct
TRSSGSIASNYVQ

SEQ ID NO: 117 - protein, 7 aa, synthetic construct
EDNQRPS

SEQ ID NO: 118 - protein, 10 aa, synthetic construct
QSFDNSNHWV

SEQ ID NO: 119 - protein, 118 aa, synthetic construct
EVQLAESGGGLVQPGGSLRLSCAASGFTFSSYSMNWVRQAPGKCLEWVLYINSRSSTKYYADSVKGRFTISRDNAKNSLYLQMNSLRDADTAVYYCARVMRHYGMDVWGQGTTVTVSS

SEQ ID NO: 120 - protein, 111 aa, synthetic construct
NFMLTQPHSVSESPEKTVTISCTRSSGSIASNYVQWYQQRPGNSPTTLIYEDNQRPSGVPDRFSGSIDSSSNSASLTISGLKTEDEADYYCQSFDNSNHWVFGCGTKLTVL

SEQ ID NO: 121 - protein, 5 aa, synthetic construct
RYSMN

SEQ ID NO: 122 - protein, 17 aa, synthetic construct
TVSSGSRYRYYADSVKG

SEQ ID NO: 123 - protein, 11 aa, synthetic construct
AGASTHDSFDI

SEQ ID NO: 124 - protein, 13 aa, synthetic construct
TRSRGSIASNYVQ

SEQ ID NO: 125 - protein, 7 aa, synthetic construct
EDNKRPS

SEQ ID NO: 126 - protein, 9 aa, synthetic construct
QSYDNSNRV

SEQ ID NO: 127 - protein, 120 aa, synthetic construct
EVQLVESGGGLVKPGGSLTLSCEASGFTFNRYSMNWVRQGPGKCLEWVSTVSSGSRYRYYADSVKGRFTISRDNAKNSLYLQMNSLRAEDTAVYYCARAGASTHDSFDIWGQGTMVTVPS

SEQ ID NO: 128 - protein, 110 aa, synthetic construct
NFMLTQSHSVSESPGKTVTISCTRSRGSIASNYVQWYQQRPGSSPTTVIYEDNKRPSGVPDRFSGSIDSSSNSASLTISGLKTEDEADYYCQSYDNSNRVFGCGTKLTVL

SEQ ID NO: 129 - protein, 5 aa, synthetic construct
SYSMN

SEQ ID NO: 130 - protein, 17 aa, synthetic construct
SISRSSSYIYYADSVKG

SEQ ID NO: 131 - protein, 10 aa, synthetic construct
EKWELLYFDA

SEQ ID NO: 132 - protein, 13 aa, synthetic construct
TRSSGSIASNYVQ

SEQ ID NO: 133 - protein, 7 aa, synthetic construct
EDNQRPS

SEQ ID NO: 134 - protein, 9 aa, synthetic construct
QSYDRSNRV

SEQ ID NO: 135 - protein, 119 aa, synthetic construct
EVQLVESGGGLVKPGGSLRLSCAASGFTVSSYSMNWVRQAPGKCLEWVSSISRSSSYIYYADSVKGRFTISRDNAKNSLYLQMNSLRAEDTAVYYCAREKWELLYFDAWGQGTLVTVSS

SEQ ID NO: 136 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDRSNRVFGCGTKLTVL

SEQ ID NO: 137 - protein, 5 aa, synthetic construct
SYSMN

SEQ ID NO: 138 - protein, 17 aa, synthetic construct
SSSRSSSYIYYADSVKG

SEQ ID NO: 139 - protein, 10 aa, synthetic construct
ERWELLYFDY

SEQ ID NO: 140 - protein, 13 aa, synthetic construct
TRSSGSIASNYVQ

SEQ ID NO: 141 - protein, 7 aa, synthetic construct
EDNQRPS

SEQ ID NO: 142 - protein, 9 aa, synthetic construct
QSYDSSIRV

SEQ ID NO: 143 - protein, 119 aa, synthetic construct
EVQLVESGGGLVKPGGSLRLSCAASGFTFSSYSMNWVRQAPGKCLEWVSSSSRSSSYIYYADSVKGRFTISRDNAKNSLYLQMNSLRAEDTAVYYCARERWELLYFDYWGQGTLVTVSS

SEQ ID NO: 144 - protein, 110 aa, synthetic construct; identical to SEQ ID NO: 104

SEQ ID NO: 145 - protein, 7 aa, synthetic construct
SGAYYWN

SEQ ID NO: 146 - protein, 16 aa, synthetic construct
DIYYSGTTYYNPALKS

SEQ ID NO: 147 - protein, 10 aa, synthetic construct
ERKNELFFDY

SEQ ID NO: 148 - protein, 13 aa, synthetic construct
TRSSGSIASNYVQ

SEQ ID NO: 149 - protein, 7 aa, synthetic construct
EDNQRPS

SEQ ID NO: 150 - protein, 9 aa, synthetic construct
QSYDSSNRV

SEQ ID NO: 151 - protein, 120 aa, synthetic construct
QVQLQESGPGLVKPSQTLSLTCIVSGGSISSGAYYWNWIRQHPGECLEWIGDIYYSGTTYYNPALKSRVSISVDTSKNQFSLNLISVTAADTAVYYCARERKNELFFDYWGQGTLVTVSS

SEQ ID NO: 152 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTLSGLKTEDEADYYCQSYDSSNRVFGCGTKLTVL

SEQ ID NO: 153 - protein, 5 aa, synthetic construct
NYAMN

SEQ ID NO: 154 - protein, 17 aa, synthetic construct
SISRSNSHKYYADSVKG

SEQ ID NO: 155 - protein, 10 aa, synthetic construct
EKWELLYFDS

SEQ ID NO: 156 - protein, 17 aa, synthetic construct
RSSQSLLDSDDGNTYLD

SEQ ID NO: 157 - protein, 7 aa, synthetic construct
TLSYRAS

SEQ ID NO: 158 - protein, 10 aa, synthetic construct
MQRIEFPSIT

SEQ ID NO: 159 - protein, 119 aa, synthetic construct
EVQVVESGGGLVKPGGSLRLSCAASGFTLSNYAMNWVRQAPGKCLEWISSISRSNSHKYYADSVKGRFAISRDIAKNSVYLQMNSLRAEDTAVYYCAREKWELLYFDSWGQGTLVTVSS

SEQ ID NO: 160 - protein, 114 aa, synthetic construct
DIVMTQTPLSLPVTPGEPASISCRSSQSLLDSDDGNTYLDWYLQKPGRSPQLLIYTLSYRASGVPDRFSGSGSGTDFTLKISRVEAEDVGIYYCMQRIEFPSITFGCGTRLEIK

SEQ ID NO: 161 - protein, 6 aa, synthetic construct
NYAMNW

SEQ ID NO: 162 - protein, 17 aa, synthetic construct
SISRSNSHKYYADSVKG

SEQ ID NO: 163 - protein, 10 aa, synthetic construct
EKWELLYFDS

SEQ ID NO: 164 - protein, 13 aa, synthetic construct
TRSSGSIASNYVQ

SEQ ID NO: 165 - protein, 7 aa, synthetic construct
EDDQRPS

SEQ ID NO: 166 - protein, 9 aa, synthetic construct
QSYDSSNRV

SEQ ID NO: 167 - protein, 119 aa, synthetic construct; identical to SEQ ID NO: 159

SEQ ID NO: 168 - protein, 110 aa, synthetic construct
NFMLTQPHSVSESPGKTITISCTRSSGSIASNYVQWYQQRPGSSPTTVIYEDDQRPSGVPDRFSGSIDSSSNSASLTISGLKTEDEADYFCQSYDSSNRVFGCGTKLTVL

SEQ ID NO: 169 - protein, 6 aa, synthetic construct
DYYIHW

SEQ ID NO: 170 - protein, 17 aa, synthetic construct
WINPNSGGTTYVQKFQG

SEQ ID NO: 171 - protein, 11 aa, synthetic construct
DRGTYYDYFDN

SEQ ID NO: 172 - protein, 17 aa, synthetic construct
KSSESVLYRSNNKNYLT

SEQ ID NO: 173 - protein, 7 aa, synthetic construct
WASIRES

SEQ ID NO: 174 - protein, 9 aa, synthetic construct
QQYYGTPYT

SEQ ID NO: 175 - protein, 120 aa, synthetic construct
QVQLMQSGAEVKKPGASVKVSCKASGYTFTDYYIHWVRQAPGQCLEWMGWINPNSGGTTYVQKFQGRVTMTRDTSISTAYMELSRLRSDDTAVYYCSRDRGTYYDYFDNWGQGTLVTVSS

SEQ ID NO: 176 - protein, 113 aa, synthetic construct
DIVMTQSPDSLAVSLGERATINCKSSESVLYRSNNKNYLTWYQQKPGQPPKLLIYWASIRESGVPDRFSGSGSGTDFTLTISSLQAEDVAVYFCQQYYGTPYTFGCGTKLEIK

SEQ ID NO: 177 - protein, 5 aa, synthetic construct
RNSMN

SEQ ID NO: 178 - protein, 17 aa, synthetic construct
SISTSGKYRYYADSLKD

SEQ ID NO: 179 - protein, 11 aa, synthetic construct
AGGSTHDSFDI

SEQ ID NO: 180 - protein, 13 aa, synthetic construct
TRSSGSIARNYVQ

SEQ ID NO: 181 - protein, 7 aa, synthetic construct
EDNKRPS

SEQ ID NO: 182 - protein, 9 aa, synthetic construct
QSYDNSNRV

SEQ ID NO: 183 - protein, 119 aa, synthetic construct
EVQLVESGGGLVKPGGSLRLSCAASGFTFSRNSMNWVRQAPGKCLEWVSSISTSGKYRYYADSLKDRFTISRDNAMNSLYLQMNSLRVEDTAVYYCARAGGSTHDSFDIWGQGTMVTVS

SEQ ID NO: 184 - protein, 110 aa, synthetic construct
NFMLTQPHSVSESPGKTVTISCTRSSGSIARNYVQWYQQRPGSSPKTVIYEDNKRPSGVPDRISGSIDSSSNSASLTISGLKTEDEADYYCQSYDNSNRVFGGGTKLTVL

SEQ ID NO: 185 - protein, 5 aa, synthetic construct
FYTMN

SEQ ID NO: 186 - protein, 17 aa, synthetic construct
SITTSSRYIYYADSVKG

SEQ ID NO: 187 - protein, 11 aa, synthetic construct
AGAKTHDAFDF

SEQ ID NO: 188 - protein, 13 aa, synthetic construct
SRSSGSIASNYVQ

SEQ ID NO: 189 - protein, 7 aa, synthetic construct
EDNQRPS

SEQ ID NO: 190 - protein, 9 aa, synthetic construct
QSYDSSNRV

SEQ ID NO: 191 - protein, 120 aa, synthetic construct
EVHLVESGGGLVKPGGSLRLSCAASGFTFSFYTMNWVRQAPGKCLEWVSSITTSSRYIYYADSVKGRFTVSRDNAKNSLYLQMNSLRAEDTAVYYCARAGAKTHDAFDFWGQGTMVTVSS

SEQ ID NO: 192 - protein, 110 aa, synthetic construct; identical to SEQ ID NO: 56

SEQ ID NO: 193 - protein, 5 aa, synthetic construct
RYSMN

SEQ ID NO: 194 - protein, 17 aa, synthetic construct
SISSSSYYIYYADSLKG

SEQ ID NO: 195 - protein, 8 aa, synthetic construct
ERRAGFDY

SEQ ID NO: 196 - protein, 13 aa, synthetic construct
TRSSGSIASNYVQ

SEQ ID NO: 197 - protein, 7 aa, synthetic construct
EDNQRPS

SEQ ID NO: 198 - protein, 9 aa, synthetic construct
QSYDSSNRV

SEQ ID NO: 199 - protein, 117 aa, synthetic construct
EVQLVESGGGLVKPGGSLRLSCAASGFTFSRYSMNWVRQAPGKCLEWVSSISSSSYYIYYADSLKGRFTISRDNAKSSLYLQMNSLRAEDTAVYYCARERRAGFDYWGQGTLVTVSS

SEQ ID NO: 200 - protein, 110 aa, synthetic construct; identical to SEQ ID NO: 32

SEQ ID NO: 201 - protein, 5 aa, synthetic construct
SSSMN

SEQ ID NO: 202 - protein, 17 aa, synthetic construct
SISSRSRHIYYADSVKG

SEQ ID NO: 203 - protein, 10 aa, synthetic construct
EKWELLYFDY

SEQ ID NO: 204 - protein, 11 aa, synthetic construct
GGNNIGSKSVH

SEQ ID NO: 205 - protein, 7 aa, synthetic construct
DDSDRPS

SEQ ID NO: 206 - protein, 11 aa, synthetic construct
QVWDSSSDHVV

SEQ ID NO: 207 - protein, 119 aa, synthetic construct
EVQLVESGGGLVKPGGSLRLSCAASGFTFSSSSMNWVRQAPGKCLEWVSSISSRSRHIYYADSVKGRFTISRDNAKNSLYLQMNSLRAEDTAVYYCAREKWELLYFDYWGQGTLVTVSS

SEQ ID NO: 208 - protein, 108 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHVVFGCGTKLTVL

SEQ ID NO: 209 - protein, 5 aa, synthetic construct
SSSMN

SEQ ID NO: 210 - protein, 17 aa, synthetic construct
SISSRSRHIYYADSVKG

SEQ ID NO: 211 - protein, 10 aa, synthetic construct
EKWELLYFDY

SEQ ID NO: 212 - protein, 12 aa, synthetic construct
RSSGSIASNYVQ

SEQ ID NO: 213 - protein, 7 aa, synthetic construct
EDNQRPS

SEQ ID NO: 214 - protein, 9 aa, synthetic construct
QSYDSSNRV

SEQ ID NO: 215 - protein, 119 aa, synthetic construct; identical to SEQ ID NO: 207

SEQ ID NO: 216 - protein, 110 aa, synthetic construct
NFMLTQPHSVSESPGKTVTISCTRSSGSIASNYVQWYQQRPGSSPTTLIYEDNQRPSGVPDRFSGSVDSSSNSASLTISGLKTEDEADYYCQSYDSSNRVFGCGTKLTVL

SEQ ID NO: 217 - protein, 5 aa, synthetic construct
SYSMN

SEQ ID NO: 218 - protein, 17 aa, synthetic construct
SISRSSAYIYYADSVKG

SEQ ID NO: 219 - protein, 10 aa, synthetic construct
EKWELLYFDY

SEQ ID NO: 220 - protein, 12 aa, synthetic construct
RSSGSIASNYVQ

SEQ ID NO: 221 - protein, 7 aa, synthetic construct
EDNQRPS

SEQ ID NO: 222 - protein, 9 aa, synthetic construct
QSYDSSNRV

SEQ ID NO: 223 - protein, 119 aa, synthetic construct
EVQLVESGGGLVKPGGSLRLSCAASRFTLSSYSMNWVRQAPGKCLEWVSSISRSSAYIYYADSVKGRFTISRDNAKNSVHLQMNSLRAEDTAVYYCAREKWELLYFDYWGQGTLVTVSS

SEQ ID NO: 224 - protein, 110 aa, synthetic construct
NFMLTQPHSVSESPGKTVTISCTRSSGSIASNYVQWYQRRPGSSPTTVIYEDNQRPSGVPDRFSGSIDSSSNSASLTISGLKTEDEADYYCQSYDSSNRVFGCGTKLTVL

SEQ ID NO: 225 - protein, 5 aa, synthetic construct
RYSMN

SEQ ID NO: 226 - protein, 17 aa, synthetic construct
TVSSGSRYRYYADSVKG

SEQ ID NO: 227 - protein, 11 aa, synthetic construct
AGGNTHDAFDI

SEQ ID NO: 228 - protein, 13 aa, synthetic construct
TRSRGSIASNYVQ

SEQ ID NO: 229 - protein, 7 aa, synthetic construct
EDNKRPS

SEQ ID NO: 230 - protein, 8 aa, synthetic construct
SYDNSNRV

SEQ ID NO: 231 - protein, 120 aa, synthetic construct
EVQLVESGGGLVKPGGSLRLSCAASGFTFNRYSMNWVRQAPGKCLEWVSTVSSGSRYRYYADSVKGRFTISRDNAKNSLYLQMNSLRAEDTAVYYCARAGGNTHDAFDIWGQGTMVTVSS

SEQ ID NO: 232 - protein, 110 aa, synthetic construct
NFMLTQSHSVSESPGKTVTISCTRSRGSIASNYVQWYQQRPGSSPTTVIYEDNKRPSGVPDRFSGSIDSSSNSASLTISGLKTEDEADYYCQSYDNSNRVFGCGTKVTVL

SEQ ID NO: 233 - protein, 6 aa, synthetic construct
RYSMNW

SEQ ID NO: 234 - protein, 17 aa, synthetic construct
LISSSSSYKYYVDSVKG

SEQ ID NO: 235 - protein, 9 aa, synthetic construct
DRRELVLDY

SEQ ID NO: 236 - protein, 11 aa, synthetic construct
SGDALPKKYAY

SEQ ID NO: 237 - protein, 7 aa, synthetic construct
EDSKRPS

SEQ ID NO: 238 - protein, 10 aa, synthetic construct
YSTNNSGNHW

SEQ ID NO: 239 - protein, 118 aa, synthetic construct
EVQLVESGGGLVKPGGSLRLSCAASGFTFSRYSMNWVRQAPGKCLEWVSLISSSSSYKYYVDSVKGRFTISRDNAKNSLYLEMSSLRAEDTAVYYCARDRRELVLDYWGQGTLVTVSS

SEQ ID NO: 240 - protein, 108 aa, synthetic construct
SYELTQPPSVSVSPGQTARITCSGDALPKKYAYWYQQKSGQAPVLVIYEDSKRPSGIPERLSGSSSGTMATLIISGAQVEDEADYYCYSTNNSGNHWVFGCGTKLTVL

SEQ ID NO: 241 - protein, 5 aa, synthetic construct
TYSMN

SEQ ID NO: 242 - protein, 17 aa, synthetic construct
TFSRSSSHIYYADSVKG

SEQ ID NO: 243 - protein, 10 aa, synthetic construct
EQWNLLYFDY

SEQ ID NO: 244 - protein, 13 aa, synthetic construct
TRSSGSIASNYVQ

SEQ ID NO: 245 - protein, 7 aa, synthetic construct
EDNQRPS

SEQ ID NO: 246 - protein, 9 aa, synthetic construct
QSYDRSNRV

SEQ ID NO: 247 - protein, 119 aa, synthetic construct
EVQLVESGGGLVRPGGSLRLSCAASGFTLSTYSMNWVRQAPGKCLEWVSTFSRSSSHIYYADSVKGRFTISRDNAKNSLYLQMNSLRAEDTAVYYCAREQWNLLYFDYWGQGTLVTVSS

SEQ ID NO: 248 - protein, 110 aa, synthetic construct
NLMLTQPHSVSESPGKTVTISCTRSSGSIASNYVQWYQQRPGSSPTTVIYEDNQRPSGVPDRFSGSIDSSSNSASLTISGLKTEDEADYYCQSYDRSNRVFGCGTKLTVL

SEQ ID NO: 249 - protein, 6 aa, synthetic construct
SRYSMN

SEQ ID NO: 250 - protein, 17 aa, synthetic construct
SISSGSRYIYYRDSVRG

SEQ ID NO: 251 - protein, 8 aa, synthetic construct
EPRNHFDY

SEQ ID NO: 252 - protein, 13 aa, synthetic construct
TRSSGSIASNYVQ

SEQ ID NO: 253 - protein, 7 aa, synthetic construct
EDNQRPS

SEQ ID NO: 254 - protein, 9 aa, synthetic construct
QSYDSSIRV

SEQ ID NO: 255 - protein, 117 aa, synthetic construct
EVQLVESGGGLVKPGGSLKLSCAASGFTFSRYSMNWVRQASGKCLEWVSSISSGSRYIYYRDSVRGRFTISRDNAKNSLYLQMNSLRAEDTALYYCAREPRNHFDYWGQGTLVTVSS

SEQ ID NO: 256 - protein, 110 aa, synthetic construct
NFMLIQPHSVSESPGKTVTISCTRSSGSIASNYVQWYQHHPGSSPTTVIYEDNQRPSGVPDRFSGSIDSSSNSASLTISGLKTEDEADYYCQSYDSSIRVFGCGTKLTVL

SEQ ID NO: 257 - protein, 5 aa, synthetic construct
RYSMN

SEQ ID NO: 258 - protein, 17 aa, synthetic construct
TISSSSSYRYYADSVKG

SEQ ID NO: 259 - protein, 11 aa, synthetic construct
AGGNTHDAFEI

SEQ ID NO: 260 - protein, 13 aa, synthetic construct
TRSSGSIARNYVQ

SEQ ID NO: 261 - protein, 7 aa, synthetic construct
EDNKRPS

SEQ ID NO: 262 - protein, 9 aa, synthetic construct
QSYDNSNRV

SEQ ID NO: 263 - protein, 120 aa, synthetic construct
EVQLVESGGGLVKPGGSLRLSCEASGFTFSRYSMNWVRQAPGKCLGWVSTISSSSSYRYYADSVKGRFTISRDNAKNSLYLQMNSLRAEDTAVYYCARAGGNTHDAFEIWGQGTMVTVSS

SEQ ID NO: 264 - protein, 110 aa, synthetic construct
NFMLTQPHSVSASPGKTVTISCTRSSGSIARNYVQWYQQRPGSSPTTVIFEDNKRPSGVPDRFSGSIDSSSNSASLTISGLKTEDEADYYCQSYDNSNRVFGCGTKLTVL

SEQ ID NO: 265 - protein, 5 aa, synthetic construct
SYTMN

SEQ ID NO: 266 - protein, 17 aa, synthetic construct
SITRSSRYIYYADSVKG

SEQ ID NO: 267 - protein, 11 aa, synthetic construct
AGAATHDAFDI

SEQ ID NO: 268 - protein, 13 aa, synthetic construct
TRSSGSIASNYVQ

SEQ ID NO: 269 - protein, 7 aa, synthetic construct
EDNQRPS

SEQ ID NO: 270 - protein, 9 aa, synthetic construct
QSYDGNNRV

SEQ ID NO: 271 - protein, 120 aa, synthetic construct
EVQLVESGGGLVKPGGSLRLSCAASGFTFSSYTMNWVRQAPGKCLEWVSSITRSSRYIYYADSVKGRFIISRDNAKNSLYLQMNSLRAEDTAVYYCARAGAATHDAFDIWAQGTMVTVSS

SEQ ID NO: 272 - protein, 110 aa, synthetic construct
NFMLTQPHSVSESPGKTVTISCTRSSGSIASNYVQWYQQRPGSSPATVIYEDNQRPSGVPDRFSGSIDSSSNSASLTISGLKTEDEADYYCQSYDGNNRVFGCGTKLTVL

SEQ ID NO: 273 - protein, 5 aa, synthetic construct
IYSMN

SEQ ID NO: 274 - protein, 17 aa, synthetic construct
SISSSSSYIHYADSVKG

SEQ ID NO: 275 - protein, 9 aa, synthetic construct
DRRERLLAY

SEQ ID NO: 276 - protein, 11 aa, synthetic construct
SGDALPKKYAY

SEQ ID NO: 277 - protein, 7 aa, synthetic construct
EDSKRPS

SEQ ID NO: 278 - protein, 11 aa, synthetic construct
YSTDSRGNLWV

SEQ ID NO: 279 - protein, 118 aa, synthetic construct
EVQLVESGGGLVKPGGSLRLSCAASGFTFSIYSMNWVRQAPGKCLEWVSSISSSSSYIHYADSVKGRFTISRDNAKNSMHLQLNSLRAEDTAVYYCARDRRERLLAYWGQGTLVTVSS

SEQ ID NO: 280 - protein, 108 aa, synthetic construct
SYELTQPPSVSVSPGQTARITCSGDALPKKYAYWYQQKSGQAPVLVIYEDSKRPSGIPERFSGSSSGTMATLTISGAQVEDEADYYCYSTDSRGNLWVFGCGTKVTVL

SEQ ID NO: 281 - protein, 5 aa, synthetic construct
SYSMN

SEQ ID NO: 282 - protein, 17 aa, synthetic construct
TISRSSSYIYYADSVKG

SEQ ID NO: 283 - protein, 10 aa, synthetic construct
EKWNLLYFDA

SEQ ID NO: 284 - protein, 13 aa, synthetic construct
TRSSGSIASNYVQ

SEQ ID NO: 285 - protein, 7 aa, synthetic construct
EDNQRPS

SEQ ID NO: 286 - protein, 9 aa, synthetic construct
QSYDSSNRV

SEQ ID NO: 287 - protein, 119 aa, synthetic construct
EVQLVESGGGLVKPGGSLRLSCAASGFTVSSYSMNWVRQAPGKCLEWVSTISRSSSYIYYADSVKGRFTISRDNAKNSLYLQMNSLRAEDTAVYFCAREKWNLLYFDAWGQGTLVTVSS

SEQ ID NO: 288 - protein, 110 aa, synthetic construct; identical to SEQ ID NO: 32

SEQ ID NO: 289 - protein, 5 aa, synthetic construct
SYSMN

SEQ ID NO: 290 - protein, 17 aa, synthetic construct
SISRSSNYIYYADSVKG

SEQ ID NO: 291 - protein, 10 aa, synthetic construct
EKWNLLYFDY

SEQ ID NO: 292 - protein, 13 aa, synthetic construct
TRSSGSIATNYVQ

SEQ ID NO: 293 - protein, 7 aa, synthetic construct
EDNKRPS

SEQ ID NO: 294 - protein, 9 aa, synthetic construct
QSYDSSNRV

SEQ ID NO: 295 - protein, 118 aa, synthetic construct
EVQLVESGGGLVKPGGSLRLSCAASGFTLSSYSMNWVRQAPGKCLEWVSSISRSSNYIYYADSVKGRFTISRDNAKNSLYLQMNSLRAEDTAVYYCAREKWNLLYFDYWGQGTLVTVS

SEQ ID NO: 296 - protein, 110 aa, synthetic construct
NLMLTQPHSVSGSPGKTVTISCTRSSGSIATNYVQWYQQRPGSSPTTVIYEDNKRPSGVPDRFSGSIDSSSNSASLTISGLKTEDEADYYCQSYDSSNRVFGCGTKLTVL

SEQ ID NO: 297 - protein, 5 aa, synthetic construct
RYWMT

SEQ ID NO: 298 - protein, 17 aa, synthetic construct
NIKKDGSENYYVDSVKG

SEQ ID NO: 299 - protein, 10 aa, synthetic construct
VRVIYDWLDP

SEQ ID NO: 300 - protein, 11 aa, synthetic construct
SGDKLGNKYAA

SEQ ID NO: 301 - protein, 7 aa, synthetic construct
QDSKRPS

SEQ ID NO: 302 - protein, 9 aa, synthetic construct
QAWDNNIVV

SEQ ID NO: 303 - protein, 117 aa, synthetic construct
EVQVVESGGGLVQPGGSLRLSCAASGFTFSRYWMTWVRQAPGKCLEWVANIKKDGSENYYVDSVKGRFTISRDNAKDSLYLQMNSLRAEDTAVYYCVRVIYDWLDPWGQGTLVTVSS

SEQ ID NO: 304 - protein, 106 aa, synthetic construct
SYELTQPPSVSVSPGQTASITCSGDKLGNKYAAWYQQKPGQSPVLVIYQDSKRPSGIPERFSGSNSGNTATLTISGTQAMDEADYYCQAWDNNIVVFGCGTKLTVL

SEQ ID NO: 305 - protein, 5 aa, synthetic construct
RYSMN

SEQ ID NO: 306 - protein, 17 aa, synthetic construct
SISMSSRYIYYVDSVKG

SEQ ID NO: 307 - protein, 10 aa, synthetic construct
ERGPGPGMDV

SEQ ID NO: 308 - protein, 13 aa, synthetic construct
TRSSGSIASNYVQ

SEQ ID NO: 309 - protein, 7 aa, synthetic construct
EDNQRPS

SEQ ID NO: 310 - protein, 9 aa, synthetic construct
QSYDSSNRV

SEQ ID NO: 311 - protein, 119 aa, synthetic construct
EVQLVESGGGLVKFGGSLRLSCAASGFTFSRYSMNWVRQAPGKCLDWVSSISMSSRYIYYVDSVKGRFTISRDNAKNSLYLQMNSLRADDTAVYYCARERGPGPGMDVWGQGTTVTVSS

SEQ ID NO: 312 - protein, 110 aa, synthetic construct; identical to SEQ ID NO: 32

SEQ ID NO: 313 - protein, 247 aa, synthetic construct
NFMLIQPHSVSESPGKTVTISCTRSSGSIASNYVQWYQHHPGSSPTTVIYEDNQRPSGVPDRFSGSIDSSSNSASLTISGLKTEDEADYYCQSYDSSIRVFGCGTKLTVLGGSEGKSSGSGSESKSTGGSEVQLVESGGGLVKPGGSLKLSCAASGFTFSRYSMNWVRQASGKCLEWVSSISSGSRYIYYRDSVRGRFTISRDNAKNSLYLQMNSLRAEDTALYYCAREPRNHFDYWGQGTLVTVSS

SEQ ID NO: 314 - protein, 479 aa, synthetic construct
NFMLIQPHSVSESPGKTVTISCTRSSGSIASNYVQWYQHHPGSSPTTVIYEDNQRPSGVPDRFSGSIDSSSNSASLTISGLKTEDEADYYCQSYDSSIRVFGCGTKLTVLGGSEGKSSGSGSESKSTGGSEVQLVESGGGLVKPGGSLKLSCAASGFTFSRYSMNWVRQASGKCLEWVSSISSGSRYIYYRDSVRGRFTISRDNAKNSLYLQMNSLRAEDTALYYCAREPRNHFDYWGQGTLVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 315 - protein, 5 aa, synthetic construct
NYGVH

SEQ ID NO: 316 - protein, 16 aa, synthetic construct
VIWAAGRTNYNSALMS

SEQ ID NO: 317 - protein, 10 aa, synthetic construct
DRYDYGYFDV

SEQ ID NO: 318 - protein, 10 aa, synthetic construct
SASSSVSYMY

SEQ ID NO: 319 - protein, 7 aa, synthetic construct
RTSSLAS

SEQ ID NO: 320 - protein, 9 aa, synthetic construct
QQYHSYPLT

SEQ ID NO: 321 - protein, 118 aa, synthetic construct
QVQLQESGPGLVKPSETLSITCTVSGFSLINYGVHWIRQPPGKGLEWLGVIWAAGRTNYNSALMSRLYISVDTSKNQVSLKLSSVTAADTAVYYCARDRYDYGYFDVWGQGTMVTVSS

SEQ ID NO: 322 - protein, 106 aa, synthetic construct
AIVLTQSPSSLSASTGDRVTISCSASSSVSYMYWYQQKSGKAPKLWIYRTSSLASRVPSRFSGSGSGTDYTLTISSLQSEDFATYYCQQYHSYPLTFGGGTKVEIK

SEQ ID NO: 323 - protein, 448 aa, synthetic construct
QVQLQESGPGLVKPSETLSITCTVSGFSLINYGVHWIRQPPGKGLEWLGVIWAAGRTNYNSALMSRLYISVDTSKNQVSLKLSSVTAADTAVYYCARDRYDYGYFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 324 - protein, 213 aa, synthetic construct
AIVLTQSPSSLSASTGDRVTISCSASSSVSYMYWYQQKSGKAPKLWIYRTSSLASRVPSRFSGSGSGTDYTLTISSLQSEDFATYYCQQYHSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 325 - protein, 20 aa, synthetic construct
GGSEGKSSGSGSESKSTGGS

SEQ ID NO: 326 - protein, 182 aa, synthetic construct
MEQGKGLAVLILAIIILQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 327 - protein, 171 aa, synthetic construct
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 328 - protein, 207 aa, synthetic construct
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 329 - protein, 164 aa, synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 330 - protein, 220 aa, synthetic construct
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAIIRDFYNPLVAEAQKRELGASLYLGWAASGLLLLGGGLLCCTCPSGGSQGPSHYMARYSTSAPAISRGPSEYPTKNYV

SEQ ID NO: 331 - protein, 5 aa, synthetic construct
GSEIL

SEQ ID NO: 332 - protein, 9 aa, synthetic construct
CYPRGSKPE

SEQ ID NO: 333 - protein, 6 aa, synthetic construct
TCPQYP

SEQ ID NO: 334 - protein, 6 aa, synthetic construct
QSGYYV

SEQ ID NO: 335 - protein, 10 aa, synthetic construct
DANFYLYLRA

SEQ ID NO: 336 - protein, 15 aa, synthetic construct
RLDLGKRILDPRGIY

(19) United States
(12) Patent Application Publication
(10) Pub. No.: US 2026/0103520 A1
(43) Pub. Date: Apr. 16, 2026

(54) ANTI-CD3 ANTIBODIES AND METHODS OF USE THEREOF

(71) Applicant: Third Arc Bio, Inc., Lower Gwynedd, PA (US)

(72) Inventors: Sanjaya Singh, Lower Gwynedd, PA (US); Danlin Yang, Lower Gwynedd, PA (US)

(21) Appl. No.: 19/358,785

(22) Filed: Oct. 15, 2025

Related U.S. Application Data
(60) Provisional application No. 63/707,305, filed on Oct. 15, 2024.
(60) Provisional application No. 63/791,917, filed on Apr. 21, 2025.

(51) Int. Cl. C07K 16/28; A61K 39/00; A61P 35/00

(52) U.S. Cl. CPC C07K 16/2809; A61P 35/00; C07K 16/28; A61K 2039/505; C07K 2317/31; C07K 2317/34; C07K 2317/52; C07K 2317/565; C07K 2317/732

(57) ABSTRACT

Antibodies and antigen-binding fragments thereof that specifically bind CLDN6 or CD3, and bispecific antibodies and antigen-binding domains that specifically bind CLDN6 and CD3 are described. Also described are nucleic acids encoding the antibodies, compositions comprising the antibodies, methods of producing the antibodies, and methods of using the antibodies for treating or preventing diseases, such as cancer.

### CROSS-REFERENCE TO RELATED APPLICATIONS

This application claims priority to and benefit of U.S. Provisional Application No. 63/707,305, filed Oct. 15, 2024, and U.S. Provisional Application No. 63/791,917, filed Apr. 21, 2025, the contents of each of which are hereby incorporated by reference in their entireties.

### REFERENCE TO A SEQUENCE LISTING SUBMITTED AS AN XML FILE

The present application hereby incorporates by reference the entire contents of the Sequence Listing File titled “206570-0003-00WO_SequenceListing.xml” in XML format, with a creation date of Oct. 14, 2025, and a size of 303,178 bytes.

### BACKGROUND OF THE INVENTION

The human Cluster of Differentiation 3 (CD3) T cell antigen receptor protein complex is composed of six distinct chains: a CD3γ chain (UniProt P09693), a CD3δ chain (UniProt P04234), two CD3ε chains (UniProt P07766), and one CD35 chain homodimer (UniProt P20963) (εγ: εδ:ζζ), which is associated with the T cell receptor α and β chain. This complex plays an important role in coupling antigen recognition to several intracellular signal-transduction pathways. The CD3 complex mediates signal transduction, resulting in T cell activation and proliferation. CD3 is required for immune response.

Bispecific antibodies and antibody fragments that bind CD3 have been explored as a means to recruit cytolytic T cells to kill tumor cells. However, the clinical use of many T cell-recruiting bispecific antibodies has been limited by challenges including unfavorable pharmacokinetics, potential immunogenicity, and manufacturing issues.

There thus exists a considerable need for bispecific antibodies that recruit cytolytic T cells to kill tumor cells that exhibit reduced toxicity and favorable manufacturing profiles. The present invention meets this long felt, but unmet, need.

### BRIEF SUMMARY OF THE INVENTION

In one aspect, the present invention provides an anti-CD3 antibody or antigen-binding fragment thereof comprising: a) a heavy chain complementarity determining region 1 (HCDR1) comprising the amino acid sequence of any one of SEQ ID NOs: 1, 9, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289, 297, and 305; b) an HCDR2 comprising the amino acid sequence of any one of SEQ ID NOs: 2, 10, 18, 26, 34, 42, 50, 58, 66, 74, 82, 90, 98, 106, 114, 122, 130, 138, 146, 154, 162, 170, 178, 186, 194, 202, 210, 218, 226, 234, 242, 250, 258, 266, 274, 282, 290, 298, and 306; c) an HCDR3 comprising the amino acid sequence of any one of SEQ ID NOs: 3, 11, 19, 27, 35, 43, 51, 59, 67, 75, 83, 91, 99, 107, 115, 123, 131, 139, 147, 155, 163, 171, 179, 187, 195, 203, 211, 219, 227, 235, 243, 251, 259, 267, 275, 283, 291, 299, and 307; d) a light chain complementarity determining region 1 (LCDR1) comprising the amino acid sequence of any one of SEQ ID NOs: 4, 12, 20, 28, 36, 44, 52, 60, 68, 76, 84, 92, 100, 108, 116, 124, 132, 140, 148, 156, 164, 172, 180, 188, 196, 204, 212, 220, 228, 236, 244, 252, 260, 268, 276, 284, 292, 300, and 308; e) an LCDR2 comprising the amino acid sequence of any one of SEQ ID NOs: 5, 13, 21, 29, 37, 45, 53, 61, 69, 77, 85, 93, 101, 109, 117, 125, 133, 141, 149, 157, 165, 173, 181, 189, 197, 205, 213, 221, 229, 237, 245, 253, 261, 269, 277, 285, 293, 301, and 309; and f) an LCDR3 comprising the amino acid sequence of any one of SEQ ID NOs: 6, 14, 22, 30, 38, 46, 54, 62, 70, 78, 86, 94, 102, 110, 118, 126, 134, 142, 150, 158, 166, 174, 182, 190, 198, 206, 214, 222, 230, 238, 246, 254, 262, 270, 278, 286, 294, 302, and 310.

In some embodiments, the anti-CD3 antibody or antigen-binding fragment thereof of comprises: a) an HCDR1, HCDR2, and HCDR3 comprising the amino acid sequences of: i) SEQ ID NOs: 1, 2, and 3, respectively; ii) SEQ ID NOs: 9, 10, and 11, respectively; iii) SEQ ID NOs: 17, 18, and 19, respectively; iv) SEQ ID NOs: 25, 26, and 27, respectively; v) SEQ ID NOs: 33, 34, and 35, respectively; vi) SEQ ID NOs: 41, 42, and 43, respectively; vii) SEQ ID NOs: 49, 50, and 51, respectively; viii) SEQ ID NOs: 57, 58, and 59, respectively; ix) SEQ ID NOs: 65, 66, and 67, respectively; x) SEQ ID NOs: 73, 74, and 75, respectively; xi) SEQ ID NOs: 81, 82, and 83, respectively; xii) SEQ ID NOs: 89, 90, and 91, respectively; xiii) SEQ ID NOs: 97, 98, and 99, respectively; xiv) SEQ ID NOs: 105, 106, and 107, respectively; xv) SEQ ID NOs: 113, 114, and 115, respectively; xvi) SEQ ID NOs: 121, 122, and 123, respectively; xvii) SEQ ID NOs: 129, 130, and 131, respectively; xviii) SEQ ID NOs: 137, 138, and 139, respectively; xix) SEQ ID NOs: 145, 146, and 147, respectively; xx) SEQ ID NOs: 153, 154, and 155, respectively; xxi) SEQ ID NOs: 161, 162, and 163, respectively; xxii) SEQ ID NOs: 169, 170, and 171, respectively; xxiii) SEQ ID NOs: 177, 178, and 179, respectively; xxiv) SEQ ID NOs: 185, 186, and 187, respectively; xxv) SEQ ID NOs: 193, 194, and 195, respectively; xxvi) SEQ ID NOs: 201, 202, and 203, respectively; xxvii) SEQ ID NOs: 209, 210, and 211, respectively; xxviii) SEQ ID NOs: 217, 218, and 219, respectively; xxix) SEQ ID NOs: 225, 226, and 227, respectively; xxx) SEQ ID NOs: 233, 234, and 235, respectively; xxxi) SEQ ID NOs: 241, 242, and 243, respectively; xxxii) SEQ ID NOs: 249, 250, and 251, respectively; xxxiii) SEQ ID NOs: 257, 258, and 259, respectively; xxxiv) SEQ ID NOs: 265, 266, and 267, respectively; xxxv) SEQ ID NOs: 273, 274, and 275, respectively; xxxvi) SEQ ID NOs: 281, 282, and 283, respectively; xxxvii) SEQ ID NOs: 289, 290, and 291, respectively; xxxviii) SEQ ID NOs: 297, 298, and 299, respectively; or xxxix) SEQ ID NOs: 305, 306, and 307; and b) an LCDR1, LCDR2, and LCDR3 comprising the amino acid sequences of: i) SEQ ID NOs: 4, 5, and 6, respectively; ii) SEQ ID NOs: 12, 13, and 14, respectively; iii) SEQ ID NOs: 20, 21, and 22, respectively; iv) SEQ ID NOs: 28, 29, and 30, respectively; v) SEQ ID NOs: 36, 37, and 38, respectively; vi) SEQ ID NOs: 44, 45, and 46, respectively; vii) SEQ ID NOs: 52, 53, and 54, respectively; viii) SEQ ID NOs: 60, 61, and 62, respectively; ix) SEQ ID NOs: 68, 69, and 70, respectively; x) SEQ ID NOs: 76, 77, and 78, respectively; xi) SEQ ID NOs: 84, 85, and 86, respectively; xii) SEQ ID NOs: 92, 93, and 94, respectively; xiii) SEQ ID NOs: 100, 101, and 102, respectively; xiv) SEQ ID NOs: 108, 109, 110, respectively; xv) SEQ ID NOs: 116, 117, and 118, respectively; xvi) SEQ ID NOs: 124, 125, and 126, respectively; xvii) SEQ ID NOs: 132, 133, and 134, respectively; xviii) SEQ ID NOs: 140, 141, and 142, respectively; xix) SEQ ID NOs: 148, 149, and 150, respectively; xx) SEQ ID NOs: 156, 157, and 158, respectively; xxi) SEQ ID NOs: 164, 165, and 166, respectively; xxii) SEQ ID NOs: 172, 173, and 174, respectively; xxiii) SEQ ID NOs: 180, 181, and 182, respectively; xxiv) SEQ ID NOs: 188, 189, and 190, respectively; xxv) SEQ ID NOs: 196, 197, and 198, respectively; xxvi) SEQ ID NOs: 204, 205, and 206, respectively; xxvii) SEQ ID NOs: 212, 213, and 214, respectively; xxviii) SEQ ID NOs: 220, 221, and 222, respectively; xxix) SEQ ID NOs: 228, 229, and 230, respectively; xxx) SEQ ID NOs: 236, 237, and 238, respectively; xxxi) SEQ ID NOs: 244, 245, and 246, respectively; xxxii) SEQ ID NOs: 252, 253, and 254, respectively; xxxiii) SEQ ID NOs: 260, 261, and 262, respectively; xxxiv) SEQ ID NOs: 268, 269, and 270, respectively; xxxv) SEQ ID NOs: 276, 277, and 278, respectively; xxxvi) SEQ ID NOs: 284, 285, and 286, respectively; xxxvii) SEQ ID NOs: 292, 293, and 294, respectively; xxxviii) SEQ ID NOs: 300, 301, and 302, respectively; or xxxix) SEQ ID NOs: 308, 309, and 310.

In some embodiments, the anti-CD3 antibody comprises an HCDR1, HCDR2, HCDR3, LCDR1, LCDR2, and LCDR3 comprising the amino acid sequences of: a) SEQ ID NOs: 1, 2, 3, 4, 5, and 6, respectively; b) SEQ ID NOs: 9, 10, 11, 12, 13, and 14, respectively; c) SEQ ID NOs: 17, 18, 19, 20, 21, and 22, respectively; d) SEQ ID NOs: 25, 26, 27, 28, 29, and 30, respectively; e) SEQ ID NOs: 33, 34, 35, 36, 37, and 38, respectively; f) SEQ ID NOs: 41, 42, 43, 44, 45, and 46, respectively; g) SEQ ID NOs: 49, 50, 51, 52, 53, and 54, respectively; h) SEQ ID NOs: 57, 58, 59, 60, 61, and 62, respectively; i) SEQ ID NOs: 65, 66, 67, 68, 69, and 70, respectively; j) SEQ ID NOs: 73, 74, 75, 76, 77, and 78, respectively; k) SEQ ID NOs: 81, 82, 83, 84, 85, and 86, respectively; l) SEQ ID NOs: 89, 90, 91, 92, 93, and 94, respectively; m) SEQ ID NOs: 97, 98, 99, 100, 101, and 102, respectively; n) SEQ ID NOs: 105, 106, 107, 108, 109, and 110, respectively; o) SEQ ID NOs: 113, 114, 115, 116, 117, and 118, respectively; p) SEQ ID NOs: 121, 122, 123, 124, 125, and 126, respectively; q) SEQ ID NOs: 129, 130, 131, 132, 133, and 134, respectively; r) SEQ ID NOs: 137, 138, 139, 140, 141, and 142, respectively; s) SEQ ID NOs: 145, 146, 147, 148, 149, and 150, respectively; t) SEQ ID NOs: 153, 154, 155, 156, 156, and 158, respectively; u) SEQ ID NOs: 161, 162, 263, 164, 165, and 166, respectively; v) SEQ ID NOs: 169, 170, 171, 172, 173, and 174, respectively; w) SEQ ID NOs: 177, 178, 179, 180, 181, and 182, respectively; x) SEQ ID NOs: 185, 186, 187, 188, 189, and 190, respectively; y) SEQ ID NOs: 193, 194, 195, 196, 197, and 198, respectively; z) SEQ ID NOs: 201, 202, 203, 204, 205, and 206, respectively; aa) SEQ ID NOs: 209, 210, 211, 212, 213, and 214, respectively; ab) SEQ ID NOs: 217, 218, 219, 220, 221, and 222, respectively; ac) SEQ ID NOs: 225, 226, 227, 228, 229, and 230, respectively; ad) SEQ ID NOs: 233, 234, 235, 236, 237, and 238, respectively; ae) SEQ ID NOs: 241, 242, 243, 244, 245, and 246, respectively; af) SEQ ID NOs: 249, 250, 251, 252, 253, and 254, respectively; ag) SEQ ID NOs: 257, 258, 259, 260, 261, and 262, respectively; ah) SEQ ID NOs: 265, 266, 267, 268, 269, and 270, respectively; ai) SEQ ID NOs: 273, 274, 275, 276, 277, and 278, respectively; aj) SEQ ID NOs: 281, 282, 283, 284, 285, and 286, respectively; ak) SEQ ID NOs: 289, 290, 291, 292, 293, and 294, respectively; al) SEQ ID NOs: 297, 298, 299, 300, 301, and 302, respectively; or am) SEQ ID NOs: 305, 306, 307, 308, 309, and 310, respectively.

In some embodiments, the anti-CD3 comprises: a) a heavy chain variable region (VH) comprising the amino acid sequence of any one of SEQ ID NOs: 7, 15, 23, 31, 39, 47, 55, 63, 71, 79, 87, 95, 103, 111, 119, 127, 135, 143, 151, 159, 167, 175, 183, 191, 199, 207, 215, 223, 231, 239, 247, 255, 263, 271, 279, 287, 295, 303, and 311; and b) a light chain variable region (VL) comprising the amino acid sequence of any one of SEQ ID NOs: 8, 16, 24, 32, 40, 48, 56, 64, 72, 80, 88, 96, 104, 112, 120, 128, 136, 144, 152, 160, 168, 176, 184, 192, 200, 208, 216, 224, 232, 240, 248, 256, 264, 272, 280, 288, 296, 304, and 312.

In some embodiments, the anti-CD3 antibody comprises a VH and a VL comprising the amino acid sequences of: a) SEQ ID NOs: 7 and 8, respectively; b) SEQ ID NOs: 15 and 16, respectively; c) SEQ ID NOs: 23 and 24, respectively; d) SEQ ID NOs: 31 and 32, respectively; e) SEQ ID NOs: 39 and 40, respectively; f) SEQ ID NOs: 47 and 48, respectively; g) SEQ ID NOs: 55 and 56, respectively; h) SEQ ID NOs: 63 and 64, respectively; i) SEQ ID NOs: 71 and 72, respectively; j) SEQ ID NOs: 79 and 80, respectively; k) SEQ ID NOs: 87 and 88, respectively; l) SEQ ID NOs: 95 and 96, respectively; m) SEQ ID NOs: 103 and 104, respectively; n) SEQ ID NOs: 111 and 112, respectively; o) SEQ ID NOs: 119 and 120, respectively; p) SEQ ID NOs: 127 and 128, respectively; q) SEQ ID NOs: 135 and 136, respectively; r) SEQ ID NOs: 143 and 144, respectively; s) SEQ ID NOs: 151 and 152, respectively; t) SEQ ID NOs: 159 and 160, respectively; u) SEQ ID NOs: 167 and 168, respectively; v) SEQ ID NOs: 175 and 176, respectively; w) SEQ ID NOs: 183 and 184, respectively; x) SEQ ID NOs: 191 and 192, respectively; y) SEQ ID NOs: 199 and 200, respectively; z) SEQ ID NOs: 207 and 208, respectively; aa) SEQ ID NOs: 215 and 216, respectively; ab) SEQ ID NOs: 223 and 224, respectively; ac) SEQ ID NOs: 231 and 232, respectively; ad) SEQ ID NOs: 239 and 240, respectively; ae) SEQ ID NOs: 247 and 248, respectively; af) SEQ ID NOs: 255 and 256, respectively; ag) SEQ ID NOs: 263 and 264, respectively; ah) SEQ ID NOs: 271 and 272, respectively; ai) SEQ ID NOs: 279 and 280, respectively; aj) SEQ ID NOs: 287 and 288, respectively; ak) SEQ ID NOs: 295 and 296, respectively; al) SEQ ID NOs: 303 and 304, respectively; or am) SEQ ID NOs: 311 and 312, respectively.

In some embodiments, the antibody is human or humanized. In some embodiments, the antibody comprises an Fc domain. In some embodiments, the Fc domain is a human immunoglobulin G (IgG) Fc domain. In some embodiments, the IgG Fc domain is an IgG1 Fc domain.

In some embodiments, the antibody specifically binds an epitope of CD3ε (SEQ ID NO: 328) comprising at least one amino acid in at least one of amino acid sequence selected from SEQ ID NOs: 331-335 and an epitope of CD3δ (SEQ In some embodiments, the antibody specifically binds an epitope of CD3ε (SEQ ID NO:328) comprising at least one amino acid sequence selected from SEQ ID NOs: 331-335 and an epitope of CD3δ (SEQ ID NO:327) comprising SEQ ID NO:336. In some embodiments, the antibody specifically binds an epitope of CD3ε (SEQ ID NO:328) comprising SEQ ID NOs: 331-335 and an epitope of CD3δ (SEQ ID NO: 327) comprising SEQ ID NO:336.

In some embodiments, the antibody is a bispecific or multispecific antibody.

In some embodiments, the present invention provides an isolated nucleic acid encoding an anti-CD3 antibody of the present invention. In some embodiments, the present invention provides a vector comprising an isolated nucleic acid of the present invention. In some embodiments, the present invention provides a host cell comprising a vector of the present invention.

In some embodiments, the present invention provides a composition comprising an antibody of the present invention and a pharmaceutically acceptable carrier.

In one aspect, the present invention provides a bispecific or multispecific antibody comprising a first binding domain that specifically binds CD3 and at least a second antigen binding domain that specifically binds at least a second antigen, wherein the first binding domain that specifically binds CD3 comprises: a) a heavy chain complementarity determining region 1 (HCDR1) comprising the amino acid sequence of any one of SEQ ID NOs: 1, 9, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289, 297, and 305; b) an HCDR2 comprising the amino acid sequence of any one of SEQ ID NOs: 2, 10, 18, 26, 34, 42, 50, 58, 66, 74, 82, 90, 98, 106, 114, 122, 130, 138, 146, 154, 162, 170, 178, 186, 194, 202, 210, 218, 226, 234, 242, 250, 258, 266, 274, 282, 290, 298, and 306; c) an HCDR3 comprising the amino acid sequence of any one of SEQ ID NOs: 3, 11, 19, 27, 35, 43, 51, 59, 67, 75, 83, 91, 99, 107, 115, 123, 131, 139, 147, 155, 163, 171, 179, 187, 195, 203, 211, 219, 227, 235, 243, 251, 259, 267, 275, 283, 291, 299, and 307; d) a light chain complementarity determining region 1 (LCDR1) comprising the amino acid sequence of any one of SEQ ID NOs: 4, 12, 20, 28, 36, 44, 52, 60, 68, 76, 84, 92, 100, 108, 116, 124, 132, 140, 148, 156, 164, 172, 180, 188, 196, 204, 212, 220, 228, 236, 244, 252, 260, 268, 276, 284, 292, 300, and 308; e) an LCDR2 comprising the amino acid sequence of any one of SEQ ID NOs: 5, 13, 21, 29, 37, 45, 53, 61, 69, 77, 85, 93, 101, 109, 117, 125, 133, 141, 149, 157, 165, 173, 181, 189, 197, 205, 213, 221, 229, 237, 245, 253, 261, 269, 277, 285, 293, 301, and 309; and f) an LCDR3 comprising the amino acid sequence of any one of SEQ ID NOs: 6, 14, 22, 30, 38, 46, 54, 62, 70, 78, 86, 94, 102, 110, 118, 126, 134, 142, 150, 158, 166, 174, 182, 190, 198, 206, 214, 222, 230, 238, 246, 254, 262, 270, 278, 286, 294, 302, and 310.

In some embodiments, the first binding domain that specifically binds CD3 comprises: a) an HCDR1, HCDR2, and HCDR3 comprising the amino acid sequences of: i) SEQ ID NOs: 1, 2, and 3, respectively; ii) SEQ ID NOs: 9, 10, and 11, respectively; iii) SEQ ID NOs: 17, 18, and 19, respectively; iv) SEQ ID NOs: 25, 26, and 27, respectively; v) SEQ ID NOs: 33, 34, and 35, respectively; vi) SEQ ID NOs: 41, 42, and 43, respectively; vii) SEQ ID NOs: 49, 50, and 51, respectively; viii) SEQ ID NOs: 57, 58, and 59, respectively; ix) SEQ ID NOs: 65, 66, and 67, respectively; x) SEQ ID NOs: 73, 74, and 75, respectively; xi) SEQ ID NOs: 81, 82, and 83, respectively; xii) SEQ ID NOs: 89, 90, and 91, respectively; xiii) SEQ ID NOs: 97, 98, and 99, respectively; xiv) SEQ ID NOs: 105, 106, and 107, respectively; xv) SEQ ID NOs: 113, 114, and 115, respectively; xvi) SEQ ID NOs: 121, 122, and 123, respectively; xvii) SEQ ID NOs: 129, 130, and 131, respectively; xviii) SEQ ID NOs: 137, 138, and 139, respectively; xix) SEQ ID NOs: 145, 146, and 147, respectively; xx) SEQ ID NOs: 153, 154, and 155, respectively; xxi) SEQ ID NOs: 161, 162, and 163, respectively; xxii) SEQ ID NOs: 169, 170, and 171, respectively; xxiii) SEQ ID NOs: 177, 178, and 179, respectively; xxiv) SEQ ID NOs: 185, 186, and 187, respectively; xxv) SEQ ID NOs: 193, 194, and 195, respectively; xxvi) SEQ ID NOs: 201, 202, and 203, respectively; xxvii) SEQ ID NOs: 209, 210, and 211, respectively; xxviii) SEQ ID NOs: 217, 218, and 219, respectively; xxix) SEQ ID NOs: 225, 226, and 227, respectively; xxx) SEQ ID NOs: 233, 234, and 235, respectively; xxxi) SEQ ID NOs: 241, 242, and 243, respectively; xxxii) SEQ ID NOs: 249, 250, and 251, respectively; xxxiii) SEQ ID NOs: 257, 258, and 259, respectively; xxxiv) SEQ ID NOs: 265, 266, and 267, respectively; xxxv) SEQ ID NOs: 273, 274, and 275, respectively; xxxvi) SEQ ID NOs: 281, 282, and 283, respectively; xxxvii) SEQ ID NOs: 289, 290, and 291, respectively; xxxviii) SEQ ID NOs: 297, 298, and 299, respectively; or xxxix) SEQ ID NOs: 305, 306, and 307; and b) an LCDR1, LCDR2, and LCDR3 comprising the amino acid sequences of: i) SEQ ID NOs: 4, 5, and 6, respectively; ii) SEQ ID NOs: 12, 13, and 14, respectively; iii) SEQ ID NOs: 20, 21, and 22, respectively; iv) SEQ ID NOs: 28, 29, and 30, respectively; v) SEQ ID NOs: 36, 37, and 38, respectively; vi) SEQ ID NOs: 44, 45, and 46, respectively; vii) SEQ ID NOs: 52, 53, and 54, respectively; viii) SEQ ID NOs: 60, 61, and 62, respectively; ix) SEQ ID NOs: 68, 69, and 70, respectively; x) SEQ ID NOs: 76, 77, and 78, respectively; xi) SEQ ID NOs: 84, 85, and 86, respectively; xii) SEQ ID NOs: 92, 93, and 94, respectively; xiii) SEQ ID NOs: 100, 101, and 102, respectively; xiv) SEQ ID NOs: 108, 109, 110, respectively; xv) SEQ ID NOs: 116, 117, and 118, respectively; xvi) SEQ ID NOs: 124, 125, and 126, respectively; xvii) SEQ ID NOs: 132, 133, and 134, respectively; xviii) SEQ ID NOs: 140, 141, and 142, respectively; xix) SEQ ID NOs: 148, 149, and 150, respectively; xx) SEQ ID NOs: 156, 157, and 158, respectively; xxi) SEQ ID NOs: 164, 165, and 166, respectively; xxii) SEQ ID NOs: 172, 173, and 174, respectively; xxiii) SEQ ID NOs: 180, 181, and 182, respectively; xxiv) SEQ ID NOs: 188, 189, and 190, respectively; xxv) SEQ ID NOs: 196, 197, and 198, respectively; xxvi) SEQ ID NOs: 204, 205, and 206, respectively; xxvii) SEQ ID NOs: 212, 213, and 214, respectively; xxviii) SEQ ID NOs: 220, 221, and 222, respectively; xxix) SEQ ID NOs: 228, 229, and 230, respectively; xxx) SEQ ID NOs: 236, 237, and 238, respectively; xxxi) SEQ ID NOs: 244, 245, and 246, respectively; xxxii) SEQ ID NOs: 252, 253, and 254, respectively; xxxiii) SEQ ID NOs: 260, 261, and 262, respectively; xxxiv) SEQ ID NOs: 268, 269, and 270, respectively; xxxv) SEQ ID NOs: 276, 277, and 278, respectively; xxxvi) SEQ ID NOs: 284, 285, and 286, respectively; xxxvii) SEQ ID NOs: 292, 293, and 294, respectively; xxxviii) SEQ ID NOs: 300, 301, and 302, respectively; or xxxix) SEQ ID NOs: 308, 309, and 310.

In some embodiments, the first antigen-binding domain that specifically binds CD3 comprises an HCDR1, HCDR2, HCDR3, LCDR1, LCDR2, and LCDR3 comprising the amino acid sequences of: a) SEQ ID NOs: 1, 2, 3, 4, 5, and 6, respectively; b) SEQ ID NOs: 9, 10, 11, 12, 13, and 14, respectively; c) SEQ ID NOs: 17, 18, 19, 20, 21, and 22, respectively; d) SEQ ID NOs: 25, 26, 27, 28, 29, and 30, respectively; e) SEQ ID NOs: 33, 34, 35, 36, 37, and 38, respectively; f) SEQ ID NOs: 41, 42, 43, 44, 45, and 46, respectively; g) SEQ ID NOs: 49, 50, 51, 52, 53, and 54, respectively; h) SEQ ID NOs: 57, 58, 59, 60, 61, and 62, respectively; i) SEQ ID NOs: 65, 66, 67, 68, 69, and 70, respectively; j) SEQ ID NOs: 73, 74, 75, 76, 77, and 78, respectively; k) SEQ ID NOs: 81, 82, 83, 84, 85, and 86, respectively; l) SEQ ID NOs: 89, 90, 91, 92, 93, and 94, respectively; m) SEQ ID NOs: 97, 98, 99, 100, 101, and 102, respectively; n) SEQ ID NOs: 105, 106, 107, 108, 109, and 110, respectively; o) SEQ ID NOs: 113, 114, 115, 116, 117, and 118, respectively; p) SEQ ID NOs: 121, 122, 123, 124, 125, and 126, respectively; q) SEQ ID NOs: 129, 130, 131, 132, 133, and 134, respectively; r) SEQ ID NOs: 137, 138, 139, 140, 141, and 142, respectively; s) SEQ ID NOs: 145, 146, 147, 148, 149, and 150, respectively; t) SEQ ID NOs: 153, 154, 155, 156, 156, and 158, respectively; u) SEQ ID NOs: 161, 162, 263, 164, 165, and 166, respectively; v) SEQ ID NOs: 169, 170, 171, 172, 173, and 174, respectively; w) SEQ ID NOs: 177, 178, 179, 180, 181, and 182, respectively; x) SEQ ID NOs: 185, 186, 187, 188, 189, and 190, respectively; y) SEQ ID NOs: 193, 194, 195, 196, 197, and 198, respectively; z) SEQ ID NOs: 201, 202, 203, 204, 205, and 206, respectively; aa) SEQ ID NOs: 209, 210, 211, 212, 213, and 214, respectively; ab) SEQ ID NOs: 217, 218, 219, 220, 221, and 222, respectively; ac) SEQ ID NOs: 225, 226, 227, 228, 229, and 230, respectively; ad) SEQ ID NOs: 233, 234, 235, 236, 237, and 238, respectively; ae) SEQ ID NOs: 241, 242, 243, 244, 245, and 246, respectively; af) SEQ ID NOs: 249, 250, 251, 252, 253, and 254, respectively; ag) SEQ ID NOs: 257, 258, 259, 260, 261, and 262, respectively; ah) SEQ ID NOs: 265, 266, 267, 268, 269, and 270, respectively; ai) SEQ ID NOs: 273, 274, 275, 276, 277, and 278, respectively; aj) SEQ ID NOs: 281, 282, 283, 284, 285, and 286, respectively; ak) SEQ ID NOs: 289, 290, 291, 292, 293, and 294, respectively; al) SEQ ID NOs: 297, 298, 299, 300, 301, and 302, respectively; or am) SEQ ID NOs: 305, 306, 307, 308, 309, and 310, respectively.

In some embodiments, the first antigen-binding domain that specifically binds CD3 comprises: a) a heavy chain variable region (VH) comprising the amino acid sequence of any one of SEQ ID NOs: 7, 15, 23, 31, 39, 47, 55, 63, 71, 79, 87, 95, 103, 111, 119, 127, 135, 143, 151, 159, 167, 175, 183, 191, 199, 207, 215, 223, 231, 239, 247, 255, 263, 271, 279, 287, 295, 303, and 311; and b) a light chain variable region (VL) comprising the amino acid sequence of any one of SEQ ID NOs: 8, 16, 24, 32, 40, 48, 56, 64, 72, 80, 88, 96, 104, 112, 120, 128, 136, 144, 152, 160, 168, 176, 184, 192, 200, 208, 216, 224, 232, 240, 248, 256, 264, 272, 280, 288, 296, 304, and 312.

In some embodiments, the first antigen-binding domain comprises a VH and a VL comprising the amino acid sequences of: a) SEQ ID NOs: 7 and 8, respectively; b) SEQ ID NOs: 15 and 16, respectively; c) SEQ ID NOs: 23 and 24, respectively; d) SEQ ID NOs: 31 and 32, respectively; e) SEQ ID NOs: 39 and 40, respectively; f) SEQ ID NOs: 47 and 48, respectively; g) SEQ ID NOs: 55 and 56, respectively; h) SEQ ID NOs: 63 and 64, respectively; i) SEQ ID NOs: 71 and 72, respectively; j) SEQ ID NOs: 79 and 80, respectively; k) SEQ ID NOs: 87 and 88, respectively; l) SEQ ID NOs: 95 and 96, respectively; m) SEQ ID NOs: 103 and 104, respectively; n) SEQ ID NOs: 111 and 112, respectively; o) SEQ ID NOs: 119 and 120, respectively; p) SEQ ID NOs: 127 and 128, respectively; q) SEQ ID NOs: 135 and 136, respectively; r) SEQ ID NOs: 143 and 144, respectively; s) SEQ ID NOs: 151 and 152, respectively; t) SEQ ID NOs: 159 and 160, respectively; u) SEQ ID NOs: 167 and 168, respectively; v) SEQ ID NOs: 175 and 176, respectively; w) SEQ ID NOs: 183 and 184, respectively; x) SEQ ID NOs: 191 and 192, respectively; y) SEQ ID NOs: 199 and 200, respectively; z) SEQ ID NOs: 207 and 208, respectively; aa) SEQ ID NOs: 215 and 216, respectively; ab) SEQ ID NOs: 223 and 224, respectively; ac) SEQ ID NOs: 231 and 232, respectively; ad) SEQ ID NOs: 239 and 240, respectively; ae) SEQ ID NOs: 247 and 248, respectively; af) SEQ ID NOs: 255 and 256, respectively; ag) SEQ ID NOs: 263 and 264, respectively; ah) SEQ ID NOs: 271 and 272, respectively; ai) SEQ ID NOs: 279 and 280, respectively; aj) SEQ ID NOs: 287 and 288, respectively; ak) SEQ ID NOs: 295 and 296, respectively; al) SEQ ID NOs: 303 and 304, respectively; or am) SEQ ID NOs: 311 and 312, respectively.

In some embodiments, the first antigen-binding domain of the bispecific antibody that specifically binds CD3 specifically binds an epitope of CD3ε (SEQ ID NO:328) comprising at least one amino acid in at least one of amino acid sequence selected from SEQ ID NOs: 331-335 and an epitope of CD3δ (SEQ ID NO:327) comprising at least one amino acid in SEQ ID NO: 336. In some embodiments, the first antigen-binding domain of the bispecific antibody that specifically binds CD3 specifically binds an epitope of CD3ε (SEQ ID NO:328) comprising at least one amino acid sequence selected from SEQ ID NOs: 331-335 and an epitope of CD38 (SEQ ID NO:327) comprising SEQ ID NO:336. In some embodiments, the first antigen-binding domain of the bispecific antibody that specifically binds CD3 specifically binds an epitope of CD3ε (SEQ ID NO:328) comprising SEQ ID NOs: 331-335 and an epitope of CD3δ (SEQ ID NO: 327) comprising SEQ ID NO:336.

In some embodiments, the bispecific or multispecific antibody is human or humanized. In some embodiments, the antibody comprises an Fc domain. In some embodiments, the Fc domain is a human immunoglobulin G (IgG) Fc domain. In some embodiments, the IgG Fc domain is an IgG1 Fc domain.

In some embodiments, the bispecific or multispecific antibody comprises two Fc chains. In some embodiments, one Fc domain comprises T366S, L368A, and Y407V substitutions and the second Fc domain comprises a T366W substitution, wherein numbering is according to the EU index. In some embodiments, the Fc domains further comprise an S354C and a Y349C substitution, wherein numbering is according to the EU index.

In some embodiments, the second antigen binding domain specifically binds a cell surface antigen that is expressed on a cell other than an immune effector cell. In some embodiments, the cell surface antigen is a tumor associated antigen.

In some embodiments, the present invention provides a composition comprising the bispecific or multispecific antibody of the present invention and a pharmaceutically acceptable carrier.

In some embodiments, the present invention provides an isolated nucleic acid encoding a bispecific or multispecific antibody of the present invention. In some embodiments, the present invention provides a vector comprising an isolated nucleic acid of the present invention. In some embodiments, the present invention provides a host cell comprising a vector of the present invention.

In one aspect, the present invention provides a method of treating a disease or disorder in a subject in need thereof comprising administering to the subject a bispecific or multispecific antibody of the present invention. In some embodiments, the disease or disorder is cancer. In some embodiments, the cancer is a solid tumor or a hematologic cancer.

In one aspect, the present invention provides a bispecific or multispecific antibody of the present invention for use in treating a disease or disorder in a subject in need thereof. In some embodiments, the disease or disorder is cancer. In some embodiments, the cancer is a solid tumor or a hematologic cancer.

In one aspect, the present invention provides a use of an antibody of the present invention in treating a disease or disorder in a subject in need thereof. In some embodiments, the antibody is a bispecific or multispecific antibody. In some embodiments, the disease or disorder is cancer. In some embodiments, the cancer is a solid tumor or a hematologic cancer.

In one aspect, the present invention provides a use of a bispecific or multispecific antibody of the present invention in preparation of a medicament for treating a disease or disorder in a subject in need thereof. In some embodiments, the disease or disorder is cancer. In some embodiments, the cancer is a solid tumor or a hematologic cancer.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 shows a schematic representation of a bispecific antibody described herein that specifically binds CLDN6 and CD3.

FIG. 2 depicts a schematic representation of the CD3-TCR complex indicating the binding epitopes that Ab897 binds to as determined by hydrogen-deuterium exchange (HDX)-mass spectrometry (HDX-MS) mapped onto the structure of CD3.

FIG. 3 depicts a representative diagram of CLDN6 in a membrane, with the two extracellular domain (ECD) loops (ECD-1 and ECD-2), the four transmembrane domains, and three intracellular domains. The amino acid residues that differ between CLDN6 and CLDN9 are indicated by arrows.

FIG. 4 depicts representative binding of CLDN6 binders 51H102 and 112H105 to CLDN6/CLDN9 hybrid mutants and wildtype CLDN6 and CLDN9 expressed in skov3 cells. Binding was assessed by flow cytometry, and mean fluorescence intensity of antibody binding was compared to that of wildtype CLDN6.

FIG. 5A and FIG. 5B show Ab897 specificity for T-cells and CLDN6 overexpressing cell lines. FIG. 5A depicts representative binding to human T cells. FIG. 5B depicts representative binding to CHO cells stable expressing CLDN6 or CLDN9.

FIG. 6 shows representative flow cytometry histograms of Ab897 binding to ovarian cancer cell lines OVCAR3, PA-1, and OV-90, which express CLDN6 compared to a lack of binding observed with SKOV3 cells. Shaded region=cell incubation with 300 nM Ab897; dashed line=cell incubation with 300 nM Null×CD3 antibody.

FIG. 7A shows flow cytometry plots showing median fluorescence intensity (MFI) values of Ab897 binding to CLDN6 cell lines OVARC3 and PA1.

FIG. 7B shows flow cytometry plots showing MFI values of Ab897 binding to CLDN6 cell line OV90, CLDN6-cell line SKOV3, and to HepG2 cells.

FIG. 8 shows a dose response of Ab897-mediated cytotoxicity on PA1-NLG cells co-cultured with pan-T cells from 5 individual donors as shown at 3:1 E:T ratio for 72 hours (top panel) or 96 hours (bottom panel). Target only control (no effector cell) did not show any cytotoxicity. All data points were in duplicates, mean±SD plotted. All negative lysis values were contained to 0 for the purposes of data plotting in a non-linear regression curve.

FIG. 9 shows a dose response of Ab897-mediated cytotoxicity on OV90-NLG cells co-cultured with pan-T cells from individual donors as shown at 3:1 E:T ratio for 48 hours (top panel), 72 hours (middle panel) or 96 hours (bottom panel). Target only control (no effector cell) did not show any cytotoxicity. All data points were in duplicates, mean±SD plotted. All negative lysis values were contained to 0 for the purposes of data plotting in a non-linear regression curve.

FIG. 10 shows dose response of Ab897-mediated cytotoxicity on OVCAR3, PA-1, and OV-90 cell lines co-cultured with pan-T cells from five individual donors at a 3:1 ratio for 72 hours. Data points represent duplicates of all five donors, mean±SD plotted.

FIG. 11 shows a dose response of Ab897-mediated cytotoxicity on HepG2-NLG liver tumor cells co-cultured with pan-T cells from individual donors as shown at 10:1 E:T ratio for 48 hours (top panel), 72 hours (middle panel) or 96 hours (bottom panel). Target only control (no effector cell) did not show any cytotoxicity. All data points were in duplicates, mean±SD plotted. All negative lysis values were contained to 0 for the purposes of data plotting in a non-linear regression curve.

FIG. 12 shows a dose response of Ab897-mediated lysis on CLDN6 OVARC3 cells and CLDN6-SLOV3 cells in co-cultures with pan-T cells from individual donors at 3:1 (top panel) 1:1 (middle panel) and 1:3 (bottom panel) E:T ratio for 72 hours. All data points were in duplicates, mean±SD plotted.

FIG. 13A shows a dose response of Ab897 mediated proliferation of CD4 (left panel) and CD8 (right panel) T cells in co-cultures of OVARC3 cells and pan-T cells from 5 individual donors at 3:1 E:T ratio for 72 hours. All data points are in duplicates. Mean±SD is plotted.

FIG. 13B shows a dose response of Ab897 mediated activation of CD4⁺ (top panel) and CD8⁺ (bottom panel) T cells in co-cultures of OVARC3 cells and pan-T cells from 5 individual donors at 3:1 E:T ratio for 72 hours, as measured by increased percentage (%) of CD4⁺CD25⁺ and CD4⁺CD69⁺ (top panel), or CD8⁺CD25⁺ and CD8⁺CD69⁺ (bottom panel) T cells. All data points are in duplicates. Mean±SD is plotted.

FIG. 13C shows a dose response of Ab897 mediated cytokine production (TNFα, left panel), IFNγ, middle panel, IL6 (right panel) in co-cultures of OVARC3 cells and pan-T cells from 5 individual donors at 3:1 E:T ratio for 72 hours. All data points are in duplicates. Mean±SD is plotted.

FIG. 14A shows anti-tumor activity of Ab897 in a OVCAR3 tumor xenograft NSG mouse model. Ab897 was administered weekly at various concentrations for four weeks. All data points represent eight mice, mean±SEM plotted.

FIG. 14B shows anti-tumor activity of Ab897 in a OV90 tumor xenograft NSG mouse model. Ab897 was administered weekly at 0.1 mg/kg for three weeks. Each line represents an individual mouse.

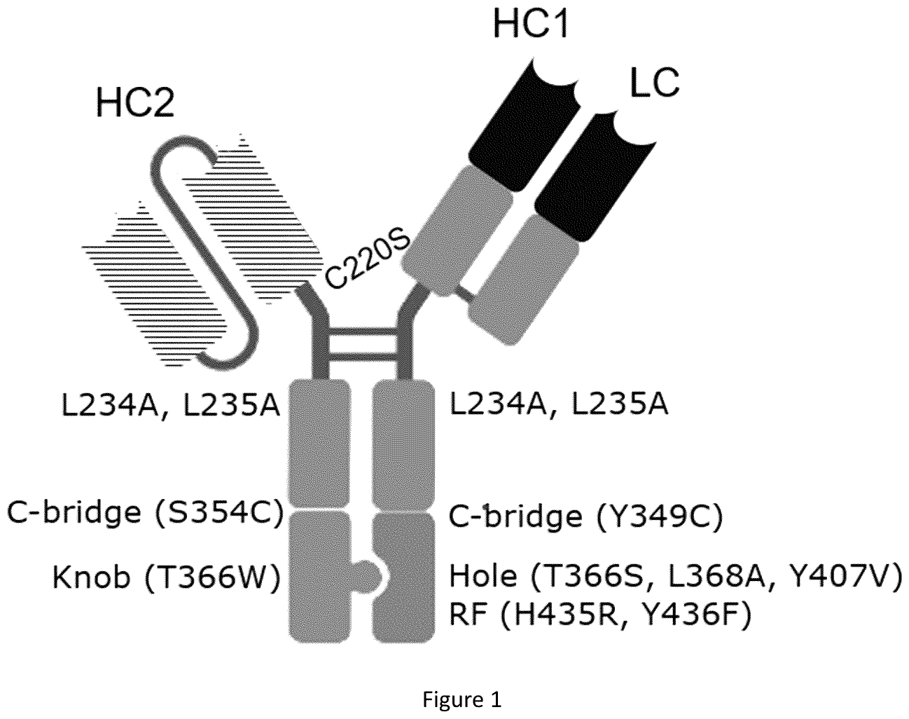

### DETAILED DESCRIPTION OF ILLUSTRATIVE EMBODIMENTS

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the disclosure provided herein. Such discussion is not an admission that any of these matters, singularly or in combination, form part of the prior art with respect to any disclosure provided herein.

Unless defined otherwise, all technical and scientific terms used herein have the same meaning as commonly understood to one of ordinary skill in the art to which the disclosure provided herein pertains. Otherwise, certain terms used herein have the meanings as set forth in the specification.

The numbering of amino acid residues of the antibody constant regions throughout the specification is according to the EU numbering as described in Kabat et al., Sequences of Proteins of Immunological Interest, 5th Ed. Public Health Service, National Institutes of Health, Bethesda, Md. (1991), unless otherwise explicitly stated. EU numbering and its correspondence to other numbering systems are available at IMGT (ImMunoGeneTics) within IMGT scientific charts.

It must be noted that as used herein and in the appended claims, the singular forms “a,” “an,” and “the” include plural reference unless the context clearly dictates otherwise.

It should also be understood that the terms “about,” “approximately,” “generally,” “substantially,” and like terms, used herein when referring to a dimension or characteristic of a component described herein, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments described herein. Such equivalents are intended to be encompassed by the disclosure provided herein.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of” indicate the inclusion of any recited integer or group of integers, but that no additional integer or group of integers can be added to the specified method, structure, or composition.

As used herein, the term “consists essentially of,” or variations such as “consist essentially of” or “consisting essentially of” indicate the inclusion of any recited integer or group of integers, and the optional inclusion of any recited integer or group of integers that do not materially change the basic or novel properties of the specified method, structure or composition. See US Manual of Patent Examining Procedure (M.P.E.P) § 2111.03.

As used herein, “subject” means a mammal, such as a human. The term “mammal” as used herein, encompasses any mammal.

The terms “identical” or percent “identity,” in the context of two or more nucleic acids or amino acid sequences (e.g., antibodies that specifically bind CD3 and polynucleotides that encode them or the bispecific or multispecific antibodies that specifically bind CD3 and one or more additional antigens and polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated.

The sequence comparison algorithm then calculates the percent sequence identity for the test sequence(s) relative to the reference sequence, based on the designated program parameters.

Alignment of sequences for comparison can be conducted, e.g., by the local homology algorithm of Smith & Waterman, Adv. Appl. Math. 1981; 2:482-9, by the homology alignment algorithm of Needleman & Wunsch, J Mol. Biol. 1970; 48:443-53, by the search for similarity method of Pearson & Lipman, Proc. Nat'l. Acad. Sci. USA 1988; 85:2444-8, by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement)).

Examples of algorithms that are suitable for determining percent sequence identity and sequence similarity are the BLAST and BLAST 2.0 algorithms, which are described in Altschul et al. J Mol. Biol. 1990; 215:403-10 and Altschul et al. Nucleic Acids Res. 1997; 25:3389-402, respectively. Software for performing BLAST analyses is publicly available through the National Center for Biotechnology Information.

A further indication that two nucleic acid sequences or amino acid sequences are substantially identical is that the amino acid sequence encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions, as described, e.g., in Molecular Diagnostics, Edition 1, 2019, E. van Pelt-Verkuil, W. B. van Leeuwen, R. the Witt eds., Springer Singapore (ISBN 978-981-13-1603-6).

As used herein, “bispecific antigen-binding molecules” include any molecule that exhibits specific binding to two distinct antigens. A bispecific antigen-binding molecule may comprise one or more first binding domains that specifically bind a first antigen and one or more second binding domains that specifically bind a second antigen. Binding domains may include, but are not limited to, amino acid sequences, nucleic acid sequences, carbohydrates, and combinations thereof. Exemplary bispecific antigen-binding molecules include, but are not limited to, bispecific antibodies, bispecific aptamers, bispecific antibody-aptamer conjugates, bispecific lectins, bispecific antibody-lectin conjugates, and bispecific aptamer-lectin conjugates.

As used herein, “multispecific antigen-binding molecules” include any molecule that exhibits specific binding to two, three, four, five, or more distinct antigens. A multispecific antigen-binding molecule may comprise one or more first binding domains that specifically bind a first antigen, one or more second binding domains that specifically bind a second antigen (i.e., bispecific). A multispecific antigen-binding molecule may further comprise one or more third binding domains that specifically bind a third antigen (i.e., trispecific). A multispecific antigen-binding molecule may further comprise one or more fourth binding domains that specifically bind a fourth antigen (i.e., tetraspecific). A multispecific antigen-binding molecule may further comprise one or more fifth, sixth, seventh, etc. binding domains that specifically bind a fifth, sixth, seventh, etc. antigen (i.e., tetraspecific, pentaspecific, hexaspecific, heptaspecific, etc.). Binding domains may include, but are not limited to, amino acid sequences, nucleic acid sequences, carbohydrates, and combinations thereof. Exemplary antigen-binding domains include, but are not limited to, antibodies, aptamers, lectins, etc.

The term “CD3” refers to the human Cluster of Differentiation 3 protein multi-subunit complex and includes any CD3, variant and/or isoform which is endogenously expressed (including expressed by T cells) or can be overexpressed on cells transfected with genes or cDNA encoding CD3. CD3 can also be referred to as “cluster of differentiation 3.” The CD3 protein multi-subunit complex is composed of six (6) distinctive polypeptide chains, which include a CD3G chain (UniProt P09693) (SEQ ID NO:326), a CD3D chain (UniProt P04234) (SEQ ID NO:327), two CD3E chains (UniProt P07766) (SEQ ID NO:328), and a CD3Z chain homodimer (UniProt P20963) (SEQ ID NO:329). These CD3 protein subunits are part of the T-cell receptor (TCR)-CD3 complex present on T-lymphocytes and play an essential role in the adaptive immune response. When antigen presenting cells (APCs) activate TCR, TCR-mediated signals are transmitted across the cell membrane by the CD3 chains CD3D, CD3E, CD3G and CD3Z. All CD3 chains contain immunoreceptor tyrosine-based activation motifs (ITAMs) in their cytoplasmic domain. Upon TCR engagement, these motifs become phosphorylated by Src family protein tyrosine kinases LCK and FYN, resulting in the activation of downstream signaling pathways.

CD3E antibodies have been described. SP34 (Yang S J, J. Immunol. 1986; 137:1097-100) reacts with both primate and human CD3 and is available commercially from Pharmingen. Additional anti-CD3 antibodies include UCHT-1, see PCT Intl. Publ. No. WO2000/041474 and Anasetti et al., Transplantation 1992; 54:844-50 and BC3 (available from Abcam). SP34 differs from UCHT-1 and BC3 in that SP34 recognizes an epitope present solely on the CD3E subunit (see Salmeron et al., J. Immunol. 1991; 147:3047-52) whereas UCHT-1 and BC3 recognize an epitope contributed by both the CD3E and CD3D subunits. An antibody sequence that is 96% identical to the SP34 V_{H} is described in PCT Intl. Publ. No. WO2007/042261.

The CD3 specific antibodies provided herein are novel and exhibit different properties as compared to those described above. The novel CD3 specific antibodies provided herein exhibit moderate binding to human CD3 and do not bind cynomolgus CD3.

As used herein, the term “antibody” is used in a broad sense and includes immunoglobulin or antibody molecules including human, humanized and chimeric antibodies and antibody fragments, including monospecific, bispecific, or multispecific antibodies with single or multiple valency. Antibodies described herein can take a number of formats, including traditional antibodies as well as antibody derivatives, fragments and mimetics, including a number of bispecific or multispecific formats and antigen-binding domains described herein. In general, antibodies are proteins or peptide chains that exhibit binding specificity to a specific antigen.

Antibody structures are well known. Traditional antibodies are “Y” shaped tetramers comprised of two heavy chains (HC) and two light chains (LC). Each heavy chain is comprised of a heavy chain variable region (VH) and a heavy chain constant region (comprised of domains CH1, hinge, CH2 and CH3), and each light chain is comprised of a light chain variable region (VL and a light chain constant region (CL1). Other useful antibody formats are those described herein and those known in the art and include antibody formats that incorporate single chain antigen-binding domains such as single chain Fv (scFv).

Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Further diversity of IgG1 is found in natural variations and polymorphisms known as allotypes (described, for example at imgt.org/IMGTrepertoire/Proteins/allotypes/human/IGH/IGHC/G1m_allotypes.html).

In one embodiment, bispecific antibodies that specifically bind CLDN6 and CD3 and are of the IgG1 subclass are provided.

In one embodiment, antibodies of the IgG1 subclass, Glm17, 1 allotype are provided.

As used herein, the term “Fc”, “Fc domain” or “Fc domain” refers to a portion of an immunoglobulin molecule that correlates to a crystallizable fragment obtained by papain digestion of an Ig molecule. Within an Fc domain, there are two “Fc chains” (e.g., a “first Fc chain” and a “second Fc chain”), each Fc chain excluding the first constant region immunoglobulin domain (CH1), and optionally a hinge or a portion of the hinge. Although the boundaries of each Fc chain may vary, the human IgG1 heavy chain Fc chain is usually defined to comprise residues C226 or P230 in its N-terminus. In some embodiments, the Fc chain comprises from about amino acid residue T225 to about K447 of the human IgG1 heavy chain. The C-terminal lysine (residue 447) of the Fc domain may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. The Fc domain mediates effector functions, such as antibody-dependent cellular cytotoxicity (ADCC) and complement-dependent cytotoxicity (CDC). In ADCC, the Fc domain of an antibody binds to Fc receptors (FcRs) on the surface of immune effector cells such as natural killer cells and macrophages, leading to the phagocytosis or lysis of the targeted cells. In CDC, the antibodies kill the targeted cells by triggering the complement cascade on the cell surface. The Fc domains may be engineered to modify the Fc-mediated effector functions using known mutations.

For many applications of therapeutic antibodies, Fc-mediated effector functions are not desired as they may be detrimental and potentially pose a safety risk by causing off-mechanism toxicity. Modifying Fc-mediated effector functions can be achieved by engineering the Fc domains to reduce their binding to FcRs or complement factors while maintaining the stability, pharmacokinetics and half-life of the engineered antibody. The binding of IgG to the activating (FcγRI, FcγRIIa, FcγRIIIa and FcγRIIIb) and inhibitory (FcγRIIb) FcRs or the first component of complement (Clq) depends on residues located in the hinge region and the CH2 domain. Mutations have been introduced in IgG1, IgG2 and IgG4 to reduce or silence Fc functionalities. In some embodiments, the IgG1 antibodies and bispecific antibodies provided herein include L234A and L235A substitutions in the Fc domain to reduce their binding to FcRs and reduce Fc-mediated effector functions.

In one embodiment, the antibodies or the bispecific antibodies provided herein comprise a modified Fc domain with one or more of the following properties: (a) reduced effector function when compared to the parent (i.e., unmodified) Fc domain; (b) reduced affinity to FcγRI, FcγRIla, FcγRIIb, FcγRIIIb and/or FcγRIIIa, (c) reduced affinity to FcγRI, (d) reduced affinity to FcγRIla (e) reduced affinity to FcγRIIb, (f) reduced affinity to FcγRIIIb or (g) reduced affinity to FcγRIIIa. Reduced effector function and reduced affinity can be measured using known methods. “Reduced” may be a reduction from about 10% to 100%, or a statistically significant reduction when compared to a test molecule, such as a bispecific antigen-binding molecule with a native unmodified Fc domain.

In some embodiments, the Fc domain comprises one or more mutations that increase antibody half-life, such as M252Y/S254T/T256E (YTE) mutations, according to EU numbering.

As used herein, the term “antigen-binding fragment” or “antigen-binding domain” is any proteinaceous structure that exhibits binding affinity for a particular antigen. Antigen-binding fragments include those provided by any known technique, such as enzymatic cleavage, peptide synthesis, and recombinant techniques. Some antigen-binding fragments are composed of portions of intact antibodies that retain antigen-binding specificity of the parent antibody molecule. For example, antigen-binding fragments may comprise at least one variable region (either a heavy chain or light chain variable region) or one or more CDRs of an antibody known to bind a particular antigen. Examples of suitable antigen-binding fragments include, without limitation, a diabody, a Fab, a Fab′, a F(ab′) 2, an Fv fragment, a single chain Fv (scFv), optionally a disulfide stabilized single chain Fv fragment, a (scFv) 2, a bispecific scFv (scFv-scFv′), a disulfide stabilized diabody (ds diabody), a single-chain antibody, a single domain antibody (sdab), a camelized single domain antibody, a nanobody, a domain antibody, a bivalent domain antibody, or any other antibody fragment that binds to an antigen but does not comprise a traditional Y-shaped antibody structure.

Additionally, antigen-binding fragments may include non-antibody proteinaceous frameworks that may successfully incorporate polypeptide segments in an orientation that confers affinity for a given antigen of interest, such as protein scaffolds.

As used herein, the term “single-chain antibody” refers to a conventional single-chain antibody such as a single-chain variable fragment (scFv). “scFv” refers to a single chain protein comprising a VH, a VL and a linker between the VH and the VL. The scFv may comprise the VL and VH in either orientation, e.g., with respect to the N- to C-terminal order of the VH and the VL. The scFv may thus be in the orientation VL-linker-VH or VH-linker-VL. The VH and VL may be connected by a short flexible peptide linker of about 11 to about 20 amino acids to allow the VH and the VL to properly fold to retain antigen-binding capabilities of the scFv. An scFv may be engineered to comprise disulfide bonds between the VH and the VL, or between the VH, VL and the linker. scFv constructs can be linked to an antibody constant chain or Fc domain at either the N- or C-terminus, directly or through a linker.

“Isolated” refers to a homogenous population of molecules (such as synthetic polynucleotides or proteins such as an antibodies or bispecific antibodies) which have been substantially separated and/or purified away from other components of the system the molecules are produced in, such as a recombinant cell as well as proteins that have been subjected to at least one purification or isolation step. “Isolated antibody” refers to an antibody that is substantially free of other cellular material and/or chemicals and encompasses antibodies that are isolated to a higher purity, such as to at least 80% purity, for example 90%-100% purity.

As used herein, the term “monoclonal antibody” refers to a preparation of antibody molecules of single molecular composition, i.e., the individual antibodies comprising the composition are identical except for possible naturally occurring mutations that may be present in minor amounts, or post-translational modifications that occur during production and storage. The monoclonal antibodies can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “human antibody” refers to an antibody that has minimal immune response when administered to a human subject. Variable regions of human antibodies are derived from human germline immunoglobulin sequences. If the antibody contains a constant region or a portion of the constant region (such as the Fc domain), the constant region is also derived from human germline immunoglobulin sequences. A human antibody comprises heavy or light chain variable regions that are “derived from” human germline immunoglobulin sequences if the variable regions of the antibody are obtained from a system that uses human germline immunoglobulin genes. Exemplary systems are human immunoglobulin gene libraries displayed on phage or mammalian cells, and transgenic non-human animals such as mice, rats or chickens carrying human immunoglobulin loci. A human antibody typically contains amino acid differences when compared to the immunoglobulins expressed in humans due to differences between the systems used to obtain the antibody and human immunoglobulin loci, introduction of naturally occurring somatic mutations, intentional introduction of substitutions into the frameworks or CDRs. A human antibody is typically about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin sequences. In some cases, a human antibody may contain consensus framework sequences derived from human framework sequence analyses, for example as described in (Knappik et al., J. Mol. Biol. 2000; 296:57-86), or synthetic HCDR3 incorporated into human immunoglobulin gene libraries displayed on phage, for example as described in (Shi et al., J. Mol. Biol. 2010; 397:385-96), and in PCT Int. Publ. No. WO2009/085462. Antibodies in which CDRs are derived from a non-human species are not included in the definition of human antibody.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable regions of both the light and heavy chains often correspond to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

As used herein, the term “multispecific antibody” refers to an antibody that specifically binds to two or more distinct antigens or epitopes. Multispecific antibodies may bind two (bispecific), three (trispecific), four (tetraspecific), five (pentaspecific), or more distinct antigens. In some embodiments, the multispecific antibodies of the disclosure bind to at least two, three, four, five, or more distinct antigens, wherein at least one antigen is CD3.

As used herein, the term “bispecific antibody” refers to an antibody that specifically binds two distinct antigens or epitopes. The bispecific antibody may comprise one or more antigen-binding domains that specifically bind each antigen or epitope. In one embodiment, the bispecific antibody of the disclosure binds two distinct antigens, wherein at least one antigen is CD3.

As used herein, an antibody or a bispecific antibody that “specifically binds CD3” refers to an antibody, a bispecific antibody, or a multispecific antibody that binds human CD3, with a K_{D} of about 1×10⁻⁷ M or less, 1× 10⁻⁸ M or less, 5×10⁻⁹ M or less, 1×10⁻⁹ M or less, 5×10⁻¹⁰ M or less or 1×10⁻¹⁰ M or less, typically with a K_{D} that is at least one hundred-fold less that its K_{D} for binding to a non-specific antigen (e.g., casein or bovine serum albumin (BSA)). The term “K_{D}” refers to the dissociation constant and is expressed as a molar concentration (M). The smaller the K_{D} value, the higher the binding affinity of the antibody for its target antigen. K_{D} values for antibodies can be determined using well known methods and those described herein. For example, the K_{D} of an antibody can be determined by using surface plasmon resonance, such as by using a biosensor system, e.g., a Biacore® system, or by using bio-layer interferometry technology, such as an Octet RED96 system. Antibodies that specifically bind to the antigen may, however, have cross-reactivity to other related antigens, for example to the same antigen from other species (homologs), such as human or monkey, for example, Macaca fascicularis (cynomolgus monkey, cyno). While a monospecific antibody specifically binds one antigen, a bispecific antibody specifically binds two distinct antigens (or two distinct epitopes on the same antigen).

### Antibodies

Provided herein are antibodies that specifically bind CD3, nucleic acids and expression vectors encoding the antibodies, recombinant cells containing the vectors, pharmaceutical compositions comprising the antibodies, methods of manufacturing the antibodies, and methods of use thereof. In certain aspects, the CD3 binding domain of the antibodies is useful for activating T-cells.

Also provided herein are bispecific antibodies comprising a first antigen-binding domain that specifically binds CD3 and a second antigen-binding domain that specifically binds a second antigen. Simultaneous binding of an antigen on a tumor cell and CD3 on a T-cell facilitates directed killing (cell lysis) of the targeted tumor cell by the activated T-cell. The bispecific antibodies that specifically bind an antigen on a tumor cell and CD3 described herein are therefore useful, inter alia, for treating diseases and disorders related to or caused by antigen-expressing tumors.

Also provided herein are multispecific antibodies comprising a first antigen-binding domain that specifically binds CD3 and at least one additional antigen-binding domain that specifically binds at least one additional antigen. In some embodiments, the multispecific antibodies comprise a first antigen-binding domain that specifically binds CD3 and second additional antigen-binding domain that specifically binds a second antigen. In some embodiments, the multispecific antibodies comprise a first antigen-binding domain that specifically binds CD3, as second antigen-binding domain that specifically binds a second antigen, and a third antigen-binding domain that specifically binds a third antigen. In some embodiments, the multispecific antibodies comprise a first antigen-binding domain that specifically binds CD3, as second antigen-binding domain that specifically binds a second antigen, a third antigen-binding domain that specifically binds a third antigen, and a fourth antigen-binding domain that specifically binds a fourth antigen.

In some embodiments, the present disclosure provides anti-CD3 antibodies, wherein the antigen-binding domain that specifically binds CD3 comprises an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to a sequence shown in Table 1. In some embodiments, the antigen-binding domain that specifically binds CD3 comprises an amino acid sequence shown in Table 1. The CDR, VH, and VL sequences of the antibodies provided herein are shown in Table 1 and Table 2. In the event of any discrepancy between the sequence of these tables and the sequences in the sequence listing or as provided elsewhere in this application, the sequences in Table 1 and Table 2 should prevail.

TABLE 1
CD3 Binding Kabat CDR Sequences

| | Variable | | | |
|---|---|---|---|---|
| Antibody | Region | CDR1 (SEQ ID NO) | CDR2 (SEQ ID NO) | CDR3 (SEQ ID NO) |
| 01C03A | VH | SGGYYWT (1) | SIYYSGSTYYNPALK | ERKNELFFDY (3) |
| | | | S (2) | |
| | VL | TRSSGSIASNYVQ (4) | EDKQRPS (5) | QSFDSSNRV (6) |
| 01G24A | VH | FYTMN (9) | SITTSSRYIYYADSVK | AGTKTHDAFDF (11) |
| | | | G (10) | |
| | VL | SRSSGSIASNYVQ (12) | EDNQRPS (13) | QSYDNSNRV (14) |
| 01M02A | VH | SYSMN (17) | TISRSSSYIYYADSVK | EKWNLLYFDS (19) |
| | | | G (18) | |
| | VL | TRSSGSIASNYVQ | EDNQRPS (21) | QSYDSSNRV (22) |
| | | (20) | | |
| 01P14N | VH | SYSMN (25) | AISRSSTYIYYADSVK | ENWDLLYFEF (27) |
| | | TRSSGSIASNYVQ | G (26) | |
| | VL | (28) | EDNQRPS (29) | QSYDSSNRV (30) |
| 02I07A | VH | SYSMN (33) | YISSSGSYKYYADSV | DRKQLLLDY (35) |
| | | | KG (34) | |
| | VL | SGDALPKKYAY (36) | EDSKRPS (37) | YSTDSSGNHWV (38) |
| 03J04A | VH | SYTMN (41) | SITRSSRYIYYADSVK | AGAATHDAFDI (43) |
| | | TRSSGSIASNYVQ | G (42) | |
| | VL | (44) | EDNQRPS (45) | QSYDSNNRV (46) |
| 03L07A | VH | FYTMN (49) | SITTSSRYIYYADSVK | AGAKTHDAFDF (51) |
| | | | G (50) | |
| | VL | SRSSGSIASNYVQ (52) | EDNQRPS (53) | QSYDSSNRV (54) |
| 03015A | VH | SYSMN (57) | SISRSTAYIYYADSVK | EKWELLYFDS (59) |
| | | | G (58) | |
| | VL | TRNSGSIASNYVQ | EDNQRPS (61) | QSYDSSNRV (62) |
| | | (60) | | |
| 03P01A | VH | TSPIN (65) | SISRISSHKYYADSVK | EKWELLYFDY (67) |
| | | | G (66) | |
| | VI | RSSQSLLDSDDGNTY | TLSYRAS (69) | MQRIEFPSIT (70) |
| | | LD (68) | | |
| 03P01Z | VH | TSPIN (73) | SISRISSHKYYADSVK | EKWELLYFDY (75) |
| | | | G (74) | |
| | VL | TRSSGSIASNYVQ | EDNQRPS (77) | QSYDSSNRV (78) |
| | | (76) | | |
| 04C03A | VH | NYILN (81) | SISSSSRYIFYADSVK | ESKWELLSFDY (83) |
| | | | G (82) | |
| | VL | TRSSGSIASNYVQ | EDNQRPS (85) | QSYDRSNRV (86) |
| | | (84) | | |
| 04J18A | VH | SGGYSWS (89) | SIYYSGRTYYNPALK | ERKNELFFDY (91) |
| | | | S (90) | |
| | VL | TRSSGSIASNYVQ | EDNQRPS (93) | QSYDSSNRV (94) |
| | | (92) | | |
| 04K01A | VH | SSLMN (97) | SISSSSNYIYYADSVR | ERWVLLYFDY (99) |
| | | | G (98) | |
| | VL | TRSSGSIASNYVQ | EDNQRPS (101) | QSYDSSIRV (102) |
| | | (100) | | |
| 04M22A | VH | SSLMN (105) | SISSSSNYIYYADSVR | ERWVLLYFDY (107) |
| | | | G (106) | |
| | VL | TRSSGSIASNYVQ | EDNQRPS (109) | QSYDSSIRV (110) |
| | | (108) | | |
| 04P20A | VH | SYSMN (113) | YINSRSSTKYYADSV | VMRHYGMDV (115) |
| | | | KG (114) | |
| | VL | TRSSGSIASNYVQ | EDNQRPS (117) | QSFDNSNHWV (118) |
| | | (116) | | |
| 05B02A | VH | RYSMN (121) | TVSSGSRYRYYADSV | AGASTHDSFDI (123) |
| | | | KG (122) | |
| | VL | TRSRGSIASNYVQ | EDNKRPS (125) | QSYDNSNRV (126) |
| | | (124) | | |
| 05L13A | VH | SYSMN (129) | SISRSSSYIYYADSVK | EKWELLYFDA (131) |
| | | | G (130) | |
| | VL | TRSSGSIASNYVQ | EDNQRPS (133) | QSYDRSNRV (134) |
| | | (132) | | |
| 05N13A | VH | SYSMN (137) | SSSRSSSYIYYADSVK | ERWELLYFDY (139) |
| | | | G (138) | |
| | VL | TRSSGSIASNYVQ | EDNQRPS (141) | QSYDSSIRV (142) |
| | | (140) | | |
| 06A06A | VH | SGAYYWN (145) | DIYYSGTTYYNPALK | ERKNELFFDY (147) |
| | | | S (146) | |
| | VL | TRSSGSIASNYVQ | EDNQRPS (149) | QSYDSSNRV (150) |
| | | (148) | | |
| 07B06A | VH | NYAMN (153) | SISRSNSHKYYADSV | EKWELLYFDS (155) |
| | | | KG (154) | |
| | VL | RSSQSLLDSDDGNTY | TLSYRAS (157) | MQRIEFPSIT (158) |
| | | LD (156) | | |
| 07B06Z | VH | NYAMNW (161) | SISRSNSHKYYADSV | EKWELLYFDS (163) |
| | | | KG (162) | |
| | VL | TRSSGSIASNYVQ | EDDQRPS (165) | QSYDSSNRV (166) |
| | | (164) | | |
| 07H10A | VH | DYYIHW (169) | WINPNSGGTTYVQKF | DRGTYYDYFDN (171) |
| | | | QG (170) | |
| | VL | KSSESVLYRSNNKNY | WASIRES (173) | QQYYGTPYT (174) |
| | | LT (172) | | |
| 07L12A | VH | RNSMN (177) | SISTSGKYRYYADSL | AGGSTHDSFDI (179) |
| | | | KD (178) | |
| | VL | TRSSGSIARNYVQ | EDNKRPS (181) | QSYDNSNRV (182) |
| | | (180) | | |
| 07P07A | VH | FYTMN (185) | SITTSSRYIYYADSVK | AGAKTHDAFDF (187) |
| | | | G (186) | |
| | VL | SRSSGSIASNYVQ | EDNQRPS (189) | QSYDSSNRV (190) |
| | | (188) | | |
| 07P15A | VH | RYSMN (193) | SISSSSYYYYADSLK | ERRAGFDY (195) |
| | | | G (194) | |
| | VL | TRSSGSIASNYVQ | EDNQRPS (197) | QSYDSSNRV (198) |
| | | (196) | | |
| 08M15A | VH | SSSMN (201) | SISSRSRHIYYADSVK | EKWELLYFDY (203) |
| | | | G (202) | |
| | VL | GGNNIGSKSVH (204) | DDSDRPS (205) | QVWDSSSDHVV |
| | | | | (206) |
| 08M15Z | VH | SSSMN (209) | SISSRSRHIYYADSVK | EKWELLYFDY (211) |
| | | | G (210) | |
| | VL | RSSGSIASNYVQ (212) | EDNQRPS (213) | QSYDSSNRV (214) |
| 09B13A | VH | SYSMN (217) | SISRSSAYIYYADSVK | EKWELLYFDY (219) |
| | | | G (218) | |
| | VL | RSSGSIASNYVQ (220) | EDNQRPS (221) | QSYDSSNRV (222) |
| 09D16A | VH | RYSMN (225) | TVSSGSRYRYYADSV | AGGNTHDAFDI (227) |
| | | | KG (226) | |
| | VL | TRSRGSIASNYVQ | EDNKRPS (229) | SYDNSNRV (230) |
| | | (228) | | |
| 10E16A | VH | RYSMNW (233) | LISSSSSYKYYVDSVK | DRRELVLDY (235) |
| | | | G (234) | |
| | VL | SGDALPKKYAY (236) | EDSKRPS (237) | YSTNNSGNHW (238) |
| 14A15A | VH | TYSMN (241) | TFSRSSSHIYYADSVK | EQWNLLYFDY (243) |
| | | | G (242) | |
| | VL | TRSSGSIASNYVQ | EDNQRPS (245) | QSYDRSNRV (246) |
| | | (244) | | |
| 14C10A | VH | RYSMN (249) | SISSGSRYIYYRDSVR | EPRNHFDY (251) |
| | | | G (250) | |
| | VL | TRSSGSIASNYVQ | EDNQRPS (253) | QSYDSSIRV (254) |
| | | (252) | | |
| 04D18A | VH | RYSMN (257) | TISSSSSYRYYADSVK | AGGNTHDAFEI (259) |
| | | | G (258) | |
| | VL | TRSSGSIARNYVQ | EDNKRPS (261) | QSYDNSNRV (262) |
| | | (260) | | |
| 02J12A | VH | SYTMN (265) | SITRSSRYIYYADSVK | AGAATHDAFDI (267) |
| | | | G (266) | |
| | VL | TRSSGSIASNYVQ | EDNQRPS (269) | QSYDGNNRV (270) |
| | | (268) | | |
| 07N22A | VH | IYSMN (273) | SISSSSSYIHYADSVK | DRRERLLAY (275) |
| | | | G (274) | |
| | VL | SGDALPKKYAY (276) | EDSKRPS (277) | YSTDSRGNLWV (278) |
| 08M01A | VH | SYSMN (281) | TISRSSSYIYYADSVK | EKWNLLYFDA (283) |
| | | | G (282) | |
| | VL | TRSSGSIASNYVQ | EDNQRPS (285) | QSYDSSNRV (286) |
| | | (284) | | |
| 10B09A | VH | SYSMN (289) | SISRSSNYIYYADSVK | EKWNLLYFDY (291) |
| | | | G (290) | |
| | VL | TRSSGSIATNYVQ | EDNKRPS (293) | QSYDSSNRV (294) |
| | | (292) | | |
| 14E03A | VH | RYWMT (297) | NIKKDGSENYYVDSV | VRVIYDWLDP (299) |
| | | | KG (298) | |
| | VL | SGDKLGNKYAA | QDSKRPS (301) | QAWDNNIVV (302) |
| | | (300) | | |
| 14018A | VH | RYSMN (305) | SISMSSRYIYYVDSVK | ERGPGPGMDV (307) |
| | | | G (306) | |
| | VL | TRSSGSIASNYVQ | EDNQRPS (309) | QSYDSSNRV (310) |
| | | (308) | | |

In some embodiments, the present disclosure provides anti-CD3 antibodies, wherein the antigen-binding domain that specifically binds CD3 comprises:
a) a heavy chain complementarity determining region 1 (HCDR1) comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to an amino acid sequence of any one of SEQ ID NOs: 1, 9, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289, 297, and 305;
b) an HCDR2 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to an amino acid sequence of any one of SEQ ID NOs: 2, 10, 18, 26, 34, 42, 50, 58, 66, 74, 82, 90, 98, 106, 114, 122, 130, 138, 146, 154, 162, 170, 178, 186, 194, 202, 210, 218, 226, 234, 242, 250, 258, 266, 274, 282, 290, 298, and 306;
c) an HCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to an amino acid sequence of any one of SEQ ID NOs: 3, 11, 19, 27, 35, 43, 51, 59, 67, 75, 83, 91, 99, 107, 115, 123, 131, 139, 147, 155, 163, 171, 179, 187, 195, 203, 211, 219, 227, 235, 243, 251, 259, 267, 275, 283, 291, 299, and 307;
d) a light chain complementarity determining region 1 (LCDR1) comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to an amino acid sequence of any one of SEQ ID NOs: 4, 12, 20, 28, 36, 44, 52, 60, 68, 76, 84, 92, 100, 108, 116, 124, 132, 140, 148, 156, 164, 172, 180, 188, 196, 204, 212, 220, 228, 236, 244, 252, 260, 268, 276, 284, 292, 300, and 308;
e) an LCDR2 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to an amino acid sequence of any one of SEQ ID NOs: 5, 13, 21, 29, 37, 45, 53, 61, 69, 77, 85, 93, 101, 109, 117, 125, 133, 141, 149, 157, 165, 173, 181, 189, 197, 205, 213, 221, 229, 237, 245, 253, 261, 269, 277, 285, 293, 301, and 309; and
f) an LCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to an amino acid sequence of any one of SEQ ID NOs: 6, 14, 22, 30, 38, 46, 54, 62, 70, 78, 86, 94, 102, 110, 118, 126, 134, 142, 150, 158, 166, 174, 182, 190, 198, 206, 214, 222, 230, 238, 246, 254, 262, 270, 278, 286, 294, 302, and 310.

In some embodiments, the present disclosure provides anti-CD3 antibodies, wherein the antigen-binding domain that specifically binds CD3 comprises:
a) a heavy chain complementarity determining region 1 (HCDR1) comprising an amino acid sequence of any one of SEQ ID NOs: 1, 9, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289, 297, and 305;
b) an HCDR2 comprising an amino acid sequence of any one of SEQ ID NOs: 2, 10, 18, 26, 34, 42, 50, 58, 66, 74, 82, 90, 98, 106, 114, 122, 130, 138, 146, 154, 162, 170, 178, 186, 194, 202, 210, 218, 226, 234, 242, 250, 258, 266, 274, 282, 290, 298, and 306;
c) an HCDR3 comprising an amino acid sequence of any one of SEQ ID NOs: 3, 11, 19, 27, 35, 43, 51, 59, 67, 75, 83, 91, 99, 107, 115, 123, 131, 139, 147, 155, 163, 171, 179, 187, 195, 203, 211, 219, 227, 235, 243, 251, 259, 267, 275, 283, 291, 299, and 307;
d) a light chain complementarity determining region 1 (LCDR1) comprising an amino acid sequence of any one of SEQ ID NOs: 4, 12, 20, 28, 36, 44, 52, 60, 68, 76, 84, 92, 100, 108, 116, 124, 132, 140, 148, 156, 164, 172, 180, 188, 196, 204, 212, 220, 228, 236, 244, 252, 260, 268, 276, 284, 292, 300, and 308;
e) an LCDR2 comprising an amino acid sequence of any one of SEQ ID NOs: 5, 13, 21, 29, 37, 45, 53, 61, 69, 77, 85, 93, 101, 109, 117, 125, 133, 141, 149, 157, 165, 173, 181, 189, 197, 205, 213, 221, 229, 237, 245, 253, 261, 269, 277, 285, 293, 301, and 309; and
f) an LCDR3 comprising an amino acid sequence of any one of SEQ ID NOs: 6, 14, 22, 30, 38, 46, 54, 62, 70, 78, 86, 94, 102, 110, 118, 126, 134, 142, 150, 158, 166, 174, 182, 190, 198, 206, 214, 222, 230, 238, 246, 254, 262, 270, 278, 286, 294, 302, and 310.

In some embodiments, the anti-CD3 antibodies comprise:
a) an HCDR1, HCDR2, and HCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of SEQ ID NOS:
   i) 1, 2, and 3, respectively;
   ii) 9, 10, and 11, respectively;
   iii) 17, 18, and 19, respectively;
   iv) 25, 26, and 27, respectively;
   v) 33, 34, and 35, respectively;
   vi) 41, 42, and 43, respectively;
   vii) 49, 50, and 51, respectively;
   viii) 57, 58, and 59, respectively;
   ix) 65, 66, and 67, respectively;
   x) 73, 74, and 75, respectively;
   xi) 81, 82, and 83, respectively;
   xii) 89, 90, and 91, respectively;
   xiii) 97, 98, and 99, respectively;
   xiv) 105, 106, and 107, respectively;
   xv) 113, 114, and 115, respectively;
   xvi) 121, 122, and 123, respectively;
   xvii) 129, 130, and 131, respectively;
   xviii) 137, 138, and 139, respectively;
   xix) 145, 146, and 147, respectively;
   xx) 153, 154, and 155, respectively;
   xxi) 161, 162, and 163, respectively;
   xxii) 169, 170, and 171, respectively;
   xxiii) 177, 178, and 179, respectively;
   xxiv) 185, 186, and 187, respectively;
   xxv) 193, 194, and 195, respectively;
   xxvi) 201, 202, and 203, respectively;
   xxvii) 209, 210, and 211, respectively;
   xxviii) 217, 218, and 219, respectively;
   xxix) 225, 226, and 227, respectively;
   xxx) 233, 234, and 235, respectively;
   xxxi) 241, 242, and 243, respectively;
   xxxii) 249, 250, and 251, respectively;
   xxxiii) 257, 258, and 259, respectively;
   xxxiv) 265, 266, and 267, respectively;
   xxxv) 273, 274, and 275, respectively;
   xxxvi) 281, 282, and 283, respectively;
   xxxvii) 289, 290, and 291, respectively;
   xxxviii) 297, 298, and 299, respectively; or
   xxxix) 305, 306, and 307, respectively; and
b) an LCDR1, LCDR2, and LCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of SEQ ID NOs:
   i) 4, 5, and 6, respectively;
   ii) 12, 13, and 14, respectively;
   iii) 20, 21, and 22, respectively;
   iv) 28, 29, and 30, respectively;
   v) 36, 37, and 38, respectively;
   vi) 44, 45, and 46, respectively;
   vii) 52, 53, and 54, respectively;
   viii) 60, 61, and 62, respectively;
   ix) 68, 69, and 70, respectively;
   x) 76, 77, and 78, respectively;
   xi) 84, 85, and 86, respectively;
   xii) 92, 93, and 94, respectively;
   xiii) 100, 101, and 102, respectively;
   xiv) 108, 109, and 110, respectively;
   xv) 116, 117, and 118, respectively;
   xvi) 124, 125, and 126, respectively;
   xvii) 132, 133, and 134, respectively;
   xviii) 140, 141, and 142, respectively;
   xix) 148, 149, and 150, respectively;
   xx) 156, 157, and 158, respectively;
   xxi) 164, 165, and 166, respectively;
   xxii) 172, 173, and 174, respectively;
   xxiii) 180, 181, and 182, respectively;
   xxiv) 188, 189, and 190, respectively;
   xxv) 196, 197, and 198, respectively;
   xxvi) 204, 205, and 206, respectively;
   xxvii) 212, 213, and 214, respectively;
   xxviii) 220, 221, and 222, respectively;
   xxix) 228, 229, and 230, respectively;
   xxx) 236, 237, and 238, respectively;
   xxxi) 244, 245, and 246, respectively;
   xxxii) 252, 253, and 254, respectively;
   xxxiii) 260, 261, and 262, respectively;
   xxxiv) 268, 269, and 270, respectively;
   xxxv) 276, 277, and 278, respectively;
   xxxvi) 284, 285, and 286, respectively;
   xxxvii) 292, 293, and 294, respectively;
   xxxviii) 300, 301, and 302, respectively; or
   xxxix) 308, 309, and 310, or respectively.

In some embodiments, the anti-CD3 antibodies comprise:
a) an HCDR1, HCDR2, and HCDR3 comprising the sequences of SEQ ID NOs:
   i) 1, 2, and 3, respectively;
   ii) 9, 10, and 11, respectively;
   iii) 17, 18, and 19, respectively;
   iv) 25, 26, and 27, respectively;
   v) 33, 34, and 35, respectively;
   vi) 41, 42, and 43, respectively;
   vii) 49, 50, and 51, respectively;
   viii) 57, 58, and 59, respectively;
   ix) 65, 66, and 67, respectively;
   x) 73, 74, and 75, respectively;
   xi) 81, 82, and 83, respectively;
   xii) 89, 90, and 91, respectively;
   xiii) 97, 98, and 99, respectively;
   xiv) 105, 106, and 107, respectively;
   xv) 113, 114, and 115, respectively;
   xvi) 121, 122, and 123, respectively;
   xvii) 129, 130, and 131, respectively;
   xviii) 137, 138, and 139, respectively;
   xix) 145, 146, and 147, respectively;
   xx) 153, 154, and 155, respectively;
   xxi) 161, 162, and 163, respectively;
   xxii) 169, 170, and 171, respectively;
   xxiii) 177, 178, and 179, respectively;
   xxiv) 185, 186, and 187, respectively;
   xxv) 193, 194, and 195, respectively;
   xxvi) 201, 202, and 203, respectively;
   xxvii) 209, 210, and 211, respectively;
   xxviii) 217, 218, and 219, respectively;
   xxix) 225, 226, and 227, respectively;
   xxx) 233, 234, and 235, respectively;
   xxxi) 241, 242, and 243, respectively;
   xxxii) 249, 250, and 251, respectively;
   xxxiii) 257, 258, and 259, respectively;
   xxxiv) 265, 266, and 267, respectively;
   xxxv) 273, 274, and 275, respectively;
   xxxvi) 281, 282, and 283, respectively;
   xxxvii) 289, 290, and 291, respectively;
   xxxviii) 297, 298, and 299, respectively; or
   xxxix) 305, 306, and 307, respectively; and
b) an LCDR1, LCDR2, and LCDR3 comprising the sequences of SEQ ID NOs:
   i) 4, 5, and 6, respectively;
   ii) 12, 13, and 14, respectively;
   iii) 20, 21, and 22, respectively;
   iv) 28, 29, and 30, respectively;
   v) 36, 37, and 38, respectively;
   vi) 44, 45, and 46, respectively;
   vii) 52, 53, and 54, respectively;
   viii) 60, 61, and 62, respectively;
   ix) 68, 69, and 70, respectively;
   x) 76, 77, and 78, respectively;
   xi) 84, 85, and 86, respectively;
   xii) 92, 93, and 94, respectively;
   xiii) 100, 101, and 102, respectively;
   xiv) 108, 109, and 110, respectively;
   xv) 116, 117, and 118, respectively;
   xvi) 124, 125, and 126, respectively;
   xvii) 132, 133, and 134, respectively;
   xviii) 140, 141, and 142, respectively;
   xix) 148, 149, and 150, respectively;
   xx) 156, 157, and 158, respectively;
   xxi) 164, 165, and 166, respectively;
   xxii) 172, 173, and 174, respectively;
   xxiii) 180, 181, and 182, respectively;
   xxiv) 188, 189, and 190, respectively;
   xxv) 196, 197, and 198, respectively;
   xxvi) 204, 205, and 206, respectively;
   xxvii) 212, 213, and 214, respectively;
   xxviii) 220, 221, and 222, respectively;
   xxix) 228, 229, and 230, respectively;
   xxx) 236, 237, and 238, respectively;
   xxxi) 244, 245, and 246, respectively;
   xxxii) 252, 253, and 254, respectively;
   xxxiii) 260, 261, and 262, respectively;
   xxxiv) 268, 269, and 270, respectively;
   xxxv) 276, 277, and 278, respectively;
   xxxvi) 284, 285, and 286, respectively;
   xxxvii) 292, 293, and 294, respectively;
   xxxviii) 300, 301, and 302, respectively; or
   xxxix) 308, 309, and 310, or respectively.

In some embodiments, the anti-CD3 antibodies comprise an HCDR1, HCDR2, HCDR3, LCDR1, LCDR2, and LCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of SEQ ID NOs:
a) 1, 2, 3, 4, 5, and 6, respectively;
b) 9, 10, 11, 12, 13, and 14, respectively;
c) 17, 18, 19, 20, 21, and 22, respectively;
d) 25, 26, 27, 28, 29, and 30, respectively;
e) 33, 34, 35, 36, 37, and 38, respectively;
f) 41, 42, 43, 44, 45, and 46, respectively;
g) 49, 50, 51, 52, 53, and 54, respectively;
h) 57, 58, 59, 60, 61, and 62, respectively;
i) 65, 66, 67, 68, 69, and 70, respectively;
j) 73, 74, 75, 76, 77, and 78, respectively;
k) 81, 82, 83, 84, 85, and 86, respectively;
l) 89, 90, 91, 92, 93, and 94, respectively;
m) 97, 98, 99, 100, 101, and 102, respectively;
n) 105, 106, 107, 108, 109, and 110, respectively;
o) 113, 114, 115, 116, 117, and 118, respectively;
p) 121, 122, 123, 124, 125, and 126, respectively;
q) 129, 130, 131, 132, 133, and 134, respectively;
r) 137, 138, 139, 140, 141, and 142, respectively;
s) 145, 146, 147, 148, 149, and 150, respectively;
t) 153, 154, 155, 156, 157, and 158, respectively;
u) 161, 162, 163, 164, 165, and 166, respectively;
v) 169, 170, 171, 172, 173, and 174, respectively;
w) 177, 178, 179, 180, 181, and 182, respectively;
x) 185, 186, 187, 188, 189, and 190, respectively;
y) 193, 194, 195, 196, 197, and 198, respectively;
z) 201, 202, 203, 204, 205, and 206, respectively;
aa) 209, 210, 211, 212, 213, and 214, respectively;
ab) 217, 218, 219, 220, 221, and 222, respectively;
ac) 225, 226, 227, 228, 229, and 230, respectively;
ad) 233, 234, 235, 236, 237, and 238, respectively;
ae) 241, 242, 243, 244, 245, and 246, respectively;
af) 249, 250, 251, 252, 253, and 254, respectively;
ag) 257, 258, 259, 260, 261, and 262, respectively;
ah) 265, 266, 267, 268, 269, and 270, respectively;
ai) 273, 274, 275, 276, 277, and 278, respectively;
aj) 281, 282, 283, 284, 285, and 286, respectively;
ak) 289, 290, 291, 292, 293, and 294, respectively;
al) 297, 298, 299, 300, 301, and 302, respectively; or
am) 305, 306, 307, 308, 309, and 310, respectively.

In some embodiments, the anti-CD3 antibodies comprise an HCDR1, HCDR2, HCDR3, LCDR1, LCDR2, and LCDR3 comprising the sequences of SEQ ID NOs:
a) 1, 2, 3, 4, 5, and 6, respectively;
b) 9, 10, 11, 12, 13, and 14, respectively;
c) 17, 18, 19, 20, 21, and 22, respectively;
d) 25, 26, 27, 28, 29, and 30, respectively;
e) 33, 34, 35, 36, 37, and 38, respectively;
f) 41, 42, 43, 44, 45, and 46, respectively;
g) 49, 50, 51, 52, 53, and 54, respectively;
h) 57, 58, 59, 60, 61, and 62, respectively;
i) 65, 66, 67, 68, 69, and 70, respectively;
j) 73, 74, 75, 76, 77, and 78, respectively;
k) 81, 82, 83, 84, 85, and 86, respectively;
l) 89, 90, 91, 92, 93, and 94, respectively;
m) 97, 98, 99, 100, 101, and 102, respectively;
n) 105, 106, 107, 108, 109, and 110, respectively;
o) 113, 114, 115, 116, 117, and 118, respectively;
p) 121, 122, 123, 124, 125, and 126, respectively;
q) 129, 130, 131, 132, 133, and 134, respectively;
r) 137, 138, 139, 140, 141, and 142, respectively;
s) 145, 146, 147, 148, 149, and 150, respectively;
t) 153, 154, 155, 156, 157, and 158, respectively;
u) 161, 162, 163, 164, 165, and 166, respectively;
v) 169, 170, 171, 172, 173, and 174, respectively;
w) 177, 178, 179, 180, 181, and 182, respectively;
x) 185, 186, 187, 188, 189, and 190, respectively;
y) 193, 194, 195, 196, 197, and 198, respectively;
z) 201, 202, 203, 204, 205, and 206, respectively;
aa) 209, 210, 211, 212, 213, and 214, respectively;
ab) 217, 218, 219, 220, 221, and 222, respectively;
ac) 225, 226, 227, 228, 229, and 230, respectively;
ad) 233, 234, 235, 236, 237, and 238, respectively;
ae) 241, 242, 243, 244, 245, and 246, respectively;
af) 249, 250, 251, 252, 253, and 254, respectively;
ag) 257, 258, 259, 260, 261, and 262, respectively;
ai) 273, 274, 275, 276, 277, and 278, respectively;
aj) 281, 282, 283, 284, 285, and 286, respectively;
ak) 289, 290, 291, 292, 293, and 294, respectively;
al) 297, 298, 299, 300, 301, and 302, respectively; or
am) 305, 306, 307, 308, 309, and 310, respectively.

In some embodiments, the anti-CD3 antibodies comprise an HCDR1, HCDR2, HCDR3, LCDR1, LCDR2, and LCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of SEQ ID NOs:
a) 1, 2, 3, 4, 5, and 6, respectively;
b) 201, 202, 203, 204, 205, and 206, respectively;
c) 249, 250, 251, 252, 253, and 254, respectively; or
d) 297, 298, 299, 300, 301, and 302, respectively.

In some embodiments, the anti-CD3 antibodies comprise an HCDR1, HCDR2, HCDR3, LCDR1, LCDR2, and LCDR3 comprising the sequences of SEQ ID NOs:
a) 1, 2, 3, 4, 5, and 6, respectively;
b) 201, 202, 203, 204, 205, and 206, respectively;
c) 249, 250, 251, 252, 253, and 254, respectively; or
d) 297, 298, 299, 300, 301, and 302, respectively.

In some embodiments, the anti-CD3 antibodies comprise a sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to a sequence listed in Table 2. In some embodiments, the anti-CD3 antibodies comprises a sequence listed in Table 2.

TABLE 2
CD3 Binding Variable Region Sequences

| | Variable | |
|---|---|---|
| Antibody | Region | Sequence (SEQ ID NO) |
| 01C03A | VH | QVQLQESGPGLVKPSQTLSLTCTVSGGSISSGGYYWTWIRQHPGKCLEW |
| | | IGSIYYSGSTYYNPALKSRLTISVDTSKNQFSLKLSSVTAADTAVYYCAR |
| | | ERKNELFFDYWGQGTLVTVSS (7) |
| | VL | NFMLTQPHSVSESPGKTVTISCTRSSGSIASNYVQWYQQRPDSSPTTVIY |
| | | EDKQRPSGVPDRFSGSIDRSSNSASLTISGLKTEDEADYYCQSFDSSNRV |
| | | FGCGTKLTVL (8) |
| 01G24A | VH | EVQLVESGGGLVKPGGSLRLSCAASGFTFNFYTMNWVRQAPGKCLEW |
| | | VSSITTSSRYIYYADSVKGRFTISRDNAKNSLYLQMNSLRAEDTAVYYC |
| | | ARAGTKTHDAFDFWGQGTMVTVSS (15) |
| | VL | NFMLTQPHSVSESPGKTVTISCSRSSGSIASNYVQWYQQRPGSSPTTVIY |
| | | EDNQRPSGVPDRFSGSIDSSSNSASLTISGLKTEDEADYYCQSYDNSNRV |
| | | FGCGTKLTVL (16) |
| 01M02A | VH | EVQLVESGGGLVKPGGSLRLSCAASGFTVSSYSMNWVRQAPGKCLEW |
| | | VSTISRSSSYIYYADSVKGRFTISRDNAKNSLYLQMNSLRAEDTAVYFCA |
| | | REKWNLLYFDSWGQGTLVTVSS (23) |
| | VL | NFMLTQSHSVSESPGKTVTISCTRSSGSIASNYVQWYQQRPGSSPTTVIY |
| | | EDNQRPSGVPDRFSGSIDSSSNSASLTISGLKTEDEADYYCQSYDSSNRV |
| | | FGCGTKLTVL (24) |
| 01P14N | VH | EVQLVESGGGLVKPGGSLRLSCAASGFTLSSYSMNWVRQAPGRCLEWV |
| | | SAISRSSTYIYYADSVKGRFTISRDNAKNSVHLQMSSLRAEDTAVYYCA |
| | | RENWDLLYFEFWGQGTLVTVSS (31) |
| | VL | NFMLTQPHSVSESPGKTVTISCTRSSGSIASNYVQWYQQRPGSSPTTVIY |
| | | EDNQRPSGVPDRFSGSIDSSSNSASLTISGLKTEDEADYYCQSYDSSNRV |
| | | FGCGTKLTVL (32) |
| 02I07A | VH | EVQLVESGGGLVKPGGSLRLSCAASGFTFSSYSMNWVRQAPGKGLEWV |
| | | SYISSSGSYKYYADSVKGRFTISRDNAKNSLYLQMNSLRAEDTAVFYCA |
| | | RDRKQLLLDYWGQGTLVTVSS (39) |
| | VL | SYELTQPPSVSVSPGQTARITCSGDALPKKYAYWYQQKSGQAPVLVIYE |
| | | DSKRPSGIPERFSGSSSGTMATLTISGAQVEDEADYYCYSTDSSGNHWV |
| | | FACGTKLTVL (40) |
| 03J04A | VH | EVQLVESGGGLVTPGGSLRLSCAASGFTFSSYTMNWVRQAPGKCLEWV |
| | | SSITRSSRYIYYADSVKGRFTISRDNAKNSLYLQMNSLRAEDTAVYYCA |
| | | RAGAATHDAFDIWAQGTMVTVSS (47) |
| | VL | NFMLTQPHSVSESPGKTVTISCTRSSGSIASNYVQWYQQRPGSSPATVIY |
| | | EDNQRPSGVPDRFSGSIDSSSNSASLTISGLKTEDEADYYCQSYDSNNRV |
| | | FGCGTKLTVL (48) |
| 03L07A | VH | EVQLVESGGGLVKPGGSLRLSCAASGFTFSFYTMNWVRQAPGKCLEWV |
| | | SSITTSSRYIYYADSVKGRFTISRDNAKNSLYLQMNSLRAEDTAVYYCA |
| | | RAGAKTHDAFDFWGQGTMVTVSS (55) |
| | VL | NFMLTQPHSVSESPGKTVTISCSRSSGSIASNYVQWYQQRPGSSPTTVIY |
| | | EDNQRPSGVPDRFSGSIDSSSNSASLTISGLKTEDEADYYCQSYDSSNRV |
| | | FGCGTKLTVL (56) |
| 03O15A | VH | EVQLVESGGGLVKPGGSLRLSCAASGFTLSSYSMNWVRQAPGKCLEWV |
| | | SSISRSTAYIYYADSVKGRFTISRDNAKNSVHLQMNSLRAEDTAVYFCA |
| | | REKWELLYFDSWGQGTLVTVSS (63) |
| | VL | NFMLTQPHSVSESPGKTVTISCTRNSGSIASNYVQWYQQRPGSSPTTVIY |
| | | EDNQRPSGVPDRFSGSIDSSSNSASLTISGLKTEDEADYYCQSYDSSNRV |
| | | FGCGTKLTVL (64) |
| 03P01A | VH | EVQVVESGGGLVKPGGSLRLSCAASGFTLSTSPINWVRQAPGKCLEWVS |
| | | SISRISSHKYYADSVKGRFTISRDNAKNSVYLQMNSLRVEDTAVYYCAR |
| | | EKWELLYFDYWGQGTLVTVSS (71) |
| | VL | DIVMTQTPLSLPVTPGEPASISCRSSQSLLDSDDGNTYLDWYLQKPGQSP |
| | | QLLIYTLSYRASGVPDRFSGSGSGTDFTLKISRVEAEDVGIYYCMQRIEFP |
| | | SITFGCGTRLVIK (72) |
| 03P01Z | VH | EVQVVESGGGLVKPGGSLRLSCAASGFTLSTSPINWVRQAPGKCLEWVS |
| | | SISRISSHKYYADSVKGRFTISRDNAKNSVYLQMNSLRVEDTAVYYCAR |
| | | EKWELLYFDYWGQGTLVTVSS (79) |
| | VL | NFMLTQPHSVSESPGKTVTISCTRSSGSIASNYVQWYQQRPGSSPTAVIY |
| | | EDNQRPSGVPDRFSGSIDSSSNSASLTISGLKTEDEADYYCQSYDSSNRV |
| | | FGCGTKLTVL (80) |
| 04C03A | VH | EVQLVESGGGLVKPGGSLRLSCAASGFTFNNYILNWVRQAPGKCLEWV |
| | | SSISSSSRYIFYADSVKGRFTISRDNAKNSLYLQMNSLRAEDTGVYYCAR |
| | | ESKWELLSFDYWGQGTLVTVSS (87) |
| | VL | NFMLTQPHSVSESPGKTVTISCTRSSGSIASNYVQWYQQRPGSSPINVIYE |
| | | DNQRPSGVPDRFSGSIDSSSNSASLTISGLKTEDEADYYCQSYDRSNRVF |
| | | GCGTKLTVL (88) |
| 04J18A | VH | QVQLQESGPGLVKPSQTLSLTCTVSGGSISSGGYSWSWIRQHPGKCLEWI |
| | | GSIYYSGRTYYNPALKSRITISVDTSKNQFSLMLSSVPAADTAVYYCARE |
| | | RKNELFFDYWGQGTLVTVSS (95) |
| | VL | NFMLTQPHSVSESPGKTITISCTRSSGSIASNYVQWYQQRPGSSPTAVIYE |
| | | DNQRPSGVPDRFSGSIDSSSNSASLTISGLKTEDEADYYCQSYDSSNRVF |
| | | GCGTKLTVL (96) |
| 04K01A | VH | EVHLVESGGGLVKPGGSLRLSCAASGFTFSSSLMNWVRQAPGKCLEWV |
| | | SSISSSSNYIYYADSVRGRFTISRDNAKNSLYLQMHSLRAEDTAVYYCAR |
| | | ERWVLLYFDYWGQGTLVTVSS (103) |
| | VL | NFMLTQPHSVSESPGKTVTISCTRSSGSIASNYVQWYQQRPGSSPTTVIY |
| | | EDNQRPSGVPDRFSGSIDSSSNSASLTISGLKTEDEADYYCQSYDSSIRVF |
| | | GCGTKLTVL (104) |
| 04M22A | VH | EVHLVESGGGLVKPGGSLRLSCAASGFTFSSSLMNWVRQAPGKCLEWV |
| | | SSISSSSNYIYYADSVRGRFTISRDNAKNSLYLQMHSLRAEDTAVYYCAR |
| | | ERWVLLYFDYWGQGTLVTVSS (111) |
| | VL | NFMLTQPHSVSESPGKTVTISCTRSSGSIASNYVQWYQQRPGSSPTTVIY |
| | | EDNQRPSGVPDRFSGSIDSSSNSASLTISGLKTEDEADYYCQSYDSSIRVF |
| | | GCGTKLTVL (112) |
| 04P20A | VH | EVQLAESGGGLVQPGGSLRLSCAASGFTFSSYSMNWVRQAPGKCLEWV |
| | | LYINSRSSTKYYADSVKGRFTISRDNAKNSLYLQMNSLRDADTAVYYC |
| | | ARVMRHYGMDVWGQGTTVTVSS (119) |
| | VL | NFMLTQPHSVSESPEKTVTISCTRSSGSIASNYVQWYQQRPGNSPTTLIY |
| | | EDNQRPSGVPDRFSGSIDSSSNSASLTISGLKTEDEADYYCQSFDNSNHW |
| | | VFGCGTKLTVL (120) |
| 05B02A | VH | EVQLVESGGGLVKPGGSLTLSCEASGFTFNRYSMNWVRQGPGKCLEWV |
| | | STVSSGSRYRYYADSVKGRFTISRDNAKNSLYLQMNSLRAEDTAVYYC |
| | | ARAGASTHDSFDIWGQGTMVTVPS (127) |
| | VL | NFMLTQSHSVSESPGKTVTISCTRSRGSIASNYVQWYQQRPGSSPTTVIY |
| | | EDNKRPSGVPDRFSGSIDSSSNSASLTISGLKTEDEADYYCQSYDNSNRV |
| | | FGCGTKLTVL (128) |
| 05L13A | VH | EVQLVESGGGLVKPGGSLRLSCAASGFTVSSYSMNWVRQAPGKCLEW |
| | | VSSISRSSSYIYYADSVKGRFTISRDNAKNSLYLQMNSLRAEDTAVYYC |
| | | AREKWELLYFDAWGQGTLVTVSS (135) |
| | VL | NFMLTQPHSVSESPGKTVTISCTRSSGSIASNYVQWYQQRPGSSPTTVIY |
| | | EDNQRPSGVPDRFSGSIDSSSNSASLTISGLKTEDEADYYCQSYDRSNRV |
| | | FGCGTKLTVL (136) |
| 05N13A | VH | EVQLVESGGGLVKPGGSLRLSCAASGFTFSSYSMNWVRQAPGKCLEWV |
| | | SSSSRSSSYIYYADSVKGRFTISRDNAKNSLYLQMNSLRAEDTAVYYCA |
| | | RERWELLYFDYWGQGTLVTVSS (143) |
| | VL | NFMLTQPHSVSESPGKTVTISCTRSSGSIASNYVQWYQQRPGSSPTTVIY |
| | | EDNQRPSGVPDRFSGSIDSSSNSASLTISGLKTEDEADYYCQSYDSSIRVF |
| | | GCGTKLTVL (144) |
| 06A06A | VH | QVQLQESGPGLVKPSQTLSLTCIVSGGSISSGAYYWNWIRQHPGECLEWI |
| | | GDIYYSGTTYYNPALKSRVSISVDTSKNQFSLNLISVTAADTAVYYCARE |
| | | RKNELFFDYWGQGTLVTVSS (151) |
| | VL | NFMLTQPHSVSESPGKTVTISCTRSSGSIASNYVQWYQQRPGSSPTTVIY |
| | | EDNQRPSGVPDRFSGSIDSSSNSASLTLSGLKTEDEADYYCQSYDSSNRV |
| | | FGCGTKLTVL (152) |
| 07B06A | VH | EVQVVESGGGLVKPGGSLRLSCAASGFTLSNYAMNWVRQAPGKCLEWI |
| | | SSISRSNSHKYYADSVKGRFAISRDIAKNSVYLQMNSLRAEDTAVYYCA |
| | | REKWELLYFDSWGQGTLVTVSS (159) |
| | VL | DIVMTQTPLSLPVTPGEPASISCRSSQSLLDSDDGNTYLDWYLQKPGRSP |
| | | QLLIYTLSYRASGVPDRFSGSGSGTDFTLKISRVEAEDVGIYYCMQRIEFP |
| | | SITFGCGTRLEIK (160) |
| 07B06Z | VH | EVQVVESGGGLVKPGGSLRLSCAASGFTLSNYAMNWVRQAPGKCLEWI |
| | | SSISRSNSHKYYADSVKGRFAISRDIAKNSVYLQMNSLRAEDTAVYYCA |
| | | REKWELLYFDSWGQGTLVTVSS (167) |
| | VL | NFMLTQPHSVSESPGKTITISCTRSSGSIASNYVQWYQQRPGSSPTTVIYE |
| | | DDQRPSGVPDRFSGSIDSSSNSASLTISGLKTEDEADYFCQSYDSSNRVF |
| | | GCGTKLTVL (168) |
| 07H10A | VH | QVQLMQSGAEVKKPGASVKVSCKASGYTFTDYYIHWVRQAPGQCLEW |
| | | MGWINPNSGGTTYVQKFQGRVTMTRDTSISTAYMELSRLRSDDTAVYY |
| | | CSRDRGTYYDYFDNWGQGTLVTVSS (175) |
| | VL | DIVMTQSPDSLAVSLGERATINCKSSESVLYRSNNKNYLTWYQQKPGQP |
| | | PKLLIYWASIRESGVPDRFSGSGSGTDFTLTISSLQAEDVAVYFCQQYYG |
| | | TPYTFGCGTKLEIK (176) |
| 07L12A | VH | EVQLVESGGGLVKPGGSLRLSCAASGFTFSRNSMNWVRQAPGKCLEW |
| | | VSSISTSGKYRYYADSLKDRFTISRDNAMNSLYLQMNSLRVEDTAVYYC |
| | | ARAGGSTHDSFDIWGQGTMVTVS (183) |
| | VL | NFMLTQPHSVSESPGKTVTISCTRSSGSIARNYVQWYQQRPGSSPKTVIY |
| | | EDNKRPSGVPDRISGSIDSSSNSASLTISGLKTEDEADYYCQSYDNSNRV |
| | | FGGGTKLTVL (184) |
| 07P07A | VH | EVHLVESGGGLVKPGGSLRLSCAASGFTFSFYTMNWVRQAPGKCLEWV |
| | | SSITTSSRYIYYADSVKGRFTVSRDNAKNSLYLQMNSLRAEDTAVYYCA |
| | | RAGAKTHDAFDFWGQGTMVTVSS (191) |
| | VL | NFMLTQPHSVSESPGKTVTISCSRSSGSIASNYVQWYQQRPGSSPTTVIY |
| | | EDNQRPSGVPDRFSGSIDSSSNSASLTISGLKTEDEADYYCQSYDSSNRV |
| | | FGCGTKLTVL (192) |
| 07P15A | VH | EVQLVESGGGLVKPGGSLRLSCAASGFTFSRYSMNWVRQAPGKCLEW |
| | | VSSISSSSYYIYYADSLKGRFTISRDNAKSSLYLQMNSLRAEDTAVYYCA |
| | | RERRAGFDYWGQGTLVTVSS (199) |
| | VL | NFMLTQPHSVSESPGKTVTISCTRSSGSIASNYVQWYQQRPGSSPTTVIY |
| | | EDNQRPSGVPDRFSGSIDSSSNSASLTISGLKTEDEADYYCQSYDSSNRV |
| | | FGCGTKLTVL (200) |
| 08M15A | VH | EVQLVESGGGLVKPGGSLRLSCAASGFTFSSSSMNWVRQAPGKCLEWV |
| | | SSISSRSRHIYYADSVKGRFTISRDNAKNSLYLQMNSLRAEDTAVYYCA |
| | | REKWELLYFDYWGQGTLVTVSS (207) |
| | VL | SYVLTQPPSVSVAPGQTARITCGGNNIGSKSVHWYQQKPGQAPVLVVY |
| | | DDSDRPSGIPERFSGSNSGNTATLTISRVEAGDEADYYCQVWDSSSDHV |
| | | VFGCGTKLTVL (208) |
| 08M15Z | VH | EVQLVESGGGLVKPGGSLRLSCAASGFTFSSSSMNWVRQAPGKCLEWV |
| | | SSISSRSRHIYYADSVKGRFTISRDNAKNSLYLQMNSLRAEDTAVYYCA |
| | | REKWELLYFDYWGQGTLVTVSS (215) |
| | VL | NFMLTQPHSVSESPGKTVTISCTRSSGSIASNYVQWYQQRPGSSPTTLIYE |
| | | DNQRPSGVPDRFSGSVDSSSNSASLTISGLKTEDEADYYCQSYDSSNRVF |
| | | GCGTKLTVL (216) |
| 09B13A | VH | EVQLVESGGGLVKPGGSLRLSCAASRFTLSSYSMNWVRQAPGKCLEWV |
| | | SSISRSSAYIYYADSVKGRFTISRDNAKNSVHLQMNSLRAEDTAVYYCA |
| | | REKWELLYFDYWGQGTLVTVSS (223) |
| | VL | NFMLTQPHSVSESPGKTVTISCTRSSGSIASNYVQWYQRRPGSSPTTVIY |
| | | EDNQRPSGVPDRFSGSIDSSSNSASLTISGLKTEDEADYYCQSYDSSNRV |
| | | FGCGTKLTVL (224) |
| 09D16A | VH | EVQLVESGGGLVKPGGSLRLSCAASGFTFNRYSMNWVRQAPGKCLEW |
| | | VSTVSSGSRYRYYADSVKGRFTISRDNAKNSLYLQMNSLRAEDTAVYY |
| | | CARAGGNTHDAFDIWGQGTMVTVSS (231) |
| | VL | NFMLTQSHSVSESPGKTVTISCTRSRGSIASNYVQWYQQRPGSSPTTVIY |
| | | EDNKRPSGVPDRFSGSIDSSSNSASLTISGLKTEDEADYYCQSYDNSNRV |
| | | FGCGTKVTVL (232) |
| 10E16A | VH | EVQLVESGGGLVKPGGSLRLSCAASGFTFSRYSMNWVRQAPGKCLEW |
| | | VSLISSSSSYKYYVDSVKGRFTISRDNAKNSLYLEMSSLRAEDTAVYYC |
| | | ARDRRELVLDYWGQGTLVTVSS (239) |
| | VL | SYELTQPPSVSVSPGQTARITCSGDALPKKYAYWYQQKSGQAPVLVIYE |
| | | DSKRPSGIPERLSGSSSGTMATLIISGAQVEDEADYYCYSTNNSGNHWVF |
| | | GCGTKLTVL (240) |
| 14A15A | VH | EVQLVESGGGLVRPGGSLRLSCAASGFTLSTYSMNWVRQAPGKCLEWV |
| | | STFSRSSSHIYYADSVKGRFTISRDNAKNSLYLQMNSLRAEDTAVYYCA |
| | | REQWNLLYFDYWGQGTLVTVSS (247) |
| | VL | NLMLTQPHSVSESPGKTVTISCTRSSGSIASNYVQWYQQRPGSSPTTVIY |
| | | EDNQRPSGVPDRFSGSIDSSSNSASLTISGLKTEDEADYYCQSYDRSNRV |
| | | FGCGTKLTVL (248) |
| 14C10A | VH | EVQLVESGGGLVKPGGSLKLSCAASGFTFSRYSMNWVRQASGKCLEW |
| | | VSSISSGSRYIYYRDSVRGRFTISRDNAKNSLYLQMNSLRAEDTALYYCA |
| | | REPRNHFDYWGQGTLVTVSS (255) |
| | VL | NFMLIQPHSVSESPGKTVTISCTRSSGSIASNYVQWYQHHPGSSPTTVIYE |
| | | DNQRPSGVPDRFSGSIDSSSNSASLTISGLKTEDEADYYCQSYDSSIRVFG |
| | | CGTKLTVL (256) |
| 04D18A | VH | EVQLVESGGGLVKPGGSLRLSCEASGFTFSRYSMNWVRQAPGKCLGW |
| | | VSTISSSSSYRYYADSVKGRFTISRDNAKNSLYLQMNSLRAEDTAVYYC |
| | | ARAGGNTHDAFEIWGQGTMVTVSS (263) |
| | VL | NFMLTQPHSVSASPGKTVTISCTRSSGSIARNYVQWYQQRPGSSPTTVIF |
| | | EDNKRPSGVPDRFSGSIDSSSNSASLTISGLKTEDEADYYCQSYDNSNRV |
| | | FGCGTKLTVL (264) |
| 02J12A | VH | EVQLVESGGGLVKPGGSLRLSCAASGFTFSSYTMNWVRQAPGKCLEWV |
| | | SSITRSSRYIYYADSVKGRFIISRDNAKNSLYLQMNSLRAEDTAVYYCAR |
| | | AGAATHDAFDIWAQGTMVTVSS (271) |
| | VL | NFMLTQPHSVSESPGKTVTISCTRSSGSIASNYVQWYQQRPGSSPATVIY |
| | | EDNQRPSGVPDRFSGSIDSSSNSASLTISGLKTEDEADYYCQSYDGNNRV |
| | | FGCGTKLTVL (272) |
| 07N22A | VH | EVQLVESGGGLVKPGGSLRLSCAASGFTFSIYSMNWVRQAPGKCLEWV |
| | | SSISSSSSYIHYADSVKGRFTISRDNAKNSMHLQLNSLRAEDTAVYYCAR |
| | | DRRERLLAYWGQGTLVTVSS (279) |
| | VL | SYELTQPPSVSVSPGQTARITCSGDALPKKYAYWYQQKSGQAPVLVIYE |
| | | DSKRPSGIPERFSGSSSGTMATLTISGAQVEDEADYYCYSTDSRGNLWV |
| | | FGCGTKVTVL (280) |
| 08M01A | VH | EVQLVESGGGLVKPGGSLRLSCAASGFTVSSYSMNWVRQAPGKCLEW |
| | | VSTISRSSSYIYYADSVKGRFTISRDNAKNSLYLQMNSLRAEDTAVYFCA |
| | | REKWNLLYFDAWGQGTLVTVSS (287) |
| | VL | NFMLTQPHSVSESPGKTVTISCTRSSGSIASNYVQWYQQRPGSSPTTVIY |
| | | EDNQRPSGVPDRFSGSIDSSSNSASLTISGLKTEDEADYYCQSYDSSNRV |
| | | FGCGTKLTVL (288) |
| 10B09A | VH | EVQLVESGGGLVKPGGSLRLSCAASGFTLSSYSMNWVRQAPGKCLEWV |
| | | SSISRSSNYIYYADSVKGRFTISRDNAKNSLYLQMNSLRAEDTAVYYCA |
| | | REKWNLLYFDYWGQGTLVTVS (295) |
| | VL | NLMLTQPHSVSGSPGKTVTISCTRSSGSIATNYVQWYQQRPGSSPTTVIY |
| | | EDNKRPSGVPDRFSGSIDSSSNSASLTISGLKTEDEADYYCQSYDSSNRV |
| | | FGCGTKLTVL (296) |
| 14E03A | VH | EVQVVESGGGLVQPGGSLRLSCAASGFTFSRYWMTWVRQAPGKCLEW |
| | | VANIKKDGSENYYVDSVKGRFTISRDNAKDSLYLQMNSLRAEDTAVYY |
| | | CVRVIYDWLDPWGQGTLVTVSS (303) |
| | VL | SYELTQPPSVSVSPGQTASITCSGDKLGNKYAAWYQQKPGQSPVLVIYQ |
| | | DSKRPSGIPERFSGSNSGNTATLTISGTQAMDEADYYCQAWDNNIVVFG |
| | | CGTKLTVL (304) |
| 14O18A | VH | EVQLVESGGGLVKFGGSLRLSCAASGFTFSRYSMNWVRQAPGKCLDW |
| | | VSSISMSSRYIYYVDSVKGRFTISRDNAKNSLYLQMNSLRADDTAVYYC |
| | | ARERGPGPGMDVWGQGTTVTVSS (311) |
| | VL | NFMLTQPHSVSESPGKTVTISCTRSSGSIASNYVQWYQQRPGSSPTTVIY |
| | | EDNQRPSGVPDRFSGSIDSSSNSASLTISGLKTEDEADYYCQSYDSSNRV |
| | | FGCGTKLTVL (312) |

In some embodiments, the anti-CD3 antibodies comprise:
a) a heavy chain variable region (VH) comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 7, 15, 23, 31, 39, 47, 55, 63, 71, 79, 87, 95, 103, 111, 119, 127, 135, 143, 151, 159, 167, 175, 183, 191, 199, 207, 215, 223, 231, 239, 247, 255, 263, 271, 279, 287, 295, 303, and 311; and
b) a light chain variable region (VL) comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 8, 16, 24, 32, 40, 48, 56, 64, 72, 80, 88, 96, 104, 112, 120, 128, 136, 144, 152, 160, 168, 176, 184, 192, 200, 208, 216, 224, 232, 240, 248, 256, 264, 272, 280, 288, 296, 304, and 312.

In some embodiments, the anti-CD3 antibodies comprise:
a) a heavy chain variable region (VH) comprising an amino acid sequence of any one of SEQ ID NOs: 7, 15, 23, 31, 39, 47, 55, 63, 71, 79, 87, 95, 103, 111, 119, 127, 135, 143, 151, 159, 167, 175, 183, 191, 199, 207, 215, 223, 231, 239, 247, 255, 263, 271, 279, 287, 295, 303, and 311; and
b) a light chain variable region (VL) comprising an amino acid sequence of SEQ ID NOs: 8, 16, 24, 32, 40, 48, 56, 64, 72, 80, 88, 96, 104, 112, 120, 128, 136, 144, 152, 160, 168, 176, 184, 192, 200, 208, 216, 224, 232, 240, 248, 256, 264, 272, 280, 288, 296, 304, and 312.

In some embodiments, the anti-CD3 antibodies comprise a VH and VL comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs:
a) 7 and 8, respectively;
b) 15 and 16, respectively;
c) 23 and 24, respectively;
d) 31 and 32, respectively;
e) 39 and 40, respectively;
f) 47 and 48, respectively;
g) 55 and 56, respectively;
h) 63 and 64, respectively;
i) 71 and 72, respectively;
j) 79 and 80, respectively;
k) 87 and 88, respectively;
l) 95 and 96, respectively;
m) 103 and 104, respectively;
n) 111 and 112, respectively;
o) 119 and 120, respectively;
p) 127 and 128, respectively;
q) 135 and 136, respectively;
r) 143 and 144, respectively;
s) 151 and 152, respectively;
t) 159 and 160, respectively;
u) 167 and 168, respectively;
v) 175 and 176, respectively;
w) 183 and 184, respectively;
x) 191 and 192, respectively;
y) 199 and 200, respectively;
z) 207 and 208, respectively;
aa) 215 and 216, respectively;
ab) 223 and 224, respectively;
ac) 231 and 232, respectively;
ad) 239 and 240, respectively;
ae) 247 and 248, respectively;
af) 255 and 256, respectively;
ag) 263 and 264, respectively;
ah) 271 and 272, respectively;
ai) 279 and 280, respectively;
aj) 287 and 288, respectively;
ak) 295 and 296, respectively;
al) 303 and 304, respectively; or
am) 311 and 312, respectively.

In some embodiments, the anti-CD3 antibodies comprise a VH and VL comprising the sequences of any one of SEQ ID NOs:
a) 7 and 8, respectively;
b) 15 and 16, respectively;
c) 23 and 24, respectively;
d) 31 and 32, respectively;
e) 39 and 40, respectively;
f) 47 and 48, respectively;
g) 55 and 56, respectively;
h) 63 and 64, respectively;
i) 71 and 72, respectively;
j) 79 and 80, respectively;
k) 87 and 88, respectively;
l) 95 and 96, respectively;
m) 103 and 104, respectively;
n) 111 and 112, respectively;
o) 119 and 120, respectively;
p) 127 and 128, respectively;
q) 135 and 136, respectively;
r) 143 and 144, respectively;
s) 151 and 152, respectively;
t) 159 and 160, respectively;
u) 167 and 168, respectively;
v) 175 and 176, respectively;
w) 183 and 184, respectively;
x) 191 and 192, respectively;
y) 199 and 200, respectively;
z) 207 and 208, respectively;
aa) 215 and 216, respectively;
ab) 223 and 224, respectively;
ac) 231 and 232, respectively;
ad) 239 and 240, respectively;
ae) 247 and 248, respectively;
af) 255 and 256, respectively;
ag) 263 and 264, respectively;
ah) 271 and 272, respectively;
ai) 279 and 280, respectively;
aj) 287 and 288, respectively;
ak) 295 and 296, respectively;
al) 303 and 304, respectively; or
am) 311 and 312, respectively.

In some embodiments, the anti-CD3 antibodies comprise a VH and VL comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of SEQ ID NOs:
a) 7 and 8, respectively;
b) 207 and 208, respectively;
c) 255 and 256, respectively; or
d) 303 and 304, respectively.

In some embodiments, the anti-CD3 antibodies comprise a VH and VL comprising the sequences of SEQ ID NOs:
a) 7 and 8, respectively;
b) 207 and 208, respectively;
c) 255 and 256, respectively; or
d) 303 and 304, respectively.

In some embodiments, the antibodies are bispecific or multispecific antibodies.

Provided herein are bispecific antibodies comprising a first antigen-binding domain that specifically binds CD3 and a second antigen-binding domain that specifically binds a second antigen, wherein the first antigen-binding domain that specifically binds CD3 comprises:
a) heavy chain complementarity determining region 1 (HCDR1) comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 1, 9, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289, 297, and 305;
b) an HCDR2 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 2, 10, 18, 26, 34, 42, 50, 58, 66, 74, 82, 90, 98, 106, 114, 122, 130, 138, 146, 154, 162, 170, 178, 186, 194, 202, 210, 218, 226, 234, 242, 250, 258, 266, 274, 282, 290, 298, and 306;
c) an HCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 3, 11, 19, 27, 35, 43, 51, 59, 67, 75, 83, 91, 99, 107, 115, 123, 131, 139, 147, 155, 163, 171, 179, 187, 195, 203, 211, 219, 227, 235, 243, 251, 259, 267, 275, 283, 291, 299, and 307;
d) a light chain complementarity determining region 1 (LCDR1) comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 4, 12, 20, 28, 36, 44, 52, 60, 68, 76, 84, 92, 100, 108, 116, 124, 132, 140, 148, 156, 164, 172, 180, 188, 196, 204, 212, 220, 228, 236, 244, 252, 260, 268, 276, 284, 292, 300, and 308;
e) an LCDR2 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 5, 13, 21, 29, 37, 45, 53, 61, 69, 77, 85, 93, 101, 109, 117, 125, 133, 141, 149, 157, 165, 173, 181, 189, 197, 205, 213, 221, 229, 237, 245, 253, 261, 269, 277, 285, 293, 301, and 309; and
f) an LCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 6, 14, 22, 30, 38, 46, 54, 62, 70, 78, 86, 94, 102, 110, 118, 126, 134, 142, 150, 158, 166, 174, 182, 190, 198, 206, 214, 222, 230, 238, 246, 254, 262, 270, 278, 286, 294, 302, and 310.

In some embodiments, the bispecific antibodies comprising a first antigen-binding domain that specifically binds CD3 and a second antigen-binding domain that specifically binds a second antigen, wherein the first antigen-binding domain that specifically binds CD3 comprises:
a) heavy chain complementarity determining region 1 (HCDR1) comprising the amino acid sequence of any one of SEQ ID NOs: 1, 9, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289, 297, and 305;
b) an HCDR2 comprising the amino acid sequence of any one of SEQ ID NOs: 2, 10, 18, 26, 34, 42, 50, 58, 66, 74, 82, 90, 98, 106, 114, 122, 130, 138, 146, 154, 162, 170, 178, 186, 194, 202, 210, 218, 226, 234, 242, 250, 258, 266, 274, 282, 290, 298, and 306;
c) an HCDR3 comprising the amino acid sequence of any one of SEQ ID NOs: 3, 11, 19, 27, 35, 43, 51, 59, 67, 75, 83, 91, 99, 107, 115, 123, 131, 139, 147, 155, 163, 171, 179, 187, 195, 203, 211, 219, 227, 235, 243, 251, 259, 267, 275, 283, 291, 299, and 307;
d) a light chain complementarity determining region 1 (LCDR1) comprising the amino acid sequence of any one of SEQ ID NOs: 4, 12, 20, 28, 36, 44, 52, 60, 68, 76, 84, 92, 100, 108, 116, 124, 132, 140, 148, 156, 164, 172, 180, 188, 196, 204, 212, 220, 228, 236, 244, 252, 260, 268, 276, 284, 292, 300, and 308;
e) an LCDR2 comprising the amino acid sequence of any one of SEQ ID NOs: 5, 13, 21, 29, 37, 45, 53, 61, 69, 77, 85, 93, 101, 109, 117, 125, 133, 141, 149, 157, 165, 173, 181, 189, 197, 205, 213, 221, 229, 237, 245, 253, 261, 269, 277, 285, 293, 301, and 309; and
f) an LCDR3 comprising the amino acid sequence of any one of SEQ ID NOs: 6, 14, 22, 30, 38, 46, 54, 62, 70, 78, 86, 94, 102, 110, 118, 126, 134, 142, 150, 158, 166, 174, 182, 190, 198, 206, 214, 222, 230, 238, 246, 254, 262, 270, 278, 286, 294, 302, and 310.

In some embodiments, the first antigen-binding domain that specifically binds CD3 comprises:
a) an HCDR1, HCDR2, and HCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs:
   i) 1, 2, and 3, respectively;
   ii) 9, 10, and 11, respectively;
   iii) 17, 18, and 19, respectively;
   iv) 25, 26, and 27, respectively;
   v) 33, 34, and 35, respectively;
   vi) 41, 42, and 43, respectively;
   vii) 49, 50, and 51, respectively;
   viii) 57, 58, and 59, respectively;
   ix) 65, 66, and 67, respectively;
   x) 73, 74, and 75, respectively;
   xi) 81, 82, and 83, respectively;
   xii) 89, 90, and 91, respectively;
   xiii) 97, 98, and 99, respectively;
   xiv) 105, 106, and 107, respectively;
   xv) 113, 114, and 115, respectively;
   xvi) 121, 122, and 123, respectively;
   xvii) 129, 130, and 131, respectively;
   xviii) 137, 138, and 139, respectively;
   xix) 145, 146, and 147, respectively;
   xx) 153, 154, and 155, respectively;
   xxi) 161, 162, and 163, respectively;
   xxii) 169, 170, and 171, respectively;
   xxiii) 177, 178, and 179, respectively;
   xxiv) 185, 186, and 187, respectively;
   xxv) 193, 194, and 195, respectively;
   xxvi) 201, 202, and 203, respectively;
   xxvii) 209, 210, and 211, respectively;
   xxviii) 217, 218, and 219, respectively;
   xxix) 225, 226, and 227, respectively;
   xxx) 233, 234, and 235, respectively;
   xxxi) 241, 242, and 243, respectively;
   xxxii) 249, 250, and 251, respectively;
   xxxiii) 257, 258, and 259, respectively;
   xxxiv) 265, 266, and 267, respectively;
   xxxv) 273, 274, and 275, respectively;
   xxxvi) 281, 282, and 283, respectively;
   xxxvii) 289, 290, and 291, respectively;
   xxxviii) 297, 298, and 299, respectively; or
   xxxix) 305, 306, and 307, respectively; and
b) an LCDR1, LCDR2, and LCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs:
   i) 4, 5, and 6, respectively;
   ii) 12, 13, and 14, respectively;
   iii) 20, 21, and 22, respectively;
   iv) 28, 29, and 30, respectively;
   v) 36, 37, and 38, respectively;
   vi) 44, 45, and 46, respectively;
   vii) 52, 53, and 54, respectively;
   viii) 60, 61, and 62, respectively;
   ix) 68, 69, and 70, respectively;
   x) 76, 77, and 78, respectively;
   xi) 84, 85, and 86, respectively;
   xii) 92, 93, and 94, respectively;
   xiii) 100, 101, and 102, respectively;
   xiv) 108, 109, and 110, respectively;
   xv) 116, 117, and 118, respectively;
   xvi) 124, 125, and 126, respectively;
   xvii) 132, 133, and 134, respectively;
   xviii) 140, 141, and 142, respectively;
   xix) 148, 149, and 150, respectively;
   xx) 156, 157, and 158, respectively;
   xxi) 164, 165, and 166, respectively;
   xxii) 172, 173, and 174, respectively;
   xxiii) 180, 181, and 182, respectively;
   xxiv) 188, 189, and 190, respectively;
   xxv) 196, 197, and 198, respectively;
   xxvi) 204, 205, and 206, respectively;
   xxvii) 212, 213, and 214, respectively;
   xxviii) 220, 221, and 222, respectively;
   xxix) 228, 229, and 230, respectively;
   xxx) 236, 237, and 238, respectively;
   xxxi) 244, 245, and 246, respectively;
   xxxii) 252, 253, and 254, respectively;
   xxxiii) 260, 261, and 262, respectively;
   xxxiv) 268, 269, and 270, respectively;
   xxxv) 276, 277, and 278, respectively;
   xxxvi) 284, 285, and 286, respectively;
   xxxvii) 292, 293, and 294, respectively;
   xxxviii) 300, 301, and 302, respectively; or
   xxxix) 308, 309, and 310, or respectively.

In some embodiments, the first antigen-binding domain that specifically binds CD3 comprises:
a) an HCDR1, HCDR2, and HCDR3 comprising the amino acid sequences of SEQ ID NOS:
   i) 1, 2, and 3, respectively;
   ii) 9, 10, and 11, respectively;
   iii) 17, 18, and 19, respectively;
   iv) 25, 26, and 27, respectively;
   v) 33, 34, and 35, respectively;
   vi) 41, 42, and 43, respectively;
   vii) 49, 50, and 51, respectively;
   viii) 57, 58, and 59, respectively;
   ix) 65, 66, and 67, respectively;
   x) 73, 74, and 75, respectively;
   xi) 81, 82, and 83, respectively;
   xii) 89, 90, and 91, respectively;
   xiii) 97, 98, and 99, respectively;
   xiv) 105, 106, and 107, respectively;
   xv) 113, 114, and 115, respectively;
   xvi) 121, 122, and 123, respectively;
   xvii) 129, 130, and 131, respectively;
   xviii) 137, 138, and 139, respectively;
   xix) 145, 146, and 147, respectively;
   xx) 153, 154, and 155, respectively;
   xxi) 161, 162, and 163, respectively;
   xxii) 169, 170, and 171, respectively;
   xxiii) 177, 178, and 179, respectively;
   xxiv) 185, 186, and 187, respectively;
   xxv) 193, 194, and 195, respectively;
   xxvi) 201, 202, and 203, respectively;
   xxvii) 209, 210, and 211, respectively;
   xxviii) 217, 218, and 219, respectively;
   xxix) 225, 226, and 227, respectively;
   xxx) 233, 234, and 235, respectively;
   xxxi) 241, 242, and 243, respectively;
   xxxii) 249, 250, and 251, respectively;
   xxxiii) 257, 258, and 259, respectively;
   xxxiv) 265, 266, and 267, respectively;
   xxxv) 273, 274, and 275, respectively;
   xxxvi) 281, 282, and 283, respectively;
   xxxvii) 289, 290, and 291, respectively;
   xxxviii) 297, 298, and 299, respectively; or
   xxxix) 305, 306, and 307, respectively; and
b) an LCDR1, LCDR2, and LCDR3 comprising the amino acid sequence of SEQ ID NOs:
   i) 4, 5, and 6, respectively;
   ii) 12, 13, and 14, respectively;
   iii) 20, 21, and 22, respectively;
   iv) 28, 29, and 30, respectively;
   v) 36, 37, and 38, respectively;
   vi) 44, 45, and 46, respectively;
   vii) 52, 53, and 54, respectively;
   viii) 60, 61, and 62, respectively;
   ix) 68, 69, and 70, respectively;
   x) 76, 77, and 78, respectively;
   xi) 84, 85, and 86, respectively;
   xii) 92, 93, and 94, respectively;
   xiii) 100, 101, and 102, respectively;
   xiv) 108, 109, and 110, respectively;
   xv) 116, 117, and 118, respectively;
   xvi) 124, 125, and 126, respectively;
   xvii) 132, 133, and 134, respectively;
   xviii) 140, 141, and 142, respectively;
   xix) 148, 149, and 150, respectively;
   xx) 156, 157, and 158, respectively;
   xxi) 164, 165, and 166, respectively;
   xxii) 172, 173, and 174, respectively;
   xxiii) 180, 181, and 182, respectively;
   xxiv) 188, 189, and 190, respectively;
   xxv) 196, 197, and 198, respectively;
   xxvi) 204, 205, and 206, respectively;
   xxvii) 212, 213, and 214, respectively;
   xxviii) 220, 221, and 222, respectively;
   xxix) 228, 229, and 230, respectively;
   xxx) 236, 237, and 238, respectively;
   xxxi) 244, 245, and 246, respectively;
   xxxii) 252, 253, and 254, respectively;
   xxxiii) 260, 261, and 262, respectively;
   xxxiv) 268, 269, and 270, respectively;
   xxxv) 276, 277, and 278, respectively;
   xxxvi) 284, 285, and 286, respectively;
   xxxvii) 292, 293, and 294, respectively;
   xxxviii) 300, 301, and 302, respectively; or
   xxxix) 308, 309, and 310, or respectively.

In some embodiments, the first antigen-binding domain that specifically binds CD3 comprises an HCDR1, HCDR2, HCDR3, LCDR1, LCDR2, and LCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs:
a) 1, 2, 3, 4, 5, and 6, respectively;
b) 9, 10, 11, 12, 13, and 14, respectively;
c) 17, 18, 19, 20, 21, and 22, respectively;
d) 25, 26, 27, 28, 29, and 30, respectively;
e) 33, 34, 35, 36, 37, and 38, respectively;
f) 41, 42, 43, 44, 45, and 46, respectively;
g) 49, 50, 51, 52, 53, and 54, respectively;
h) 57, 58, 59, 60, 61, and 62, respectively;
i) 65, 66, 67, 68, 69, and 70, respectively;
j) 73, 74, 75, 76, 77, and 78, respectively;
k) 81, 82, 83, 84, 85, and 86, respectively;
l) 89, 90, 91, 92, 93, and 94, respectively;
m) 97, 98, 99, 100, 101, and 102, respectively;
n) 105, 106, 107, 108, 109, and 110, respectively;
o) 113, 114, 115, 116, 117, and 118, respectively;
p) 121, 122, 123, 124, 125, and 126, respectively;
q) 129, 130, 131, 132, 133, and 134, respectively;
r) 137, 138, 139, 140, 141, and 142, respectively;
s) 145, 146, 147, 148, 149, and 150, respectively;
t) 153, 154, 155, 156, 157, and 158, respectively;
u) 161, 162, 163, 164, 165, and 166, respectively;
v) 169, 170, 171, 172, 173, and 174, respectively;
w) 177, 178, 179, 180, 181, and 182, respectively;
x) 185, 186, 187, 188, 189, and 190, respectively;
y) 193, 194, 195, 196, 197, and 198, respectively;
z) 201, 202, 203, 204, 205, and 206, respectively;
aa) 209, 210, 211, 212, 213, and 214, respectively;
ab) 217, 218, 219, 220, 221, and 222, respectively;
ac) 225, 226, 227, 228, 229, and 230, respectively;
ad) 233, 234, 235, 236, 237, and 238, respectively;
ae) 241, 242, 243, 244, 245, and 246, respectively;
af) 249, 250, 251, 252, 253, and 254, respectively;
ag) 257, 258, 259, 260, 261, and 262, respectively;
ah) 265, 266, 267, 268, 269, and 270, respectively;
ai) 273, 274, 275, 276, 277, and 278, respectively;
aj) 281, 282, 283, 284, 285, and 286, respectively;
ak) 289, 290, 291, 292, 293, and 294, respectively;
al) 297, 298, 299, 300, 301, and 302, respectively; or
am) 305, 306, 307, 308, 309, and 310, respectively.

In some embodiments, the first antigen-binding domain that specifically binds CD3 comprises an HCDR1, HCDR2, HCDR3, LCDR1, LCDR2, and LCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of SEQ ID NOs:
a) 1, 2, 3, 4, 5, and 6, respectively;
b) 201, 202, 203, 204, 205, and 206, respectively;
c) 249, 250, 251, 252, 253, and 254, respectively; or
d) 297, 298, 299, 300, 301, and 302, respectively.

In some embodiments, the first antigen-binding domain that specifically binds CD3 comprises an HCDR1, HCDR2, HCDR3, LCDR1, LCDR2, and LCDR3 comprising the sequences of SEQ ID NOs:
a) 1, 2, 3, 4, 5, and 6, respectively;
b) 201, 202, 203, 204, 205, and 206, respectively;
c) 249, 250, 251, 252, 253, and 254, respectively; or
d) 297, 298, 299, 300, 301, and 302, respectively.

In some embodiments, the first antigen-binding domain that specifically binds CD3 comprises an HCDR1, HCDR2, HCDR3, LCDR1, LCDR2, and LCDR3 comprising the amino acid sequences of SEQ ID NOS:
a) 1, 2, 3, 4, 5, and 6, respectively;
b) 9, 10, 11, 12, 13, and 14, respectively;
c) 17, 18, 19, 20, 21, and 22, respectively;
d) 25, 26, 27, 28, 29, and 30, respectively;
e) 33, 34, 35, 36, 37, and 38, respectively;
f) 41, 42, 43, 44, 45, and 46, respectively;
g) 49, 50, 51, 52, 53, and 54, respectively;
h) 57, 58, 59, 60, 61, and 62, respectively;
i) 65, 66, 67, 68, 69, and 70, respectively;
j) 73, 74, 75, 76, 77, and 78, respectively;
k) 81, 82, 83, 84, 85, and 86, respectively;
l) 89, 90, 91, 92, 93, and 94, respectively;
m) 97, 98, 99, 100, 101, and 102, respectively;
n) 105, 106, 107, 108, 109, and 110, respectively;
o) 113, 114, 115, 116, 117, and 118, respectively;
p) 121, 122, 123, 124, 125, and 126, respectively;
q) 129, 130, 131, 132, 133, and 134, respectively;
r) 137, 138, 139, 140, 141, and 142, respectively;
s) 145, 146, 147, 148, 149, and 150, respectively;
t) 153, 154, 155, 156, 157, and 158, respectively;
u) 161, 162, 163, 164, 165, and 166, respectively;
v) 169, 170, 171, 172, 173, and 174, respectively;
w) 177, 178, 179, 180, 181, and 182, respectively;
x) 185, 186, 187, 188, 189, and 190, respectively;
y) 193, 194, 195, 196, 197, and 198, respectively;
z) 201, 202, 203, 204, 205, and 206, respectively;
aa) 209, 210, 211, 212, 213, and 214, respectively;
ab) 217, 218, 219, 220, 221, and 222, respectively;
ac) 225, 226, 227, 228, 229, and 230, respectively;
ad) 233, 234, 235, 236, 237, and 238, respectively;
ae) 241, 242, 243, 244, 245, and 246, respectively;
af) 249, 250, 251, 252, 253, and 254, respectively;
ag) 257, 258, 259, 260, 261, and 262, respectively;
ah) 265, 266, 267, 268, 269, and 270, respectively;
ai) 273, 274, 275, 276, 277, and 278, respectively;
aj) 281, 282, 283, 284, 285, and 286, respectively;
ak) 289, 290, 291, 292, 293, and 294, respectively;
al) 297, 298, 299, 300, 301, and 302, respectively; or
am) 305, 306, 307, 308, 309, and 310, respectively.

In some embodiments, the first antigen-binding domain that specifically binds CD3 comprises:
a) a heavy chain variable region (VH) comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 7, 15, 23, 31, 39, 47, 55, 63, 71, 79, 87, 95, 103, 111, 119, 127, 135, 143, 151, 159, 167, 175, 183, 191, 199, 207, 215, 223, 231, 239, 247, 255, 263, 271, 279, 287, 295, 303, and 311; and
b) a light chain variable region (VL) comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 8, 16, 24, 32, 40, 48, 56, 64, 72, 80, 88, 96, 104, 112, 120, 128, 136, 144, 152, 160, 168, 176, 184, 192, 200, 208, 216, 224, 232, 240, 248, 256, 264, 272, 280, 288, 296, 304, and 312.

In some embodiments, the first antigen-binding domain that specifically binds CD3 comprises:
a) a heavy chain variable region (VH) comprising the amino acid sequence of any one of SEQ ID NOs: 7, 15, 23, 31, 39, 47, 55, 63, 71, 79, 87, 95, 103, 111, 119, 127, 135, 143, 151, 159, 167, 175, 183, 191, 199, 207, 215, 223, 231, 239, 247, 255, 263, 271, 279, 287, 295, 303, and 311; and
b) a light chain variable region (VL) comprising the amino acid sequence of any one of SEQ ID NOs: 8, 16, 24, 32, 40, 48, 56, 64, 72, 80, 88, 96, 104, 112, 120, 128, 136, 144, 152, 160, 168, 176, 184, 192, 200, 208, 216, 224, 232, 240, 248, 256, 264, 272, 280, 288, 296, 304, and 312.

In some embodiments, the first antigen-binding domain that specifically binds CD3 comprises a VH and VL comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOS:
a) 7 and 8, respectively;
b) 15 and 16, respectively;
c) 23 and 24, respectively;
d) 31 and 32, respectively;
e) 39 and 40, respectively;
f) 47 and 48, respectively;
g) 55 and 56, respectively;
h) 63 and 64, respectively;
i) 71 and 72, respectively;
j) 79 and 80, respectively;
k) 87 and 88, respectively;
l) 95 and 96, respectively;
m) 103 and 104, respectively;
n) 111 and 112, respectively;
o) 119 and 120, respectively;
p) 127 and 128, respectively;
q) 135 and 136, respectively;
r) 143 and 144, respectively;
s) 151 and 152, respectively;
t) 159 and 160, respectively;
u) 167 and 168, respectively;
v) 175 and 176, respectively;
w) 183 and 184, respectively;
x) 191 and 192, respectively;
y) 199 and 200, respectively;
z) 207 and 208, respectively;
aa) 215 and 216, respectively;
ab) 223 and 224, respectively;
ac) 231 and 232, respectively;
ad) 239 and 240, respectively;
ae) 247 and 248, respectively;
af) 255 and 256, respectively;
ag) 263 and 264, respectively;
ah) 271 and 272, respectively;
ai) 279 and 280, respectively;
aj) 287 and 288, respectively;
ak) 295 and 296, respectively;
al) 303 and 304, respectively; or am) 311 and 312, respectively.

In some embodiments, the first antigen-binding domain that specifically binds CD3 comprises a VH and VL comprising the amino acid sequences of SEQ ID NO:
a) 7 and 8, respectively;
b) 15 and 16, respectively;
c) 23 and 24, respectively;
d) 31 and 32, respectively;
e) 39 and 40, respectively;
f) 47 and 48, respectively;
g) 55 and 56, respectively;
h) 63 and 64, respectively;
i) 71 and 72, respectively;
j) 79 and 80, respectively;
k) 87 and 88, respectively;
l) 95 and 96, respectively;
m) 103 and 104, respectively;
n) 111 and 112, respectively;
o) 119 and 120, respectively;
p) 127 and 128, respectively;
q) 135 and 136, respectively;
r) 143 and 144, respectively;
s) 151 and 152, respectively;
t) 159 and 160, respectively;
u) 167 and 168, respectively;
v) 175 and 176, respectively;
w) 183 and 184, respectively;
x) 191 and 192, respectively;
y) 199 and 200, respectively;
z) 207 and 208, respectively;
aa) 215 and 216, respectively;
ab) 223 and 224, respectively;
ac) 231 and 232, respectively;
ad) 239 and 240, respectively;
ae) 247 and 248, respectively;
af) 255 and 256, respectively;
ag) 263 and 264, respectively;
ah) 271 and 272, respectively;
ai) 279 and 280, respectively;
aj) 287 and 288, respectively;
ak) 295 and 296, respectively;
al) 303 and 304, respectively; or
am) 311 and 312, respectively.

In some embodiments, the first antigen-binding domain that specifically binds CD3 comprises a VH and VL comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of SEQ ID NOs:
a) 7 and 8, respectively;
b) 207 and 208, respectively;
c) 255 and 256, respectively; or
d) 303 and 304, respectively.

In some embodiments, the first antigen-binding domain that specifically binds CD3 comprises a VH and VL comprising the sequences of SEQ ID NOs:
a) 7 and 8, respectively;
b) 207 and 208, respectively;
c) 255 and 256, respectively; or
d) 303 and 304, respectively.

Provided herein are multispecific antibodies comprising a first antigen-binding domain that specifically binds CD3 and at least one, two, three, four, five, or more antigen-binding domains that specifically bind a second, third, fourth, fifth, sixth, or more antigen, wherein the first antigen-binding domain that specifically binds CD3 comprises:
a) heavy chain complementarity determining region 1 (HCDR1) comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 1, 9, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289, 297, and 305;
b) an HCDR2 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 2, 10, 18, 26, 34, 42, 50, 58, 66, 74, 82, 90, 98, 106, 114, 122, 130, 138, 146, 154, 162, 170, 178, 186, 194, 202, 210, 218, 226, 234, 242, 250, 258, 266, 274, 282, 290, 298, and 306;
c) an HCDR3 comprising an amino acid sequence having at least 85% identity, at least 90% identity, at least 95% identity, at least 96% identity, at least 97% identity, at least 98% identity, or at least 99% identity to the sequences of any one of SEQ ID NOs: 3, 11, 19, 27, 35, 43, 51, 59, 67, 75, 83, 91, 99, 107, 115, 123, 131, 139, 147, 155, 163, 171, 179, 187, 195, 203, 211, 219, 227, 235, 243, 251, 259, 267, 275, 283, 291, 299, and 307;
d) a light chain complementarity determining region 1 (LCDR1) comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 4, 12, 20, 28, 36, 44, 52, 60, 68, 76, 84, 92, 100, 108, 116, 124, 132, 140, 148, 156, 164, 172, 180, 188, 196, 204, 212, 220, 228, 236, 244, 252, 260, 268, 276, 284, 292, 300, and 308;
e) an LCDR2 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 5, 13, 21, 29, 37, 45, 53, 61, 69, 77, 85, 93, 101, 109, 117, 125, 133, 141, 149, 157, 165, 173, 181, 189, 197, 205, 213, 221, 229, 237, 245, 253, 261, 269, 277, 285, 293, 301, and 309; and
f) an LCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 6, 14, 22, 30, 38, 46, 54, 62, 70, 78, 86, 94, 102, 110, 118, 126, 134, 142, 150, 158, 166, 174, 182, 190, 198, 206, 214, 222, 230, 238, 246, 254, 262, 270, 278, 286, 294, 302, and 310.

In some embodiments, the first antigen-binding domain that specifically binds CD3 and at least one, two, three, four, five, or more antigen-binding domains that specifically bind a second, third, fourth, fifth, sixth, or more antigen, wherein the first antigen-binding domain that specifically binds CD3 comprises:
a) heavy chain complementarity determining region 1 (HCDR1) comprising the amino acid sequences of any one of SEQ ID NOs: 1, 9, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289, 297, and 305;
b) an HCDR2 comprising the amino acid sequence of any one of SEQ ID NOs: 2, 10, 18, 26, 34, 42, 50, 58, 66, 74, 82, 90, 98, 106, 114, 122, 130, 138, 146, 154, 162, 170, 178, 186, 194, 202, 210, 218, 226, 234, 242, 250, 258, 266, 274, 282, 290, 298, and 306;
c) an HCDR3 comprising the amino acid sequence of any one of SEQ ID NOs: 3, 11, 19, 27, 35, 43, 51, 59, 67, 75, 83, 91, 99, 107, 115, 123, 131, 139, 147, 155, 163, 171, 179, 187, 195, 203, 211, 219, 227, 235, 243, 251, 259, 267, 275, 283, 291, 299, and 307;
d) a light chain complementarity determining region 1 (LCDR1) comprising the amino acid sequence of any one of SEQ ID NOs: 4, 12, 20, 28, 36, 44, 52, 60, 68, 76, 84, 92, 100, 108, 116, 124, 132, 140, 148, 156, 164, 172, 180, 188, 196, 204, 212, 220, 228, 236, 244, 252, 260, 268, 276, 284, 292, 300, and 308;
e) an LCDR2 comprising the amino acid sequence of any one of SEQ ID NOs: 5, 13, 21, 29, 37, 45, 53, 61, 69, 77, 85, 93, 101, 109, 117, 125, 133, 141, 149, 157, 165, 173, 181, 189, 197, 205, 213, 221, 229, 237, 245, 253, 261, 269, 277, 285, 293, 301, and 309; and
f) an LCDR3 comprising the amino acid sequence of any one of SEQ ID NOs: 6, 14, 22, 30, 38, 46, 54, 62, 70, 78, 86, 94, 102, 110, 118, 126, 134, 142, 150, 158, 166, 174, 182, 190, 198, 206, 214, 222, 230, 238, 246, 254, 262, 270, 278, 286, 294, 302, and 310.

In some embodiments, the first antigen-binding domain that specifically binds CD3 comprises:
a) an HCDR1, HCDR2, and HCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs:
   i) 1, 2, and 3, respectively;
   ii) 9, 10, and 11, respectively;
   iii) 17, 18, and 19, respectively;
   iv) 25, 26, and 27, respectively;
   v) 33, 34, and 35, respectively;
   vi) 41, 42, and 43, respectively;
   vii) 49, 50, and 51, respectively;
   viii) 57, 58, and 59, respectively;
   ix) 65, 66, and 67, respectively;
   x) 73, 74, and 75, respectively;
   xi) 81, 82, and 83, respectively;
   xii) 89, 90, and 91, respectively;
   xiii) 97, 98, and 99, respectively;
   xiv) 105, 106, and 107, respectively;
   xv) 113, 114, and 115, respectively;
   xvi) 121, 122, and 123, respectively;
   xvii) 129, 130, and 131, respectively;
   xviii) 137, 138, and 139, respectively;
   xix) 145, 146, and 147, respectively;
   xx) 153, 154, and 155, respectively;
   xxi) 161, 162, and 163, respectively;
   xxii) 169, 170, and 171, respectively;
   xxiii) 177, 178, and 179, respectively;
   xxiv) 185, 186, and 187, respectively;
   xxv) 193, 194, and 195, respectively;
   xxvi) 201, 202, and 203, respectively;
   xxvii) 209, 210, and 211, respectively;
   xxviii) 217, 218, and 219, respectively;
   xxix) 225, 226, and 227, respectively;
   xxx) 233, 234, and 235, respectively;
   xxxi) 241, 242, and 243, respectively;
   xxxii) 249, 250, and 251, respectively;
   xxxiii) 257, 258, and 259, respectively;
   xxxiv) 265, 266, and 267, respectively;
   xxxv) 273, 274, and 275, respectively;
   xxxvi) 281, 282, and 283, respectively;
   xxxvii) 289, 290, and 291, respectively;
   xxxviii) 297, 298, and 299, respectively; or
   xxxix) 305, 306, and 307, respectively; and
b) an LCDR1, LCDR2, and LCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs:
   i) 4, 5, and 6, respectively;
   ii) 12, 13, and 14, respectively;
   iii) 20, 21, and 22, respectively;
   iv) 28, 29, and 30, respectively;
   v) 36, 37, and 38, respectively;
   vi) 44, 45, and 46, respectively;
   vii) 52, 53, and 54, respectively;
   viii) 60, 61, and 62, respectively;
   ix) 68, 69, and 70, respectively;
   x) 76, 77, and 78, respectively;
   xi) 84, 85, and 86, respectively;
   xii) 92, 93, and 94, respectively;
   xiii) 100, 101, and 102, respectively;
   xiv) 108, 109, and 110, respectively;
   xv) 116, 117, and 118, respectively;
   xvi) 124, 125, and 126, respectively;
   xvii) 132, 133, and 134, respectively;
   xviii) 140, 141, and 142, respectively;
   xix) 148, 149, and 150, respectively;
   xx) 156, 157, and 158, respectively;
   xxi) 164, 165, and 166, respectively;
   xxii) 172, 173, and 174, respectively;
   xxiii) 180, 181, and 182, respectively;
   xxiv) 188, 189, and 190, respectively;
   xxv) 196, 197, and 198, respectively;
   xxvi) 204, 205, and 206, respectively;
   xxvii) 212, 213, and 214, respectively;
   xxviii) 220, 221, and 222, respectively;
   xxix) 228, 229, and 230, respectively;
   xxx) 236, 237, and 238, respectively;
   xxxi) 244, 245, and 246, respectively;
   xxxii) 252, 253, and 254, respectively;
   xxxiii) 260, 261, and 262, respectively;
   xxxiv) 268, 269, and 270, respectively;
   xxxv) 276, 277, and 278, respectively;
   xxxvi) 284, 285, and 286, respectively;
   xxxvii) 292, 293, and 294, respectively;
   xxxviii) 300, 301, and 302, respectively; or
   xxxix) 308, 309, and 310, or respectively.

In some embodiments, the first antigen-binding domain that specifically binds CD3 comprises:
a) an HCDR1, HCDR2, and HCDR3 comprising the amino acid sequences of SEQ ID NOs:
   i) 1, 2, and 3, respectively;
   ii) 9, 10, and 11, respectively;
   iii) 17, 18, and 19, respectively;
   iv) 25, 26, and 27, respectively;
   v) 33, 34, and 35, respectively;
   vi) 41, 42, and 43, respectively;
   vii) 49, 50, and 51, respectively;
   viii) 57, 58, and 59, respectively;
   ix) 65, 66, and 67, respectively;
   x) 73, 74, and 75, respectively;
   xi) 81, 82, and 83, respectively;
   xii) 89, 90, and 91, respectively;
   xiii) 97, 98, and 99, respectively;
   xiv) 105, 106, and 107, respectively;
   xv) 113, 114, and 115, respectively;
   xvi) 121, 122, and 123, respectively;
   xvii) 129, 130, and 131, respectively;
   xviii) 137, 138, and 139, respectively;
   xix) 145, 146, and 147, respectively;
   xx) 153, 154, and 155, respectively;
   xxi) 161, 162, and 163, respectively;
   xxii) 169, 170, and 171, respectively;
   xxiii) 177, 178, and 179, respectively;
   xxiv) 185, 186, and 187, respectively;
   xxv) 193, 194, and 195, respectively;
   xxvi) 201, 202, and 203, respectively;
   xxvii) 209, 210, and 211, respectively;
   xxviii) 217, 218, and 219, respectively;
   xxix) 225, 226, and 227, respectively;
   xxx) 233, 234, and 235, respectively;
   xxxi) 241, 242, and 243, respectively;
   xxxii) 249, 250, and 251, respectively;
   xxxiii) 257, 258, and 259, respectively;
   xxxiv) 265, 266, and 267, respectively;
   xxxv) 273, 274, and 275, respectively;
   xxxvi) 281, 282, and 283, respectively;
   xxxvii) 289, 290, and 291, respectively;
   xxxviii) 297, 298, and 299, respectively; or
   xxxix) 305, 306, and 307, respectively; and
b) an LCDR1, LCDR2, and LCDR3 comprising the amino acid sequence of SEQ ID NOS:
   i) 4, 5, and 6, respectively;
   ii) 12, 13, and 14, respectively;
   iii) 20, 21, and 22, respectively;
   iv) 28, 29, and 30, respectively;
   v) 36, 37, and 38, respectively;
   vi) 44, 45, and 46, respectively;
   vii) 52, 53, and 54, respectively;
   viii) 60, 61, and 62, respectively;
   ix) 68, 69, and 70, respectively;
   x) 76, 77, and 78, respectively;
   xi) 84, 85, and 86, respectively;
   xii) 92, 93, and 94, respectively;
   xiii) 100, 101, and 102, respectively;
   xiv) 108, 109, and 110, respectively;
   xv) 116, 117, and 118, respectively;
   xvi) 124, 125, and 126, respectively;
   xvii) 132, 133, and 134, respectively;
   xviii) 140, 141, and 142, respectively;
   xix) 148, 149, and 150, respectively;
   xx) 156, 157, and 158, respectively;
   xxi) 164, 165, and 166, respectively;
   xxii) 172, 173, and 174, respectively;
   xxiii) 180, 181, and 182, respectively;
   xxiv) 188, 189, and 190, respectively;
   xxv) 196, 197, and 198, respectively;
   xxvi) 204, 205, and 206, respectively;
   xxvii) 212, 213, and 214, respectively;
   xxviii) 220, 221, and 222, respectively;
   xxix) 228, 229, and 230, respectively;
   xxx) 236, 237, and 238, respectively;
   xxxi) 244, 245, and 246, respectively;
   xxxii) 252, 253, and 254, respectively;
   xxxiii) 260, 261, and 262, respectively;
   xxxiv) 268, 269, and 270, respectively;
   xxxv) 276, 277, and 278, respectively;
   xxxvi) 284, 285, and 286, respectively;
   xxxvii) 292, 293, and 294, respectively;
   xxxviii) 300, 301, and 302, respectively; or
   xxxix) 308, 309, and 310, or respectively.

In some embodiments, the first antigen-binding domain that specifically binds CD3 comprises an HCDR1, HCDR2, HCDR3, LCDR1, LCDR2, and LCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs:
a) 1, 2, 3, 4, 5, and 6, respectively;
b) 9, 10, 11, 12, 13, and 14, respectively;
c) 17, 18, 19, 20, 21, and 22, respectively;
d) 25, 26, 27, 28, 29, and 30, respectively;
e) 33, 34, 35, 36, 37, and 38, respectively;
f) 41, 42, 43, 44, 45, and 46, respectively;
g) 49, 50, 51, 52, 53, and 54, respectively;
h) 57, 58, 59, 60, 61, and 62, respectively;
i) 65, 66, 67, 68, 69, and 70, respectively;
j) 73, 74, 75, 76, 77, and 78, respectively;
k) 81, 82, 83, 84, 85, and 86, respectively;
l) 89, 90, 91, 92, 93, and 94, respectively;
m) 97, 98, 99, 100, 101, and 102, respectively;
n) 105, 106, 107, 108, 109, and 110, respectively;
o) 113, 114, 115, 116, 117, and 118, respectively;
p) 121, 122, 123, 124, 125, and 126, respectively;
q) 129, 130, 131, 132, 133, and 134, respectively;
1) 137, 138, 139, 140, 141, and 142, respectively;
s) 145, 146, 147, 148, 149, and 150, respectively;
t) 153, 154, 155, 156, 157, and 158, respectively;
u) 161, 162, 163, 164, 165, and 166, respectively;
v) 169, 170, 171, 172, 173, and 174, respectively;
w) 177, 178, 179, 180, 181, and 182, respectively;
x) 185, 186, 187, 188, 189, and 190, respectively;
y) 193, 194, 195, 196, 197, and 198, respectively;
z) 201, 202, 203, 204, 205, and 206, respectively;
aa) 209, 210, 211, 212, 213, and 214, respectively;
ab) 217, 218, 219, 220, 221, and 222, respectively;
ac) 225, 226, 227, 228, 229, and 230, respectively;
ad) 233, 234, 235, 236, 237, and 238, respectively;
ae) 241, 242, 243, 244, 245, and 246, respectively;
af) 249, 250, 251, 252, 253, and 254, respectively;
ag) 257, 258, 259, 260, 261, and 262, respectively;
ah) 265, 266, 267, 268, 269, and 270, respectively;
ai) 273, 274, 275, 276, 277, and 278, respectively;
aj) 281, 282, 283, 284, 285, and 286, respectively;
ak) 289, 290, 291, 292, 293, and 294, respectively;
al) 297, 298, 299, 300, 301, and 302, respectively; or
am) 305, 306, 307, 308, 309, and 310, respectively.

In some embodiments, the first antigen-binding domain that specifically binds CD3 comprises an HCDR1, HCDR2, HCDR3, LCDR1, LCDR2, and LCDR3 comprising the amino acid sequences of SEQ ID NOS:
a) 1, 2, 3, 4, 5, and 6, respectively;
b) 9, 10, 11, 12, 13, and 14, respectively;
c) 17, 18, 19, 20, 21, and 22, respectively;
d) 25, 26, 27, 28, 29, and 30, respectively;
e) 33, 34, 35, 36, 37, and 38, respectively;
f) 41, 42, 43, 44, 45, and 46, respectively;
g) 49, 50, 51, 52, 53, and 54, respectively;
h) 57, 58, 59, 60, 61, and 62, respectively;
i) 65, 66, 67, 68, 69, and 70, respectively;
j) 73, 74, 75, 76, 77, and 78, respectively;
k) 81, 82, 83, 84, 85, and 86, respectively;
l) 89, 90, 91, 92, 93, and 94, respectively;
m) 97, 98, 99, 100, 101, and 102, respectively;
n) 105, 106, 107, 108, 109, and 110, respectively;
o) 113, 114, 115, 116, 117, and 118, respectively;
p) 121, 122, 123, 124, 125, and 126, respectively;
q) 129, 130, 131, 132, 133, and 134, respectively;
r) 137, 138, 139, 140, 141, and 142, respectively;
s) 145, 146, 147, 148, 149, and 150, respectively;
t) 153, 154, 155, 156, 157, and 158, respectively;
u) 161, 162, 163, 164, 165, and 166, respectively;
v) 169, 170, 171, 172, 173, and 174, respectively;
w) 177, 178, 179, 180, 181, and 182, respectively;
x) 185, 186, 187, 188, 189, and 190, respectively;
y) 193, 194, 195, 196, 197, and 198, respectively;
z) 201, 202, 203, 204, 205, and 206, respectively;
aa) 209, 210, 211, 212, 213, and 214, respectively;
ab) 217, 218, 219, 220, 221, and 222, respectively;
ac) 225, 226, 227, 228, 229, and 230, respectively;
ad) 233, 234, 235, 236, 237, and 238, respectively;
ae) 241, 242, 243, 244, 245, and 246, respectively;
af) 249, 250, 251, 252, 253, and 254, respectively;
ag) 257, 258, 259, 260, 261, and 262, respectively;
ai) 273, 274, 275, 276, 277, and 278, respectively;
aj) 281, 282, 283, 284, 285, and 286, respectively;
ak) 289, 290, 291, 292, 293, and 294, respectively;
al) 297, 298, 299, 300, 301, and 302, respectively; or
am) 305, 306, 307, 308, 309, and 310, respectively.

In some embodiments, the first antigen-binding domain that specifically binds CD3 comprises an HCDR1, HCDR2, HCDR3, LCDR1, LCDR2, and LCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of SEQ ID NOS:

a) 1, 2, 3, 4, 5, and 6, respectively;
b) 201, 202, 203, 204, 205, and 206, respectively;
c) 249, 250, 251, 252, 253, and 254, respectively; or
d) 297, 298, 299, 300, 301, and 302, respectively.

In some embodiments, the first antigen-binding domain that specifically binds CD3 comprises an HCDR1, HCDR2, HCDR3, LCDR1, LCDR2, and LCDR3 comprising the sequences of SEQ ID NOs:
a) 1, 2, 3, 4, 5, and 6, respectively;
b) 201, 202, 203, 204, 205, and 206, respectively;
c) 249, 250, 251, 252, 253, and 254, respectively; or
d) 297, 298, 299, 300, 301, and 302, respectively.

In some embodiments, the first antigen-binding domain that specifically binds CD3 comprises:
a) a heavy chain variable region (VH) comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 7, 15, 23, 31, 39, 47, 55, 63, 71, 79, 87, 95, 103, 111, 119, 127, 135, 143, 151, 159, 167, 175, 183, 191, 199, 207, 215, 223, 231, 239, 247, 255, 263, 271, 279, 287, 295, 303, and 311; and
b) a light chain variable region (VL) comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 8, 16, 24, 32, 40, 48, 56, 64, 72, 80, 88, 96, 104, 112, 120, 128, 136, 144, 152, 160, 168, 176, 184, 192, 200, 208, 216, 224, 232, 240, 248, 256, 264, 272, 280, 288, 296, 304, and 312.

In some embodiments, the first antigen-binding domain that specifically binds CD3 comprises:
a) a heavy chain variable region (VH) comprising the amino acid sequence of any one of SEQ ID NOs: 7, 15, 23, 31, 39, 47, 55, 63, 71, 79, 87, 95, 103, 111, 119, 127, 135, 143, 151, 159, 167, 175, 183, 191, 199, 207, 215, 223, 231, 239, 247, 255, 263, 271, 279, 287, 295, 303, and 311; and
b) a light chain variable region (VL) comprising the amino acid sequence of any one of SEQ ID NOs: 8, 16, 24, 32, 40, 48, 56, 64, 72, 80, 88, 96, 104, 112, 120, 128, 136, 144, 152, 160, 168, 176, 184, 192, 200, 208, 216, 224, 232, 240, 248, 256, 264, 272, 280, 288, 296, 304, and 312.

In some embodiments, the first antigen-binding domain that specifically binds CD3 comprises a VH and VL comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs:
a) 7 and 8, respectively;
b) 15 and 16, respectively;
c) 23 and 24, respectively;
d) 31 and 32, respectively;
e) 39 and 40, respectively;
f) 47 and 48, respectively;
g) 55 and 56, respectively;
h) 63 and 64, respectively;
i) 71 and 72, respectively;
j) 79 and 80, respectively;
k) 87 and 88, respectively;
l) 95 and 96, respectively;
m) 103 and 104, respectively;
n) 111 and 112, respectively;
o) 119 and 120, respectively;
p) 127 and 128, respectively;
q) 135 and 136, respectively;
r) 143 and 144, respectively;
s) 151 and 152, respectively;
t) 159 and 160, respectively;
u) 167 and 168, respectively;
v) 175 and 176, respectively;
w) 183 and 184, respectively;
x) 191 and 192, respectively;
y) 199 and 200, respectively;
z) 207 and 208, respectively;
aa) 215 and 216, respectively;
ab) 223 and 224, respectively;
ac) 231 and 232, respectively;
ad) 239 and 240, respectively;
ae) 247 and 248, respectively;
af) 255 and 256, respectively;
ag) 263 and 264, respectively;
ah) 271 and 272, respectively;
ai) 279 and 280, respectively;
aj) 287 and 288, respectively;
ak) 295 and 296, respectively;
al) 303 and 304, respectively; or am) 311 and 312, respectively.

In some embodiments, the first antigen-binding domain that specifically binds CD3 comprises a VH and VL comprising the amino acid sequences of SEQ ID NO:
a) 7 and 8, respectively;
b) 15 and 16, respectively;
c) 23 and 24, respectively;
d) 31 and 32, respectively;
e) 39 and 40, respectively;
f) 47 and 48, respectively;
g) 55 and 56, respectively;
h) 63 and 64, respectively;
i) 71 and 72, respectively;
j) 79 and 80, respectively;
k) 87 and 88, respectively;
l) 95 and 96, respectively;
m) 103 and 104, respectively;
n) 111 and 112, respectively;
o) 119 and 120, respectively;
p) 127 and 128, respectively;
q) 135 and 136, respectively;
r) 143 and 144, respectively;
s) 151 and 152, respectively;
t) 159 and 160, respectively;
u) 167 and 168, respectively;
v) 175 and 176, respectively;
w) 183 and 184, respectively;
x) 191 and 192, respectively;
y) 199 and 200, respectively;
z) 207 and 208, respectively;
aa) 215 and 216, respectively;
ab) 223 and 224, respectively;
ac) 231 and 232, respectively;
ad) 239 and 240, respectively;
ae) 247 and 248, respectively;
af) 255 and 256, respectively;
ag) 263 and 264, respectively;
ah) 271 and 272, respectively;
ai) 279 and 280, respectively;
aj) 287 and 288, respectively;
ak) 295 and 296, respectively;
al) 303 and 304, respectively; or
am) 311 and 312, respectively.

In some embodiments, the first antigen-binding domain that specifically binds CD3 comprises a VH and VL comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of SEQ ID NOs:
a) 7 and 8, respectively;
b) 207 and 208, respectively;
c) 255 and 256, respectively; or
d) 303 and 304, respectively.

In some embodiments, the first antigen-binding domain that specifically binds CD3 comprises a VH and VL comprising the sequences of SEQ ID NOs:
a) 7 and 8, respectively;
b) 207 and 208, respectively;
c) 255 and 256, respectively; or
d) 303 and 304, respectively.

Also provided herein are bispecific and multispecific antibodies comprising a first antigen-binding domain that specifically binds CD3 and at least a second antigen-binding domain that specifically binds a second antigen, where the first antigen-binding domain does not bind cells expressing cynomolgus CD3.

Also provided herein are bispecific and multispecific antibodies comprising a first antigen-binding domain that specifically binds CD3 and at least a second antigen-binding domain that specifically binds a second antigen, wherein the second antigen-binding domain binds CD3 with a binding dissociation constant (K_{D})) value of less than about 1.2× 10⁻⁸ M as measured in a surface plasmon resonance assay at 25° C.

Also provided herein are bispecific and multispecific antibodies comprising a first antigen-binding domain that specifically binds CD3 and at least a second antigen-binding domain that specifically binds a second antigen, wherein the first antigen-binding domain comprises a single-chain antibody or a Fab.

Also provided herein are bispecific and multispecific antibodies comprising a first antigen-binding domain that specifically binds CD3 and at least a second antigen-binding domain that specifically binds CD3, wherein the bispecific antibodies are IgG1 subtype.

Also provided herein are bispecific and multispecific antibodies comprising a first antigen-binding domain that specifically binds CD3 and at least a second antigen-binding domain that specifically binds a second antigen, wherein the bispecific antibodies comprise an Fc domain, comprising one or more substitutions in the Fc domain. In some embodiments, the one or more substitutions in the Fc domain reduce Fc domain-mediated effector function, reduce binding to protein A, or improve bispecific antibody production.

Also provided herein are bispecific and multispecific antibodies comprising a first antigen-binding domain that specifically binds CD3 and at least a second antigen-binding domain that specifically binds a second antibody, wherein the bispecific and multispecific antibodies comprise an Fc domain comprising one or more substitutions in the Fc domain. In some embodiments, the one or more substitutions in the Fc domain, using residue numbering according to EU numbering, comprise H435R, Y436F, T366S/L368A/Y407V, T366W, S354C, Y349C, L234A, and/or L235A. In some embodiments, one Fc domain comprises L234A, L235A, S354C, Y349C, T366S, L368A, Y407V, H435R, and Y436F substitutions and the second Fc domain comprises L234A, L235A, S354C, Y349C, and T366W substitutions.

In some embodiments, the antibodies, bispecific antibodies, and multispecific antibodies described herein induce T-cell activation and proliferation.

In some embodiments, the antibodies, bispecific antibodies, and multispecific antibodies described herein activate cytokine release.

In other embodiments, variant antibodies, antigen-binding fragments, scFv or bispecific antibodies comprising one or more conservative mutations in the antigen-binding domain. The conservative mutations may reside in the well-known framework regions or any of the CDRs, as long as the variant antibodies, antigen-binding fragments or scFv retain the desired functional properties of the parent molecules, such as those described herein.

Those of skill in the art are aware that various amino acids have similar properties. One or more such amino acids of an antibody can often be substituted by one or more other such amino acids without eliminating a desired activity of that antibody. For example, the amino acids glycine, alanine, valine, leucine, and isoleucine can often be substituted for one another (conservative modifications).

“Conservative modifications” refer to amino acid modifications that do not significantly affect or alter the binding characteristics of the antibody or an antigen-binding fragment containing the amino acid modifications. Conservative modifications include amino acid substitutions, insertions and deletions. Conservative amino acid substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis (MacLennan et al., Acta Physiol. Scand. Suppl. 1988; 643:55-67; Sasaki et al., Adv. Biophys. 1988; 35:1-24).

TABLE 3
Conservative Amino Acid Substitutions

| | | 3 Letter | 1 Letter |
|---|---|---|---|
| Group | Amino Acid | Abbreviation | Abbreviation |
| Aromatic | Phenylalanine | Phe | F |
| | Tryptophan | Trp | W |
| | Tyrosine | Tyr | Y |
| Negatively Charged | Aspartate | Asp | D |
| | Glutamate | Glu | E |
| Non-Polar Aliphatic | Alanine | Ala | A |
| | Glycine | Gly | G |
| | Isoleucine | Ile | I |
| | Leucine | Leu | L |
| | Methionine | Met | M |
| | Valine | Val | V |
| Polar Uncharged | Asparagine | Asn | N |
| | Cysteine | Cys | C |
| | Glutamine | Gln | Q |
| | Proline | Pro | P |
| | Serine | Ser | S |
| | Threonine | Thr | T |
| Positively Charged | Arginine | Arg | R |
| | Histidine | His | H |
| | Lysine | Lys | K |

Within a group of amino acids, some substitutions may be preferred over others. For example, glycine and alanine may preferably be used to substitute for one another (since they have relatively short side chains) and valine, leucine and isoleucine may be used to substitute for one another (since they have larger aliphatic side chains which are hydrophobic). Amino acid substitutions to the antibodies described herein may be made by known methods for example by PCR mutagenesis (U.S. Pat. No. 4,683,195). Alternatively, libraries of variants may be generated for example using random (NNK) or non-random codons, for example DVK codons, which encode 11 amino acids (Ala, Cys, Asp, Glu, Gly, Lys, Asn, Arg, Ser, Tyr, Trp). The resulting antibody variants may be tested for their characteristics using assays described herein.

Amino acid deletions or insertions can also be made relative to the amino acid sequences provided for the antibodies described herein. Thus, for example, amino acids which do not have a substantial effect on the activity of the polypeptide, or at least which do not eliminate such activity, can be deleted. Such deletions can be advantageous since the overall length and the molecular weight of a polypeptide can be reduced whilst still retaining activity. This can enable the amount of polypeptide required for a particular purpose to be reduced—for example, dosage levels can be reduced.

Various embodiments comprising optional amino acid substitutions in the antibody sequences are provided herein. In some embodiments, the antibody comprises up to 10, up to 5, or up to 2 amino acid substitutions in the antigen-binding domain. For example, anti-CD3 antibodies or antigen-binding domains comprising the 6 CDRs of 01C03A, 01G24A, 01M02A, 01P14N, 02107A, 02107A, 03J04A, 03L07A, 03O15A, 03P01A, 03P01Z, 04C03A, 04J18A, 04K01A, 04M22A, 04P20A, 05B02A, 05L13A, 05N13A, 06a06A, 07B06A, 07B06A, 07B06Z, 07H10A, 07L12A, 07P07A, 07P15A, 08M15A, 08M15Z, 09B13A, 09D16A, 10E16A, 14C10A, 04D18A, 02J12A, 07N22A, 08M01A, 10B09A, 14E03A, or 14018A may comprise up to 10 amino acid substitutions across all of its CDR regions, up to 5 amino acid substitutions, or up to 2 amino acid substitutions. In another example, anti-CD3 antibodies or antigen-binding domains comprising the VH and VL of 01C03A, 01G24A, 01M02A, 01P14N, 02107A, 02107A, 03J04A, 03L07A, 03015A, 03P01A, 03P01Z, 04C03A, 04J18A, 04K01A, 04M22A, 04P20A, 05B02A, 05L13A, 05N13A, 06a06A, 07B06A, 07B06A, 07B06Z, 07H10A, 07L12A, 07P07A, 07P15A, 08M15A, 08M15Z, 09B13A, 09D16A, 10E16A, 14C10A, 04D18A, 02J12A, 07N22A, 08M01A, 10B09A, 14E03A, or 14O18A may comprise up to 10 amino acid substitutions across all of the VH and VL regions, up to 5 amino acid substitutions, or up to 2 amino acid substitutions.

In some embodiments, the one or more amino acid substitutions are in the CDR region or regions. In other embodiments, the one or more amino acid substitutions are in the framework regions, i.e. in the variable heavy and light chains but not in the CDR region or regions. In other embodiments, the one or more amino acid substitutions may be at any position in the variable heavy and/or variable light regions. In some embodiments, the amino acid substitutions do not occur in a CDR sequence. In some embodiments, the amino acid substitutions do not adversely affect the binding specificity and/or affinity of the antibody. Accordingly, the variant antibody may have the same (or substantially the same) or a superior functional profile as the antibody from which is it derived.

### Bispecific Format

Different formats of bispecific antibodies have been described and reviewed by Chames and Baty in Curr. Opin. Drug Disc. Devel. 2009; 12:276-83.

In some embodiments, the present disclosure provides bispecific antibodies that are IgG-like molecules with CH3 domains with asymmetric mutations to drive heterodimerization (as compared to homodimerization) between two parental antibodies, forming recombinant IgG-like bispecific antibodies, wherein the two sides of the antibody each contain a Fab fragment, or part of a Fab fragment, or an scFv of at least two different antibodies.

“Homodimerization” as used herein refers to an interaction of two heavy chains having identical CH3 amino acid sequences.

“Heterodimerization” as used herein refers to an interaction of two heavy chains having non-identical CH3 amino acid sequences.

In some embodiments, the bispecific antibodies are of IgG1 subtype.

In some embodiments, the bispecific antibodies comprise a first antigen-binding domain that specifically binds CD3, a second antigen-binding domain that specifically binds a second antigen, and an Fc domain, wherein the Fc domain comprises one or more substitutions in the Fc domain that reduces Fc domain-mediated effector functions.

In some embodiments, the second antigen that the second antigen-binding domain specifically binds is a cell surface antigen that is expressed on a cell other than an immune T-cell. In some embodiments, the second antigen is a cell surface antigen that is expressed on a cell other than an immune effector T-cell. In some embodiments, the second antigen is a cell surface antigen that is expressed on a diseased cell. In some embodiments the cell surface antigen expressed on a diseased cell is associated with the diseased cell (e.g., expressed at a higher level on a diseased cell than a non-diseased cell, expressed only on the diseased cell, an isoform expressed on a diseased cell that is different from that expressed on a non-diseased cell, expressed only on a diseased cell that has been infected with a pathogen, etc.). In some embodiments, the cell surface antigen associated with the disease is a cancer-associated or tumor-associated antigen.

In some embodiments, bispecific antibodies comprise a first antigen-binding domain that specifically binds CD3, a second antigen-binding domain that specifically binds a second antigen, and an Fc domain, wherein the Fc domain is of IgG1 isotype. In some embodiments, the IgG1 isotype Fc domain has one or more substitutions in the Fc domain that reduce effector function and knob-in-hole mutations. The Fc domain, with reduced effector function and knob-in-hole mutations, IgG1 platform is designed to create bispecific antibodies with reduced or eliminated Fc-mediated effector functions while maintaining the stability, pharmacokinetics, and half-life of the bispecific antibody. This is particularly useful for bispecific antibodies where Fc-mediated effector functions are not desired or could cause unintended side effects.

The “knob-in-hole” approach involves the introduction of specific mutations in the constant (CH) region of the heavy chains to promote the preferential pairing of two different heavy chains, typically of different antigenic specificity. (Sun et al., Science Translational Medicine 2015, 7: 287ra70; PCT Intl. Publ. No. WO2006/028936). The “knob” mutation creates a protruding amino acid side chain on one heavy chain, while the “hole” mutation generates a cavity on the other heavy chain. The knob fits into the hole, promoting the correct heterodimerization of the two heavy chains. In some embodiments, the “knob” mutation may comprise a mutation of threonine (T) to tyrosine (Y) or T to tryptophan (W) at position 366 (T366Y or T366W) on one heavy chain to create the knob. In some embodiments, the “hole” mutation may comprise mutations of threonine (T) to serine(S) at position 366 (T366S), leucine (L) to alanine (A) at position 368 (L368A), and tyrosine (Y) to serine(S) at position 407 (Y407S) or Y to valine (V) at position 407 (Y407V) on the other heavy chain to create the hole. In some embodiments, T366W functions as a knob mutation in the CD3 binding heavy chain and T366S, L368A and Y407V function as hole mutations in the second antigen binding heavy chain in the bispecific antibodies described herein. It is expected that the functionality of the bispecific antibodies is not altered if the knob-hole mutations were reversed in the two heavy chains. In some embodiments, an additional mutation of serine(S) to cysteine (C) at position 354 on one heavy chain and a mutation of tyrosine (Y) to cysteine (C) at position 349 may improve the heterodimerization ratio. In some embodiments, the additional mutations of S354C in the CD3 binding heavy chain and Y349C in the second antigen binding heavy chain are also present in the antibodies described herein and may provide for an improved heterodimerization ratio of up to 95%. (Nat. Biotechnol. 1998; 16 (7): 677-81). It is expected that functionality of the resulting bispecific antibody is not altered if the S354C and Y349C mutations were reversed in the two heavy chains.

Further, in some embodiments, the bispecific antibody comprising a first antigen-binding domain that specifically binds CD3 and a second antigen-binding domain that specifically binds a second antigen comprise one or more substitutions in the Fc domain that reduces its binding to protein A.

In some embodiments, H435R and Y436F substitutions are incorporated into the CH3 domain of an antibody heavy chain of the present invention in an asymmetric manner (e.g., mutations in one heavy chain only). In some embodiments the substitutions H435R and Y436F are in the CH3 domain of a heavy chain comprising a “knob” mutation. In some embodiments, H435R and Y436F substitutions are in a heavy chain comprising a T366W substitution. In some embodiments, H435R and Y436 substitutions are in the CD3 binding heavy chain, ablating protein A binding of the bispecific antibody incorporating these mutations. The H435R and Y436F mutations may also be introduced in the CH3 domain of the second antigen binding heavy chain with the same effect. By using this combination of mutations, highly pure monovalent bispecific antibodies can be rapidly purified directly from combined culture media using standard protein A purification. (Pharmaceutics 2019 Dec. 18; 12 (1): 3).

Other strategies, such as promoting heavy chain heterodimerization using electrostatic interactions by substituting positively charged residues at one CH3 surface and negatively charged residues at a second CH3 surface, may be used, as described in US Pat. Publ. No. US2010/0015133; US Pat. Publ. No. US2009/0182127; US Pat. Publ. No. US2010/028637 or US Pat. Publ. No. US2011/0123532, to promote heterodimerization of two heavy chains of different binding specificity.

In other such strategies, heterodimerization may be promoted using asymmetric substitutions in the heavy chains as described in U.S. Pat. Publ. No. US2012/0149876 or U.S. Pat. Publ. No. US2013/0195849.

Another embodiment incorporates a VH44-VL100 interchain disulfide bond to increase the stability of the scFv portion of the bispecific antibodies described herein. See Prot. Eng. Des. Sel. 2012; 25 (7): 321-9.

In some embodiments, the bispecific antibody comprising a first antigen-binding domain that specifically binds CD3 and a second antigen-binding domain that specifically binds a second antigen comprises a first heavy chain (HCl) that comprises substitutions L234A, L235A, Y349C, S354C, T366S, L368A, Y407V, H435R and Y436F and a second heavy chain (HC2) that comprises substitutions L234A, L235A, T366W, Y349C, and S354C, according to EU numbering. In some embodiments, HCl and/or HC2 further comprise M252Y/S254T/T256E (YTE) mutations, according to EU numbering.

In addition to the methods described above, the bispecific antibody comprising a first antigen-binding domain that specifically binds CD3 and a second antigen-binding domain that specifically binds a second antigen described herein may be generated in vitro in a cell-free environment by introducing asymmetrical mutations in the CH3 regions of two mono specific homodimeric antibodies and forming the bispecific heterodimeric antibody from two parent monospecific homodimeric antibodies in reducing conditions to allow disulfide bond isomerization according to methods described in PCT Intl. Pat. Publ. No. WO2011/131746. In the methods, the first monospecific antigen-binding molecule (e.g., antibody that specifically binds CD3) and the second monospecific antigen-binding molecule (e.g., antibody that specifically binds a second antigen) are engineered to have certain substitutions at the CH3 domain that promotes heterodimer stability; the antigen-binding molecules are incubated together under reducing conditions sufficient to allow the cysteines in the hinge region to undergo disulfide bond isomerization; thereby generating the bispecific antibody by Fab arm exchange. The incubation conditions may optimally be restored to non-reducing conditions. Exemplary reducing agents that may be used are 2-mercaptoethylamine (2-MEA), dithiothreitol (DTT), dithioerythritol (DTE), glutathione, tris (2-carboxyethyl) phosphine (TCEP), L-cysteine and beta-mercaptoethanol, preferably a reducing agent selected from the group consisting of: 2-mercaptoethylamine, dithiothreitol and tris(2-carboxyethyl) phosphine. For example, incubation for at least 90 minutes at a temperature of at least 20° C. in the presence of at least 25 mM 2-MEA or in the presence of at least 0.5 mM dithiothreitol at a pH from 5-8, for example at pH of 7.0 or at pH of 7.4 may be used.

Also provided herein are isolated nucleic acids encoding the antibody or antigen-binding fragment thereof described herein. “Nucleic acid” refers to a synthetic molecule comprising a chain of nucleotides covalently linked by a sugar-phosphate backbone or other equivalent covalent chemistry. cDNA is an exemplary synthetic polynucleotide. In another general aspect, the disclosure provided herein relates to an isolated nucleic acid encoding a bispecific antibody described herein. It will be appreciated by those skilled in the art that the coding sequence can be altered or codon optimization may be performed without changing the amino acid sequence of the expressed protein, such as an antibody. Accordingly, it will be understood by those skilled in the art that nucleic acid sequences encoding the bispecific antibodies, antibodies or antigen-binding fragments thereof described herein can be altered or codon optimized without changing the amino acid sequences of the expressed antibodies.

Also provided herein are vectors comprising an isolated nucleic acid encoding an antibody or antigen-binding fragment thereof described herein.

Further provided herein are vectors comprising an isolated nucleic acid encoding a bispecific antibody comprising a first antigen-binding domain that specifically binds CD3 and a second antigen-binding domain that specifically binds a second antigen described herein. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and/origin of replication. The promoter can be a constitutive, inducible or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to embodiments disclosed herein. Such techniques are well known to those skilled in the art in view of the present disclosure.

Also provided herein are host cells comprising the vector described herein.

Further provided herein is a host cell comprising a vector comprising an isolated nucleic acid encoding a bispecific antibody comprising a first antigen-binding domain that specifically binds CD3 and a second antigen-binding domain that specifically binds a second antigen described herein. Any host cell known to those skilled in the art in view of the present disclosure can be used for recombinant expression of the bispecific antibodies, antibodies or antigen-binding fragments thereof described herein. In some embodiments, the host cells are E. coli TG1 or BL21 cells, CHO-DG44, CHO-K1 or HEK293 cells. According to particular embodiments, the recombinant expression vector is transformed into host cells by conventional methods such as chemical transfection, heat shock, or electroporation, where it is stably integrated into the host cell genome such that the recombinant nucleic acid is effectively expressed.

Also provided herein are methods of producing an antibody or antigen-binding fragment thereof described herein, comprising culturing a host cell described herein under conditions to produce an antibody or antigen-binding fragment thereof described herein, and purifying the antibody or antigen-binding fragment thereof.

Further provided herein are methods of producing a bispecific antibody comprising a first antigen-binding domain that specifically binds CD3 and a second antigen-binding domain that specifically binds a second antigen, comprising culturing a host cell comprising a vector comprising an isolated nucleic acid encoding a bispecific antibody comprising a first antigen-binding domain that specifically binds CD3 and a second antigen-binding domain that specifically binds a second antigen under conditions to produce the bispecific antibody, and purifying the bispecific antibody. Expressed bispecific antibodies or antigen-binding fragments thereof can be harvested from the cells and purified according to conventional techniques known in the art.

### Pharmaceutical Compositions

Also provided herein is a pharmaceutical composition comprising an isolated antibody or antigen-binding fragment thereof described herein and a pharmaceutically acceptable carrier. In another general aspect, the pharmaceutical composition comprises an antibody described herein and a pharmaceutically acceptable carrier. In some embodiments, the antibody is a bispecific or multispecific antibody.

The term “pharmaceutical composition” as used herein means a product comprising an antibody described herein together with a pharmaceutically acceptable carrier. Antibodies described herein and compositions comprising them are also useful in the manufacture of a medicament for therapeutic applications mentioned herein. In some embodiments, the antibody is a bispecific or multispecific antibody.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition or the biological activity of a pharmaceutical composition as provided herein. In particular embodiments, any pharmaceutically acceptable carrier suitable for use in an antibody pharmaceutical composition can be used.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include buffers, diluents, solvents, tonicity regulating agents, preservatives, stabilizers, and chelating agents. One or more pharmaceutically acceptable carriers may be used in formulating the pharmaceutical compositions described herein.

Another embodiment described herein provides a pharmaceutical composition formulated as a liquid formulation. An embodiment of a liquid formulation is an aqueous formulation, i.e., a formulation comprising water. The liquid formulation may comprise a solution, a suspension, an emulsion, a microemulsion, a gel, and the like. An aqueous formulation typically comprises at least 50% w/w water, or at least 60%, 70%, 75%, 80%, 85%, 90%, or at least 95% w/w of water.

Another embodiment described herein provides a pharmaceutical composition as non-aqueous formulation. Non-aqueous antibody formulations are described in U.S. Pat. No. 11,795,429.

Another embodiment described herein provides a pharmaceutical composition formulated as an injectable which can be injected, for example, via an injection device (e.g., a syringe or an infusion pump). The injection may be delivered subcutaneously, intramuscularly, intraperitoneally, intravitreally, or intravenously, for example.

In another embodiment, the pharmaceutical composition is a solid formulation, e.g., a freeze-dried or spray-dried composition, which may be used as is, or whereto the physician or the patient adds solvents, and/or diluents prior to use.

The dosage forms may be immediate release, sustained release, or modified release.

The pH in an aqueous formulation can be between pH 3 and pH 10. In one embodiment described herein, the pH of the pharmaceutical composition is from about 7.0 to about 9.5. In another embodiment described herein, the pH of the pharmaceutical composition is from about 3.0 to about 7.0.

In another embodiment described herein, the pharmaceutical composition comprises a buffer. Non-limiting examples of buffers include: arginine, aspartic acid, bicine, citrate, disodium hydrogen phosphate, fumaric acid, glycine, glycylglycine, histidine, lysine, maleic acid, malic acid, sodium acetate, sodium carbonate, sodium dihydrogen phosphate, sodium phosphate, succinate, tartaric acid, tricine, and tris(hydroxymethyl)-aminomethane, and mixtures thereof. The buffer may be present individually or in the aggregate, in a concentration from about 0.01 mg/mL to about 50 mg/mL, for example from about 0.1 mg/mL to about 20 mg/mL. Pharmaceutical compositions comprising any of these specific buffers, alone or in combination, constitute alternative embodiments described herein.

Another embodiment described herein provides a pharmaceutical composition comprising a preservative. Non-limiting examples of preservatives include: benzethonium chloride, benzoic acid, benzyl alcohol, bronopol, butyl 4-hydroxybenzoate, chlorobutanol, chlorocresol, chlorohexidine, chlorphenesin, o-cresol, m-cresol, p-cresol, ethyl 4-hydroxybenzoate, imidurea, methyl 4-hydroxybenzoate, phenol, 2-phenoxyethanol, 2-phenylethanol, propyl 4-hydroxybenzoate, sodium dehydroacetate, thiomerosal, and mixtures thereof. The preservative may be present individually or in the aggregate, in a concentration from about 0.01 mg/mL to about 50 mg/mL, for example from about 0.1 mg/mL to about 20 mg/mL. Pharmaceutical compositions comprising any of these specific preservatives, alone or in combination, constitute alternative embodiments described herein.

Another embodiment described herein provides a pharmaceutical composition comprising an isotonic agent. Non-limiting examples of the embodiment include a salt (such as sodium chloride), an amino acid (such as glycine, histidine, arginine, lysine, isoleucine, aspartic acid, tryptophan, and threonine), an alditol (such as glycerol, 1,2-propanediol propyleneglycol), 1,3-propanediol, and 1,3-butanediol), polyethyleneglycol (e.g., PEG400), and mixtures thereof. Another example of an isotonic agent includes a sugar. Non-limiting examples of sugars may be mono-, di-, or polysaccharides, or water-soluble glucans, including for example fructose, glucose, mannose, sorbose, xylose, maltose, lactose, sucrose, trehalose, dextran, pullulan, dextrin, cyclodextrin, alpha- and beta-HPCD, soluble starch, hydroxyethyl starch, and sodium carboxymethyl-cellulose. Another example of an isotonic agent is a sugar alcohol, wherein the term “sugar alcohol” is defined as a C (4-8) hydrocarbon having at least one —OH group. Non-limiting examples of sugar alcohols include mannitol, sorbitol, inositol, galactitol, dulcitol, xylitol, and arabitol. Pharmaceutical compositions comprising any of the isotonic agents alone or in combination constitute alternative embodiments described herein. The isotonic agent may be present individually or in the aggregate, in a concentration from about 0.01 mg/mL to about 50 mg/mL, for example from about 0.1 mg/mL to about 20 mg/mL. Pharmaceutical compositions comprising any of these specific isotonic agents, alone or in combination, constitute alternative embodiments described herein.

Another embodiment described herein provides a pharmaceutical composition comprising a chelating agent. Non-limiting examples of chelating agents include citric acid, aspartic acid, salts of ethylenediaminetetraacetic acid (EDTA) and mixtures thereof. The chelating agent may be present individually or in the aggregate, in a concentration from about 0.01 mg/mL to about 50 mg/mL, for example from about 0.1 mg/mL to about 20 mg/mL. Pharmaceutical compositions comprising any of these specific chelating agents, alone or in combination, constitute alternative embodiments described herein.

Another embodiment described herein provides a pharmaceutical composition comprising a stabilizer. Non-limiting examples of stabilizers include one or more aggregation inhibitors, one or more oxidation inhibitors, one or more surfactants, and/or one or more protease inhibitors.

Another embodiment described herein provides a pharmaceutical composition comprising a stabilizer, wherein the stabilizer is carboxy-hydroxycellulose and derivates thereof (such as HPC, HPC-SL, HPC-L and HPMC), cyclodextrins, 2-methylthioethanol, polyethylene glycol (such as PEG 3350), polyvinyl alcohol (PVA), polyvinylpyrrolidone, salts (such as sodium chloride), sulfur-containing substances such as monothioglycerol), or thioglycolic acid. The stabilizer may be present individually or in the aggregate, in a concentration from about 0.01 mg/mL to about 50 mg/mL, for example from about 0.1 mg/mL to about 20 mg/mL. Pharmaceutical compositions comprising any of these specific stabilizers, alone or in combination, constitute alternative embodiments described herein.

Another embodiment described herein provides a pharmaceutical composition comprising one or more surfactants. The term “surfactant” refers to any molecules or ions that are comprised of a water-soluble (hydrophilic) part, and a fat-soluble (lipophilic) part. The surfactant may, for example, be selected from the group consisting of anionic surfactants, cationic surfactants, nonionic surfactants, and/or zwitterionic surfactants. The surfactant may be present individually or in the aggregate, in a concentration from about 0.1 mg/mL to about 20 mg/mL. Pharmaceutical compositions comprising any of these specific surfactants, alone or in combination, constitute alternative embodiments described herein.

Another embodiment described herein provides a pharmaceutical composition comprising one or more protease inhibitors, such as, e.g., EDTA, and/or benzamidine hydrochloric acid (HCl). The protease inhibitor may be present individually or in the aggregate, in a concentration from about 0.1 mg/mL to about 20 mg/mL. Pharmaceutical compositions comprising any of these specific protease inhibitors, alone or in combination, constitute alternative embodiments described herein.

Another embodiment described herein provides a method of producing a pharmaceutical composition comprising a bispecific antibody, antibody or antigen-binding fragment thereof described herein, comprising combining the bispecific antibody, antibody or antigen-binding fragment thereof with a pharmaceutically acceptable carrier to generate the pharmaceutical composition. In another general aspect, an embodiment described herein relates to a method of producing a pharmaceutical composition comprising a bispecific antibody described herein, comprising combining a bispecific antibody described herein with a pharmaceutically acceptable carrier to generate the pharmaceutical composition.

### Methods of Use

Provided herein are methods comprising administering to a subject a therapeutically effective amount of a pharmaceutical composition comprising an anti-CD3 antibody or a bispecific or multispecific antibody that specifically binds CD3 and at least one second agent of the invention. In some embodiments, the subject is a subject in need thereof.

Further provided herein are methods comprising administering to a subject a therapeutically effective amount of a pharmaceutical composition comprising a bispecific or multispecific antibody comprising a first antigen-binding domain that specifically binds CD3 and a second antigen-binding domain that specifically binds a second antigen. In some embodiments, the subject is a subject in need thereof.

The therapeutically effective amount of a pharmaceutical composition of the disclosure can comprise any of the antibodies or bispecific antibodies as disclosed herein and a pharmaceutically acceptable carrier or diluent. As used herein, the expression “a subject in need thereof” means a human or non-human animal that exhibits one or more symptoms or indicia of cancer (e.g., a subject expressing a tumor or suffering from any of the cancers mentioned herein), or who otherwise would benefit from an activation or recruitment of T cells. The bispecific antibodies of the disclosure demonstrated robust T cell activation in vitro. It is expected that the bispecific and multispecific antibodies of the disclosure will also demonstrate T cell activation in vivo.

The antibodies, bispecific antibodies, and multispecific antibodies described herein (and pharmaceutical compositions comprising the same) are useful, inter alia, for treating any disease or disorder in which stimulation, activation and/or targeting of an immune response would be beneficial. In particular, the bispecific antibodies or multispecific antibodies comprising a first antigen-binding domain that specifically binds CD3 and at least a second antigen-binding domain that specifically binds a second antigen described herein may be used for the treatment, prevention and/or amelioration of any disease or disorder that may be treated by activation or recruitment of T cells.

Also provided herein are methods for treating advanced cancer in a subject. As used herein, “advanced cancer” refers to a cancer which has grown beyond its original location. An advanced cancer may be “locally advanced,” in which the cancer has spread outside the body part it originated in, but has not yet spread to other parts of the body, or “metastatic,” in which the cancer has spread to other parts of the body.

Also provided herein are methods for treating refractory cancer in a subject. As used herein, the term “refractory cancer” refers to a cancer that has not responded to a treatment or has become resistant to said treatment.

Also provided herein are methods for treating unresectable cancer in a subject. As used herein, the term “unresectable cancer” refers to a cancer that cannot be removed with surgery.

Also provided herein are methods for treating residual cancer in a subject. As used herein, the term “residual cancer” means the existence or persistence of one or more cancerous cells in a subject following treatment with an anti-cancer therapy.

As used herein, the terms “treat,” “treating,” and “treatment” are all intended to refer to an amelioration or reversal of at least one measurable physical parameter related to a disease or disorder, e.g., cancer.

According to particular embodiments described herein, the therapeutically effective amount of pharmaceutical compositions administered to treat a subject are sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The dosage of the therapeutically effective amount of the pharmaceutical composition can vary according to various factors, such as the disease, disorder or condition to be treated, the means of administration, the target site, the physiological state of the subject (including, e.g., age, body weight, health), other medications administered, and whether the treatment is prophylactic or therapeutic. Treatment dosages are optimally titrated to optimize safety and efficacy.

### Administration Regimens

According to certain embodiments provided herein, multiple doses of antibodies that specifically bind CD3, or bispecific antibodies or multispecific antibodies comprising a first antigen-binding domain that specifically binds CD3 and at least a second antigen-binding domain that specifically binds a second antigen, described herein may be administered to a subject over a defined time course. The methods according to this aspect comprise sequentially administering to a subject multiple doses of an antibody as described herein. As used herein, “sequentially administering” means that each dose of an antibody is administered to the subject at a different point in time, e.g., on different days separated by a predetermined interval (e.g., hours, days, weeks or months). Also provided herein are methods which comprise sequentially administering to the patient a single initial dose of an antibody, followed by one or more secondary doses of the antibody and optionally followed by one or more tertiary doses of the antibody.

The terms “initial dose,” “secondary doses,” and “tertiary doses,” refer to the temporal sequence of administration of the bispecific antibody of the disclosure. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the antigen-binding molecule, but generally may differ from one another in terms of frequency of administration. In other embodiments, the amount of an antibody contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

The phrase “the immediately preceding dose,” as used herein, means, in a sequence of multiple administrations, the dose of an antibody which is administered to a patient prior to the administration of the very next dose in the sequence with no intervening doses.

The methods according to this aspect may comprise administering to a patient any number of secondary and/or tertiary doses of an antibody that specifically binds CD3, or a bispecific antibody or multispecific antibody comprising a first antigen-binding domain that specifically binds CD3 and at least a second antigen-binding domain that specifically binds a second antigen, described herein.

The frequency of administration may also be adjusted during the course of treatment by a physician depending on the needs of the individual patient following clinical examination.

### Diagnostic Use

The antibodies and antigen-binding fragments thereof, and the bispecific antibodies described herein may also be used for diagnostic purposes, such as detecting CD3 expressing cells in vitro or in vivo using known methods.

### EMBODIMENTS

This disclosure provided herein provides the following non-limiting embodiments.

Embodiment 1 is an anti-CD3 antibody or antigen-binding fragment thereof comprising:
a) a heavy chain complementarity determining region 1 (HCDR1) comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 1, 9, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289, 297, and 305;
b) an HCDR2 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 2, 10, 18, 26, 34, 42, 50, 58, 66, 74, 82, 90, 98, 106, 114, 122, 130, 138, 146, 154, 162, 170, 178, 186, 194, 202, 210, 218, 226, 234, 242, 250, 258, 266, 274, 282, 290, 298, and 306;
c) an HCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 3, 11, 19, 27, 35, 43, 51, 59, 67, 75, 83, 91, 99, 107, 115, 123, 131, 139, 147, 155, 163, 171, 179, 187, 195, 203, 211, 219, 227, 235, 243, 251, 259, 267, 275, 283, 291, 299, and 307;
d) a light chain complementarity determining region 1 (LCDR1) comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity of the sequences of any one of SEQ ID NOs: 4, 12, 20, 28, 36, 44, 52, 60, 68, 76, 84, 92, 100, 108, 116, 124, 132, 140, 148, 156, 164, 172, 180, 188, 196, 204, 212, 220, 228, 236, 244, 252, 260, 268, 276, 284, 292, 300, and 308;
e) an LCDR2 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 5, 13, 21, 29, 37, 45, 53, 61, 69, 77, 85, 93, 101, 109, 117, 125, 133, 141, 149, 157, 165, 173, 181, 189, 197, 205, 213, 221, 229, 237, 245, 253, 261, 269, 277, 285, 293, 301, and 309; and
f) an LCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 6, 14, 22, 30, 38, 46, 54, 62, 70, 78, 86, 94, 102, 110, 118, 126, 134, 142, 150, 158, 166, 174, 182, 190, 198, 206, 214, 222, 230, 238, 246, 254, 262, 270, 278, 286, 294, 302, and 310.

Embodiment 2 the anti-CD3 antibody or antigen-binding fragment thereof of embodiment 1 comprising:
a) a heavy chain complementarity determining region 1 (HCDR1) comprising the amino acid sequence of any one of SEQ ID NOs: 1, 9, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289, 297, and 305;
b) an HCDR2 comprising the amino acid sequence of any one of SEQ ID NOs: 2, 10, 18, 26, 34, 42, 50, 58, 66, 74, 82, 90, 98, 106, 114, 122, 130, 138, 146, 154, 162, 170, 178, 186, 194, 202, 210, 218, 226, 234, 242, 250, 258, 266, 274, 282, 290, 298, and 306; c) an HCDR3 comprising the amino acid sequence of any one of SEQ ID NOs: 3, 11, 19, 27, 35, 43, 51, 59, 67, 75, 83, 91, 99, 107, 115, 123, 131, 139, 147, 155, 163, 171, 179, 187, 195, 203, 211, 219, 227, 235, 243, 251, 259, 267, 275, 283, 291, 299, and 307;
d) a light chain complementarity determining region 1 (LCDR1) comprising the amino acid sequence of any one of SEQ ID NOs: 4, 12, 20, 28, 36, 44, 52, 60, 68, 76, 84, 92, 100, 108, 116, 124, 132, 140, 148, 156, 164, 172, 180, 188, 196, 204, 212, 220, 228, 236, 244, 252, 260, 268, 276, 284, 292, 300, and 308;
e) an LCDR2 comprising the amino acid sequence of any one of SEQ ID NOs: 5, 13, 21, 29, 37, 45, 53, 61, 69, 77, 85, 93, 101, 109, 117, 125, 133, 141, 149, 157, 165, 173, 181, 189, 197, 205, 213, 221, 229, 237, 245, 253, 261, 269, 277, 285, 293, 301, and 309; and
f) an LCDR3 comprising the amino acid sequence of any one of SEQ ID NOs: 6, 14, 22, 30, 38, 46, 54, 62, 70, 78, 86, 94, 102, 110, 118, 126, 134, 142, 150, 158, 166, 174, 182, 190, 198, 206, 214, 222, 230, 238, 246, 254, 262, 270, 278, 286, 294, 302, and 310.

Embodiment 3 is the anti-CD3 antibody or antigen-binding fragment thereof of embodiment 1 or 2, comprising:
a) an HCDR1, HCDR2, and HCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of:
   i) SEQ ID NOs: 1, 2, and 3, respectively;
   ii) SEQ ID NOs: 9, 10, and 11, respectively;
   iii) SEQ ID NOs: 17, 18, and 19, respectively;
   iv) SEQ ID NOs: 25, 26, and 27, respectively;
   v) SEQ ID NOs: 33, 34, and 35, respectively;
   vi) SEQ ID NOs: 41, 42, and 43, respectively;
   vii) SEQ ID NOs: 49, 50, and 51, respectively;
   viii) SEQ ID NOs: 57, 58, and 59, respectively;
   ix) SEQ ID NOs: 65, 66, and 67, respectively;
   x) SEQ ID NOs: 73, 74, and 75, respectively;
   xi) SEQ ID NOs: 81, 82, and 83, respectively;
   xii) SEQ ID NOs: 89, 90, and 91, respectively;
   xiii) SEQ ID NOs: 97, 98, and 99, respectively;
   xiv) SEQ ID NOs: 105, 106, and 107, respectively;
   xv) SEQ ID NOs: 113, 114, and 115, respectively;
   xvi) SEQ ID NOs: 121, 122, and 123, respectively;
   xvii) SEQ ID NOs: 129, 130, and 131, respectively;
   xviii) SEQ ID NOs: 137, 138, and 139, respectively;
   xix) SEQ ID NOs: 145, 146, and 147, respectively;
   xx) SEQ ID NOs: 153, 154, and 155, respectively;
   xxi) SEQ ID NOs: 161, 162, and 163, respectively;
   xxii) SEQ ID NOs: 169, 170, and 171, respectively;
   xxiii) SEQ ID NOs: 177, 178, and 179, respectively;
   xxiv) SEQ ID NOs: 185, 186, and 187, respectively;
   xxv) SEQ ID NOs: 193, 194, and 195, respectively;
   xxvi) SEQ ID NOs: 201, 202, and 203, respectively;
   xxvii) SEQ ID NOs: 209, 210, and 211, respectively;
   xxviii) SEQ ID NOs: 217, 218, and 219, respectively;
   xxix) SEQ ID NOs: 225, 226, and 227, respectively;
   xxx) SEQ ID NOs: 233, 234, and 235, respectively;
   xxxi) SEQ ID NOs: 241, 242, and 243, respectively;
   xxxii) SEQ ID NOs: 249, 250, and 251, respectively;
   xxxiii) SEQ ID NOs: 257, 258, and 259, respectively;
   xxxiv) SEQ ID NOs: 265, 266, and 267, respectively;
   xxxv) SEQ ID NOs: 273, 274, and 275, respectively;
   xxxvi) SEQ ID NOs: 281, 282, and 283, respectively;
   xxxvii) SEQ ID NOs: 289, 290, and 291, respectively;
   xxxviii) SEQ ID NOs: 297, 298, and 299, respectively; or
   xxxix) SEQ ID NOs: 305, 306, and 307; and
b) an LCDR1, LCDR2, and LCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of:
   i) SEQ ID NOs: 4, 5, and 6, respectively;
   ii) SEQ ID NOs: 12, 13, and 14, respectively;
   iii) SEQ ID NOs: 20, 21, and 22, respectively;
   iv) SEQ ID NOs: 28, 29, and 30, respectively;
   v) SEQ ID NOs: 36, 37, and 38, respectively;
   vi) SEQ ID NOs: 44, 45, and 46, respectively;
   vii) SEQ ID NOs: 52, 53, and 54, respectively;
   viii) SEQ ID NOs: 60, 61, and 62, respectively;
   ix) SEQ ID NOs: 68, 69, and 70, respectively;
   x) SEQ ID NOs: 76, 77, and 78, respectively;
   xi) SEQ ID NOs: 84, 85, and 86, respectively;
   xii) SEQ ID NOs: 92, 93, and 94, respectively;
   xiii) SEQ ID NOs: 100, 101, and 102, respectively;
   xiv) SEQ ID NOs: 108, 109, 110, respectively;
   xv) SEQ ID NOs: 116, 117, and 118, respectively;
   xvi) SEQ ID NOs: 124, 125, and 126, respectively;
   xvii) SEQ ID NOs: 132, 133, and 134, respectively;
   xviii) SEQ ID NOs: 140, 141, and 142, respectively;
   xix) SEQ ID NOs: 148, 149, and 150, respectively;
   xx) SEQ ID NOs: 156, 157, and 158, respectively;
   xxi) SEQ ID NOs: 164, 165, and 166, respectively;
   xxii) SEQ ID NOs: 172, 173, and 174, respectively;
   xxiii) SEQ ID NOs: 180, 181, and 182, respectively;
   xxiv) SEQ ID NOs: 188, 189, and 190, respectively;
   xxv) SEQ ID NOs: 196, 197, and 198, respectively;
   xxvi) SEQ ID NOs: 204, 205, and 206, respectively;
   xxvii) SEQ ID NOs: 212, 213, and 214, respectively;
   xxviii) SEQ ID NOs: 220, 221, and 222, respectively;
   xxix) SEQ ID NOs: 228, 229, and 230, respectively;
   xxx) SEQ ID NOs: 236, 237, and 238, respectively;
   xxxi) SEQ ID NOs: 244, 245, and 246, respectively;
   xxxii) SEQ ID NOs: 252, 253, and 254, respectively;
   xxxiii) SEQ ID NOs: 260, 261, and 262, respectively;
   xxxiv) SEQ ID NOs: 268, 269, and 270, respectively;
   xxxv) SEQ ID NOs: 276, 277, and 278, respectively;
   xxxvi) SEQ ID NOs: 284, 285, and 286, respectively;
   xxxvii) SEQ ID NOs: 292, 293, and 294, respectively;
   xxxviii) SEQ ID NOs: 300, 301, and 302, respectively; or
   xxxix) SEQ ID NOs: 308, 309, and 310.

Embodiment 4 is the anti-CD3 antibody or antigen-binding fragment thereof of any one of embodiments 1-3, comprising:
a) an HCDR1, HCDR2, and HCDR3 comprising the amino acid sequences of:
   i) SEQ ID NOs: 1, 2, and 3, respectively;
   ii) SEQ ID NOs: 9, 10, and 11, respectively;
   iii) SEQ ID NOs: 17, 18, and 19, respectively;
   iv) SEQ ID NOs: 25, 26, and 27, respectively;
   v) SEQ ID NOs: 33, 34, and 35, respectively;
   vi) SEQ ID NOs: 41, 42, and 43, respectively;
   vii) SEQ ID NOs: 49, 50, and 51, respectively;
   viii) SEQ ID NOs: 57, 58, and 59, respectively;
   ix) SEQ ID NOs: 65, 66, and 67, respectively;
   x) SEQ ID NOs: 73, 74, and 75, respectively;
   xi) SEQ ID NOs: 81, 82, and 83, respectively;
   xii) SEQ ID NOs: 89, 90, and 91, respectively;
   xiii) SEQ ID NOs: 97, 98, and 99, respectively;
   xiv) SEQ ID NOs: 105, 106, and 107, respectively;
   xv) SEQ ID NOs: 113, 114, and 115, respectively;
   xvi) SEQ ID NOs: 121, 122, and 123, respectively;
   xvii) SEQ ID NOs: 129, 130, and 131, respectively;
   xviii) SEQ ID NOs: 137, 138, and 139, respectively;
   xix) SEQ ID NOs: 145, 146, and 147, respectively;
   xx) SEQ ID NOs: 153, 154, and 155, respectively;
   xxi) SEQ ID NOs: 161, 162, and 163, respectively;
   xxii) SEQ ID NOs: 169, 170, and 171, respectively;
   xxiii) SEQ ID NOs: 177, 178, and 179, respectively;
   xxiv) SEQ ID NOs: 185, 186, and 187, respectively;
   xxv) SEQ ID NOs: 193, 194, and 195, respectively;
   xxvi) SEQ ID NOs: 201, 202, and 203, respectively;
   xxvii) SEQ ID NOs: 209, 210, and 211, respectively;
   xxviii) SEQ ID NOs: 217, 218, and 219, respectively;
   xxix) SEQ ID NOs: 225, 226, and 227, respectively;
   xxx) SEQ ID NOs: 233, 234, and 235, respectively;
   xxxi) SEQ ID NOs: 241, 242, and 243, respectively;
   xxxii) SEQ ID NOs: 249, 250, and 251, respectively;
   xxxiii) SEQ ID NOs: 257, 258, and 259, respectively;
   xxxiv) SEQ ID NOs: 265, 266, and 267, respectively;
   xxxv) SEQ ID NOs: 273, 274, and 275, respectively;
   xxxvi) SEQ ID NOs: 281, 282, and 283, respectively;
   xxxvii) SEQ ID NOs: 289, 290, and 291, respectively;
   xxxviii) SEQ ID NOs: 297, 298, and 299, respectively; or
   xxxix) SEQ ID NOs: 305, 306, and 307; and
b) an LCDR1, LCDR2, and LCDR3 comprising the amino acid sequences of:
   i) SEQ ID NOs: 4, 5, and 6, respectively;
   ii) SEQ ID NOs: 12, 13, and 14, respectively;
   iii) SEQ ID NOs: 20, 21, and 22, respectively;
   iv) SEQ ID NOs: 28, 29, and 30, respectively;
   v) SEQ ID NOs: 36, 37, and 38, respectively;
   vi) SEQ ID NOs: 44, 45, and 46, respectively;
   vii) SEQ ID NOs: 52, 53, and 54, respectively;
   viii) SEQ ID NOs: 60, 61, and 62, respectively;
   ix) SEQ ID NOs: 68, 69, and 70, respectively;
   x) SEQ ID NOs: 76, 77, and 78, respectively;
   xi) SEQ ID NOs: 84, 85, and 86, respectively;
   xii) SEQ ID NOs: 92, 93, and 94, respectively;
   xiii) SEQ ID NOs: 100, 101, and 102, respectively;
   xiv) SEQ ID NOs: 108, 109, 110, respectively;
   xv) SEQ ID NOs: 116, 117, and 118, respectively;
   xvi) SEQ ID NOs: 124, 125, and 126, respectively;
   xvii) SEQ ID NOs: 132, 133, and 134, respectively;
   xviii) SEQ ID NOs: 140, 141, and 142, respectively;
   xix) SEQ ID NOs: 148, 149, and 150, respectively;
   xx) SEQ ID NOs: 156, 157, and 158, respectively;
   xxi) SEQ ID NOs: 164, 165, and 166, respectively;
   xxii) SEQ ID NOs: 172, 173, and 174, respectively;
   xxiii) SEQ ID NOs: 180, 181, and 182, respectively;
   xxiv) SEQ ID NOs: 188, 189, and 190, respectively;
   xxv) SEQ ID NOs: 196, 197, and 198, respectively;
   xxvi) SEQ ID NOs: 204, 205, and 206, respectively;
   xxvii) SEQ ID NOs: 212, 213, and 214, respectively;
   xxviii) SEQ ID NOs: 220, 221, and 222, respectively;
   xxix) SEQ ID NOs: 228, 229, and 230, respectively;
   xxx) SEQ ID NOs: 236, 237, and 238, respectively;
   xxxi) SEQ ID NOs: 244, 245, and 246, respectively;
   xxxii) SEQ ID NOs: 252, 253, and 254, respectively;
   xxxiii) SEQ ID NOs: 260, 261, and 262, respectively;
   xxxiv) SEQ ID NOs: 268, 269, and 270, respectively;
   xxxv) SEQ ID NOs: 276, 277, and 278, respectively;
   xxxvi) SEQ ID NOs: 284, 285, and 286, respectively;
   xxxvii) SEQ ID NOs: 292, 293, and 294, respectively;
   xxxviii) SEQ ID NOs: 300, 301, and 302, respectively; or
   xxxix) SEQ ID NOs: 308, 309, and 310.

Embodiment 5 is the anti-CD3 antibody of any one of embodiments 1-4, comprising an HCDR1, HCDR2, HCDR3, LCDR1, LCDR2, and LCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of:
a) SEQ ID NOs: 1, 2, 3, 4, 5, and 6, respectively;
b) SEQ ID NOs: 9, 10, 11, 12, 13, and 14, respectively;
c) SEQ ID NOs: 17, 18, 19, 20, 21, and 22, respectively;
d) SEQ ID NOs: 25, 26, 27, 28, 29, and 30, respectively;
e) SEQ ID NOs: 33, 34, 35, 36, 37, and 38, respectively;
f) SEQ ID NOs: 41, 42, 43, 44, 45, and 46, respectively;
g) SEQ ID NOs: 49, 50, 51, 52, 53, and 54, respectively;
h) SEQ ID NOs: 57, 58, 59, 60, 61, and 62, respectively;
i) SEQ ID NOs: 65, 66, 67, 68, 69, and 70, respectively;
j) SEQ ID NOs: 73, 74, 75, 76, 77, and 78, respectively;
k) SEQ ID NOs: 81, 82, 83, 84, 85, and 86, respectively;
l) SEQ ID NOs: 89, 90, 91, 92, 93, and 94, respectively;
m) SEQ ID NOs: 97, 98, 99, 100, 101, and 102, respectively;
n) SEQ ID NOs: 105, 106, 107, 108, 109, and 110, respectively;
o) SEQ ID NOs: 113, 114, 115, 116, 117, and 118, respectively;
p) SEQ ID NOs: 121, 122, 123, 124, 125, and 126, respectively;
q) SEQ ID NOs: 129, 130, 131, 132, 133, and 134, respectively;
r) SEQ ID NOs: 137, 138, 139, 140, 141, and 142, respectively;
s) SEQ ID NOs: 145, 146, 147, 148, 149, and 150, respectively;
t) SEQ ID NOs: 153, 154, 155, 156, 156, and 158, respectively;
u) SEQ ID NOs: 161, 162, 263, 164, 165, and 166, respectively;
v) SEQ ID NOs: 169, 170, 171, 172, 173, and 174, respectively;
w) SEQ ID NOs: 177, 178, 179, 180, 181, and 182, respectively;
x) SEQ ID NOs: 185, 186, 187, 188, 189, and 190, respectively;
y) SEQ ID NOs: 193, 194, 195, 196, 197, and 198, respectively;
z) SEQ ID NOs: 201, 202, 203, 204, 205, and 206, respectively;
aa) SEQ ID NOs: 209, 210, 211, 212, 213, and 214, respectively;
ab) SEQ ID NOs: 217, 218, 219, 220, 221, and 222, respectively;
ac) SEQ ID NOs: 225, 226, 227, 228, 229, and 230, respectively;
ad) SEQ ID NOs: 233, 234, 235, 236, 237, and 238, respectively;
ae) SEQ ID NOs: 241, 242, 243, 244, 245, and 246, respectively;
af) SEQ ID NOs: 249, 250, 251, 252, 253, and 254, respectively;
ag) SEQ ID NOs: 257, 258, 259, 260, 261, and 262, respectively;
ah) SEQ ID NOs: 265, 266, 267, 268, 269, and 270, respectively;
ai) SEQ ID NOs: 273, 274, 275, 276, 277, and 278, respectively;
aj) SEQ ID NOs: 281, 282, 283, 284, 285, and 286, respectively;
ak) SEQ ID NOs: 289, 290, 291, 292, 293, and 294, respectively;
al) SEQ ID NOs: 297, 298, 299, 300, 301, and 302, respectively; or
am) SEQ ID NOs: 305, 306, 307, 308, 309, and 310, respectively.

Embodiment 6 is the anti-CD3 antibody of any one of embodiments 1-5, comprising an HCDR1, HCDR2, HCDR3, LCDR1, LCDR2, and LCDR3 comprising the amino acid sequences of:
a) SEQ ID NOs: 1, 2, 3, 4, 5, and 6, respectively;
b) SEQ ID NOs: 9, 10, 11, 12, 13, and 14, respectively;
c) SEQ ID NOs: 17, 18, 19, 20, 21, and 22, respectively;
d) SEQ ID NOs: 25, 26, 27, 28, 29, and 30, respectively;
e) SEQ ID NOs: 33, 34, 35, 36, 37, and 38, respectively;
f) SEQ ID NOs: 41, 42, 43, 44, 45, and 46, respectively;
g) SEQ ID NOs: 49, 50, 51, 52, 53, and 54, respectively;
h) SEQ ID NOs: 57, 58, 59, 60, 61, and 62, respectively;
i) SEQ ID NOs: 65, 66, 67, 68, 69, and 70, respectively;
j) SEQ ID NOs: 73, 74, 75, 76, 77, and 78, respectively;
k) SEQ ID NOs: 81, 82, 83, 84, 85, and 86, respectively;
l) SEQ ID NOs: 89, 90, 91, 92, 93, and 94, respectively;
m) SEQ ID NOs: 97, 98, 99, 100, 101, and 102, respectively;
n) SEQ ID NOs: 105, 106, 107, 108, 109, and 110, respectively;
o) SEQ ID NOs: 113, 114, 115, 116, 117, and 118, respectively;
p) SEQ ID NOs: 121, 122, 123, 124, 125, and 126, respectively;
q) SEQ ID NOs: 129, 130, 131, 132, 133, and 134, respectively;
r) SEQ ID NOs: 137, 138, 139, 140, 141, and 142, respectively;
s) SEQ ID NOs: 145, 146, 147, 148, 149, and 150, respectively;
t) SEQ ID NOs: 153, 154, 155, 156, 156, and 158, respectively;
u) SEQ ID NOs: 161, 162, 263, 164, 165, and 166, respectively;
v) SEQ ID NOs: 169, 170, 171, 172, 173, and 174, respectively;
w) SEQ ID NOs: 177, 178, 179, 180, 181, and 182, respectively;
x) SEQ ID NOs: 185, 186, 187, 188, 189, and 190, respectively;
y) SEQ ID NOs: 193, 194, 195, 196, 197, and 198, respectively;
z) SEQ ID NOs: 201, 202, 203, 204, 205, and 206, respectively;
aa) SEQ ID NOs: 209, 210, 211, 212, 213, and 214, respectively;
ab) SEQ ID NOs: 217, 218, 219, 220, 221, and 222, respectively;
ac) SEQ ID NOs: 225, 226, 227, 228, 229, and 230, respectively;
ad) SEQ ID NOs: 233, 234, 235, 236, 237, and 238, respectively;
ae) SEQ ID NOs: 241, 242, 243, 244, 245, and 246, respectively;
af) SEQ ID NOs: 249, 250, 251, 252, 253, and 254, respectively;
ag) SEQ ID NOs: 257, 258, 259, 260, 261, and 262, respectively;
ah) SEQ ID NOs: 265, 266, 267, 268, 269, and 270, respectively;
ai) SEQ ID NOs: 273, 274, 275, 276, 277, and 278, respectively;
aj) SEQ ID NOs: 281, 282, 283, 284, 285, and 286, respectively;
ak) SEQ ID NOs: 289, 290, 291, 292, 293, and 294, respectively;
al) SEQ ID NOs: 297, 298, 299, 300, 301, and 302, respectively; or
am) SEQ ID NOs: 305, 306, 307, 308, 309, and 310, respectively.

Embodiment 7 is the anti-CD3 antibody of any one of embodiments 1-6, comprising: an HCDR1, HCDR2, HCDR3, LCDR1, LCDR2, and LCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of SEQ ID NOs:
a) 1, 2, 3, 4, 5, and 6, respectively;
b) 201, 202, 203, 204, 205, and 206, respectively;
c) 249, 250, 251, 252, 253, and 254, respectively; or
d) 297, 298, 299, 300, 301, and 302, respectively.

Embodiment 8 is the anti-CD3 of any one of antibodies 1-7, comprising: an HCDR1, HCDR2, HCDR3, LCDR1, LCDR2, and LCDR3 comprising the sequences of SEQ ID NOs:
a) 1, 2, 3, 4, 5, and 6, respectively;
b) 201, 202, 203, 204, 205, and 206, respectively;
c) 249, 250, 251, 252, 253, and 254, respectively; or
d) 297, 298, 299, 300, 301, and 302, respectively.

Embodiment 9 is the anti-CD3 antibody of any one of embodiments 1-8, comprising:
a) a heavy chain variable region (VH) comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 7, 15, 23, 31, 39, 47, 55, 63, 71, 79, 87, 95, 103, 111, 119, 127, 135, 143, 151, 159, 167, 175, 183, 191, 199, 207, 215, 223, 231, 239, 247, 255, 263, 271, 279, 287, 295, 303, and 311; and
b) a light chain variable region (VL) comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 8, 16, 24, 32, 40, 48, 56, 64, 72, 80, 88, 96, 104, 112, 120, 128, 136, 144, 152, 160, 168, 176, 184, 192, 200, 208, 216, 224, 232, 240, 248, 256, 264, 272, 280, 288, 296, 304, and 312.

Embodiment 10 is the anti-CD3 antibody of any one of embodiments 1-9, comprising:
a) a heavy chain variable region (VH) comprising the amino acid sequence of any one of SEQ ID NOs: 7, 15, 23, 31, 39, 47, 55, 63, 71, 79, 87, 95, 103, 111, 119, 127, 135, 143, 151, 159, 167, 175, 183, 191, 199, 207, 215, 223, 231, 239, 247, 255, 263, 271, 279, 287, 295, 303, and 311; and
b) a light chain variable region (VL) comprising the amino acid sequence of any one of SEQ ID NOs: 8, 16, 24, 32, 40, 48, 56, 64, 72, 80, 88, 96, 104, 112, 120, 128, 136, 144, 152, 160, 168, 176, 184, 192, 200, 208, 216, 224, 232, 240, 248, 256, 264, 272, 280, 288, 296, 304, and 312.

Embodiment 11 is the anti-CD3 antibody of any one of embodiments 1-10, comprising a VH and a VL comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of:
a) SEQ ID NOs: 7 and 8, respectively;
b) SEQ ID NOs: 15 and 16, respectively;
c) SEQ ID NOs: 23 and 24, respectively;
d) SEQ ID NOs: 31 and 32, respectively;
e) SEQ ID NOs: 39 and 40, respectively;
f) SEQ ID NOs: 47 and 48, respectively;
g) SEQ ID NOs: 55 and 56, respectively;
h) SEQ ID NOs: 63 and 64, respectively;
i) SEQ ID NOs: 71 and 72, respectively;
j) SEQ ID NOs: 79 and 80, respectively;
k) SEQ ID NOs: 87 and 88, respectively;
l) SEQ ID NOs: 95 and 96, respectively;
m) SEQ ID NOs: 103 and 104, respectively;
n) SEQ ID NOs: 111 and 112, respectively;
o) SEQ ID NOs: 119 and 120, respectively;
p) SEQ ID NOs: 127 and 128, respectively;
q) SEQ ID NOs: 135 and 136, respectively;
r) SEQ ID NOs: 143 and 144, respectively;
s) SEQ ID NOs: 151 and 152, respectively;
t) SEQ ID NOs: 159 and 160, respectively;
u) SEQ ID NOs: 167 and 168, respectively;
v) SEQ ID NOs: 175 and 176, respectively;
w) SEQ ID NOs: 183 and 184, respectively;
x) SEQ ID NOs: 191 and 192, respectively;
y) SEQ ID NOs: 199 and 200, respectively;
z) SEQ ID NOs: 207 and 208, respectively;
aa) SEQ ID NOs: 215 and 216, respectively;
ab) SEQ ID NOs: 223 and 224, respectively;
ac) SEQ ID NOs: 231 and 232, respectively;
ad) SEQ ID NOs: 239 and 240, respectively;
ae) SEQ ID NOs: 247 and 248, respectively;
af) SEQ ID NOs: 255 and 256, respectively;
ag) SEQ ID NOs: 263 and 264, respectively;
ah) SEQ ID NOs: 271 and 272, respectively;
ai) SEQ ID NOs: 279 and 280, respectively;
aj) SEQ ID NOs: 287 and 288, respectively;
ak) SEQ ID NOs: 295 and 296, respectively;
al) SEQ ID NOs: 303 and 304, respectively; or
am) SEQ ID NOs: 311 and 312, respectively.

Embodiment 12 is the anti-CD3 antibody of any one of embodiments 1-11, comprising a VH and a VL comprising the amino acid sequences of:
a) SEQ ID NOs: 7 and 8, respectively;
b) SEQ ID NOs: 15 and 16, respectively;
c) SEQ ID NOs: 23 and 24, respectively;
d) SEQ ID NOs: 31 and 32, respectively;
e) SEQ ID NOs: 39 and 40, respectively;
f) SEQ ID NOs: 47 and 48, respectively;
g) SEQ ID NOs: 55 and 56, respectively;
h) SEQ ID NOs: 63 and 64, respectively;
i) SEQ ID NOs: 71 and 72, respectively;
j) SEQ ID NOs: 79 and 80, respectively;
k) SEQ ID NOs: 87 and 88, respectively;
l) SEQ ID NOs: 95 and 96, respectively;
m) SEQ ID NOs: 103 and 104, respectively;
n) SEQ ID NOs: 111 and 112, respectively;
o) SEQ ID NOs: 119 and 120, respectively;
p) SEQ ID NOs: 127 and 128, respectively;
q) SEQ ID NOs: 135 and 136, respectively;
r) SEQ ID NOs: 143 and 144, respectively;
s) SEQ ID NOs: 151 and 152, respectively;
t) SEQ ID NOs: 159 and 160, respectively;
u) SEQ ID NOs: 167 and 168, respectively;
v) SEQ ID NOs: 175 and 176, respectively;
w) SEQ ID NOs: 183 and 184, respectively;
x) SEQ ID NOs: 191 and 192, respectively;
y) SEQ ID NOs: 199 and 200, respectively;
z) SEQ ID NOs: 207 and 208, respectively;
aa) SEQ ID NOs: 215 and 216, respectively;
ab) SEQ ID NOs: 223 and 224, respectively;
ac) SEQ ID NOs: 231 and 232, respectively;
ad) SEQ ID NOs: 239 and 240, respectively;
ae) SEQ ID NOs: 247 and 248, respectively;
af) SEQ ID NOs: 255 and 256, respectively;
ag) SEQ ID NOs: 263 and 264, respectively;
ah) SEQ ID NOs: 271 and 272, respectively;
ai) SEQ ID NOs: 279 and 280, respectively;
aj) SEQ ID NOs: 287 and 288, respectively;
ak) SEQ ID NOs: 295 and 296, respectively;
al) SEQ ID NOs: 303 and 304, respectively; or
am) SEQ ID NOs: 311 and 312, respectively.

Embodiment 13 is the anti-CD3 antibody of any one of embodiments 1-12, comprising a VH and VL comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of SEQ ID NOs:
a) 7 and 8, respectively;
b) 207 and 208, respectively;
c) 255 and 256, respectively; or
d) 303 and 304, respectively.

Embodiment 14 is the anti-CD3 antibody of any one of embodiments 1-13, comprising a VH and VL comprising the sequences of SEQ ID NOs:
a) 7 and 8, respectively;
b) 207 and 208, respectively;
c) 255 and 256, respectively; or
d) 303 and 304, respectively.

Embodiment 15 is the anti-CD3 antibody of any one of embodiments 1-14, wherein the antibody comprises from 1 to 10, from 1 to 5, or from 1 to 2 amino acid substitutions across all framework regions.

Embodiment 16 is the anti-CD3 antibody of any one of embodiments 1-15, wherein the antibody comprises 1 or 2 amino acid substitutions across all framework regions.

Embodiment 17 is the anti-CD3 antibody of any one of embodiments 1-16, wherein the antibody comprises from 1 to 10, from 1 to 5, or from 1 to 2 amino acid substitutions across all six CDR regions.

Embodiment 18 is the anti-CD3 antibody of any one of embodiments 1-17, wherein the antibody comprises 1 or 2 amino acid substitution across all six CDR regions.

Embodiment 19 is the anti-CD3 antibody of embodiment 15 or 16, wherein the amino acid substitutions are not in a CDR region.

Embodiment 20 is the anti-CD3 antibody of any one of embodiments 1-19, wherein the amino acid substitutions are all conservative amino acid substitutions.

Embodiment 21 is the anti-CD3 antibody of any one of embodiments 1-20, wherein the antibody is human or humanized.

Embodiment 22 is the anti-CD3 antibody of any one of embodiments 1-21, wherein the antibody comprises an Fc domain.

Embodiment 23 is the anti-CD3 antibody of any one of embodiments 1-22, wherein the Fc domain is a human immunoglobulin G (IgG) Fc domain.

Embodiment 24 is the anti-CD3 antibody of any one of embodiments 1-23, wherein the IgG Fc domain is an IgG1 Fc domain.

Embodiment 25 is the anti-CD3 antibody of any one of embodiments 1-24, wherein the Fc domain comprises M252Y, S254T, and T256E (YTE) substitutions, wherein numbering is according to the EU index.

Embodiment 26 is the anti-CD3 antibody of any one of embodiments 1-25, wherein the antibody is a bispecific or multispecific antibody.

Embodiment 27 is an isolated nucleic acid encoding the anti-CD3 antibody of any one of embodiments 1-26.

Embodiment 28 is a vector comprising the isolated nucleic acid of embodiment 27.

Embodiment 29 is a host cell comprising the isolated the nucleic acid of embodiment 27 or the vector of embodiment 28.

Embodiment 30 is a composition comprising the anti-CD3 antibody of any one of embodiments 1-26, the isolated nucleic acid of embodiment 27, or the vector of embodiment 28 and a pharmaceutically acceptable carrier.

Embodiment 31 is a method of producing the anti-CD3 antibody of any one of embodiments 1-26 comprising: culturing the host cell of embodiment 29 in an environment conducive to the expression of the anti-CD3 antibody of any one of embodiments 1-26; and isolating the anti-CD3 antibody of any one of embodiments 1-26.

Embodiment 32 is a bispecific or multispecific antibody comprising a first binding domain that specifically binds CD3 and at least a second antigen binding domain that specifically binds at least a second antigen, wherein the first binding domain that specifically binds CD3 comprises:
a) a heavy chain complementarity determining region 1 (HCDR1) comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 1, 9, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289, 297, and 305;
b) an HCDR2 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 2, 10, 18, 26, 34, 42, 50, 58, 66, 74, 82, 90, 98, 106, 114, 122, 130, 138, 146, 154, 162, 170, 178, 186, 194, 202, 210, 218, 226, 234, 242, 250, 258, 266, 274, 282, 290, 298, and 306;
c) an HCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 3, 11, 19, 27, 35, 43, 51, 59, 67, 75, 83, 91, 99, 107, 115, 123, 131, 139, 147, 155, 163, 171, 179, 187, 195, 203, 211, 219, 227, 235, 243, 251, 259, 267, 275, 283, 291, 299, and 307;
d) a light chain complementarity determining region 1 (LCDR1) comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 4, 12, 20, 28, 36, 44, 52, 60, 68, 76, 84, 92, 100, 108, 116, 124, 132, 140, 148, 156, 164, 172, 180, 188, 196, 204, 212, 220, 228, 236, 244, 252, 260, 268, 276, 284, 292, 300, and 308;
e) an LCDR2 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 5, 13, 21, 29, 37, 45, 53, 61, 69, 77, 85, 93, 101, 109, 117, 125, 133, 141, 149, 157, 165, 173, 181, 189, 197, 205, 213, 221, 229, 237, 245, 253, 261, 269, 277, 285, 293, 301, and 309; and
f) an LCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 6, 14, 22, 30, 38, 46, 54, 62, 70, 78, 86, 94, 102, 110, 118, 126, 134, 142, 150, 158, 166, 174, 182, 190, 198, 206, 214, 222, 230, 238, 246, 254, 262, 270, 278, 286, 294, 302, and 310.

Embodiment 33 is the bispecific or multispecific antibody of embodiment 32, comprising a first binding domain that specifically binds CD3 and at least a second antigen binding domain that specifically binds at least a second antigen, wherein the first binding domain that specifically binds CD3 comprises:
a) a heavy chain complementarity determining region 1 (HCDR1) comprising the amino acid sequence of any one of SEQ ID NOs: 1, 9, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289, 297, and 305;
b) an HCDR2 comprising the amino acid sequence of any one of SEQ ID NOs: 2, 10, 18, 26, 34, 42, 50, 58, 66, 74, 82, 90, 98, 106, 114, 122, 130, 138, 146, 154, 162, 170, 178, 186, 194, 202, 210, 218, 226, 234, 242, 250, 258, 266, 274, 282, 290, 298, and 306;
c) an HCDR3 comprising the amino acid sequence of any one of SEQ ID NOs: 3, 11, 19, 27, 35, 43, 51, 59, 67, 75, 83, 91, 99, 107, 115, 123, 131, 139, 147, 155, 163, 171, 179, 187, 195, 203, 211, 219, 227, 235, 243, 251, 259, 267, 275, 283, 291, 299, and 307;
d) a light chain complementarity determining region 1 (LCDR1) comprising the amino acid sequence of any one of SEQ ID NOs: 4, 12, 20, 28, 36, 44, 52, 60, 68, 76, 84, 92, 100, 108, 116, 124, 132, 140, 148, 156, 164, 172, 180, 188, 196, 204, 212, 220, 228, 236, 244, 252, 260, 268, 276, 284, 292, 300, and 308;
e) an LCDR2 comprising the amino acid sequence of any one of SEQ ID NOs: 5, 13, 21, 29, 37, 45, 53, 61, 69, 77, 85, 93, 101, 109, 117, 125, 133, 141, 149, 157, 165, 173, 181, 189, 197, 205, 213, 221, 229, 237, 245, 253, 261, 269, 277, 285, 293, 301, and 309; and
f) an LCDR3 comprising the amino acid sequence of any one of SEQ ID NOs: 6, 14, 22, 30, 38, 46, 54, 62, 70, 78, 86, 94, 102, 110, 118, 126, 134, 142, 150, 158, 166, 174, 182, 190, 198, 206, 214, 222, 230, 238, 246, 254, 262, 270, 278, 286, 294, 302, and 310.

Embodiment 34 is the bispecific or multispecific antibody of embodiment 32 or 33, wherein the first binding domain that specifically binds CD3 comprises:
a) an HCDR1, HCDR2, and HCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of:
   i) SEQ ID NOs: 1, 2, and 3, respectively;
   ii) SEQ ID NOs: 9, 10, and 11, respectively;
   iii) SEQ ID NOs: 17, 18, and 19, respectively;
   iv) SEQ ID NOs: 25, 26, and 27, respectively;
   v) SEQ ID NOs: 33, 34, and 35, respectively;
   vi) SEQ ID NOs: 41, 42, and 43, respectively;
   vii) SEQ ID NOs: 49, 50, and 51, respectively;
   viii) SEQ ID NOs: 57, 58, and 59, respectively;
   ix) SEQ ID NOs: 65, 66, and 67, respectively;
   x) SEQ ID NOs: 73, 74, and 75, respectively;
   xi) SEQ ID NOs: 81, 82, and 83, respectively;
   xii) SEQ ID NOs: 89, 90, and 91, respectively;
   xiii) SEQ ID NOs: 97, 98, and 99, respectively;
   xiv) SEQ ID NOs: 105, 106, and 107, respectively;
   xv) SEQ ID NOs: 113, 114, and 115, respectively;
   xvi) SEQ ID NOs: 121, 122, and 123, respectively;
   xvii) SEQ ID NOs: 129, 130, and 131, respectively;
   xviii) SEQ ID NOs: 137, 138, and 139, respectively;
   xix) SEQ ID NOs: 145, 146, and 147, respectively;
   xx) SEQ ID NOs: 153, 154, and 155, respectively;
   xxi) SEQ ID NOs: 161, 162, and 163, respectively;
   xxii) SEQ ID NOs: 169, 170, and 171, respectively;
   xxiii) SEQ ID NOs: 177, 178, and 179, respectively;
   xxiv) SEQ ID NOs: 185, 186, and 187, respectively;
   xxv) SEQ ID NOs: 193, 194, and 195, respectively;
   xxvi) SEQ ID NOs: 201, 202, and 203, respectively;
   xxvii) SEQ ID NOs: 209, 210, and 211, respectively;
   xxviii) SEQ ID NOs: 217, 218, and 219, respectively;
   xxix) SEQ ID NOs: 225, 226, and 227, respectively;
   xxx) SEQ ID NOs: 233, 234, and 235, respectively;
   xxxi) SEQ ID NOs: 241, 242, and 243, respectively;
   xxxii) SEQ ID NOs: 249, 250, and 251, respectively;
   xxxiii) SEQ ID NOs: 257, 258, and 259, respectively;
   xxxiv) SEQ ID NOs: 265, 266, and 267, respectively;
   XXXV) SEQ ID NOs: 273, 274, and 275, respectively;
   xxxvi) SEQ ID NOs: 281, 282, and 283, respectively;
   xxxvii) SEQ ID NOs: 289, 290, and 291, respectively;
   xxxviii) SEQ ID NOs: 297, 298, and 299, respectively; or
   xxxix) SEQ ID NOs: 305, 306, and 307; and
b) an LCDR1, LCDR2, and LCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of:
   i) SEQ ID NOs: 4, 5, and 6, respectively;
   ii) SEQ ID NOs: 12, 13, and 14, respectively;
   iii) SEQ ID NOs: 20, 21, and 22, respectively;
   iv) SEQ ID NOs: 28, 29, and 30, respectively;
   v) SEQ ID NOs: 36, 37, and 38, respectively;
   vi) SEQ ID NOs: 44, 45, and 46, respectively;
   vii) SEQ ID NOs: 52, 53, and 54, respectively;
   viii) SEQ ID NOs: 60, 61, and 62, respectively;
   ix) SEQ ID NOs: 68, 69, and 70, respectively;
   x) SEQ ID NOs: 76, 77, and 78, respectively;
   xi) SEQ ID NOs: 84, 85, and 86, respectively;
   xii) SEQ ID NOs: 92, 93, and 94, respectively;
   xiii) SEQ ID NOs: 100, 101, and 102, respectively;
   xiv) SEQ ID NOs: 108, 109, 110, respectively;
   xv) SEQ ID NOs: 116, 117, and 118, respectively;
   xvi) SEQ ID NOs: 124, 125, and 126, respectively;
   xvii) SEQ ID NOs: 132, 133, and 134, respectively;
   xviii) SEQ ID NOs: 140, 141, and 142, respectively;
   xix) SEQ ID NOs: 148, 149, and 150, respectively;
   xx) SEQ ID NOs: 156, 157, and 158, respectively;
   xxi) SEQ ID NOs: 164, 165, and 166, respectively;
   xxii) SEQ ID NOs: 172, 173, and 174, respectively;
   xxiii) SEQ ID NOs: 180, 181, and 182, respectively;
   xxiv) SEQ ID NOs: 188, 189, and 190, respectively;
   xxv) SEQ ID NOs: 196, 197, and 198, respectively;
   xxvi) SEQ ID NOs: 204, 205, and 206, respectively;
   xxvii) SEQ ID NOs: 212, 213, and 214, respectively;
   xxviii) SEQ ID NOs: 220, 221, and 222, respectively;
   xxix) SEQ ID NOs: 228, 229, and 230, respectively;
   xxx) SEQ ID NOs: 236, 237, and 238, respectively;
   xxxi) SEQ ID NOs: 244, 245, and 246, respectively;
   xxxii) SEQ ID NOs: 252, 253, and 254, respectively;
   xxxiii) SEQ ID NOs: 260, 261, and 262, respectively;
   xxxiv) SEQ ID NOs: 268, 269, and 270, respectively;
   xxxv) SEQ ID NOs: 276, 277, and 278, respectively;
   xxxvi) SEQ ID NOs: 284, 285, and 286, respectively;
   xxxvii) SEQ ID NOs: 292, 293, and 294, respectively;
   xxxviii) SEQ ID NOs: 300, 301, and 302, respectively; or
   xxxix) SEQ ID NOs: 308, 309, and 310.

Embodiment 35 is the bispecific or multispecific antibody of any one of embodiments 32-34, wherein the first binding domain that specifically binds CD3 comprises:
a) an HCDR1, HCDR2, and HCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of:
   i) SEQ ID NOs: 1, 2, and 3, respectively;
   ii) SEQ ID NOs: 9, 10, and 11, respectively;
   iii) SEQ ID NOs: 17, 18, and 19, respectively;
   iv) SEQ ID NOs: 25, 26, and 27, respectively;
   v) SEQ ID NOs: 33, 34, and 35, respectively;
   vi) SEQ ID NOs: 41, 42, and 43, respectively;
   vii) SEQ ID NOs: 49, 50, and 51, respectively;
   viii) SEQ ID NOs: 57, 58, and 59, respectively;
   ix) SEQ ID NOs: 65, 66, and 67, respectively;
   x) SEQ ID NOs: 73, 74, and 75, respectively;
   xi) SEQ ID NOs: 81, 82, and 83, respectively;
   xii) SEQ ID NOs: 89, 90, and 91, respectively;
   xiii) SEQ ID NOs: 97, 98, and 99, respectively;
   xiv) SEQ ID NOs: 105, 106, and 107, respectively;
   xv) SEQ ID NOs: 113, 114, and 115, respectively;
   xvi) SEQ ID NOs: 121, 122, and 123, respectively;
   xvii) SEQ ID NOs: 129, 130, and 131, respectively;
   xviii) SEQ ID NOs: 137, 138, and 139, respectively;
   xix) SEQ ID NOs: 145, 146, and 147, respectively;
   xx) SEQ ID NOs: 153, 154, and 155, respectively;
   xxi) SEQ ID NOs: 161, 162, and 163, respectively;
   xxii) SEQ ID NOs: 169, 170, and 171, respectively;
   xxiii) SEQ ID NOs: 177, 178, and 179, respectively;
   xxiv) SEQ ID NOs: 185, 186, and 187, respectively;
   xxv) SEQ ID NOs: 193, 194, and 195, respectively;
   xxvi) SEQ ID NOs: 201, 202, and 203, respectively;
   xxvii) SEQ ID NOs: 209, 210, and 211, respectively;
   xxviii) SEQ ID NOs: 217, 218, and 219, respectively;
   xxix) SEQ ID NOs: 225, 226, and 227, respectively;
   xxx) SEQ ID NOs: 233, 234, and 235, respectively;
   xxxi) SEQ ID NOs: 241, 242, and 243, respectively;
   xxxii) SEQ ID NOs: 249, 250, and 251, respectively;
   xxxiii) SEQ ID NOs: 257, 258, and 259, respectively;
   xxxiv) SEQ ID NOs: 265, 266, and 267, respectively;
   xxxv) SEQ ID NOs: 273, 274, and 275, respectively;
   xxxvi) SEQ ID NOs: 281, 282, and 283, respectively;
   xxxvii) SEQ ID NOs: 289, 290, and 291, respectively;
   xxxviii) SEQ ID NOs: 297, 298, and 299, respectively; or
   xxxix) SEQ ID NOs: 305, 306, and 307; and
b) an LCDR1, LCDR2, and LCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of:
   i) SEQ ID NOs: 4, 5, and 6, respectively;
   ii) SEQ ID NOs: 12, 13, and 14, respectively;
   iii) SEQ ID NOs: 20, 21, and 22, respectively;
   iv) SEQ ID NOs: 28, 29, and 30, respectively;
   v) SEQ ID NOs: 36, 37, and 38, respectively;
   vi) SEQ ID NOs: 44, 45, and 46, respectively;
   vii) SEQ ID NOs: 52, 53, and 54, respectively;
   viii) SEQ ID NOs: 60, 61, and 62, respectively;
   ix) SEQ ID NOs: 68, 69, and 70, respectively;
   x) SEQ ID NOs: 76, 77, and 78, respectively;
   xi) SEQ ID NOs: 84, 85, and 86, respectively;
   xii) SEQ ID NOs: 92, 93, and 94, respectively;
   xiii) SEQ ID NOs: 100, 101, and 102, respectively;
   xiv) SEQ ID NOs: 108, 109, 110, respectively;
   xv) SEQ ID NOs: 116, 117, and 118, respectively;
   xvi) SEQ ID NOs: 124, 125, and 126, respectively;
   xvii) SEQ ID NOs: 132, 133, and 134, respectively;
   xviii) SEQ ID NOs: 140, 141, and 142, respectively;
   xix) SEQ ID NOs: 148, 149, and 150, respectively;
   xx) SEQ ID NOs: 156, 157, and 158, respectively;
   xxi) SEQ ID NOs: 164, 165, and 166, respectively;
   xxii) SEQ ID NOs: 172, 173, and 174, respectively;
   xxiii) SEQ ID NOs: 180, 181, and 182, respectively;
   xxiv) SEQ ID NOs: 188, 189, and 190, respectively;
   xxv) SEQ ID NOs: 196, 197, and 198, respectively;
   xxvi) SEQ ID NOs: 204, 205, and 206, respectively;
   xxvii) SEQ ID NOs: 212, 213, and 214, respectively;
   xxviii) SEQ ID NOs: 220, 221, and 222, respectively;
   xxix) SEQ ID NOs: 228, 229, and 230, respectively;
   xxx) SEQ ID NOs: 236, 237, and 238, respectively;
   xxxi) SEQ ID NOs: 244, 245, and 246, respectively;
   xxxii) SEQ ID NOs: 252, 253, and 254, respectively;
   xxxiii) SEQ ID NOs: 260, 261, and 262, respectively;
   xxxiv) SEQ ID NOs: 268, 269, and 270, respectively;
   xxxv) SEQ ID NOs: 276, 277, and 278, respectively;
   xxxvi) SEQ ID NOs: 284, 285, and 286, respectively;
   xxxvii) SEQ ID NOs: 292, 293, and 294, respectively;
   xxxviii) SEQ ID NOs: 300, 301, and 302, respectively; or
   xxxix) SEQ ID NOs: 308, 309, and 310.

Embodiment 36 is the bispecific or multispecific antibody of any one of embodiments 32-35, wherein the first antigen-binding domain that specifically binds CD3 comprises an HCDR1, HCDR2, HCDR3, LCDR1, LCDR2, and LCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of:
a) SEQ ID NOs: 1, 2, 3, 4, 5, and 6, respectively;
b) SEQ ID NOs: 9, 10, 11, 12, 13, and 14, respectively;
c) SEQ ID NOs: 17, 18, 19, 20, 21, and 22, respectively;
d) SEQ ID NOs: 25, 26, 27, 28, 29, and 30, respectively;
e) SEQ ID NOs: 33, 34, 35, 36, 37, and 38, respectively;
f) SEQ ID NOs: 41, 42, 43, 44, 45, and 46, respectively;
g) SEQ ID NOs: 49, 50, 51, 52, 53, and 54, respectively;
h) SEQ ID NOs: 57, 58, 59, 60, 61, and 62, respectively;
i) SEQ ID NOs: 65, 66, 67, 68, 69, and 70, respectively;
j) SEQ ID NOs: 73, 74, 75, 76, 77, and 78, respectively;
k) SEQ ID NOs: 81, 82, 83, 84, 85, and 86, respectively;
l) SEQ ID NOs: 89, 90, 91, 92, 93, and 94, respectively;
m) SEQ ID NOs: 97, 98, 99, 100, 101, and 102, respectively;
n) SEQ ID NOs: 105, 106, 107, 108, 109, and 110, respectively;
o) SEQ ID NOs: 113, 114, 115, 116, 117, and 118, respectively;
p) SEQ ID NOs: 121, 122, 123, 124, 125, and 126, respectively;
q) SEQ ID NOs: 129, 130, 131, 132, 133, and 134, respectively;
r) SEQ ID NOs: 137, 138, 139, 140, 141, and 142, respectively;
s) SEQ ID NOs: 145, 146, 147, 148, 149, and 150, respectively;
t) SEQ ID NOs: 153, 154, 155, 156, 156, and 158, respectively;
u) SEQ ID NOs: 161, 162, 263, 164, 165, and 166, respectively;
v) SEQ ID NOs: 169, 170, 171, 172, 173, and 174, respectively;
w) SEQ ID NOs: 177, 178, 179, 180, 181, and 182, respectively;
x) SEQ ID NOs: 185, 186, 187, 188, 189, and 190, respectively;
y) SEQ ID NOs: 193, 194, 195, 196, 197, and 198, respectively;
z) SEQ ID NOs: 201, 202, 203, 204, 205, and 206, respectively;
aa) SEQ ID NOs: 209, 210, 211, 212, 213, and 214, respectively;
ab) SEQ ID NOs: 217, 218, 219, 220, 221, and 222, respectively;
ac) SEQ ID NOs: 225, 226, 227, 228, 229, and 230, respectively;
ad) SEQ ID NOs: 233, 234, 235, 236, 237, and 238, respectively;
ae) SEQ ID NOs: 241, 242, 243, 244, 245, and 246, respectively;
af) SEQ ID NOs: 249, 250, 251, 252, 253, and 254, respectively;
ag) SEQ ID NOs: 257, 258, 259, 260, 261, and 262, respectively;
ah) SEQ ID NOs: 265, 266, 267, 268, 269, and 270, respectively;
ai) SEQ ID NOs: 273, 274, 275, 276, 277, and 278, respectively;
aj) SEQ ID NOs: 281, 282, 283, 284, 285, and 286, respectively;
ak) SEQ ID NOs: 289, 290, 291, 292, 293, and 294, respectively;
al) SEQ ID NOs: 297, 298, 299, 300, 301, and 302, respectively; or
am) SEQ ID NOs: 305, 306, 307, 308, 309, and 310, respectively.

Embodiment 37 is the bispecific or multispecific antibody of any one of embodiments 32-36, wherein the first antigen-binding domain that specifically binds CD3 comprises an HCDR1, HCDR2, HCDR3, LCDR1, LCDR2, and LCDR3 comprising the amino acid sequences of:
a) SEQ ID NOs: 1, 2, 3, 4, 5, and 6, respectively;
b) SEQ ID NOs: 9, 10, 11, 12, 13, and 14, respectively;
c) SEQ ID NOs: 17, 18, 19, 20, 21, and 22, respectively;
d) SEQ ID NOs: 25, 26, 27, 28, 29, and 30, respectively;
e) SEQ ID NOs: 33, 34, 35, 36, 37, and 38, respectively;
f) SEQ ID NOs: 41, 42, 43, 44, 45, and 46, respectively;
g) SEQ ID NOs: 49, 50, 51, 52, 53, and 54, respectively;
h) SEQ ID NOs: 57, 58, 59, 60, 61, and 62, respectively;
i) SEQ ID NOs: 65, 66, 67, 68, 69, and 70, respectively;
j) SEQ ID NOs: 73, 74, 75, 76, 77, and 78, respectively;
k) SEQ ID NOs: 81, 82, 83, 84, 85, and 86, respectively;
l) SEQ ID NOs: 89, 90, 91, 92, 93, and 94, respectively;
m) SEQ ID NOs: 97, 98, 99, 100, 101, and 102, respectively;
n) SEQ ID NOs: 105, 106, 107, 108, 109, and 110, respectively;
o) SEQ ID NOs: 113, 114, 115, 116, 117, and 118, respectively;
p) SEQ ID NOs: 121, 122, 123, 124, 125, and 126, respectively;
q) SEQ ID NOs: 129, 130, 131, 132, 133, and 134, respectively;
r) SEQ ID NOs: 137, 138, 139, 140, 141, and 142, respectively;
s) SEQ ID NOs: 145, 146, 147, 148, 149, and 150, respectively;
t) SEQ ID NOs: 153, 154, 155, 156, 156, and 158, respectively;
u) SEQ ID NOs: 161, 162, 263, 164, 165, and 166, respectively;
v) SEQ ID NOs: 169, 170, 171, 172, 173, and 174, respectively;
w) SEQ ID NOs: 177, 178, 179, 180, 181, and 182, respectively;
x) SEQ ID NOs: 185, 186, 187, 188, 189, and 190, respectively;
y) SEQ ID NOs: 193, 194, 195, 196, 197, and 198, respectively;
z) SEQ ID NOs: 201, 202, 203, 204, 205, and 206, respectively;
aa) SEQ ID NOs: 209, 210, 211, 212, 213, and 214, respectively;
ab) SEQ ID NOs: 217, 218, 219, 220, 221, and 222, respectively;
ac) SEQ ID NOs: 225, 226, 227, 228, 229, and 230, respectively;
ad) SEQ ID NOs: 233, 234, 235, 236, 237, and 238, respectively;
ae) SEQ ID NOs: 241, 242, 243, 244, 245, and 246, respectively;
af) SEQ ID NOs: 249, 250, 251, 252, 253, and 254, respectively;
ag) SEQ ID NOs: 257, 258, 259, 260, 261, and 262, respectively;
ah) SEQ ID NOs: 265, 266, 267, 268, 269, and 270, respectively;
ai) SEQ ID NOs: 273, 274, 275, 276, 277, and 278, respectively;
aj) SEQ ID NOs: 281, 282, 283, 284, 285, and 286, respectively;
ak) SEQ ID NOs: 289, 290, 291, 292, 293, and 294, respectively;
al) SEQ ID NOs: 297, 298, 299, 300, 301, and 302, respectively; or
am) SEQ ID NOs: 305, 306, 307, 308, 309, and 310, respectively.

Embodiment 38 is the bispecific or multispecific antibody of any one of embodiments 32-37, wherein the first antigen-binding domain that specifically binds CD3 comprises an HCDR1, HCDR2, HCDR3, LCDR1, LCDR2, and LCDR3 comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of SEQ ID NOS:
a) 1, 2, 3, 4, 5, and 6, respectively;
b) 201, 202, 203, 204, 205, and 206, respectively;
c) 249, 250, 251, 252, 253, and 254, respectively; or
d) 297, 298, 299, 300, 301, and 302, respectively.

Embodiment 39 is the bispecific or multispecific antibody of any one of embodiments 32-38, wherein the first antigen-binding domain that specifically binds CD3 comprises an HCDR1, HCDR2, HCDR3, LCDR1, LCDR2, and LCDR3 comprising the sequences of SEQ ID NOs:
a) 1, 2, 3, 4, 5, and 6, respectively;
b) 201, 202, 203, 204, 205, and 206, respectively;
c) 249, 250, 251, 252, 253, and 254, respectively; or
d) 297, 298, 299, 300, 301, and 302, respectively.

Embodiment 40 is the bispecific or multispecific antibody of any one of embodiments 32-39, wherein the first antigen-binding domain that specifically binds CD3 comprises:
a) a heavy chain variable region (VH) comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 7, 15, 23, 31, 39, 47, 55, 63, 71, 79, 87, 95, 103, 111, 119, 127, 135, 143, 151, 159, 167, 175, 183, 191, 199, 207, 215, 223, 231, 239, 247, 255, 263, 271, 279, 287, 295, 303, and 311; and
b) a light chain variable region (VL) comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of any one of SEQ ID NOs: 8, 16, 24, 32, 40, 48, 56, 64, 72, 80, 88, 96, 104, 112, 120, 128, 136, 144, 152, 160, 168, 176, 184, 192, 200, 208, 216, 224, 232, 240, 248, 256, 264, 272, 280, 288, 296, 304, and 312.

Embodiment 41 is the bispecific or multispecific antibody of any one of embodiments 32-40, wherein the first antigen-binding domain that specifically binds CD3 comprises:
a) a heavy chain variable region (VH) comprising the amino acid sequence of any one of SEQ ID NOs: 7, 15, 23, 31, 39, 47, 55, 63, 71, 79, 87, 95, 103, 111, 119, 127, 135, 143, 151, 159, 167, 175, 183, 191, 199, 207, 215, 223, 231, 239, 247, 255, 263, 271, 279, 287, 295, 303, and 311; and
b) a light chain variable region (VL) comprising the amino acid sequence of any one of SEQ ID NOs: 8, 16, 24, 32, 40, 48, 56, 64, 72, 80, 88, 96, 104, 112, 120, 128, 136, 144, 152, 160, 168, 176, 184, 192, 200, 208, 216, 224, 232, 240, 248, 256, 264, 272, 280, 288, 296, 304, and 312.

Embodiment 42 is the bispecific or multispecific antibody of any one of embodiments 32-41, wherein the first antigen-binding domain comprises a VH and a VL comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of:
a) SEQ ID NOs: 7 and 8, respectively;
b) SEQ ID NOs: 15 and 16, respectively;
c) SEQ ID NOs: 23 and 24, respectively;
d) SEQ ID NOs: 31 and 32, respectively;
e) SEQ ID NOs: 39 and 40, respectively;
f) SEQ ID NOs: 47 and 48, respectively;
g) SEQ ID NOs: 55 and 56, respectively;
h) SEQ ID NOs: 63 and 64, respectively;
i) SEQ ID NOs: 71 and 72, respectively;
j) SEQ ID NOs: 79 and 80, respectively;
k) SEQ ID NOs: 87 and 88, respectively;
l) SEQ ID NOs: 95 and 96, respectively;
m) SEQ ID NOs: 103 and 104, respectively;
n) SEQ ID NOs: 111 and 112, respectively;
o) SEQ ID NOs: 119 and 120, respectively;
p) SEQ ID NOs: 127 and 128, respectively;
q) SEQ ID NOs: 135 and 136, respectively;
r) SEQ ID NOs: 143 and 144, respectively;
s) SEQ ID NOs: 151 and 152, respectively;
t) SEQ ID NOs: 159 and 160, respectively;
u) SEQ ID NOs: 167 and 168, respectively;
v) SEQ ID NOs: 175 and 176, respectively;
w) SEQ ID NOs: 183 and 184, respectively;
x) SEQ ID NOs: 191 and 192, respectively;
y) SEQ ID NOs: 199 and 200, respectively;
z) SEQ ID NOs: 207 and 208, respectively;
aa) SEQ ID NOs: 215 and 216, respectively;
ab) SEQ ID NOs: 223 and 224, respectively;
ac) SEQ ID NOs: 231 and 232, respectively;
ad) SEQ ID NOs: 239 and 240, respectively;
ae) SEQ ID NOs: 247 and 248, respectively;
af) SEQ ID NOs: 255 and 256, respectively;
ag) SEQ ID NOs: 263 and 264, respectively;
ah) SEQ ID NOs: 271 and 272, respectively;
ai) SEQ ID NOs: 279 and 280, respectively;
aj) SEQ ID NOs: 287 and 288, respectively;
ak) SEQ ID NOs: 295 and 296, respectively;
al) SEQ ID NOs: 303 and 304, respectively; or
am) SEQ ID NOs: 311 and 312, respectively.

Embodiment 43 is the bispecific or multispecific antibody of any one of embodiments 32-42, wherein the first antigen-binding domain comprises a VH and a VL comprising the amino acid sequences of:
a) SEQ ID NOs: 7 and 8, respectively;
b) SEQ ID NOs: 15 and 16, respectively;
c) SEQ ID NOs: 23 and 24, respectively;
d) SEQ ID NOs: 31 and 32, respectively;
e) SEQ ID NOs: 39 and 40, respectively;
f) SEQ ID NOs: 47 and 48, respectively;
g) SEQ ID NOs: 55 and 56, respectively;
h) SEQ ID NOs: 63 and 64, respectively;
i) SEQ ID NOs: 71 and 72, respectively;
j) SEQ ID NOs: 79 and 80, respectively;
k) SEQ ID NOs: 87 and 88, respectively;
l) SEQ ID NOs: 95 and 96, respectively;
m) SEQ ID NOs: 103 and 104, respectively;
n) SEQ ID NOs: 111 and 112, respectively;
o) SEQ ID NOs: 119 and 120, respectively;
p) SEQ ID NOs: 127 and 128, respectively;
q) SEQ ID NOs: 135 and 136, respectively;
r) SEQ ID NOs: 143 and 144, respectively;
s) SEQ ID NOs: 151 and 152, respectively;
t) SEQ ID NOs: 159 and 160, respectively;
u) SEQ ID NOs: 167 and 168, respectively;
v) SEQ ID NOs: 175 and 176, respectively;
w) SEQ ID NOs: 183 and 184, respectively;
x) SEQ ID NOs: 191 and 192, respectively;
y) SEQ ID NOs: 199 and 200, respectively;
z) SEQ ID NOs: 207 and 208, respectively;
aa) SEQ ID NOs: 215 and 216, respectively;
ab) SEQ ID NOs: 223 and 224, respectively;
ac) SEQ ID NOs: 231 and 232, respectively;
ad) SEQ ID NOs: 239 and 240, respectively;
ae) SEQ ID NOs: 247 and 248, respectively;
af) SEQ ID NOs: 255 and 256, respectively;
ag) SEQ ID NOs: 263 and 264, respectively;
ah) SEQ ID NOs: 271 and 272, respectively;
ai) SEQ ID NOs: 279 and 280, respectively;
aj) SEQ ID NOs: 287 and 288, respectively;
ak) SEQ ID NOs: 295 and 296, respectively;
al) SEQ ID NOs: 303 and 304, respectively; or
am) SEQ ID NOs: 311 and 312, respectively.

Embodiment 44 is the bispecific or multispecific antibody of any one of embodiments 32-43, wherein the first antigen-binding domain comprises a VH and a VL comprising an amino acid sequence having at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to the sequences of:
a) 7 and 8, respectively;
b) 207 and 208, respectively;
c) 255 and 256, respectively; or
d) 303 and 304, respectively.

Embodiment 45 is the bispecific or multispecific antibody of any one of embodiments 32-44, wherein the first antigen-binding domain comprises a VH and a VL comprising the amino acid sequences of:
a) 7 and 8, respectively;
b) 207 and 208, respectively;
c) 255 and 256, respectively; or
d) 303 and 304, respectively.

Embodiment 46 is the anti-CD3 antibody of any one of embodiments 32-45, wherein the antibody comprises from 1 to 10, from 1 to 5, or from 1 to 2 amino acid substitutions across all framework regions.

Embodiment 47 is the anti-CD3 antibody of any one of embodiments 32-46, wherein the antibody comprises 1 or 2 amino acid substitutions across all framework regions.

Embodiment 48 is the anti-CD3 antibody of any one of embodiments 32-47, wherein the antibody comprises from 1 to 10, from 1 to 5, or from 1 to 2 amino acid substitutions across all six CDR regions.

Embodiment 49 is the anti-CD3 antibody of any one of embodiments 32-48, wherein the antibody comprises 1 or 2 amino acid substitution across all six CDR regions.

Embodiment 50 is the anti-CD3 antibody of embodiment 46 or 47, wherein the amino acid substitutions are not in a CDR region.

Embodiment 51 is the anti-CD3 antibody of any one of embodiments 32-50, wherein the amino acid substitutions are all conservative amino acid substitutions.

Embodiment 52 is the bispecific or multispecific antibody of any one of embodiments 32-51, wherein the antibody is human or humanized.

Embodiment 53 is the bispecific or multispecific antibody of any one of embodiments 32-52, wherein the antibody comprises an Fc domain.

Embodiment 54 is the bispecific or multispecific antibody of any one of embodiments 32-53, wherein the Fc domain is a human immunoglobulin G (IgG) Fc domain.

Embodiment 55 is the bispecific or multispecific antibody of any one of embodiments 32-54, wherein the IgG Fc domain is an IgG1 Fc domain.

Embodiment 56 is the bispecific or multispecific antibody of any one of embodiments 28-55, wherein the Fc domain comprises M252Y, S254T, and T256E (YTE) substitutions, wherein numbering is according to the EU index.

Embodiment 57 is the bispecific or multispecific antibody of any one of embodiments 32-56, wherein the bispecific or multispecific antibody comprises two Fc chains.

Embodiment 58 is the bispecific or multispecific antibody of any one of embodiments 32-57, wherein one Fc domain comprises T366S, L368A, and Y407V substitutions and the second Fc domain comprises a T366W substitution, wherein numbering is according to the EU index.

Embodiment 59 is the bispecific or multispecific antibody of any one of embodiments 32-58, wherein the Fc domains further comprise an S354C and a Y349C substitution, wherein numbering is according to the EU index.

Embodiment 60 is the bispecific or multispecific antibody of any one of embodiments 32-59, wherein the second antigen binding domain specifically binds a cell surface antigen that is expressed on a cell other than an immune effector cell.

Embodiment 61 is the bispecific or multispecific antibody of any one of embodiments 32-60, wherein the cell surface antigen is a tumor associated antigen.

Embodiment 62 is an isolated nucleic acid encoding the bispecific or multispecific antibody of any one of embodiments 32-61.

Embodiment 63 is a vector comprising the isolated nucleic acid of embodiment 62.

Embodiment 64 is a host cell comprising the vector of embodiment 63.

Embodiment 65 is a method of producing the bispecific or multispecific antibody of any one of embodiments 32-61 comprising: culturing the host cell of embodiment 64 in an environment conducive to the expression of the bispecific or multispecific antibody of any one of embodiments 32-61; and collecting the bispecific or multispecific antibody of any one of embodiments 32-61.

Embodiment 66 is a composition comprising the bispecific or multispecific antibody of any one of claims 32-61, the isolated nucleic acid of embodiment 62, or the vector of embodiment 63 and a pharmaceutically acceptable carrier.

Embodiment 67 is a method of treating a disease or disorder in a subject in need thereof comprising administering to the subject the bispecific antibody of any one of embodiments 32-61, the isolated nucleic acid of embodiment 62, the vector of embodiment 63, or the composition of embodiment 66.

Embodiment 68 is the method of embodiment 67, wherein the disease or disorder is cancer.

Embodiment 69 is the method of embodiment 68, wherein the cancer is a solid tumor or a hematologic cancer.

Embodiment 70 is the bispecific or multispecific antibody of any one of embodiments 32-61, the isolated nucleic acid of embodiment 62, the vector of embodiment 63, or the composition of embodiment 66 for use in treating a disease or disorder in a subject in need thereof.

Embodiment 71 is the bispecific or multispecific antibody, isolated nucleic acid, vector, or composition for use of embodiment 70, wherein the disease or disorder is cancer.

Embodiment 72 is the bispecific or multispecific antibody, isolated nucleic acid, vector, or composition for use of embodiment 71, wherein the cancer is a solid tumor or a hematologic cancer.

Embodiment 73 is a use of the bispecific or multispecific antibody of any one of embodiments 32-61, the isolated nucleic acid of embodiment 62, the vector of embodiment 63, or the composition of embodiment 66 in treating a disease or disorder in a subject in need thereof.

Embodiment 74 is the use of embodiment 73, wherein the disease or disorder is cancer.

Embodiment 75 is the use of embodiment 74, wherein the cancer is a solid tumor or a hematologic cancer.

Embodiment 76 is a use of the bispecific or multispecific antibody of any one of claims 32-61, the isolated nucleic acid of embodiment 62, the vector of 63, the host cell of 64, or the composition of embodiment 66 in preparation of a medicament for treating a disease or disorder in a subject in need thereof.

Embodiment 77 is the use of embodiment 76, wherein the disease or disorder is cancer.

Embodiment 78 is the use of claim 77, wherein the cancer is a solid tumor or a hematologic cancer.

### EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Generation of CD3 Binders

Human immunoglobulin Kappa-Lambda (Ablexis®; AlivaMab, LLC.) transgenic mice were immunized with human CD3 epsilon/delta heterodimer protein and primary human T cells. Titers were assessed by flow cytometry with titration of individual animal immune serum on parental Jurkat and Jurkat KO cells throughout the course of the immunization. The animals with the best test bleed titers were selected for hybridoma generation in which the lymph nodes and spleen were pooled and processed for B-cell enrichment prior to electrofusion hybridoma generation.

The plated hybridomas were screened by flow cytometry on Jurkat parental and Jurkat KO cells in a primary screen. 93 hybridomas were advanced to confirmatory screening via binding to human pan T cells, ELISA binding to human CD3 epsilon/delta, human CD3 epsilon/gamma, and cyno CD3 epsilon/delta heterodimer proteins, as well as off-target analysis by Octet using the same proteins. 52 representative hybridomas were selected for subcloning, and 39 subclones with the most promising preliminary results were obtained for molecular recovery and sequencing of heavy and light variable regions (Table 2).

Binding Screening on Jurkat and Pan T Cells: Multiplex screening on Jurkat WT (ATCC), Jurkat RT3-T3.5 CD3-KO (ATCC), and human pan T cells (Stemcell, Cat #70024) were performed on an iQue3 cytometer. 50 μL of neat hybridoma supernatant from each clone was added to cells seeded at 50K per well and incubated for 30 minutes at 4° C., followed by washing twice with FACS buffer and incubation with a secondary goat anti-mouse IgG Fc-647 (Jackson, Cat #115-606-071) detection antibody for 30 minutes at 4° C. Upon final washing with the FACS buffer, the data was acquired on the cytometer. Cell binding results are displayed in Table 4.

TABLE 4
Cell Binding Screen of CD3 Binders

| | Binding to Jurkat cells | | | Binding to Pan T cells | |
|---|---|---|---|---|---|
| | Jurkat | Jurkat | | Pan | Fold |
| | WT | TCR KO | Fold | T cells | (pan |
| Clone name | gMFI | gMFI | (WT/KO) | gMFI | T/buffer) |
| 01C03A | 27475 | 3122 | 9 | 98281 | 31 |
| 01G24A | 70365 | 5647 | 12 | 390845 | 122 |
| 01M02A | 20664 | 2882 | 7 | 70950 | 22 |
| 01P14N | 15412 | 2333 | 7 | 79383 | 25 |
| 02I07A | 68123 | 4740 | 14 | 276302 | 86 |
| 02J12A | 16174 | 2515 | 6 | 42934 | 13 |
| 03J04A | 68046 | 5121 | 13 | 356439 | 112 |
| 03L07A | 36744 | 4386 | 8 | 485550 | 152 |
| 03O15A | 22127 | 2902 | 8 | 60697 | 19 |
| 03P01Z | 4763 | 2092 | 2 | 10017 | 3 |
| 04C03A | 14940 | 2777 | 5 | 92114 | 29 |
| 04D18A | 8291 | 2369 | 3 | 9420 | 3 |
| 04J18A | 37018 | 3311 | 11 | 181754 | 57 |
| 04K01A | 3835 | 1939 | 2 | 7809 | 2 |
| 04M22A | 18789 | 2788 | 7 | 220661 | 69 |
| 04P20A | 57967 | 4231 | 14 | 179357 | 56 |
| 05B02A | 82765 | 5126 | 16 | 389697 | 122 |
| 05L13A | 35725 | 3661 | 10 | 73230 | 23 |
| 05N13A | 13273 | 2548 | 5 | 42183 | 13 |
| 06A06A | 59122 | 4621 | 13 | 323363 | 101 |
| 07B06Z | 25493 | 3161 | 8 | 83849 | 26 |
| 07H10A | 61166 | 5220 | 12 | 286226 | 90 |
| 07L12A | 12682 | 2588 | 5 | 68016 | 21 |
| 07N22A | 11471 | 3143 | 4 | 23282 | 7 |
| 07P07A | 31769 | 3441 | 9 | 87366 | 27 |
| 07P15A | 45322 | 4393 | 10 | 229033 | 72 |
| 08M01A | 73364 | 5914 | 12 | 314059 | 98 |
| 08M15Z | 6374 | 2698 | 2 | 13519 | 4 |
| 09B13A | 42941 | 4078 | 11 | 130709 | 41 |
| 09D16A | 35170 | 4116 | 9 | 61333 | 19 |
| 10B09A | 14679 | 3691 | 4 | 35695 | 11 |
| 10E16A | 74413 | 4733 | 16 | 192427 | 60 |
| 14A15A | 130929 | 8289 | 16 | 412135 | 129 |
| 14C10A | 59259 | 5525 | 11 | 175044 | 55 |
| 14E03A | 27590 | 4013 | 7 | 74095 | 23 |
| 14O18A | 45805 | 6332 | 7 | 195203 | 61 |
| FACS buffer | 2869 | 2650 | 1 | 3197 | 1 |

CD3 Protein Binding Screen: ELISA assays for direct protein binding were performed using a capture format coated with 1 μg/mL anti-huFc (Jakson, Cat #109-007-008). Each 1 μg/mL in-house produced human CD3 E/D (epsilon delta)-Fc, human CD3 E/G (epsilon gamma)-Fc, cyno CD3 E/D (epsilon delta)-Fc, and hulgG Fc protein (Jackson, Cat #009-000-008) was added to the well containing the anti-huFc surface, followed by the addition of hybridoma supernatants, and the secondary HRP-Goat Anti-Mouse IgG (Jackson, Cat #115-035-164) for detection. Protein binding results are depicted in Table 5. For lower cytotoxicity, antibodies that demonstrated minimal binding to cyno CD3 were favored over those which displayed significant binding to cyno CD3.

The CD3-TCR complex is made of multiple protein chains, in addition to TCR chains, it comprises epsilon, delta, and gamma chains. While the TCR confers the binding specificity for targets, the CD3 subunits facilitate signal transduction necessary for T cell activation. While the mechanisms through which antigen sensing and signal transduction occur in the CD3-TCR complex are still under debate, recent revelations regarding the intricate 3D structure of the CD3-TCR complex open the possibility of modulating its activity by designing targeted drugs and tools. Aptamers and other CD3 binders that can activate this complex can bind to one of these chains or multiple chains. It is believed that different epitopes of CD3 can confer different functionality (Menon, A. P., et al., 2023, Cancers, 15 (4): 1189). Currently almost all the CD3 engagers target only one epitope identified by CD3 antibody SP34 on the epsilon chain (Martin, G., et al., 2020, J Immunother Cancer, 8 (Suppl 3): A7-A8) The various CD3 binders generated here recognize different epitopes on the CD3 complex. To identify the epitope diversity, proteins that either contained epsilon and delta chain or epsilon and gamma chain were used. It was reasoned that if a binder binds both proteins, it is most likely identifying only the epsilon chain. If, however, it recognizes only one of the proteins the majority of its binding epitope is being contributed by the non-epsilon chain (either delta or gamma).

TABLE 5
CD3 Binding

| | | | | Hu Fc | | | |
|---|---|---|---|---|---|---|---|
| | Hu | Hu | Cy | counter- | Fold | Fold | Fold |
| Clone | CD3 E-D | CD3 E-G | CD 3 E-G | screen | (hu CD3 | (hu CD3 | (Cyno CD3 |
| name | RLU | RLU | RLU | RLU | E-D/Hu Fc) | E-G/Hu Fc) | E-D/Hu Fc) |
| 01C03A | 462884 | 454098 | 276021 | 64433 | 7 | 7 | 4 |
| 01G24A | 831340 | 786593 | 3098 | 1356 | 613 | 580 | 2 |
| 01M02A | 505789 | 542679 | 5700 | 3152 | 160 | 172 | 2 |
| 01P14N | 936215 | 949392 | 1001004 | 536327 | 2 | 2 | 2 |
| 02I07A | 553818 | 447252 | 91991 | 16205 | 34 | 28 | 6 |
| 02J12A | 118006 | 129089 | 5877 | 2489 | 47 | 52 | 2 |
| 03J04A | 468354 | 425382 | 3874 | 3089 | 152 | 138 | 1 |
| 03L07A | 1136426 | 1008684 | 2500 | 1234 | 921 | 818 | 2 |
| 03O15A | 323255 | 168526 | 3763 | 2454 | 132 | 69 | 2 |
| 03P01Z | 26188 | 31836 | 10815 | 3270 | 8 | 10 | 3 |
| 04C03A | 315145 | 278073 | 8375 | 2026 | 156 | 137 | 4 |
| 04D18A | 5649 | 6003 | 2465 | 1489 | 4 | 4 | 2 |
| 04J18A | 655518 | 434162 | 11926 | 1411 | 465 | 308 | 8 |
| 04K01A | 448109 | 447416 | 455103 | 85917 | 5 | 5 | 5 |
| 04M22A | 645417 | 609537 | 67681 | 34540 | 19 | 18 | 2 |
| 04P20A | 400746 | 298606 | 2388 | 1099 | 365 | 272 | 2 |
| 05B02A | 546667 | 480127 | 1928 | 1281 | 427 | 375 | 2 |
| 05L13A | 684278 | 627777 | 2275 | 811 | 844 | 774 | 3 |
| 05N13A | 336945 | 87827 | 4051 | 951 | 354 | 92 | 4 |
| 06A06A | 618879 | 471341 | 1053 | 2256 | 274 | 209 | 0 |
| 07B06Z | 611783 | 459938 | 2243 | 364 | 1683 | 1265 | 6 |
| 07H10A | 672863 | 680011 | 584651 | 4138 | 163 | 164 | 141 |
| 07L12A | 69644 | 80692 | 6931 | 8314 | 8 | 10 | 1 |
| 07N22A | 62799 | 64261 | 1450 | 559 | 112 | 115 | 3 |
| 07P07A | 505590 | 569003 | 92027 | 46741 | 11 | 12 | 2 |
| 07P15A | 419361 | 322416 | 1215 | 631 | 665 | 511 | 2 |
| 08M01A | 1098014 | 1065161 | 1708 | 1637 | 671 | 651 | 1 |
| 08M15Z | 66901 | 50627 | 948 | 541 | 124 | 94 | 2 |
| 09B13A | 464075 | 270814 | 1625 | 844 | 550 | 321 | 2 |
| 09D16A | 149437 | 172131 | 800 | 799 | 187 | 216 | 1 |
| 10B09A | 224688 | 205807 | 965 | 304 | 740 | 678 | 3 |
| 10E16A | 452253 | 402312 | 510 | 644 | 703 | 625 | 1 |
| 14A15A | 727115 | 735876 | 15045 | 554 | 1314 | 1329 | 27 |
| 14C10A | 309644 | 335927 | 555 | 586 | 528 | 573 | 1 |
| 14E03A | 100829 | 111604 | 738 | 301 | 335 | 371 | 2 |
| 14O18A | 644514 | 697976 | 3103 | 796 | 810 | 877 | 4 |
| FACS buffer | 964 | 563 | 623 | 561 | 2 | 1 | 1 |

Binding Affinity for Human CD3: Binding kinetics of the identified CD3 binders to human CD3 proteins were determined on an Octet using anti-mouse Fc capture (AMC) sensors (Sartorius, Cat #18-5088). Each hybridoma supernatant was diluted 1:3 in kinetics buffer and loaded onto the AMC sensor, followed by the binding measurement to 200 nM human CD3 E/D (epsilon delta)-Fc or human CD3 E/G (epsilon gamma)-Fc protein in solution. The experimental setup included a 180s sensor equilibration step, a 120s baseline step, a 300s loading step, a 240s association step, and a 360s dissociation step with a 120s regeneration step in between each loading-binding cycle. The collected data were referenced using a parallel buffer blank subtraction, and the baseline was aligned to the y-axis prior to fitting using a 1:1 Langmuir kinetic model. The kinetic parameters include kₒₙ the association rate constant for the antibody-antigen binding reaction, k_{off} the dissociation rate constant of the antibody-antigen complex, and KD the equilibrium dissociation constant defined by k_{off}/kₒₙ. Binding affinity data for the identified CD3 binders is shown in Table 6.

TABLE 6
Human CD3 Binding Affinity

| Clone | Hu CD3 E/D | | | Hu CD3 E/G | | |
|---|---|---|---|---|---|---|
| name | KD (M) | k_{off} (1/s) | kₒₙ (1/Ms) | KD (M) | k_{off} (1/s) | kₒₙ (1/Ms) |
| 01C03A | 4.4E−10 | 2.5E−04 | 5.7E+05 | 1.1E−09 | 4.7E−04 | 4.3E+05 |
| 01G24A | 1.5E−08 | 2.0E−03 | 1.3E+05 | 4.7E−08 | 5.3E−03 | 1.1E+05 |
| 01M02A | 2.6E−10 | 1.9E−04 | 7.2E+05 | 3.3E−13 | 1.0E−07 | 3.1E+05 |
| 01P14N | 4.6E−13 | 1.00E−07 | 2.20E+05 | 6.8E−13 | 1.00E−07 | 1.47E+05 |
| 02I07A | 8.4E−09 | 1.3E−03 | 1.5E+05 | 3.2E−08 | 4.3E−03 | 1.4E+05 |
| 02J12A | No binding | | | No binding | | |
| 03J04A | 3.0E−08 | 7.2E−03 | 2.4E+05 | 5.4E−08 | 1.1E−02 | 2.0E+05 |
| 03L07A | 2.0E−09 | 3.3E−04 | 1.6E+05 | 7.0E−09 | 8.1E−04 | 1.2E+05 |
| 03O15A | 1.1E−08 | 2.9E−03 | 2.7E+05 | 1.2E−08 | 2.5E−03 | 2.2E+05 |
| 03P01Z | 3.0E−09 | 6.9E−04 | 2.3E+05 | 5.6E−09 | 3.8E−03 | 6.8E+05 |
| 04C03A | 6.1E−08 | 4.9E−03 | 8.0E+04 | 1.5E−09 | 1.0E−03 | 7.1E+05 |
| 04D18A | 4.4E−13 | 1.0E−07 | 2.3E+05 | No binding | | |
| 04J18A | 3.0E−09 | 6.9E−04 | 2.3E+05 | 5.6E−09 | 3.8E−03 | 6.8E+05 |
| 04K01A | 5.0E−10 | 5.5E−04 | 1.1E+06 | No binding | | |
| 04M22A | 4.3E−13 | 1.0E−07 | 2.3E+05 | No binding | | |
| 04P20A | No binding | | | No binding | | |
| 05B02A | 4.8E−09 | 1.2E−03 | 2.5E+05 | 1.4E−08 | 2.6E−03 | 1.8E+05 |
| 05L13A | 4.1E−13 | 1.0E−07 | 2.4E+05 | 6.2E−09 | 2.3E−03 | 3.7E+05 |
| 05N13A | 3.1E−08 | 2.8E−02 | 9.0E+05 | 6.9E−06 | 5.3E−01 | 7.7E+04 |
| 06A06A | 1.5E−09 | 3.4E−04 | 2.2E+05 | 1.1E−08 | 1.9E−03 | 1.8E+05 |
| 07B06Z | 6.5E−13 | 1.0E−07 | 1.6E+05 | 6.4E−10 | 8.5E−05 | 1.3E+05 |
| 07H10A | 1.6E−09 | 4.0E−04 | 2.5E+05 | 9.7E−10 | 2.3E−04 | 2.4E+05 |
| 07L12A | 3.5E−13 | 1.0E−07 | 2.8E+05 | 2.7E−10 | 6.1E−04 | 2.3E+06 |
| 07N22A | 1.3E−09 | 1.4E−03 | 1.1E+06 | No binding | | |
| 07P07A | 1.4E−13 | 1.0E−07 | 7.1E+05 | 7.8E−10 | 9.3E−04 | 1.2E+06 |
| 07P15A | 2.8E−08 | 4.2E−02 | 1.5E+06 | No binding | | |
| 08M01A | 2.1E−09 | 3.7E−04 | 1.7E+05 | 4.0E−09 | 5.0E−04 | 1.2E+05 |
| 08M15Z | 9.3E−08 | 9.3E−02 | 1.0E+06 | No binding | | |
| 09B13A | 1.5E−08 | 3.8E−03 | 2.6E+05 | 4.9E−09 | 2.5E−03 | 5.1E+05 |
| 09D16A | 5.6E−13 | 1.0E−07 | 1.8E+05 | 2.3E−13 | 1.0E−07 | 4.3E+05 |
| 10B09A | 6.3E−10 | 2.9E−04 | 4.7E+05 | 1.8E−13 | 1.0E−07 | 5.4E+05 |
| 10E16A | 7.2E−09 | 3.6E−03 | 5.0E+05 | 8.4E−09 | 4.7E−03 | 5.6E+05 |
| 14A15A | 6.5E−13 | 1.0E−07 | 1.5E+05 | 1.1E−12 | 1.0E−07 | 8.9E+04 |
| 14C10A | 4.8E−09 | 4.1E−03 | 8.5E+05 | 1.9E−09 | 1.4E−03 | 7.2E+05 |
| 14E03A | No binding | | | No binding | | |
| 14O18A | 1.2E−13 | 1.0E−07 | 8.4E+05 | 6.6E−10 | 6.9E−04 | 1.0E+06 |

Binding sites on CD3 engaged by 14C10A were subsequently mapped by hydrogen-deuterium exchange mass spectrometry (HDX-MS), A mixture containing Ab897 and human CD3εδ (ACROBiosystems, Cat #CDD-H52W1) was prepared at a 1:1.3 molar ratio, with concentrations of 10.5 μM and 8.1 μM, respectively. The deuterium exchange reaction was initiated by mixing 10 μl of the mixture solution or 10 μL of CD3 alone into 90 μL of deuterium oxide labeling buffer (50 mM sodium phosphate, 100 mM sodium chloride, pH 7.0). The reaction was incubated for 0 s, 15 s, 60 s, 600 s, and 3600 s at 20° C., and then quenched by adding 100 μL of 4 M Guanidine HCl, 0.85 M TCEP buffer to the fully deuterated samples. The quenched samples were subjected to on-column pepsin and prolyl endopeptidase digestion, followed by LC-MS analysis using a UPLC-MS system consisting of a Waters Acquity UPLC coupled to an Orbitrap Exploris™ 240 Mass Spectrometer (Thermo Scientific). Peptide identification was performed by searching the MS/MS data against the sequences of human CD3 using Protein Metrics. The mass tolerance for the precursor and product ions was set to 10 ppm and 0.02 Da, respectively. For the analysis of H/D exchange MS data, HDX WorkBench was used to calculate the deuterium uptake level of individual peptides by determining the mass difference between the deuterated peptide and its native form. Peptides exhibiting a significantly reduced average percentage of deuterium uptake in the presence of Ab897 were considered the epitope.

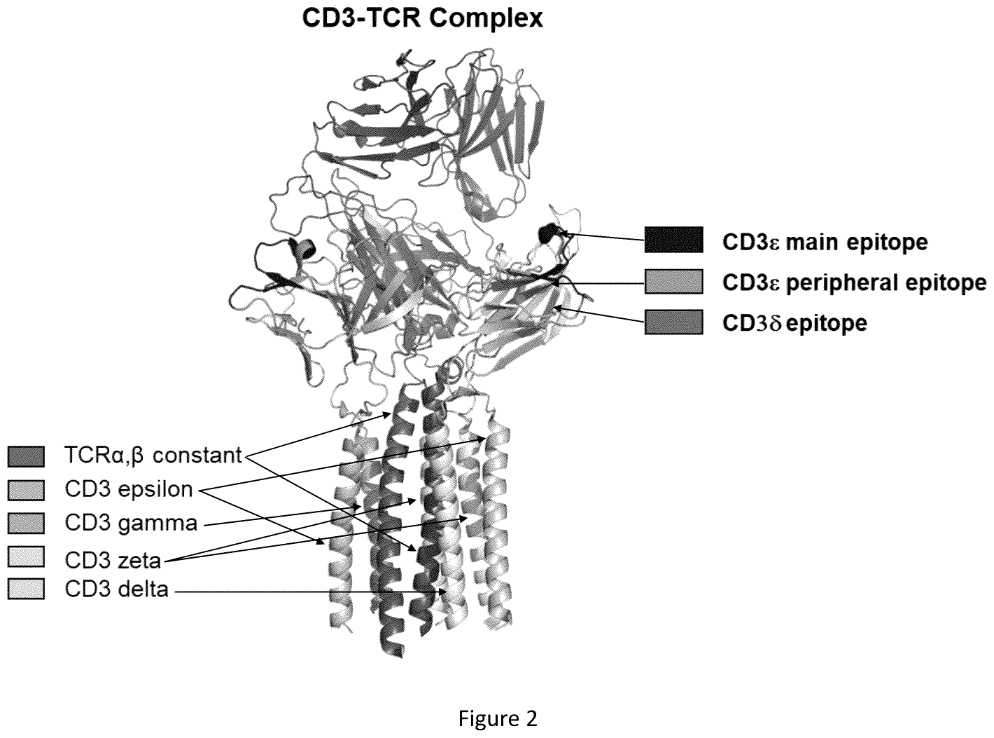

HDX revealed interactions with both the CD3ε and CD3δ subunits (FIG. 2). Comparative deuterium uptake analysis between the 14C10A/CD3εδ complex and CD3εδ alone identified the primary binding regions on CD3ε [Uniprot ID: P07766] as residues 54-58 (GSEIL, SEQ ID NO:331) and 98-106 (CYPRGSKPE, SEQ ID NO:332), marked by significant reductions in deuterium uptake. Additional residues 48-54 (TCPQYP, SEQ ID NO:333), 92-97 (QSGYYV, SEQ ID NO:334), and 107-116 (DANFYLYLRA, SEQ ID NO:335) were considered peripheral interaction regions due to moderate reductions observed. Similarity, a substantial decrease in deuterium uptake was observed on CD3δ [Uniprot ID: P04234] at residues 57-71 (RLDLGKRILDPRGIY, SEQ ID NO:336) upon 14C10A binding.

### Example 2: CD3×CLDN6 Bispecific Antibodies

CLDN6 Binder Generation: SJL and BALB/c mice were immunized with a pVAX (Invitrogen) CLDN6 vector incorporating the human CLDN6 ECD sequence (UniProt ID: P56747). Antibody titers were assessed by flow cytometry with titration of individual animal immune serum on a CHO-CLDN6 overexpressing cell line and parental cell line throughout the course of the immunization. The animals with the best test bleed titers were selected for B cell isolation and culture, followed by primary screening by flow cytometry on CHO-CLDN6 cells. Top binders were advanced for secondary screening by flow cytometry to verify CLDN6 specificity and cross-reactivity with CHO-CLDN9 overexpressing cells. The top 48 CLDN6-specific B cells with minimal binding to CLDN9 were selected for cloning and sequencing to recover the V region sequence. The recovered heavy and light variable region sequence for each antibody were transfected and expressed in CHO cells, followed by the screening of cell culture supernatants by flow cytometry to confirm recombinant antibodies retain CLDN6 specificity. The 48 CLDN6 binders were screened against closely related proteins CLDN3, CLDN4, and CLDN9, and of the 48 binders, only one binder demonstrated substantial binding to CDLN6 without any observable binding to CLDN3, CLDN4, and CLDN9. The 48 CLDN6 binders were screened by flow cytometry against CHO cells expressing closely related proteins CLDN3, CLDN4, and CLDN9. Of the 48 binders, 19 were selected for additional screening against CLDN6 vs CLDN9 (Table 7). Of these, one binder demonstrated substantial binding to CDLN6 without any observable binding to CLDN3, CLDN4, and CLDN9.

TABLE 7
Antibody Binding to CLDN6 and CLDN9

| | | | | CHO- | CHO- | |
|---|---|---|---|---|---|---|
| | CHO-K1 | CHO-K1 | CHO-K1 | CLDN6/ | CLDN9/ | CLDN6/ |
| Clone ID | CLDN6 | CLDN9 | WT | WT | WT | CLDN9 |
| 51H102/51K301 | 1981869 | 21572 | 12881 | 153.9 | 1.7 | 91.9 |
| 112H105/112K107 | 1164509 | 547379 | 12272 | 94.9 | 44.6 | 2.1 |
| 88H201/88K405 | 714482 | 438449 | 19316 | 37 | 22.7 | 1.6 |
| 54H100/54K303 | 691664 | 156199 | 12819 | 54 | 12.2 | 4.4 |
| 96H500/96K120 | 603467 | 350722 | 16188 | 37.3 | 21.7 | 1.7 |
| 94H909/94K110 | 375130 | 308405 | 13356 | 28.1 | 23.1 | 1.2 |
| 38H109/38K800 | 274363 | 120652 | 10689 | 25.7 | 11.3 | 2.3 |
| 90H501/90K904 | 272302 | 155625 | 9742 | 28 | 16.0 | 1.7 |
| 78H500/78K111 | 175733 | 126205 | 12409 | 14.2 | 10.2 | 1.4 |
| 22H201/22K609 | 151430 | 178142 | 10713 | 14.1 | 16.6 | 0.9 |
| 39H501/39K143 | 139733 | 41255 | 9890 | 14.1 | 4.2 | 3.4 |
| 80H302/80K501 | 91475 | 31865 | 9313 | 9.8 | 3.4 | 2.9 |
| 18H102/18K308 | 90241 | 48397 | 9583 | 9.4 | 5.1 | 1.9 |
| 48H105/48K501 | 83191 | 24116 | 8576 | 9.7 | 2.8 | 3.4 |
| 86H103/86K108 | 76662 | 61359 | 9652 | 7.9 | 6.4 | 1.2 |
| 40H108/40K171 | 70216 | 18683 | 9369 | 7.5 | 2.0 | 3.8 |
| 5H113/5K501 | 68946 | 10540 | 7585 | 9.1 | 1.4 | 6.5 |
| 72H400/72K202 | 64554 | 10384 | 9071 | 7.1 | 1.1 | 6.2 |
| 49H500/49K105 | 45218 | 9338 | 8220 | 5.5 | 1.1 | 4.8 |

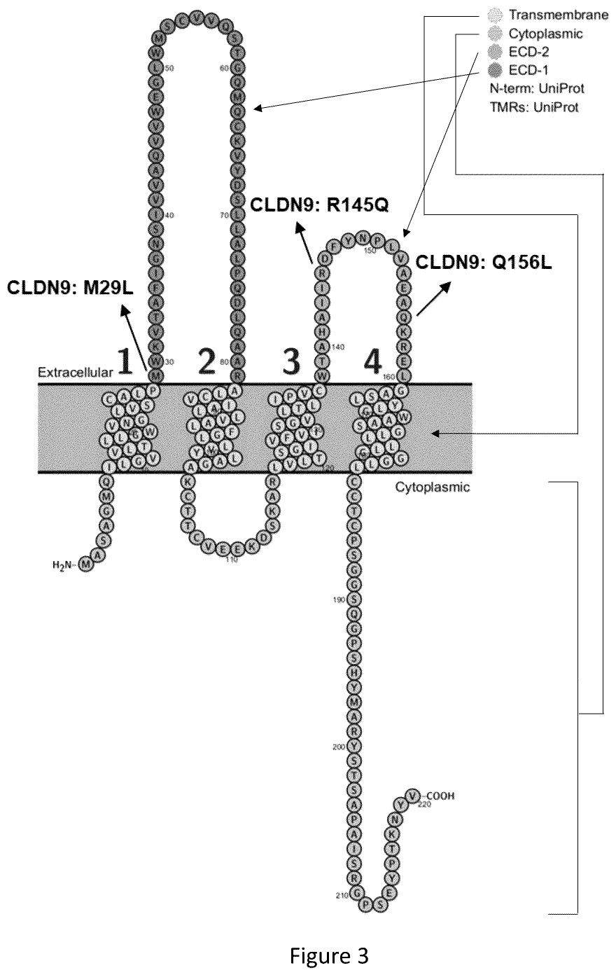

To gain further insight into the molecular features underlying the specificity of 51H102 for CLDN6 over CLDN9, site-directed mutagenesis was performed at the three amino acid positions that differ in their ECDs to enable epitope mapping at the residue level (FIG. 3). A total of six CLDN6/CLDN9 hybrid constructs were generated by substituting the CLDN6 residues with the corresponding CLDN9 residues at each differing position. These included three single-point mutants (M29L, R145Q, and Q156L) and three double-point mutants (M29L+R145Q, M29L+Q156L, and R145Q+Q156L), along with wide-type sCLDN6 and CLDN9 controls. All constructs were cloned into the pcDNA3.1 vector and transiently expressed in SKOV3 cells, which lack endogenous CLDN6 or CLDN9 expression, and antibody binding was assessed by flow cytometry. In addition to 51H102, a similarly high-affinity CLDN6 binder 112H105, which exhibits 50% cross-reactivity with CLDN9, was included to further evaluate the role of these residues in determining binding specificity. Anti-CLDN6 antibodies (100 nM) were incubated with each transfected cell line for 1 hour at 4° C. Cells were washed with PBS, and the bound antibodies were stained with PE-conjugated polyclonal goat anti-human IgG (Abcam, Cat #ab98596) for 1 hour at 4° C. Samples were acquired in the PE channel on the Novocyte Flow Cytometer (Agilent Technologies). Data were analyzed using NovoExpress software (Agilent Technologies, version 1.2.5). The antibody binding percentage to mutants relative to wild-type CLDN6 was calculated by subtracting the binding signal from untransfected cells and dividing by the binding signal from wild-type CLDN6 cells.

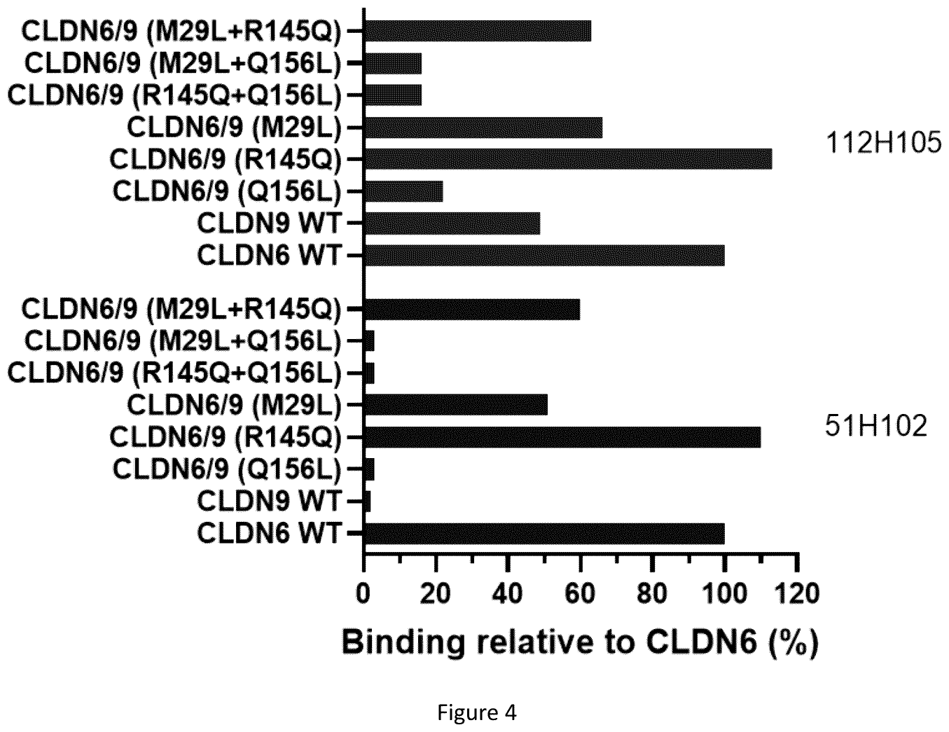

To define the molecular basis of 51H102 binding specificity, site-directed mutagenesis was performed at the three amino acid positions in the ECDs that differ between CLDN6 and CLDN9, enabling epitope mapping at the residue level (FIG. 3). Six CLDN6/CLDN9 hybrid constructs were generated by substituting CLDN6 residues with their CLDN9 counterparts, including three single-point mutants (M29L, R145Q, and Q156L) and three double-point mutants (M29L+R145Q, M29L+Q156L, and R145Q+Q156L), along with wild-type CLDN6 and CLDN9 controls. Constructs were transiently expressed in SKOV3 cells, which lack endogenous CLDN6 and CLDN9 expression, and antibody binding was assessed via flow cytometry. In addition to 51H102, a similarly high-affinity CLDN6 binder 112H105, which exhibits 50% cross-reactivity with CLDN9, was included as a comparator. The results showed that both M29L and Q156L mutations impaired 51H102 binding to CLDN6 (FIG. 4). The Q156L substitution completely abolished binding, while M29L reduced binding by an approximate 50%. R145Q alone had no effect; however, when combined with Q156L, CLDN6 binding was entirely lost. Similarly, the M29L+Q156L double abolished binding, mirroring the single Q156L effect. In contrast, the M29L+R145Q mutant reduced binding by an approximate 50%, comparable to the single M29L mutant. These findings indicate that Q156 in ECD2 is the primary contact residue for 51H102, with M29 in ECD1 also contributing to CLDN6 recognition, consistent with binding to a conformational epitope spanning both ECD domains. Data from the CLDN9-cross-reactive binder 112H105 showed a similar trend, with both M29 and Q156 contributing to CLDN6 binding but with differential effects on CLDN9. The Q156L substitution, as well as its double mutants M29L+Q156L and R145Q+Q156L, significantly impaired binding to CLDN6 (FIG. 4). Unlike 51H102, for which Q156L completely abolished CLDN6 binding, this mutation reduced 112H105 binding to CLDN6 by 70-80% while also decreasing binding to CLDN9 by an approximate 50%. In contrast, the M29L single and its M29L+R145Q mutant had no negative impact on CLDN9 but reduced CLDN6 binding by an approximate 30%. Collectively, these results indicate that Q156 is a critical determinant for interactions with both CLDN6 and CLDN9, while 51H102 achieves exquisite specificity by recognizing Q156 together with M29 in a CLDN6-specific conformational context.

Bispecific Antibody Design: For flexibility in the design of bispecific and multispecific antibodies, the CD3 binders were converted to an scFv VL-linker-VH format, with a standard linker. In order to determine which of the scFvs would be able to stably bind CD3, the scFvs were used to form a test bispecific antibody with the scFv conjugated to an Fc domain and coupled with a tumor antigen-binding Fab conjugated to an Fc. Heterodimerization was achieved by incorporation of “knob” and “hole” substitutions in Fc domains. The test bispecific antibodies were analyzed for thermostability, and of the anti-CD3 binders, four (01C03A, 08M15A, 14C10A, and 14E03A) demonstrated high thermostability (Tₘ>60° C.) scFvs in a VL-linker-VH orientation (Table 8), These four CD3 binders were then engineered to further increase stability, with a linker sequence of GGSEGKSSGSGSESKSTGGS (SEQ ID NO:325), and with the amino acids at positions VH44 and VL100 (Kabat numbering) substituted with cysteines. The C-terminus of the anti-CD3 scFv was conjugated to a human IgG1 Fc domain. Of the four anti-CD3 binders, upon formation of anti-CD3×anti-CLDN6 bispecific antibodies, antibody Ab897, comprising the VH and VL of 14C10, demonstrated moderate CD3 binding which resulted in the best targeted cell killing of the four bispecific antibodies. Ab897 comprises the CD3-binding domain of 14C10 and the CLDN6-binding domain of 51H102/51K301 of Table 7.

TABLE 8
CD3 scFv Thermostability

| Clone Name | Tₘ (° C.) | K_{D} at RT (nM) |
|---|---|---|
| 07H10A | 69.3 | No Binding |
| 01C03A | 64.1 | 3.0 |
| 14C10A | 63.9 | 6.3 |
| 08M15A | 61.6 | 12.5 |
| 14E03A | 60.6 | 4.7 |
| 04J18A | 59.9 | 1.4 |
| 07P15A | 58.6 | 10.6 |
| 05L13A | 57.6 | 2.3 |
| 10B09A | 57.2 | 2.7 |
| 08M01A | 56.3 | 3.2 |
| 03P01Z | 55.9 | 5.5 |
| 09D16A | 55.6 | 2.2 |
| 02I07A | 55.2 | 13.3 |
| 01P14N | 54.8 | >100 |
| 04K01A | 54.6 | 2.0 |
| 05B02A | 54.2 | 3.5 |
| 04D18A | 54.1 | 12.5 |
| 09B13A | 53.7 | 24.5 |
| 03O15A | 53.6 | 12.0 |
| 04P20A | 52.5 | 5.9 |
| 01G24A | 52.3 | 15.3 |
| 07B06Z | 52.0 | 3.5 |
| 07P07A | 51.9 | 3.6 |
| 08M15Z | 51.7 | 3.7 |
| 03L07A | 51.6 | 1.4 |
| 01M02A | 51.1 | 2.3 |
| 14A15A | 50.9 | 2.9 |
| 04C03A | 49.2 | 4.8 |
| 02J12A | 48.8 | >200 |
| 06A06A | 47.5 | 8.7 |
| 07L12A | 46.8 | 3.7 |

Bispecific Antibody Production: Transient transfection of Ab897 constructs was performed in ExpiCHO-S™ (Thermo-Fisher Scientific) cells at 4 L scale. Ab897 comprises a HCl comprising the amino acid sequence of SEQ ID NO: 323, a LC comprising the amino acid sequence of SEQ ID NO: 324 and a HC2 comprising the amino acid sequence of SEQ ID NO: 315.

Three plasmid DNA were co-transfected for the expression of Ab897:

TABLE 9
Plasmid DNA for Expression

| | Concentration | |
|---|---|---|
| Plasmid | (ng/μL) | |
| LC (Bsyn-1033) IPA-CLDN6-51H1-Hum2_LC | 914.1 | ng/μL |
| HC1 Hole (Bsyn-1200) _CLDN6-51H1-Hu5- | 1304.4 | ng/μL |
| G1m17-hole Fc | | |
| HC2 Knob (Bsyn-1197) _Ab133scFv_G1m17-Kb Fc | 1025.4 | ng/μL |

The three plasmid DNAs were co-transfected in the ratio of 1×Knob, 2×Hole, 3×LC. Total 3.2 mg of DNA mixture was prepared to transfect 3.2 L of cells (1 mg DNA/L).

TABLE 10
Transient Transfection Details

| | |
|---|---|
| Host cell line | ExpiCHO-S ™ |
| Passage number @ Cells | 17 |
| used for transfection | |
| Transfection Cell count | ~6.0 × 10⁶ Cells/mL |
| Cell viability | 99% |
| Flask | 2 L Flask × 8 |
| | (400 mL per flask) |
| Media | ExpiCHO ™ Expression |
| | Medium |
| Transfection Batch Number | TFB030762 |

Cells were maintained at cell density of (0.25-6.0)×10⁶ cells/mL with viability of ≥95% for routine maintenance and incubated at 37° C. with 8% CO₂ at 100 RPM.

ExpiCHO Max Titer Transfection Protocol was followed as per the manufacturer's instructions. Twenty-four hours before transfection, ExpiCHO-S™ Cells with cell viability of >99%, were centrifuged at 800 RPM for five minutes and re-suspended at ˜2.5×10⁶ cells/mL into eight 2 L Erlenmeyer shake flasks, containing 400 mL of culture media. On the day of transfection, the cell density was maintained at ˜6.0×10⁶ cells/mL.

For 3200 mL transfection volume, 3.2 mg plasmid DNA (1.0 mg/L) mix was diluted in 160 mL of OptiPRO SFM Medium and filter sterilized. In another 160 mL of OptiPRO SFM medium, 10.24 mL of transfection reagent, ExpiFectamine CHO Reagent (3.2 times of DNA) was diluted. Diluted plasmid DNA and ExpiFectamine CHO Reagent were mixed together and incubated for 2 minutes at room temperature (RT). After the incubation, the complex was added immediately to the flask containing cells and gently swirled for homogeneous mixing. Flasks were then incubated at 37° C. with 8% CO₂ at 100 RPM.

At 18-20 hours of post-transfection on Day 01, cells were fed with the kit provided ExpiCHO Feed (16% of culture volume (512 mL)) and Enhancer (0.6% of culture volume (19.2 mL). The cells were shifted to a 32° C. incubator with 5% CO₂ for the remaining expression days. At day 05, the cells were fed with ExpiCHO feed (16% of culture volume (512 mL)). The transfected culture (˜4 L) was harvested on day 12 having 9.61×10⁶ Cell Count and 58% viability. Culture medium was centrifuged at 4700 RPM for 20 minutes. Supernatant harvest was filtered through a 0.22 μm membrane capsule filter and submitted for purification.

Affinity capture was performed with a MabSelectSure XK 16/20 Protein A-Affinity column. The column was regenerated with 0.5 M NaOH and after water wash, equilibrated with 1×PBS, pH 7.4. Harvest was loaded on the column and binding was carried out at a flow rate of 3 mL/min using AKTA Pure system (Unicorn version 7.6). The antibody captured column was washed with 1×PBS, pH 7.4 and eluted first with 50 mM Sodium Citrate pH 3.5 (eluate 1) followed by 100 mM Glycine pH 2.5 (eluate 2). Both eluates were neutralized by 2 M Tris pH 8.0. Elute from pH 3.5 was taken for further processing.

Eluate 1 volume was 215 mL. The sample was concentrated to 70 mL using Amicon® Ultra 15 mL filter (30 kDa NMWL) for gel filtration chromatography. No visual precipitation was observed during the concentration step.

A HiLoad 26/600 Superdex200 (320 mL) column was used with standard 3 mL/min flow rate. The column was cleaned with 0.5 M NaOH, followed by a water wash. The column was then equilibrated to 20 mM Histidine, 150 mM NaCl pH 6.0. 14 mL of prepared sample was injected into the column and eluted with elution buffer (20 mM Histidine, 150 mM NaCl pH 6.0) at 3 mL/min and 2 mL fractions were collected. The eluted fractions were analyzed by HPLC-based SEC purity and SDS-PAGE.

A 4-12% NuPAGE Precasted Gel (Invitrogen Cat. No. 13010771) was used with LDS 4× sample buffer (Invitrogen Cat. No. LC6065). Pre-stained protein standard from Pure Gene (Cat. No. PG-PMT0772) was used. The running buffer was NuPAGE MES SDS Running buffer-20× (Invitrogen Cat. No. NP000202). Antibody expression was observed in the harvest sample showing a protein band at the expected ˜125 kDa size (calculated molecular weight is 124,858.01 Da).

Additional SDS-PAGE analysis was conducted with protein samples prepared in 4× loading dye in non-reduced and reduced conditions and post incubation at 95° C. for 5 minutes. The results showed the migration of an intact molecule of ˜124 kDa in nonreducing conditions. In reducing condition, the Ab897 heavy and light chains were observed to run separately localizing as per their molecular weight.

A TSKgel G300SWXL column with a 1×PBS (pH 7.4)+0.2M L-arginine monohydrochloride mobile phase was used to analyze gel filtration column fractions on a Shimadzu Prominence-I LC-2030C-3D plus instrument at a detection wavelength of 280 nm. Biorad standards (Cat. No. 1511901) were used. The results showed that the Ab897 protein purity was >98.7% and confirmed the protein mass.

### Example 3: Assessment of the Binding Specificity of Ab897 for Human T Cells and CLDN6 Overexpressing Cell Lines

T-cells: The binding of an exemplary bispecific antibody that specifically binds CLDN6 and CD3 (Ab897) to Pan T cells from five human peripheral blood mononuclear cells (PBMC) donors was determined.

Isolation of Pan T cell: PBMCs were thawed and centrifuged for five minutes at 1,200 RPM at room temperature. Pan T cells were isolated using EasySep human T cell isolation kit as per manufacturer's instructions (StemCell Technologies; Cat no. #17951) and counted using a hemocytometer.

T-cells were re-suspended in L/D staining (Violet fluorescent dye in 1×DPBS; 1 μL/million/mL) and were incubated at 37° C. for 20 minutes. Post incubation, double the volume of FACS buffer (2% FBS in PBS) was added to stop the reaction, and suspension was centrifuged for five minutes at 1,200 RPM. Cells were then re-suspended in FACS buffer to obtain 2 million/mL final cell concentration.

Cells were seeded in a 96 well V bottom plate at a density of 0.1 million cells in 50 μL per well. Cell suspensions were made using FACS buffer.

Primary Antibody Dilution: A working concentration of 4000 nM (2×) followed by a 3-fold, 10-point DRC was prepared in media. 50 μL of antibody were added to 50 μL of cell suspension (final top concentration is 2000 nM followed by a 3-fold, 10-point DRC). Separate wells were kept for NBS (no bispecific sample).

The plates were incubated at 37° C. for 1 hour. Post incubation, cells were washed by adding 150 μL/well FACS buffer and pelleted at 1,400 RPM for five minutes. Washing was repeated twice with 200 μL/well of FACS buffer. The supernatant was decanted completely.

Secondary Antibody Addition: 100 μL/well of Alexa Fluor 647 anti-human IgG (1:100 dilution) was used to re-suspend the pellet. The plates were incubated for 30 minutes at 4° C. Post incubation, the cells were washed with 150 μL/well FACS buffer and centrifuged at 1,400 RPM for five minutes. Washing was repeated twice with 200 μL/well 1×DPBS. Supernatant was decanted completely.

The pellets were re-suspended in 60 μL of FACS buffer per well. Data from the plates were acquired using a Novocyte flow cytometer and the data were analyzed using NovoExpress software.

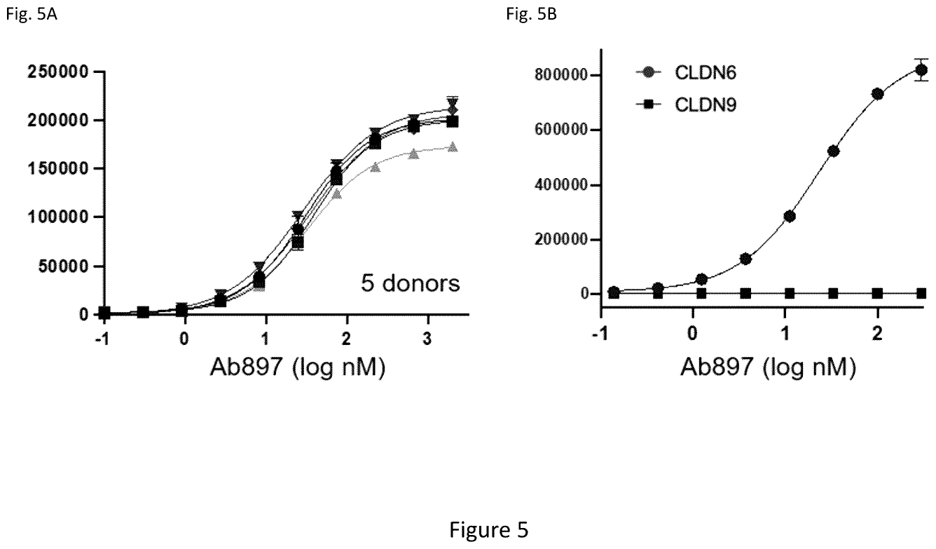

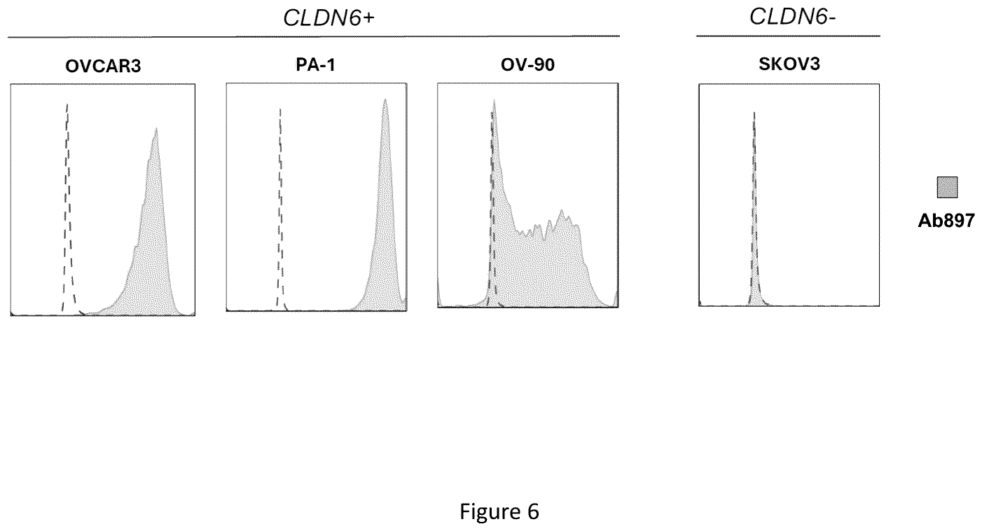

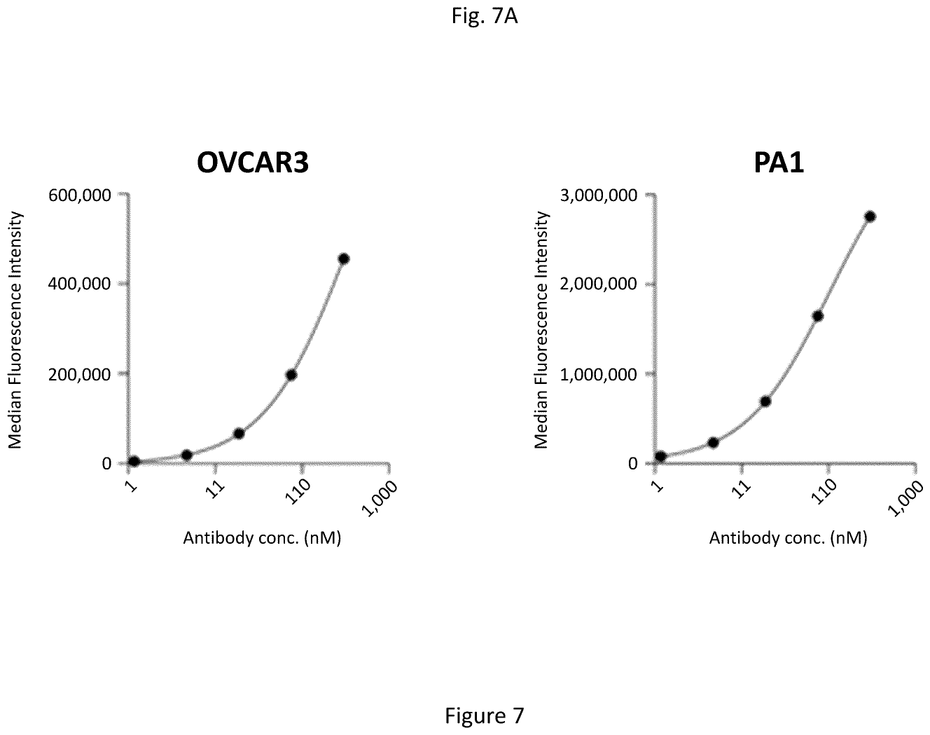

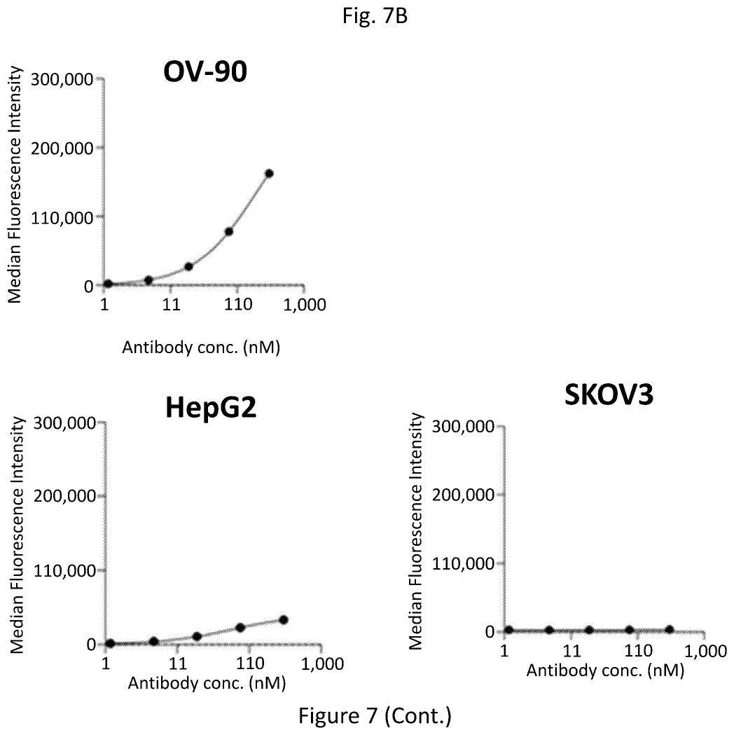

Binding Assay Calculations: The median fluorescence intensity (MFI) of each sample was obtained from the analyzed flow cytometry data file. The average MFI value of the secondary alone control wells (labelled as NBS) was calculated. The control average MFI value was subtracted from each sample MFI to remove background. The background-subtracted MFI value for each sample was plotted using GraphPad prism and fitted with a 4-parameter non-linear regression with log transformed X-axis. FIG. 5A shows flow cytometry plots showing median fluorescence intensity (MFI) values of Ab897b binding to Pan T cells from 5 donors. The results showed Ab897 binds to Pan T cells in a similar dose-dependent manner in all 5 donors with an average KD of 33 nM.

CHO Cells: The binding of Ab897 to CHO—K-CLDN6 and CHO—K-CLDN9 over expression cell lines was determined.

Assay Protocol: Cells were dissociated using 0.25% trypsin and the cell suspension was collected. Cells were centrifuged at 1,200 RPM for five minutes at room temperature, re-suspended in complete media (F12K+10% FBS) and counted. The cells were re-suspended in prepared L/D staining (violet fluorescent dye in 1×DPBS; 1 μL/million/mL) and were incubated at 37° C. for 15-20 minutes. Post incubation the cells were topped up with double the volume of FACS buffer (2% FBS in 1×PBS) to stop the reaction and centrifuged for five minutes at 1,200 RPM. The cells were re-suspended in FACS buffer. Cells were seeded in a 96 well V bottom plate at a density of 0.1 million cells in 50 μL per well. Cells were dissociated using 0.25% trypsin and the cell suspension was collected. Cells were centrifuged at 1200 RPM for five minutes at room temperature, re-suspended in complete media (F12K+10% FBS) and counted. The cells were re-suspended in prepared L/D staining (violet fluorescent dye in 1×DPBS; 1 μL/million/mL) and were incubated at 37° C. for 15-20 minutes. Post incubation the cells were topped up with double the volume of FACS buffer (2% FBS in 1×PBS) to stop the reaction and centrifuged for five minutes at 1,200 RPM. The cells were re-suspended in FACS buffer. Cells were seeded in a 96 well V bottom plate at a density of 0.1 million cells in 50 μL per well.

Primary Antibody Dilution: A working concentration of 600 nM (2×) followed by a 4-fold, 5-point DRC was prepared in media. 50 μL of antibody were added to 50 μL of cell suspension (final top concentration is 300 nM, followed by a 4-fold, 5-point DRC). Separate wells were kept for NBS (0 nM antibody). The plates were incubated at 37° C. for one hour. Post incubation the cells were washed by adding 150 μL/well FACS buffer and pelleted at 1,400 RPM for five minutes. Washing was repeated twice with 200 μL/well of FACS buffer. The supernatant was removed completely after the second wash.

Secondary Antibody Addition: 100 μL/well of Alexa Fluor 647 anti-human IgG (1:300 dilution in FACS buffer) was used to re-suspend the pellets. The plates were incubated for 30 minutes at 4° C. Post incubation, the cells were washed with 150 μL/well FACS buffer and centrifuged at 1,400 RPM for five minutes. Washing was repeated twice with 200 μL/well 1×DPBS. The supernatant was removed completely after the second wash. The pellets were re-suspended in 60 μL of FACS buffer per well. Data from the plates were acquired on a Novocyte flow cytometer and data were analyzed using NovoExpress software.

Binding Assay Calculation: The median fluorescence intensity (MFI) of each sample was obtained from the analyzed flow cytometry data file. The average MFI value of the secondary alone control wells (labelled as NBS or no bispecific sample) was calculated. The control average MFI value was subtracted from each sample MFI to remove background. The background-subtracted MFI value for each sample was plotted using GraphPad prism and fitted with a 4-parameter non-linear regression with log transformed X-axis. FIG. 5B shows flow cytometry plots showing median fluorescence intensity (MFI) values of Ab897 binding to CHO-CLDN6 and CHO-CLDN9 cells. The results showed dose-dependent binding of Ab897 on CHO-CLDN6 cell, with a K_{D} of approximately 23 nM, but does not show any binding on CHO-CLDN9 cells up to a concentration of 300 nM.

### Example 5: Assessment of the Binding Profile of Ab897 Using a Human Plasma Membrane Protein Cell Array and Flow Cytometry

The Retrogenix Cell Microarray Technology (Charles River, Derbyshire, UK) was used to screen for specific off-target binding interactions of an exemplary bispecific antibody that specifically binds CLDN6 and CD3 (Ab897) by evaluating levels of binding of Ab897. A control human IgG antibody targeting CD3 (Ab946) was used as a positive control.

The study was divided into 4 phases:
Phase 1. Pre-screens to determine the level of background binding of Ab897 to fixed untransfected HEK293 cells and cells over-expressing CD3E (plasma membrane and tethered secreted forms), CD3D+CD3E, CD3G+CD3E or CLDN6. These data were used to assess the suitability and optimal concentration for onward screening.
Phase 2. Library screen. Ab897 was screened for binding against fixed HEK293 cells over-expressing 6, 105 individual full-length human plasma membrane proteins, secreted and cell surface-tethered human secreted proteins, as well as a further 400 human heterodimers. This identified library interactions.
Phase 3. Confirmation screens. All library interactions were re-expressed, and probed with Ab897 or control, to determine which interactions, if any, were repeatable and specific to Ab897. This was performed on both fixed and live cells.
Phase 4. Flow cytometry-based follow-on study to investigate the identified Ab897b-specific interactions further on live cells.

### Methodology

### Pre-Screen:

1. Slides were spotted with expression vectors encoding both ZsGreen1 and human CD3E (plasma membrane and tethered secreted forms), CD3D+CD3E, CD3G+CD3E, CLDN6, CD20 or EGFR, and used to reverse-transfect HEK293 cells.
2. 2, 5 or 20 μg/mL of Ab897, 1 μg/mL of Rituximab biosimilar or PBS only was added to the above cells/slides after fixation.
3. Binding to target-expressing cells and untransfected cells was assessed using an AlexaFluor 647 labelled anti-human IgG Fc (AF647 anti-hIgG Fc) detection antibody, followed by fluorescence imaging.
4. This detection antibody has been validated previously for use in the Retrogenix Cell Microarray Technology system for detecting human IgGs and human Fc fusion proteins.

Library screen: For Library screening, 6,105 expression vectors, encoding both ZsGreen1 and a full-length human plasma membrane protein, secreted or a cell surface-tethered human secreted protein, plus a further 400 human heterodimers were individually arrayed in duplicate across cell microarray slides (‘slide-sets’). The test human proteins included CLDN1, CLDN2, CLDN3, CLDN4, CLDN5, CLDN6, CLDN7, CLDN8, CLDN9, CLDN10, CLDN11, CLDN12, CLDN13, CLDN14, CLDN15. CLDN16, CLDN17, CLDN18, CLDN19, CLDN20, CLDN22, CLDN23, CLDN24, CLDN25 and CLDN34.

An expression vector (pIRES-hEGFR-IRES-ZsGreen1) was spotted in quadruplicate on every slide and was used to ensure that a minimal threshold of transfection efficiency had been achieved or exceeded on every slide. Human HEK293 cells were used for reverse transfection/expression. Ab897 was added to each slide after cell fixation, giving a final concentration of 2 μg/mL.

Detection of binding was performed using the same fluorescent secondary antibody as used in the Pre-screen (AF647 anti-hIgG Fc). Ab897 was screened against 2 replicate slide-sets. Fluorescent images were analyzed and quantitated (for transfection) using ImageQuant software (GE healthcare, Version 8.2).

A protein interaction was defined as a duplicate spot showing a raised signal compared to background levels. This was achieved by visual inspection using the ImageQuant software. Interactions were classified as ‘strong, medium, weak or very weak’, depending on the intensity of the duplicate spots. A significant interaction was defined as a signal of weak intensity or greater and did not refer to a statistical significance.

Confirmation screen: Vectors encoding all interactions identified in the Library screen, plus control vectors encoding CD20 (positive control) and EGFR (transfection and negative control), were arrayed and expressed in HEK293 cells on new slides. Confirmation screen slides and analyses were carried out as for the Library screen either after cell fixation (n=2) or in the absence of fixation (n=1). Slides were treated with 2 μg/mL of Ab897b, 2 μg/mL of Ab946 (positive control), 1 μg/mL of Rituximab biosimilar (Charles River positive control) or no test molecule (secondary only; negative control). Binding to target-expressing cells and untransfected cells was again assessed by fluorescence imaging.

Follow-on flow cytometry validation study: Human HEK293 cells were transfected with expression vectors encoding ZsGreen1 only, or ZsGreen1 and CLDN6, CD3E (plasma membrane and tethered secreted forms), SIRPA (isoform 2 and isoform 4) or CD20 (assay control). Live cell transfectants were incubated with 2 μg/mL Ab897, 1 μg/mL Rituximab biosimilar (Charles River positive control) or assay buffer only, as indicated. Cells were washed and incubated with the same AF647 anti-hIgG Fc detection antibody as used in the cell microarray screens. Cells were again washed and analyzed by flow cytometry. A 7AAD live/dead dye was used to exclude dead cells, and ZsGreen+ (transfected) cells were selected for analysis.

### Results

Pre-screen: Ab897 showed low to high levels of background binding to fixed untransfected HEK293 cells when tested at 2, 5, and 20 μg/mL. Binding to over-expressed CLDN6, CD3D+CD3E, and CD3G+CD3E, the primary targets, were observed at all 3 concentrations tested. Based upon these data, Ab897 was fully profiled at a final concentration of 2 μg/mL on fixed cells.

Library screen: After screening 2 μg/mL of Ab897 for binding against fixed HEK293 cells expressing 6105 individual full-length human plasma membrane proteins, secreted and cell surface-tethered human secreted proteins, as well as a further 400 human heterodimers 15 library interactions were identified altogether. Spot intensities ranged from very weak to medium/strong.

Confirmation screens: In subsequent Confirmation screens, all 15 library interactions, vectors encoding CD3E (plasma membrane and tethered secreted forms) and 2 control receptors (CD20 and EGFR) were over-expressed in HEK293 cells. Rituximab biosimilar showed a strong intensity interaction with over-expressed CD20 on fixed cell microarrays, validating the incubation conditions and detection system.

On fixed cell microarrays, all of the library interactions were reproducibly observed with Ab897 in the Confirmation screen. Ten of the 15 library interactions were determined to be non-specific binding as at least 1 of the control treatments (Rituximab biosimilar and/or no primary test molecule) also displayed binding. These 10 interactions included Fc gamma receptor, binding occurring through Fc domain of antibodies.

Five specific interactions were identified. Four of those were interactions with the primary targets CLDN6, and CD3E (also in a heterodimer with either CD3D or CD3G. Specific interactions using this method were detected with SIPRA (isoforms 2 and 4), and were investigated further.

Flow cytometry follow up results (live cells): In order to investigate the identified interactions of Ab897 further, CLDN6, CD3E (plasma membrane and tethered secreted forms), SIRPA (isoform 2 and isoform 4) and CD20 as a control were over-expressed in HEK293 cells. Live cell transfectants, including ZsGreen1-only transfected cells, were incubated with 2 μg/mL Ab897, 1 μg/mL Rituximab biosimilar (Charles River positive control) or assay buffer alone. Interactions were then investigated by flow cytometry.

Ab897 showed an interaction measured as strong fluorescence intensity, with its primary target, CLDN6. Ab897 showed no significant interaction with CD3E (plasma membrane or tethered secreted form) or SIRPA (isoform 2 or isoform 4). The lack of interaction with CD3E is likely to be caused by CD3 being unstable and undetectable at the cell membrane in living cells in the absence of fixation.

The binding pattern of Ab897 to heterodimers CD3E+CD3D and CD3E+CD3G suggested that the binding epitope of Ab892 is contained within CD3E, as the common element of both heterodimers. Binding to CD3E alone was not observed in fixed cells, which has also been observed with other CD3E binders. Although not stabilized at the cell surface, the process of cell fixation makes the cells slightly porous and allows some binding to intracellular CD3E. The fact that no binding was observed with Ab897 suggests that the CD3E epitope is unmasked by a conformational change in CD3E upon binding to CD3D or CD3G subunits and this hypothesis is supported by published data (Salmerón et al., A conformational epitope expressed upon association of CD3-epsilon with either CD3-delta or CD3-gamma is the main target for recognition by anti-CD3 monoclonal antibodies. J. Immunol. 1991; 147:3047-52.)

In conclusion, these results indicate high specificity of Ab897b for its primary targets, CLDN6 and CD3E. Summary of the evaluated specific interactions are shown in Table 11.

TABLE 11
Specific Interactions Identified in Library and Confirmation Screens (≥weak)

| | | Confirmation | | |
|---|---|---|---|---|
| | Library Screen | Screen | | |
| | (fixed cells) | (fixed cells) | Confirmation | Confirmation |
| | (two replicate | (two replicate | Screen | Screen |
| Gene ID (Uniprot ID) | experiments) | experiments) | (live cells) | (flow cytometry) |
| SIRPA (P78324-4) | v. weak | weak | NI | NI |
| SIRPA (P78324-2) | v. weak | weak | NI | NI |
| CLDN6 (P56747-1) | medium | strong | medium | strong |
| CD3D + CD3E | medium/strong | strong | v. weak | NT |
| (P04234-1 + P07766-1) | | | | |
| CD3G + CD3E | medium/strong | strong | NI | NT |
| P09693-1 + P07766-1) | | | | |

NI: no interaction detected;
NT: not tested

### Example 6: Assessment of the Binding Specificity of Ab897 for CLDN6-Expressing Human Tumor Cell Lines and the CLDN6-Negative Human Tumor Cell Line SKOV3

The binding of an exemplary bispecific antibody that specifically binds CLDN6 and CD3 (Ab897) to OVCAR3, PA1, OV90, HepG2 and SKOV3 cell lines was determined to examine endogenous expression of, and binding to, CLDN6. Media composition for the cell lines tested is shown in Table 12.

TABLE 12
Cell Culture Media

| Cell | |
|---|---|
| Line | Media Composition |
| OVCAR3 | RPMI (ATCC Modification, Gibco Cat. No. A10491-01) + |
| | 20% FBS + 0.01 mg/mL insulin |
| SKOV3 | McCoy's 5A (ATCC Cat. No. 30-2007) + 10% FBS |
| PA1 | DMEM (Gibco Cat. No. 11995-040) + 10% FBS |
| OV90 | 1:1 mixture of MCDB 105 Medium (Sigma-Aldrich Cat. |
| | No. 117-500) containing a final concentration of |
| | 1.5 g/L sodium bicarbonate and Medium 199 (Sigma-Aldrich |
| | Cat. No. M4530) containing a final concentration of |
| | 2.2 g/L sodium bicarbonate + 15% FBS |
| HepG2 | EMEM (ATCC Cat. No. 30-2003) + 10% FBS |

### Assay Protocol

Harvesting of Target Cells: Cells were washed with DPBS twice (5 mL/flask) and dissociated using 5 mL/flask of 0.25% trypsin. The flasks were incubated at 37° C. for 3 minutes and neutralized using 10 mL of respective complete media. Cell suspensions were centrifuged at 1,200 RPM for five minutes at RT. Supernatants were discarded after centrifugation and the pellets were re-suspended in 1 mL of respective complete media. 10 μL aliquots were taken for cell counting using Neubauer chamber hemocytometer and trypan blue method.

Live Dead Staining and Seeding of Target Cells: The cells were centrifuged at 1,200 RPM for five minutes at RT. Pellets were re-suspended in 3 mL of prepared L/D staining (Violet fluorescent dye in 1×DPBS; 1 μL/million/mL). Resuspended cells in Falcon tubes were incubated at RT for 20 minutes. Post incubation, 6 mL of 2% FACS buffer (2% FBS in PBS) was added to stop the reaction. Cells suspensions were centrifuged for five minutes at 1,200 RPM. The cells were re-suspended in 1.5 mL of 2% FACS buffer. Cells were seeded in a 96 well V-bottom plate at a density of 0.1 million cells in 50 μL of 2% FACS buffer per well.

Primary Antibody Dilution: Working concentration of 600 nM (2×) followed by a 4-fold, 5-point DRC was prepared in RMPI 1640 media containing 10% FBS. 50 μL of each antibody were added to 50 μL of cell suspension in 96 well V-bottom plate (final top concentration is 300 nM followed by a 4-fold, 5-point DRC). Separate wells were kept for NBS (0 nM antibody). The plates were incubated at 37° C. for one hour. Post incubation, the cells were washed by adding 150 μL FACS buffer/well and pelleted at 1,400 RPM for five

[symbol]

The cytotoxicity of an exemplary bispecific antibody that specifically binds CLDN6 and CD3 (Ab897) on the PA1 (CLDN6⁺) ovarian tumor cell line at 72 and 96 hours in cocultures of the PA1 tumor cell line with 5 independent human Pan T-cell donors at a 3:1 E:T ratio was determined.

### Assay Protocol

NLG transduction and seeding of PA1-NLG cells: A PA1-NLG stable cell line was utilized for these studies (stably transduced with Incucyte Nuclight Green). Cells grown in flasks were trypsinized and spun at 1,200 RPM for five minutes at RT. The cell pellet was resuspended in EMEM media+10% FBS and cells were counted by hemocytometer. 96 well flat bottom plates were seeded as per the plate map with 3,000 cells/100 μL/well PA1-NLG cells. Seeded plates were incubated overnight at 37° C., 5% CO₂.

Addition of antibodies and effector cells: PBMCs from 5 donors were thawed and Pan T cells were isolated from the PBMCs using StemCell Pan T cell isolation kit as per manufacturer's instructions. Purified T cells were counted on a hemocytometer. The Pan T cells were resuspended in RPMI media+10% FBS and added to the PA1-NLG cells at a 3:1 E:T ratio (9,000 T cells/80 μL/well). In order to prepare antibody dilutions, a 10× (300 nM) intermediate stock was prepared using complete media. The 300 nM stock was further serially diluted 4-fold, 10-point DRC to prepare all intermediate stocks. From these respective 10× intermediate stocks, 20 μL was added to each well as per plate map that already contains 180 μL of cell culture media with effector and target cells to obtain final top concentration of 30 nM with a 4-fold, 10-point DRC. Ab897 (full DRC) with target cells referred to as Ab+T; 0 nM Ab897 with T cells+ target cells referred to E+T and 0 nM Ab897 with target cells alone referred to T were used as controls.

Incucyte set up: After setting up the assay, plates were placed in the Incucyte chamber at 37° C. with 5% CO₂. The plates were left undisturbed in the Incucyte chamber for 30 minutes to stabilize, and any condensation that formed was subsequently removed from the plate. Using the Incucyte S3 software, the plates were set to scan each well at every 6 or 12 hours, till the end point as per assay requirement. The time-lapse images were taken with phase and respective fluorescent channel (Green or Red) from every well and then analyzed using the same software. The fluorescent intensity captured as “Integrated Intensity Per Well (GCU×μm²/Well)” was used further to calculate the % cytotoxicity.

Cytotoxicity assay calculation: The cytotoxicity file from the Incucyte was analyzed and the raw data based on fluorescence intensity was exported. The fluorescence intensity value from the control well (E+T alone, 0 nM Ab897) was set to 100%. Percentage killing for samples with Ab897 was determined compared to E+ alone control. The relative percentages were plotted using GraphPad Prism using the formula: 100−(sample/avg control)×100.

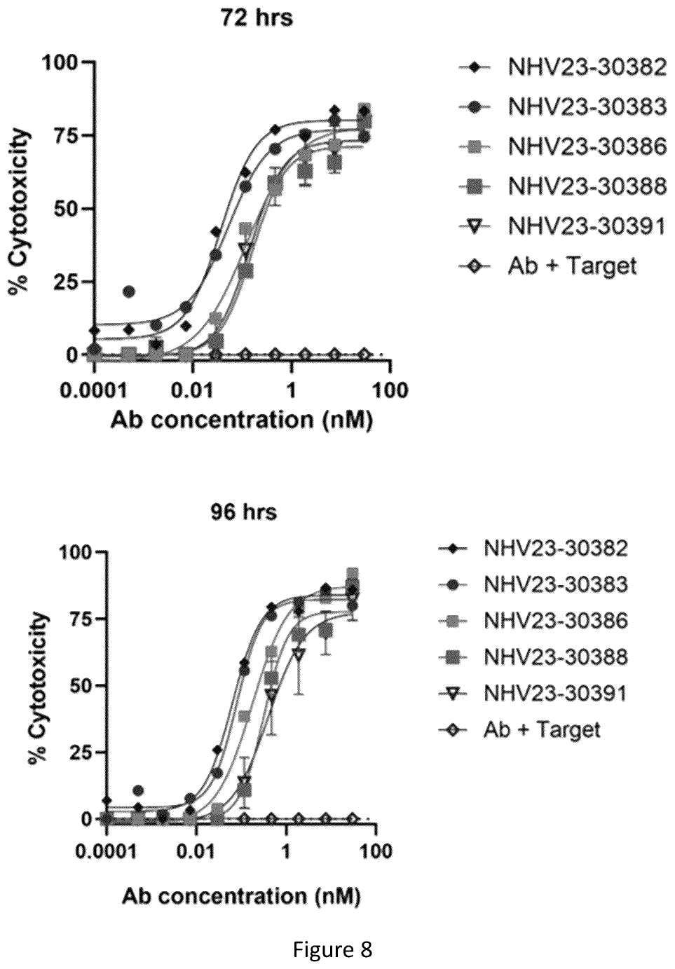

FIG. 8 plots show % lysis at 72 and 96 hr timepoints in an Incucyte based cytotoxicity assay of Ab897b on PA1-NLG cells at 3:1 E:T ratio. Target only control (no effector cells) did not show any cytotoxicity. All data points are in duplicates, mean±SD plotted. All negative lysis values were constrained to 0 for the purpose of data plotting in a non-linear regression curve.

After up to 96 hours of human Pan-T cell coculture with tumor cell lines at a fixed E:T ratio of 3:1, Ab897 shows sub-nM dose dependent cytotoxicity across all 5 human T cell donors on the PA1 tumor cell line. Maximal cytotoxicity (˜75-80%) was observed at the 3:1 ratio at both the 72- and 96-hour time points in all donors. There was clear donor dependent variability in the potency at both timepoints.

### Example 8: T-Cell Mediated Cytotoxicity of Ab897 on CLDN6+ Tumor Cell Line OV90

The cytotoxicity of an exemplary bispecific antibody that specifically binds CLDN6 and CD3 (Ab897) on the OV90 (CLDN6⁺) ovarian tumor cell line at 72 and 96 hours in cocultures of the OV90 tumor cell line with 5 independent human Pan T-cell donors at a 3:1 E:T ratio was determined.

### Assay Protocol

NLG transduction and seeding of OV90-NLG cells: OV90-NLG stable cell line was utilized for these studies (stably transduced with Incucyte Nuclight Green). Cells grown in flasks were trypsinized and spun at 1,200 RPM for five minutes at RT. The cell pellet was resuspended in complete medium (1:1 mixture of MCDB 105 media with 1.5 g/L sodium bicarbonate and Medium 199 with 2.2 g/L sodium bicarbonate+15% FBS) and cells were counted using a hemocytometer. 96 well flat bottom plates were seeded as per the plate map with 7,500 OV90-NLG cells/100 μL/well. Seeded plates were incubated overnight at 37° C., 5% CO₂.

Addition of antibodies and effector cells: PBMCs from 5 donors were thawed and Pan-T cells were isolated from the PBMCs using StemCell kit as per manufacturer's instructions. T cells were counted on a hemocytometer. The Pan T cells were resuspended in complete medium and added to the OV90-NLG cells at an E:T ratio of 3:1 (22,500 T cells/50 μL/well). In order to prepare antibody dilutions, a 10× (300 nM) intermediate stock was prepared using complete media. The 300 nM stock was further serially diluted 4-fold, 10-point DRC to prepare all intermediate stocks. From these respective 10× intermediate stocks, 20 μL was added to each well as per plate map that already contains 180 μL of cell culture media with effector and target cells to obtain final top concentration of 30 nM with a 4-fold, 10-point DRC. Ab897 (full DRC) with target cells referred to as Ab+T; 0 nM Ab897b with T cells+ target cells referred to E+T and 0 nM Ab897b with target cells alone referred to T were used as controls.

Incucyte set up: After setting up the assay, plates were placed in the Incucyte chamber at 37° C. with 5% CO₂. The plates were left undisturbed in the Incucyte chamber for 30 minutes to stabilize, and any condensation that formed was subsequently removed from the plate. Using the Incucyte S3 software, the plates were set to scan each well at every 6 or 12 hours, till the end point as per assay requirement. The time-lapse images were taken with phase and respective fluorescent channel (Green or Red) from every well and then analyzed using the same software. The fluorescent intensity captured as “Integrated Intensity Per Well (GCU×μm²/Well)” was used further to calculate the % cytotoxicity.

Cytotoxicity assay calculation: The cytotoxicity file from the Incucyte was analyzed and the raw data based on fluorescence intensity was exported. The fluorescence intensity value from the control well (E+T alone, 0 nM Ab897) was set to 100%. Percentage killing for samples with Ab897 was determined compared to E+ alone control. The relative percentages were plotted using GraphPad Prism using the formula: 100−(sample/avg control)×100.

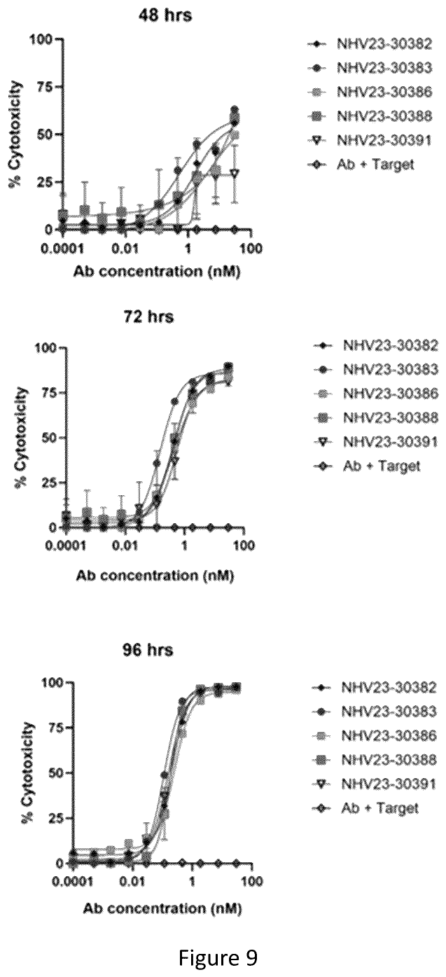

FIG. 9 plots show % lysis at 48-, 72- and 96-hour timepoints in an Incucyte based cytotoxicity assay of Ab897 on OV90-NLG cells at 3:1 E:T ratio. Target only control (no effector cells) did not show any cytotoxicity. All data points are in duplicates, mean±SD plotted. All negative lysis values were constrained to 0 for the purpose of data plotting in a non-linear regression curve.

After up to 96 hours of human Pan-T cell coculture with tumor cell lines at a fixed E:T ratio of 3:1, Ab897 shows sub-nM dose dependent cytotoxicity across all 5 human T cell donors on the OV90 tumor cell line with peak potency obtained at 96 hours. Maximal cytotoxicity (˜95%) was observed at the 3:1 ratio at the 96-hour time point only in all donors. There was donor dependent variability in the potency and maximal cytotoxicity at early time points (48 hours) which resolved by 96 hours in all donors. Ab897 showed dose dependent cytotoxicity in OV90 cells at 3:1 E:T ratio reaching a maximum of ˜95% killing.

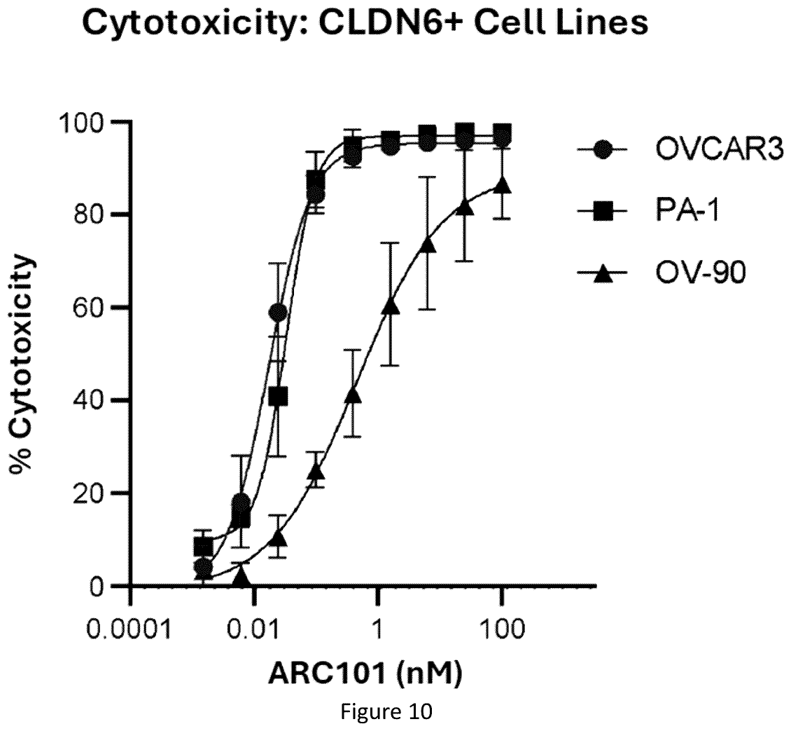

Comparative cytotoxicity against CLDN6⁺OVCAR3, PA-1, and OV-90 cell lines are shown in FIG. 10.

### Example 9: T-Cell Mediated Cytotoxicity of Ab897 on CLDN6+ Tumor Cell Line HepG2

The cytotoxicity of an exemplary bispecific antibody that specifically binds CLDN6 and CD3 (Ab897) on the HepG2 (CLDN6+) liver cancer cell line at 72 and 96 hours in cocultures of the HepG2 cell line with 5 independent human Pan T-cell donors at a 10:1 E:T ratio was determined.

### Assay Protocol

NLG transduction and seeding of HepG2-NLG cells: HepG2-NLG stable cell line was utilized for these studies (stably transduced with Incucyte Nuclight Green). Cells grown in flasks were trypsinized and spun at 1,200 RPM for five minutes at RT. The cell pellet was resuspended in complete medium (EMEM+10% FBS) and cells were counted using a hemocytometer. 96 well flat bottom plates were seeded with 3,000 HepG2-NLG cells/100 μL/well. Seeded plates were incubated overnight at 37° C., 5% CO₂.

Addition of antibodies and effector cells: PBMCs from 5 donors were thawed and Pan-T cells were isolated from the PBMCs using StemCell kit as per manufacturer's instructions. T cells were counted on a hemocytometer. The Pan T cells were resuspended in complete medium and added to the HepG2-NLG cells at an E:T ratio of 10:1 (30,000 T cells/50 μL/well). In order to prepare antibody dilutions, a 10× (300 nM) intermediate stock was prepared using complete media. The 300 nM stock was further serially diluted 4-fold, 10-point DRC to prepare all intermediate stocks. From these respective 10× intermediate stocks, 20 μL was added to each well that already contained 180 μL of cell culture media with effector and target cells to obtain final top concentration of 30 nM with a 4-fold, 10-point DRC. Ab897 (full DRC) with target cells referred to as Ab+T; 0 nM Ab897b with T cells+ target cells referred to E+T and 0 nM Ab897 with target cells alone referred to T were used as controls.

Incucyte set up: After setting up the assay, plates were placed in the Incucyte chamber at 37° C. with 5% CO₂. The plates were left undisturbed in the Incucyte chamber for 30 minutes to stabilize, and any condensation that formed was subsequently removed from the plate. Using the Incucyte S3 software, the plates were set to scan each well at every 6 or 12 hours, till the end point as per assay requirement. The time-lapse images were taken with phase and respective fluorescent channel (Green or Red) from every well and then analyzed using the same software. The fluorescent intensity captured as “Integrated Intensity Per Well (GCU×μm²/Well)” was used further to calculate the % cytotoxicity.

Cytotoxicity assay calculation: The cytotoxicity file from the Incucyte was analyzed and the raw data based on fluorescence intensity was exported. The fluorescence intensity value from the control well (E+T alone, 0 nM Ab897) was set to 100%. Percentage killing for samples with Ab897 was determined compared to E+ alone control. The relative percentages were plotted using GraphPad Prism using the formula: 100−(sample/avg control)×100.

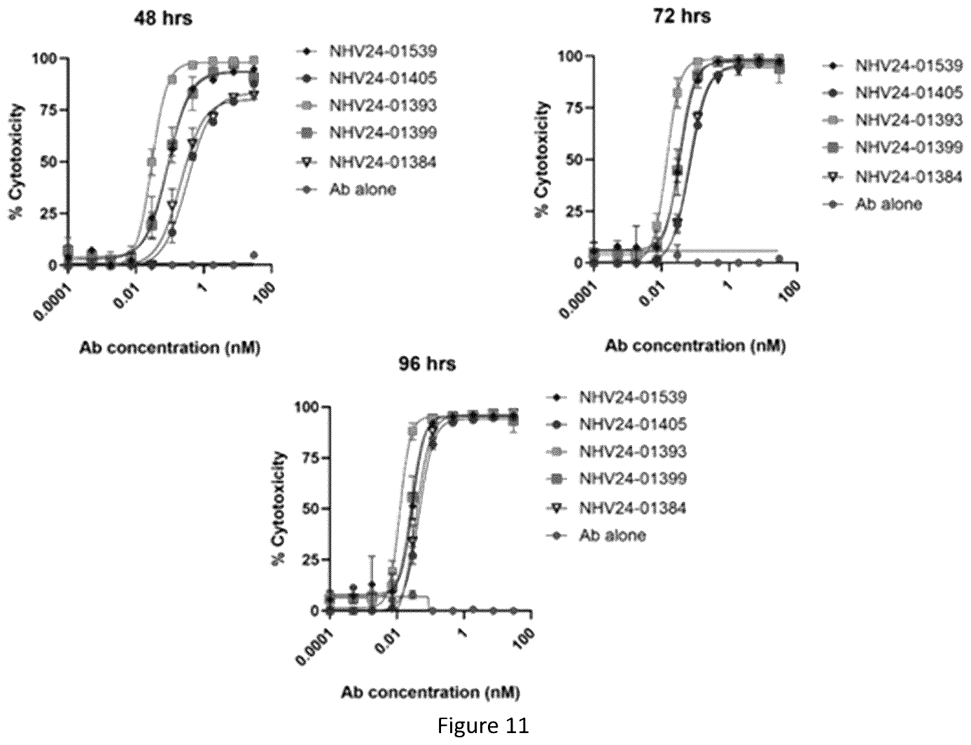

FIG. 11 plots show % lysis at 48-, 72- and 96-hour timepoints in an Incucyte based cytotoxicity assay of Ab897 on HepG2-NLG cells at 10:1 E:T ratio. Target only control (no effector cells) did not show any cytotoxicity. All data points are in duplicates, mean±SD plotted. All negative lysis values were constrained to 0 for the purpose of data plotting in a non-linear regression curve.

After up to 96 hours of human Pan-T cell coculture with tumor cell lines at a fixed E:T ratio of 10:1, Ab897 shows sub-nM dose dependent cytotoxicity across all 5 human T cell donors on the HepG2 tumor cell line with peak potency obtained by 72 hours. Maximal cytotoxicity (˜95%) was observed as early as 48 hours in the majority of donors. There was donor dependent variability in the potency and maximal cytotoxicity at early time points (48 hours) which resolved by 72 hours in all donors.

### Example 10: T-Cell Mediated Cytotoxicity of Ab897 on CLDN6+ Tumor Cell Line OVCAR-3 and CLDN6-Tumor Cell Line SKOV3

The cytotoxicity of an exemplary bispecific antibody that specifically binds CLDN6 and CD3 (Ab897) on the OVCAR3 (CLDN6+) and SKOV3 (CLDN6−) ovarian tumor cell lines at various E:T ratios (3:1, 1:1, 1:3) at 72 hours in cocultures of the cell lines with 5 independent human Pan T-cell donors was determined.

### Assay Protocol

Seeding of OVCAR3-NLG and SKOV3-NLR cells: OVCAR3-NLG stable cell line was utilized for these studies (stably transduced with Incucyte Nuclight Green). Cells grown in flasks were trypsinized and spun at 1,200 RPM for five minutes at RT. The cell pellet was resuspended in RPMI media+20% FBS and cells were counted using a hemocytometer. 96 well flat bottom plates were seeded with 5,000 OVCAR3-NLG cells/100 μL/well. Seeded plates were incubated overnight at 37° C., 5% CO₂.

SKOV3-NLR stable cell line was utilized for these studies (stably transduced with Incucyte Nuclight Red). Cells grown in flasks were trypsinized and spun at 1,200 RPM for five minutes at RT. The cell pellet was resuspended in McCoy's 5A media+10% FBS and count was obtained using a hemocytometer. 96-well flat bottom view plates were seeded with 2,500 SKOV3-NLR cells/100 μl/well. Seeded plates were incubated overnight at 37° C., 5% CO₂.

Addition of antibodies and effector cells: PBMCs from 5 donors were thawed and Pan-T cells were isolated from the PBMCs using StemCell kit as per manufacturer's instructions. T cells were counted on a hemocytometer. The Pan T cells were resuspended in RPMI media+10% FBS. The E:T ratios used were 3:1, 1:1 and 1:3. For OVCAR3 cells, 15,000 T cells/50 μL/well were added for 3:1 E:T, 5,000 T cells/50 μL/well were added for 1:1 E:T, and 1,650 T cells/50 μL/well were added for 1:3 E:T ratio. For SKOV3 cells, 7,500 T cells/50 μL/well were added for 3:1 E:T, 2,500 T cells/50 μL/well were added for 1:1 E:T, and 850 T cells/50 μL/well were added for 1:3 E:T ratio.

Antibody dilutions were prepared in RPMI+10% FBS, where 4× antibody stock was made and 50 μL of this was added to a well to give the 1× final concentration. Ab897 was used at a starting concentration of 30 nM and diluted 4-fold (9 dilutions) for a total of 10-points. Control wells (0 nM Ab897) were included for each E+T ratio and target cell alone conditions. 50 μL of 4× dilutions of antibody+50 μL cell suspension were added. For the control wells, only media was added instead of antibody.

Incucyte set up: After setting up the assay, plates were placed in the Incucyte chamber at 37° C. with 5% CO₂. The plates were left undisturbed in the Incucyte chamber for 30 minutes to stabilize, and any condensation that formed was subsequently removed from the plate. Using the Incucyte S3 software, plates were set to scan each well every 6 hours up to 24 hours and every 12 hours thereafter, until the end point was reached. The time-lapse images were taken with phase and respective fluorescent channel (Green or Red) from every well and then analyzed using the same software. The fluorescent intensity captured as “Integrated Intensity Per Well (GCU×μm²/Well)” was used further to calculate the % cytotoxicity.

Cytotoxicity assay calculation: The cytotoxicity file from the Incucyte was analyzed and the raw data based on fluorescence intensity was exported. The fluorescence intensity value from the control well (E+T alone, 0 nM Ab897) was set to 100%. Percentage killing for samples with Ab897 was determined compared to E+T alone control. The relative percentages were plotted using GraphPad Prism using the formula: 100−(sample/avg control)×100.

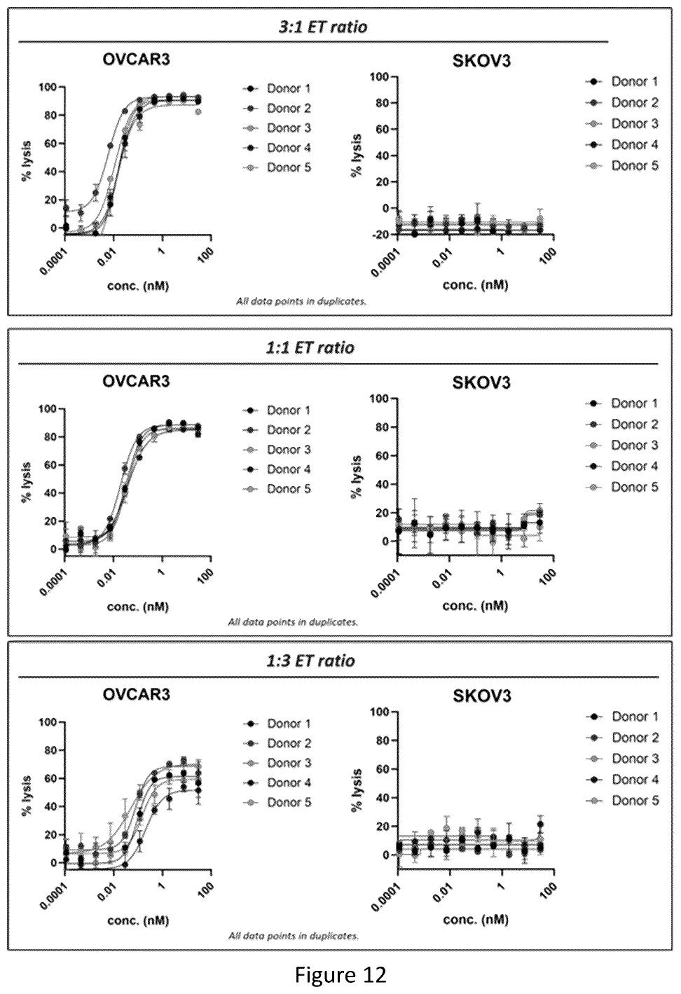

FIG. 12 plots show % lysis at the 72-hour timepoint in an Incucyte based cytotoxicity assay of Ab897 on OVCAR3 and SKOV3 cell lines at different E:T ratios. All data points are in duplicates, mean±SD plotted.

At 72 hours of human Pan-T cell coculture with tumor cell lines, Ab897b shows sub-nM dose dependent cytotoxicity across all 5 human T cell donors on OVCAR3 (CLDN6+) cells at all tested E:T ratios (3:1, 1:1 and 1:3). Maximal cytotoxicity (˜90-95%) was observed at 3:1 and 1:1 E:T ratios, with a lower maximal cytotoxicity observed (˜50-80%) at 1:3 E:T ratio. Ab897 demonstrates no/negligible cytotoxicity in all 5 donors and E:T ratios tested when assessed on the SKOV3 (CLDN6−) cell.

Cytotoxicity and T-cell activation with Ab897 on SKOV3 (CLDN6−) cells at 3:1 E:T ratio at 48 and 72 hours in human Pan-T cell cocultures (5 independent donors) was also determined. As described above, cytotoxicity was measured via Incucyte-based cell viability over time using stably transduced SKOV3 (Incucyte Nuclight Red) cells. T-cells on the plate were collected for flow-cytometry-based measurement of activation and proliferation.

At 48 and 72 hours of coculture of human Pan-T cells with tumor cell lines: Ab897b demonstrated no or minimal cytotoxicity in all 5 donors on the SKOV3 cell line at both time points. Ab897b demonstrated no stimulation of T cell proliferation or activation in all 5 donors on the SKOV3 cell line at both times (results not shown).

### Example 11: T Cell Proliferation, Activation, and Cytokine Release with Ab897 on OVCAR3 (CLDN6+) Cells

Antibody-induced T cell activation and proliferation and cytokine release at 72 hours in human Pan-T cell cocultures (5 independent donors) with the OVCAR3 ovarian tumor cell line at 3:1 E:T ratio by an exemplary bispecific antibody that specifically binds CLDN6 and CD3 (Ab897) was determined.

### Assay Protocol

Seeding of OVCAR-3 NLG cells: OVCAR3-NLG stable cell line was utilized for these studies (stably transduced with Incucyte Nuclight Green). Cells grown in flasks were trypsinized and spun at 1,200 RPM for five minutes at RT. The cell pellet was resuspended in RPMI media+20% FBS and the count was obtained using a hemocytometer. 96 well flat bottom plates were seeded as per the plate map with 5,000 cells/100 μL/well OVCAR-3 NLG cells well in RPMI1640+20% FBS+0.01 mg/mL human insulin. Seeded plates were incubated overnight at 37° C., 5% CO₂.

Addition of antibodies and effector cells: PBMCs (peripheral blood mononuclear cells) from 5 human donors were thawed and Pan-T cells were isolated from the PBMCs using StemCell kit according to manufacturer's instructions. T cell counts were obtained using a hemocytometer. To allow for measurement of T cell proliferation, T cells were incubated with 5 μM CellTrace Violet (CTV) dye in DPBS for 10 minutes at room temperature. After 10 minutes, 10 mL of complete media was used to terminate the staining. Cells were then spun down at 1700 RPM for five minutes. Cell pellets were resuspended in 1 mL of RPMI 1640 media+10% FBS, counted and added to OVCAR3-NLG cells at a 3:1 E:T ratio (15,000 T cells/50 μL/well).

Antibody dilutions were prepared in RPMI 1640+10% FBS, where 4× antibody stock was made and 50 μL of each 4× dilution was added to the well to give the 1× final concentration. Ab897 was used at a starting concentration of 30 nM and diluted 4-fold (9 dilutions) for a total of 10-points. Controls (no antibody added) included T cells+OVCAR3 cells or OVCAR3 target cells alone.

50 μL of antibody+50 μL cell suspension were added. For control wells, only media with no antibody was added. After 72 hours, supernatants were collected from the plates and assessed for TNF-α, IFN-γ and IL-6 using Luminex kits. T cells remaining in the plate were collected after centrifugation and used for flow cytometry-based assay to test for T cell activation and proliferation.

T cell activation and proliferation assay: The replica assay plates were processed to analyze T cell activation and proliferation by flow cytometry. In brief, the cells from the plates were mixed gently with multi-channel pipette and the plates were centrifuged at 1,700 RPM for three minutes. The supernatants were collected and stored at −80° C. for the Luminex assay if required. The cell pellets were further processed for flow cytometry as follows.

Cells were stained with Live/Dead stain for 20 minutes at room temperature. The cells were washed twice using FACS buffer (3% FBS in HBSS) at 1,700 RPM for 3 minutes and re-suspended in 100 μL of FACS buffer. Surface staining antibodies were added to the respective wells as a cocktail of antibodies as per the panel (see Table 13) and incubated for 30 minutes at 4° C. in the dark.

TABLE 13
T-Cell Detection

| Sample No. | Marker | Fluorochrome |
|---|---|---|
| 1 | Live/Dead | Near IR |
| 2 | CD4 | PE |
| 3 | CD8 | APC/AF647 |
| 4 | CD25 | BV785 |
| 5 | Cell trace violet dye | Pacific blue channel |

After incubation, cells were washed twice using FACS buffer (3% FBS in HBSS) and re-suspended in 100 μL of the same buffer. Data from Samples 1-5 were acquired on a Novocyte flow cytometer and the data were analyzed using NovoExpress software.

Luminex Assay: On the day of the assay, supernatants stored at −80° C. were thawed on ice and centrifuged at 16,000×g for 4 minutes prior to use. All samples, reagents, & standards were prepared as per manufacturer's instruction.

In brief, the standards provided in the kit were reconstituted using Calibrator Diluent RD6-52. The standards were serially diluted 3-fold, 6-point DRC. The samples were diluted as required using Calibrator Diluent RD6-52. The Microparticle Cocktail vial was centrifuged for 30 seconds at 1000×g, vortexed gently, and was diluted using Diluent RD2-1. 50 μL of standard or sample was added per well as per the plate map. 50 μL of the microparticle cocktail was added to each well of the microplate. The plates were securely covered with a foil plate sealer and incubated overnight at 4° C. on a horizontal orbital microplate shaker set at 800 RPM. Using a magnetic device designed to accommodate a microplate, the plates were washed using the magnet. The plates were washed three times with Wash Buffer (100 μL/well). 50 μL of diluted Biotin-Antibody Cocktail was added to each well. The plates were securely covered with a foil plate sealer and incubated for 1 hour at room temperature on a horizontal orbital microplate shaker set at 800 RPM. After incubation, the plates were washed 3 times as mentioned before. 50 μL of diluted Streptavidin-PE was then added to each well. The plates were securely covered with a foil plate sealer and incubated for 30 minutes at room temperature on a horizontal orbital microplate shaker set at 800 RPM. After incubation, the plates were washed 3 times as mentioned before. After washing, the microparticles were resuspended in 100 μL of Wash Buffer/well. The plates were incubated for 2 minutes on the shaker and read within 90 minutes using a Luminex® Analyzer.

### Calculations

T-cell proliferation and activation: For T cell proliferation and activation assays, cells were gated on live cells, then CD4 or CD8. Cells were scored for % CTV for T cell proliferation and % CD25 and % CD69 for T cell activation. For activation, graphs show % positive gated on CD4+ or CD8+ cells. For proliferation assay, E+T (no antibody control) was used for gating and decrease in the CTV intensity was collectively taken as % proliferation from the respective parent population (E+T/no antibody control).

Cytokine profiling: Data were collected using a Luminex MAGPIX system (Thermo Fisher Scientific). MFI values were converted to cytokine levels in pg/mL using the standard curve by MAGPIX software with a 5-parameter weighted logistic regression analysis.

### Results

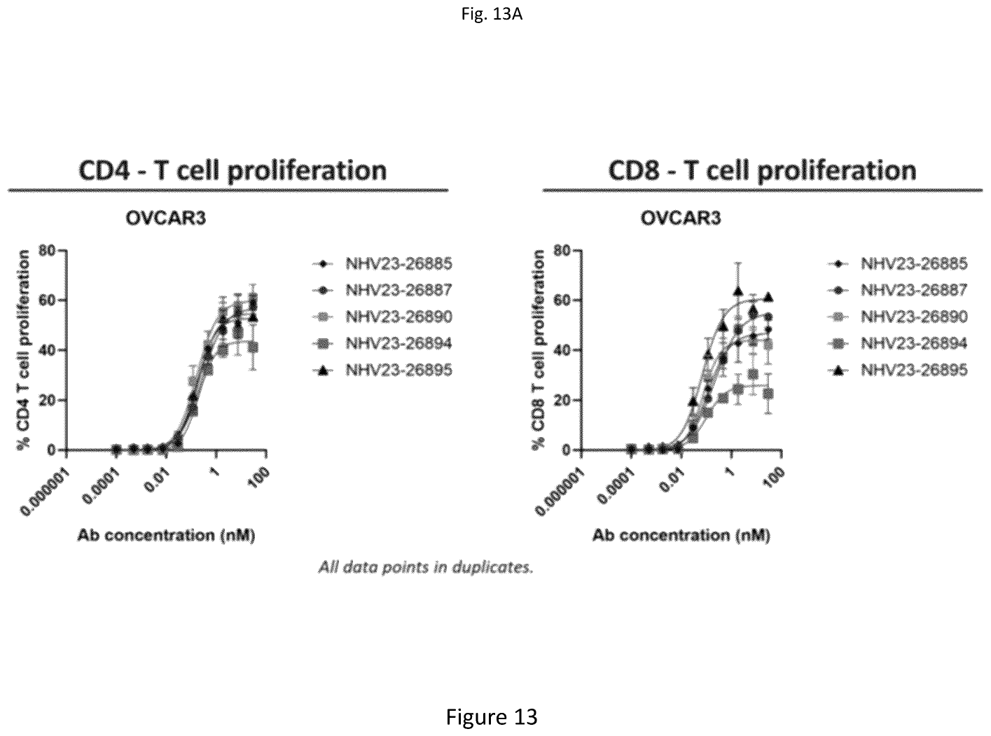

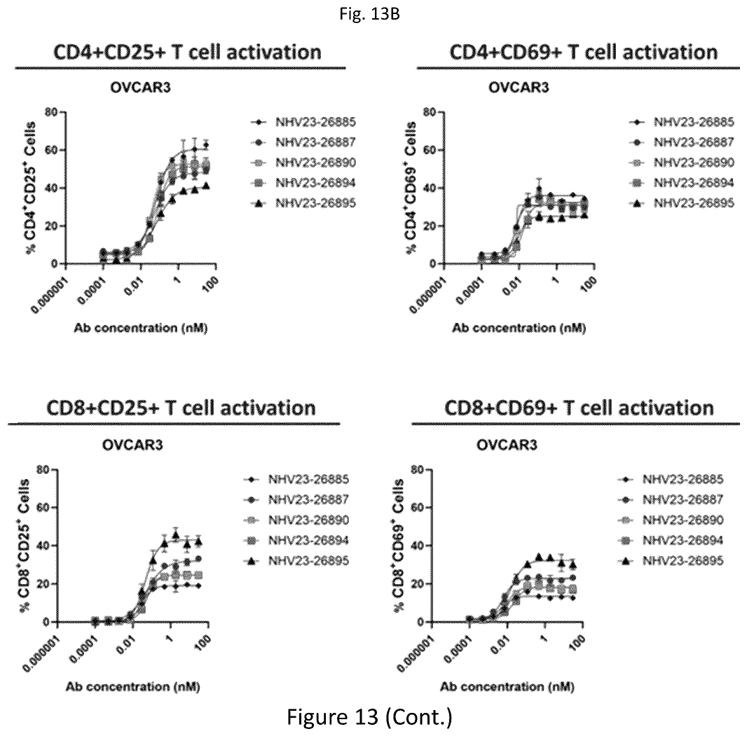

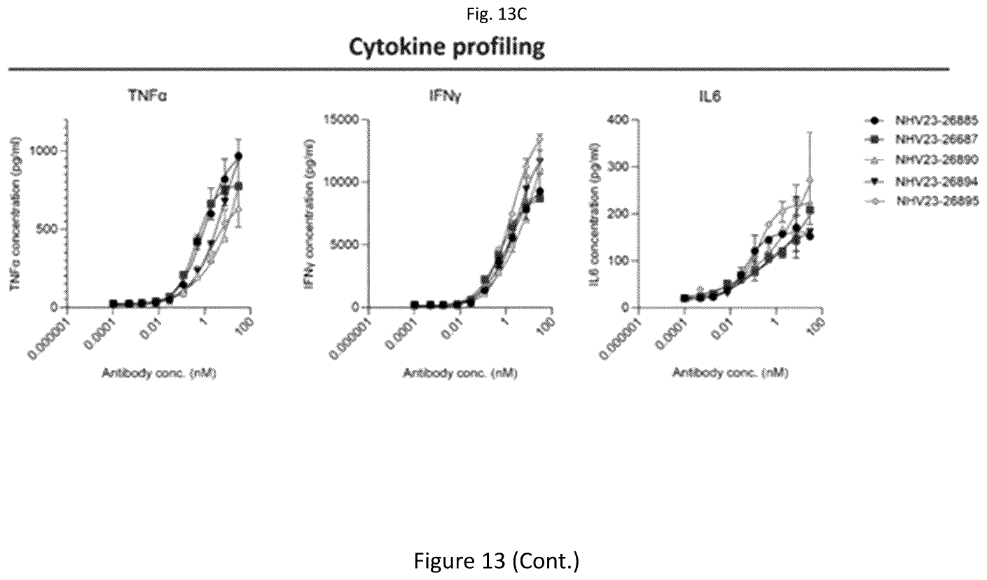

FIG. 13 shows plots of A) T cell proliferation; B) CD4+ and CD8+ T cell activation and C) cytokine profiling at a 72-hour timepoint from OVCAR3 cell cocultured with Pan-T cells from 5 donors at a 3:1 E:T ratio using Ab897b antibody. All data points in duplicates. Mean±SD is plotted.

The results with Ab897 in coculture with human Pan T cells from multiple donors with the OVCAR3 ovarian tumor cell line demonstrated:
1. Dose dependent T cell proliferation and activation after 72-hour coculture with Ab897b and OVCAR3 cells at 3:1 ET ratio. Sub-nM potency of both T cell proliferation and activation was observed in all donors. Percent maximal activity in both activation and proliferation was donor dependent.
2. Dose dependent cytokine increase (TNF-α, IFN-γ and IL-2) were observed at 72 hours with Ab897b and OVCAR3 cells at 3:1 ET ratio. Potency of cytokine release was right shifted (less potent) compared to activation and proliferation of T cells and maximal cytokine release was variable and donor dependent.

### Example 12: In Vivo Ovarian Cancer Tumor Xenograft Model

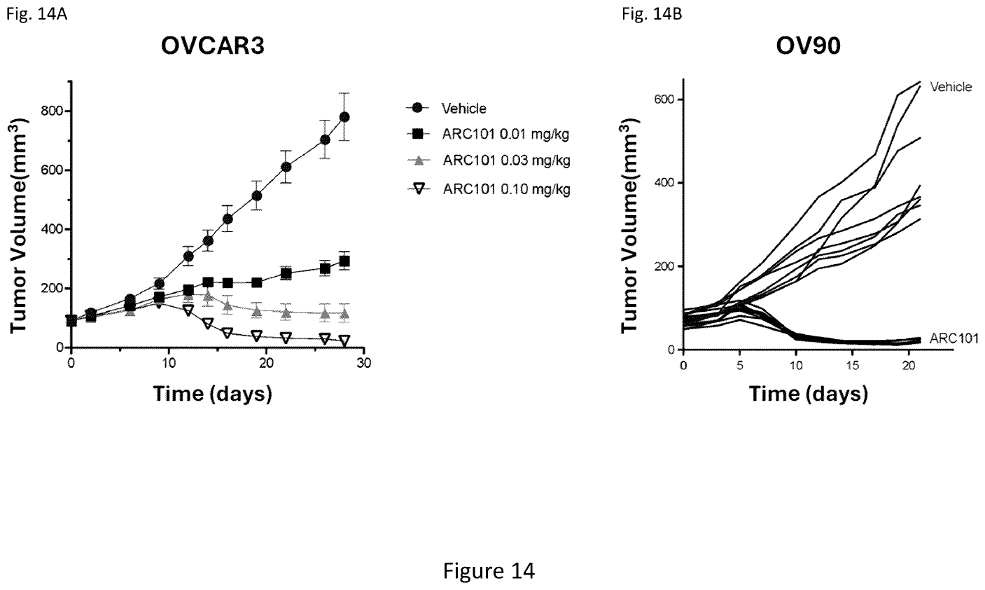

To evaluate the potential effectiveness of Ab897, OVCAR3 xenografts were transplanted into a humanized NSG mice. The mice were then treated with Ab897 weekly for four weeks at various doses, all of which demonstrated significant inhibition of tumor growth (at least 50% tumor growth inhibition (TGI), FIG. 14A).

Given the OVCAR3 results, OV90 xenografts were transplanted into humanized NSG mice and treated with Ab897 weekly at 0.1 mg/kg for three weeks, with a significant decrease in tumor volume observed after only two weeks (FIG. 14B).

It will be appreciated by those skilled in the art that changes could be made to the embodiments described above without departing from the broad inventive concept thereof It is understood, therefore, that the disclosure provided herein is not limited to the particular embodiments disclosed, but it is intended to cover modifications within the spirit and scope of the present disclosure as defined by the present description.

SEQUENCE LISTING
ANTI-CD3 BINDING DOMAINS
01C03A
HCDR1:
(SEQ ID NO: 1)
SGGYYWT
HCDR2:
(SEQ ID NO: 2)
SIYYSGSTYYNPALKS
HCDR3:
(SEQ ID NO: 3)
ERKNELFFDY
LCDR1:
(SEQ ID NO: 4)
TRSSGSIASNYVQ
LCDR2:
(SEQ ID NO: 5)
EDKQRPS
LCDR3:
(SEQ ID NO: 6)
QSFDSSNRV
VH:
(SEQ ID NO: 7)
QVQLQESGPGLVKPSQTLSLTCTVSGGSISSGGYYWTWIRQHPGKCLEW
IGSIYYSGSTYYNPALKSRLTISVDTSKNQFSLKLSSVTAADTAVYYCA
RERKNELFFDYWGQGTLVTVSS
VL:
(SEQ ID NO: 8)
NFMLTQPHSVSESPGKTVTISCTRSSGSIASNYVQWYQQRPDSSPTTVI
YEDKQRPSGVPDRFSGSIDRSSNSASLTISGLKTEDEADYYCQSFDSSN
RVFGCGTKLTVL
01G24A
HCDR1:
(SEQ ID NO: 9)
FYTMN
HCDR2:
(SEQ ID NO: 10)
SITTSSRYIYYADSVKG
HCDR3:
(SEQ ID NO: 11)
AGTKTHDAFDF
LCDR1:
(SEQ ID NO: 12)
SRSSGSIASNYVQ
LCDR2:
(SEQ ID NO: 13)
EDNQRPS
LCDR3:
(SEQ ID NO: 14)
QSYDNSNRV
VH:
(SEQ ID NO: 15)
EVQLVESGGGLVKPGGSLRLSCAASGFTFNFYTMNWVRQAPGKCLEWVS
SITTSSRYIYYADSVKGRFTISRDNAKNSLYLQMNSLRAEDTAVYYCAR
AGTKTHDAFDFWGQGTMVTVSS
VL:
(SEQ ID NO: 16)
NFMLTQPHSVSESPGKTVTISCSRSSGSIASNYVQWYQQRPGSSPTTVI
YEDNQRPSGVPDRFSGSIDSSSNSASLTISGLKTEDEADYYCQSYDNSN
RVFGCGTKLTVL
01M02A
HCDR1:
(SEQ ID NO: 17)
SYSMN
HCDR2:
(SEQ ID NO: 18)
TISRSSSYIYYADSVKG
HCDR3:
(SEQ ID NO: 19)
EKWNLLYFDS
LCDR1:
(SEQ ID NO: 20)
TRSSGSIASNYVQ
LCDR2:
(SEQ ID NO: 21)
EDNQRPS
LCDR3:
(SEQ ID NO: 22)
QSYDSSNRV
VH:
(SEQ ID NO: 23)
EVQLVESGGGLVKPGGSLRLSCAASGFTVSSYSMNWVRQAPGKCLEWVS
TISRSSSYIYYADSVKGRFTISRDNAKNSLYLQMNSLRAEDTAVYFCAR
EKWNLLYFDSWGQGTLVTVSS
VL:
(SEQ ID NO: 24)
NFMLTQSHSVSESPGKTVTISCTRSSGSIASNYVQWYQQRPGSSPTTVI
YEDNQRPSGVPDRFSGSIDSSSNSASLTISGLKTEDEADYYCQSYDSSN
RVFGCGTKLTVL
01P14N
HCDR1:
(SEQ ID NO: 25)
SYSMN
HCDR2:
(SEQ ID NO: 26)
AISRSSTYIYYADSVKG
HCDR3:
(SEQ ID NO: 27)
ENWDLLYFEF
LCDR1:
(SEQ ID NO: 28)
TRSSGSIASNYVQ
LCDR2:
(SEQ ID NO: 29)
EDNQRPS
LCDR3:
(SEQ ID NO: 30)
QSYDSSNRV
VH:
(SEQ ID NO: 31)
EVQLVESGGGLVKPGGSLRLSCAASGFTLSSYSMNWVRQAPGRCLEWVS
AISRSSTYIYYADSVKGRFTISRDNAKNSVHLQMSSLRAEDTAVYYCAR
ENWDLLYFEFWGQGTLVTVSS
VL:
(SEQ ID NO: 32)
NFMLTQPHSVSESPGKTVTISCTRSSGSIASNYVQWYQQRPGSSPTTVI
YEDNQRPSGVPDRFSGSIDSSSNSASLTISGLKTEDEADYYCQSYDSSN
RVFGCGTKLTVL
02I07A
HCDR1:
(SEQ ID NO: 33)
SYSMN
HCDR2:
(SEQ ID NO: 34)
YISSSGSYKYYADSVKG
HCDR3:
(SEQ ID NO: 35)
DRKQLLLDY
LCDR1:
(SEQ ID NO: 36)
SGDALPKKYAY
LCDR2:
(SEQ ID NO: 37)
EDSKRPS
LCDR3:
(SEQ ID NO: 38)
YSTDSSGNHWV
VH:
(SEQ ID NO: 39)
EVQLVESGGGLVKPGGSLRLSCAASGFTFSSYSMNWVRQAPGKGLEWVS
YISSSGSYKYYADSVKGRFTISRDNAKNSLYLQMNSLRAEDTAVFYCAR
DRKQLLLDYWGQGTLVTVSS
VL:
(SEQ ID NO: 40)
SYELTQPPSVSVSPGQTARITCSGDALPKKYAYWYQQKSGQAPVLVIYE
DSKRPSGIPERFSGSSSGTMATLTISGAQVEDEADYYCYSTDSSGNHWV
FACGTKLTVL
03J04A
HCDR1:
(SEQ ID NO: 41)
SYTMN
HCDR2:
(SEQ ID NO: 42)
SITRSSRYIYYADSVKG
HCDR3:
(SEQ ID NO: 43)
AGAATHDAFDI
LCDR1:
(SEQ ID NO: 44)
TRSSGSIASNYVQ
LCDR2:
(SEQ ID NO: 45)
EDNQRPS
LCDR3:
(SEQ ID NO: 46)
QSYDSNNRV
VH:
(SEQ ID NO: 47)
EVQLVESGGGLVTPGGSLRLSCAASGFTFSSYTMNWVRQAPGKCLEWVS
SITRSSRYIYYADSVKGRFTISRDNAKNSLYLQMNSLRAEDTAVYYCAR
AGAATHDAFDIWAQGTMVTVSS
VL:
(SEQ ID NO: 48)
NFMLTQPHSVSESPGKTVTISCTRSSGSIASNYVQWYQQRPGSSPATVI
YEDNQRPSGVPDRFSGSIDSSSNSASLTISGLKTEDEADYYCQSYDSNN
RVFGCGTKLTVL
03L07A
HCDR1:
(SEQ ID NO: 49)
FYTMN
HCDR2:
(SEQ ID NO: 50)
SITTSSRYIYYADSVKG
HCDR3:
(SEQ ID NO: 51)
AGAKTHDAFDF
LCDR1:
(SEQ ID NO: 52)
SRSSGSIASNYVQ
LCDR2:
(SEQ ID NO: 53)
EDNQRPS
LCDR3:
(SEQ ID NO: 54)
QSYDSSNRV
VH:
(SEQ ID NO: 55)
EVQLVESGGGLVKPGGSLRLSCAASGFTFSFYTMNWVRQAPGKCLEWVS
SITTSSRYIYYADSVKGRFTISRDNAKNSLYLQMNSLRAEDTAVYYCAR
AGAKTHDAFDFWGQGTMVTVSS
VL:
(SEQ ID NO: 56)
NFMLTQPHSVSESPGKTVTISCSRSSGSIASNYVQWYQQRPGSSPTTVI
YEDNQRPSGVPDRFSGSIDSSSNSASLTISGLKTEDEADYYCQSYDSSN
RVFGCGTKLTVL
03O15A
HCDR1:
(SEQ ID NO: 57)
SYSMN
HCDR2:
(SEQ ID NO: 58)
SISRSTAYIYYADSVKG
HCDR3:
(SEQ ID NO: 59)
EKWELLYFDS
LCDR1:
(SEQ ID NO: 60)
TRNSGSIASNYVQ
LCDR2:
(SEQ ID NO: 61)
EDNQRPS
LCDR3:
(SEQ ID NO: 62)
QSYDSSNRV
VH:
(SEQ ID NO: 63)
EVQLVESGGGLVKPGGSLRLSCAASGFTLSSYSMNWVRQAPGKCLEWVS
SISRSTAYIYYADSVKGRFTISRDNAKNSVHLQMNSLRAEDTAVYFCAR
EKWELLYFDSWGQGTLVTVSS
VL:
(SEQ ID NO: 64)
NFMLTQPHSVSESPGKTVTISCTRNSGSIASNYVQWYQQRPGSSPTTVI
YEDNQRPSGVPDRFSGSIDSSSNSASLTISGLKTEDEADYYCQSYDSSN
RVFGCGTKLTVL
03P01A
HCDR1:
(SEQ ID NO: 65)
TSPIN
HCDR2:
(SEQ ID NO: 66)
SISRISSHKYYADSVKG
HCDR3:
(SEQ ID NO: 67)
EKWELLYFDY
LCDR1:
(SEQ ID NO: 68)
RSSQSLLDSDDGNTYLD
LCDR2:
(SEQ ID NO: 69)
TLSYRAS
LCDR3:
(SEQ ID NO: 70)
MQRIEFPSIT
VH:
(SEQ ID NO: 71)
EVQVVESGGGLVKPGGSLRLSCAASGFTLSTSPINWVRQAPGKCLEWVS
SISRISSHKYYADSVKGRFTISRDNAKNSVYLQMNSLRVEDTAVYYCAR
EKWELLYFDYWGQGTLVTVSS
VL:
(SEQ ID NO: 72)
DIVMTQTPLSLPVTPGEPASISCRSSQSLLDSDDGNTYLDWYLQKPGQS
PQLLIYTLSYRASGVPDRFSGSGSGTDFTLKISRVEAEDVGIYYCMQRI
EFPSITFGCGTRLVIK
03P01Z
HCDR1:
(SEQ ID NO: 73)
TSPIN
HCDR2:
(SEQ ID NO: 74)
SISRISSHKYYADSVKG
HCDR3:
(SEQ ID NO: 75)
EKWELLYFDY
LCDR1:
(SEQ ID NO: 76)
TRSSGSIASNYVQ
LCDR2:
(SEQ ID NO: 77)
EDNQRPS
LCDR3:
(SEQ ID NO: 78)
QSYDSSNRV
VH:
(SEQ ID NO: 79)
EVQVVESGGGLVKPGGSLRLSCAASGFTLSTSPINWVRQAPGKCLEWVS
SISRISSHKYYADSVKGRFTISRDNAKNSVYLQMNSLRVEDTAVYYCAR
EKWELLYFDYWGQGTLVTVSS
VL:
(SEQ ID NO: 80)
NFMLTQPHSVSESPGKTVTISCTRSSGSIASNYVQWYQQRPGSSPTAVI
YEDNQRPSGVPDRFSGSIDSSSNSASLTISGLKTEDEADYYCQSYDSSN
RVFGCGTKLTVL
04C03A
HCDR1:
(SEQ ID NO: 81)
NYILN
HCDR2:
(SEQ ID NO: 82)
SISSSSRYIFYADSVKG
HCDR3:
(SEQ ID NO: 83)
ESKWELLSFDY
LCDR1:
(SEQ ID NO: 84)
TRSSGSIASNYVQ
LCDR2:
(SEQ ID NO: 85)
EDNQRPS
LCDR3:
(SEQ ID NO: 86)
QSYDRSNRV
VH:
(SEQ ID NO: 87)
EVQLVESGGGLVKPGGSLRLSCAASGFTFNNYILNWVRQAPGKCLEWVS
SISSSSRYIFYADSVKGRFTISRDNAKNSLYLQMNSLRAEDTGVYYCAR
ESKWELLSFDYWGQGTLVTVSS
VL:
(SEQ ID NO: 88)
NFMLTQPHSVSESPGKTVTISCTRSSGSIASNYVQWYQQRPGSSPINVI
YEDNQRPSGVPDRFSGSIDSSSNSASLTISGLKTEDEADYYCQSYDRSN
RVFGCGTKLTVL
04J18A
HCDR1:
(SEQ ID NO: 89)
SGGYSWS
HCDR2:
(SEQ ID NO: 90)
SIYYSGRTYYNPALKS
HCDR3:
(SEQ ID NO: 91)
ERKNELFFDY
LCDR1:
(SEQ ID NO: 92)
TRSSGSIASNYVQ
LCDR2:
(SEQ ID NO: 93)
EDNQRPS
LCDR3:
(SEQ ID NO: 94)
QSYDSSNRV
VH:
(SEQ ID NO: 95)
QVQLQESGPGLVKPSQTLSLTCTVSGGSISSGGYSWSWIRQHPGKCLEW
IGSIYYSGRTYYNPALKSRITISVDTSKNQFSLMLSSVPAADTAVYYCA
RERKNELFFDYWGQGTLVTVSS
VL:
(SEQ ID NO: 96)
NFMLTQPHSVSESPGKTITISCTRSSGSIASNYVQWYQQRPGSSPTAVI
YEDNQRPSGVPDRFSGSIDSSSNSASLTISGLKTEDEADYYCQSYDSSN
RVFGCGTKLTVL
04K01A
HCDR1:
(SEQ ID NO: 97)
SSLMN
HCDR2:
(SEQ ID NO: 98)
SISSSSNYIYYADSVRG
HCDR3:
(SEQ ID NO: 99)
ERWVLLYFDY
LCDR1:
(SEQ ID NO: 100)
TRSSGSIASNYVQ
LCDR2:
(SEQ ID NO: 101)
EDNQRPS
LCDR3:
(SEQ ID NO: 102)
QSYDSSIRV
VH:
(SEQ ID NO: 103)
EVHLVESGGGLVKPGGSLRLSCAASGFTFSSSLMNWVRQAPGKCLEWVS
SISSSSNYIYYADSVRGRFTISRDNAKNSLYLQMHSLRAEDTAVYYCAR
ERWVLLYFDYWGQGTLVTVSS
VL:
(SEQ ID NO: 104)
NFMLTQPHSVSESPGKTVTISCTRSSGSIASNYVQWYQQRPGSSPTTVI
YEDNQRPSGVPDRFSGSIDSSSNSASLTISGLKTEDEADYYCQSYDSSI
RVFGCGTKLTVL
04M22A
HCDR1:
(SEQ ID NO: 105)
SSLMN
HCDR2:
(SEQ ID NO: 106)
SISSSSNYIYYADSVRG
HCDR3:
(SEQ ID NO: 107)
ERWVLLYFDY
LCDR1:
(SEQ ID NO: 108)
TRSSGSIASNYVQ
LCDR2:
(SEQ ID NO: 109)
EDNQRPS
LCDR3:
(SEQ ID NO: 110)
QSYDSSIRV
VH:
(SEQ ID NO: 111)
EVHLVESGGGLVKPGGSLRLSCAASGFTFSSSLMNWVRQAPGKCLEWVS
SISSSSNYIYYADSVRGRFTISRDNAKNSLYLQMHSLRAEDTAVYYCAR
ERWVLLYFDYWGQGTLVTVSS
VL:
(SEQ ID NO: 112)
NFMLTQPHSVSESPGKTVTISCTRSSGSIASNYVQWYQQRPGSSPTTVI
YEDNQRPSGVPDRFSGSIDSSSNSASLTISGLKTEDEADYYCQSYDSSI
RVFGCGTKLTVL
04P20A
HCDR1:
(SEQ ID NO: 113)
SYSMN
HCDR2:
(SEQ ID NO: 114)
YINSRSSTKYYADSVKG
HCDR3:
(SEQ ID NO: 115)
VMRHYGMDV
LCDR1:
(SEQ ID NO: 116)
TRSSGSIASNYVQ
LCDR2:
(SEQ ID NO: 117)
EDNQRPS
LCDR3:
(SEQ ID NO: 118)
QSFDNSNHWV
VH:
(SEQ ID NO: 119)
EVQLAESGGGLVQPGGSLRLSCAASGFTFSSYSMNWVRQAPGKCLEWVL
YINSRSSTKYYADSVKGRFTISRDNAKNSLYLQMNSLRDADTAVYYCAR
VMRHYGMDVWGQGTTVTVSS
VL:
(SEQ ID NO: 120)
NFMLTQPHSVSESPEKTVTISCTRSSGSIASNYVQWYQQRPGNSPTTLI
YEDNQRPSGVPDRFSGSIDSSSNSASLTISGLKTEDEADYYCQSFDNSN
HWVFGCGTKLTVL
05B02A
HCDR1:
(SEQ ID NO: 121)
RYSMN
HCDR2:
(SEQ ID NO: 122)
TVSSGSRYRYYADSVKG
HCDR3:
(SEQ ID NO: 123)
AGASTHDSFDI
LCDR1:
(SEQ ID NO: 124)
TRSRGSIASNYVQ
LCDR2:
(SEQ ID NO: 125)
EDNKRPS
LCDR3:
(SEQ ID NO: 126)
QSYDNSNRV
VH:
(SEQ ID NO: 127)
EVQLVESGGGLVKPGGSLTLSCEASGFTFNRYSMNWVRQGPGKCLEWVS
TVSSGSRYRYYADSVKGRFTISRDNAKNSLYLQMNSLRAEDTAVYYCAR
AGASTHDSFDIWGQGTMVTVPS
VL:
(SEQ ID NO: 128)
NFMLTQSHSVSESPGKTVTISCTRSRGSIASNYVQWYQQRPGSSPTTVI
YEDNKRPSGVPDRFSGSIDSSSNSASLTISGLKTEDEADYYCQSYDNSN
RVFGCGTKLTVL
05L13A
HCDR1:
(SEQ ID NO: 129)
SYSMN
HCDR2:
(SEQ ID NO: 130)
SISRSSSYIYYADSVKG
HCDR3:
(SEQ ID NO: 131)
EKWELLYFDA
LCDR1:
(SEQ ID NO: 132)
TRSSGSIASNYVQ
LCDR2:
(SEQ ID NO: 133)
EDNQRPS
LCDR3:
(SEQ ID NO: 134)
QSYDRSNRV
VH:
(SEQ ID NO: 135)
EVQLVESGGGLVKPGGSLRLSCAASGFTVSSYSMNWVRQAPGKCLEWVS
SISRSSSYIYYADSVKGRFTISRDNAKNSLYLQMNSLRAEDTAVYYCAR
EKWELLYFDAWGQGTLVTVSS
VL:
(SEQ ID NO: 136)
NFMLTQPHSVSESPGKTVTISCTRSSGSIASNYVQWYQQRPGSSPTTVI
YEDNQRPSGVPDRFSGSIDSSSNSASLTISGLKTEDEADYYCQSYDRSN
RVFGCGTKLTVL
05N13A
HCDR1:
(SEQ ID NO: 137)
SYSMN
HCDR2:
(SEQ ID NO: 138)
SSSRSSSYIYYADSVKG
HCDR3:
(SEQ ID NO: 139)
ERWELLYFDY
LCDR1:
(SEQ ID NO: 140)
TRSSGSIASNYVQ
LCDR2:
(SEQ ID NO: 141)
EDNQRPS
LCDR3:
(SEQ ID NO: 142)
QSYDSSIRV
VH:
(SEQ ID NO: 143)
EVQLVESGGGLVKPGGSLRLSCAASGFTFSSYSMNWVRQAPGKCLEWVS
SSSRSSSYIYYADSVKGRFTISRDNAKNSLYLQMNSLRAEDTAVYYCAR
ERWELLYFDYWGQGTLVTVSS
VL:
(SEQ ID NO: 144)
NFMLTQPHSVSESPGKTVTISCTRSSGSIASNYVQWYQQRPGSSPTTVI
YEDNQRPSGVPDRFSGSIDSSSNSASLTISGLKTEDEADYYCQSYDSSI
RVFGCGTKLTVL
06A06A
HCDR1:
(SEQ ID NO: 145)
SGAYYWN
HCDR2:
(SEQ ID NO: 146)
DIYYSGTTYYNPALKS
HCDR3:
(SEQ ID NO: 147)
ERKNELFFDY
LCDR1:
(SEQ ID NO: 148)
TRSSGSIASNYVQ
LCDR2:
(SEQ ID NO: 149)
EDNQRPS
LCDR3:
(SEQ ID NO: 150)
QSYDSSNRV
VH:
(SEQ ID NO: 151)
QVQLQESGPGLVKPSQTLSLTCIVSGGSISSGAYYWNWIRQHPGECLEW
IGDIYYSGTTYYNPALKSRVSISVDTSKNQFSLNLISVTAADTAVYYCA
RERKNELFFDYWGQGTLVTVSS
VL:
(SEQ ID NO: 152)
NFMLTQPHSVSESPGKTVTISCTRSSGSIASNYVQWYQQRPGSSPTTVI
YEDNQRPSGVPDRFSGSIDSSSNSASLTLSGLKTEDEADYYCQSYDSSN
RVFGCGTKLTVL
07B06A
HCDR1:
(SEQ ID NO: 153)
NYAMN
HCDR2:
(SEQ ID NO: 154)
SISRSNSHKYYADSVKG
HCDR3:
(SEQ ID NO: 155)
EKWELLYFDS
LCDR1:
(SEQ ID NO: 156)
RSSQSLLDSDDGNTYLD
LCDR2:
(SEQ ID NO: 157)
TLSYRAS
LCDR3:
(SEQ ID NO: 158)
MQRIEFPSIT
VH:
(SEQ ID NO: 159)
EVQVVESGGGLVKPGGSLRLSCAASGFTLSNYAMNWVRQAPGKCLEWIS
SISRSNSHKYYADSVKGRFAISRDIAKNSVYLQMNSLRAEDTAVYYCAR
EKWELLYFDSWGQGTLVTVSS
VL:
(SEQ ID NO: 160)
DIVMTQTPLSLPVTPGEPASISCRSSQSLLDSDDGNTYLDWYLQKPGRS
PQLLIYTLSYRASGVPDRFSGSGSGTDFTLKISRVEAEDVGIYYCMQRI
EFPSITFGCGTRLEIK
07B06Z
HCDR1:
(SEQ ID NO: 161)
NYAMNW
HCDR2:
(SEQ ID NO: 162)
SISRSNSHKYYADSVKG
HCDR3:
(SEQ ID NO: 163)
EKWELLYFDS
LCDR1:
(SEQ ID NO: 164)
TRSSGSIASNYVQ
LCDR2:
(SEQ ID NO: 165)
EDDQRPS
LCDR3:
(SEQ ID NO: 166)
QSYDSSNRV
VH:
(SEQ ID NO: 167)
EVQVVESGGGLVKPGGSLRLSCAASGFTLSNYAMNWVRQAPGKCLEWIS
SISRSNSHKYYADSVKGRFAISRDIAKNSVYLQMNSLRAEDTAVYYCAR
EKWELLYFDSWGQGTLVTVSS
VL:
(SEQ ID NO: 168)
NFMLTQPHSVSESPGKTITISCTRSSGSIASNYVQWYQQRPGSSPTTVI
YEDDQRPSGVPDRFSGSIDSSSNSASLTISGLKTEDEADYFCQSYDSSN
RVFGCGTKLTVL
07H10A
HCDR1:
(SEQ ID NO: 169)
DYYIHW
HCDR2:
(SEQ ID NO: 170)
WINPNSGGTTYVQKFQG
HCDR3:
(SEQ ID NO: 171)
DRGTYYDYFDN
LCDR1:
(SEQ ID NO: 172)
KSSESVLYRSNNKNYLT
LCDR2:
(SEQ ID NO: 173)
WASIRES
LCDR3:
(SEQ ID NO: 174)
QQYYGTPYT
VH:
(SEQ ID NO: 175)
QVQLMQSGAEVKKPGASVKVSCKASGYTFTDYYIHWVRQAPGQCLEWMG
WINPNSGGTTYVQKFQGRVTMTRDTSISTAYMELSRLRSDDTAVYYCSR
DRGTYYDYFDNWGQGTLVTVSS
VL:
(SEQ ID NO: 176)
DIVMTQSPDSLAVSLGERATINCKSSESVLYRSNNKNYLTWYQQKPGQP
PKLLIYWASIRESGVPDRFSGSGSGTDFTLTISSLQAEDVAVYFCQQYY
GTPYTFGCGTKLEIK
07L12A
HCDR1:
(SEQ ID NO: 177)
RNSMN
HCDR2:
(SEQ ID NO: 178)
SISTSGKYRYYADSLKD
HCDR3:
(SEQ ID NO: 179)
AGGSTHDSFDI
LCDR1:
(SEQ ID NO: 180)
TRSSGSIARNYVQ
LCDR2:
(SEQ ID NO: 181)
EDNKRPS
LCDR3:
(SEQ ID NO: 182)
QSYDNSNRV
VH:
(SEQ ID NO: 183)
EVQLVESGGGLVKPGGSLRLSCAASGFTFSRNSMNWVRQAPGKCLEWVS
SISTSGKYRYYADSLKDRFTISRDNAMNSLYLQMNSLRVEDTAVYYCAR
AGGSTHDSFDIWGQGTMVTVS
VL:
(SEQ ID NO: 184)
NFMLTQPHSVSESPGKTVTISCTRSSGSIARNYVQWYQQRPGSSPKTVI
YEDNKRPSGVPDRISGSIDSSSNSASLTISGLKTEDEADYYCQSYDNSN
RVFGGGTKLTVL
07P07A
HCDR1:
(SEQ ID NO: 185)
FYTMN
HCDR2:
(SEQ ID NO: 186)
SITTSSRYIYYADSVKG
HCDR3:
(SEQ ID NO: 187)
AGAKTHDAFDF
LCDR1:
(SEQ ID NO: 188)
SRSSGSIASNYVQ
LCDR2:
(SEQ ID NO: 189)
EDNQRPS
LCDR3:
(SEQ ID NO: 190)
QSYDSSNRV
VH:
(SEQ ID NO: 191)
EVHLVESGGGLVKPGGSLRLSCAASGFTFSFYTMNWVRQAPGKCLEWVS
SITTSSRYIYYADSVKGRFTVSRDNAKNSLYLQMNSLRAEDTAVYYCAR
AGAKTHDAFDFWGQGTMVTVSS
VL:
(SEQ ID NO: 192)
NFMLTQPHSVSESPGKTVTISCSRSSGSIASNYVQWYQQRPGSSPTTVI
YEDNQRPSGVPDRFSGSIDSSSNSASLTISGLKTEDEADYYCQSYDSSN
RVFGCGTKLTVL
07P15A
HCDR1:
(SEQ ID NO: 193)
RYSMN
HCDR2:
(SEQ ID NO: 194)
SISSSSYYIYYADSLKG
HCDR3:
(SEQ ID NO: 195)
ERRAGFDY
LCDR1:
(SEQ ID NO: 196)
TRSSGSIASNYVQ
LCDR2:
(SEQ ID NO: 197)
EDNQRPS
LCDR3:
(SEQ ID NO: 198)
QSYDSSNRV
VH:
(SEQ ID NO: 199)
EVQLVESGGGLVKPGGSLRLSCAASGFTFSRYSMNWVRQAPGKCLEWVS
SISSSSYYIYYADSLKGRFTISRDNAKSSLYLQMNSLRAEDTAVYYCAR
ERRAGFDYWGQGTLVTVSS
VL:
(SEQ ID NO: 200)
NFMLTQPHSVSESPGKTVTISCTRSSGSIASNYVQWYQQRPGSSPTTVI
YEDNQRPSGVPDRFSGSIDSSSNSASLTISGLKTEDEADYYCQSYDSSN
RVFGCGTKLTVL
08M15A
HCDR1:
(SEQ ID NO: 201)
SSSMN
HCDR2:
(SEQ ID NO: 202)
SISSRSRHIYYADSVKG
HCDR3:
(SEQ ID NO: 203)
EKWELLYFDY
LCDR1:
(SEQ ID NO: 204)
GGNNIGSKSVH
LCDR2:
(SEQ ID NO: 205)
DDSDRPS
LCDR3:
(SEQ ID NO: 206)
QVWDSSSDHVV
VH:
(SEQ ID NO: 207)
EVQLVESGGGLVKPGGSLRLSCAASGFTFSSSSMNWVRQAPGKCLEWVS
SISSRSRHIYYADSVKGRFTISRDNAKNSLYLQMNSLRAEDTAVYYCAR
EKWELLYFDYWGQGTLVTVSS
VL:
(SEQ ID NO: 208)
SYVLTQPPSVSVAPGQTARITCGGNNIGSKSVHWYQQKPGQAPVLVVYD
DSDRPSGIPERFSGSNSGNTATLTISRVEAGDEADYYCQVWDSSSDHVV
FGCGTKLTVL
08M15Z
HCDR1:
(SEQ ID NO: 209)
SSSMN
HCDR2:
(SEQ ID NO: 210)
SISSRSRHIYYADSVKG
HCDR3:
(SEQ ID NO: 211)
EKWELLYFDY
LCDR1:
(SEQ ID NO: 212)
RSSGSIASNYVQ
LCDR2:
(SEQ ID NO: 213)
EDNQRPS
LCDR3:
(SEQ ID NO: 214)
QSYDSSNRV
VH:
(SEQ ID NO: 215)
EVQLVESGGGLVKPGGSLRLSCAASGFTFSSSSMNWVRQAPGKCLEWVS
SISSRSRHIYYADSVKGRFTISRDNAKNSLYLQMNSLRAEDTAVYYCAR
EKWELLYFDYWGQGTLVTVSS
VL:
(SEQ ID NO: 216)
NFMLTQPHSVSESPGKTVTISCTRSSGSIASNYVQWYQQRPGSSPTTLI
YEDNQRPSGVPDRFSGSVDSSSNSASLTISGLKTEDEADYYCQSYDSSN
RVFGCGTKLTVL
09B13A
HCDR1:
(SEQ ID NO: 217)
SYSMN
HCDR2:
(SEQ ID NO: 218)
SISRSSAYIYYADSVKG
HCDR3:
(SEQ ID NO: 219)
EKWELLYFDY
LCDR1:
(SEQ ID NO: 220)
RSSGSIASNYVQ
LCDR2:
(SEQ ID NO: 221)
EDNQRPS
LCDR3:
(SEQ ID NO: 222)
QSYDSSNRV
VH:
(SEQ ID NO: 223)
EVQLVESGGGLVKPGGSLRLSCAASRFTLSSYSMNWVRQAPGKCLEWVS
SISRSSAYIYYADSVKGRFTISRDNAKNSVHLQMNSLRAEDTAVYYCAR
EKWELLYFDYWGQGTLVTVSS
VL:
(SEQ ID NO: 224)
NFMLTQPHSVSESPGKTVTISCTRSSGSIASNYVQWYQRRPGSSPTTVI
YEDNQRPSGVPDRFSGSIDSSSNSASLTISGLKTEDEADYYCQSYDSSN
RVFGCGTKLTVL
09D16A
HCDR1:
(SEQ ID NO: 225)
RYSMN
HCDR2:
(SEQ ID NO: 226)
TVSSGSRYRYYADSVKG
HCDR3:
(SEQ ID NO: 227)
AGGNTHDAFDI
LCDR1:
(SEQ ID NO: 228)
TRSRGSIASNYVQ
LCDR2:
(SEQ ID NO: 229)
EDNKRPS
LCDR3:
(SEQ ID NO: 230)
SYDNSNRV
VH:
(SEQ ID NO: 231)
EVQLVESGGGLVKPGGSLRLSCAASGFTFNRYSMNWVRQAPGKCLEWVS
TVSSGSRYRYYADSVKGRFTISRDNAKNSLYLQMNSLRAEDTAVYYCAR
AGGNTHDAFDIWGQGTMVTVSS
VL:
(SEQ ID NO: 232)
NFMLTQSHSVSESPGKTVTISCTRSRGSIASNYVQWYQQRPGSSPTTVI
YEDNKRPSGVPDRFSGSIDSSSNSASLTISGLKTEDEADYYCQSYDNSN
RVFGCGTKVTVL
10E16A
HCDR1:
(SEQ ID NO: 233)
RYSMNW
HCDR2:
(SEQ ID NO: 234)
LISSSSSYKYYVDSVKG
HCDR3:
(SEQ ID NO: 235)
DRRELVLDY
LCDR1:
(SEQ ID NO: 236)
SGDALPKKYAY
LCDR2:
(SEQ ID NO: 237)
EDSKRPS
LCDR3:
(SEQ ID NO: 238)
YSTNNSGNHW
VH:
(SEQ ID NO: 239)
EVQLVESGGGLVKPGGSLRLSCAASGFTFSRYSMNWVRQAPGKCLEWVS
LISSSSSYKYYVDSVKGRFTISRDNAKNSLYLEMSSLRAEDTAVYYCAR
DRRELVLDYWGQGTLVTVSS
VL:
(SEQ ID NO: 240)
SYELTQPPSVSVSPGQTARITCSGDALPKKYAYWYQQKSGQAPVLVIYE
DSKRPSGIPERLSGSSSGTMATLIISGAQVEDEADYYCYSTNNSGNHWV
FGCGTKLTVL
14A15A
HCDR1:
(SEQ ID NO: 241)
TYSMN
HCDR2:
(SEQ ID NO: 242)
TFSRSSSHIYYADSVKG
HCDR3:
(SEQ ID NO: 243)
EQWNLLYFDY
LCDR1:
(SEQ ID NO: 244)
TRSSGSIASNYVQ
LCDR2:
(SEQ ID NO: 245)
EDNQRPS
LCDR3:
(SEQ ID NO: 246)
QSYDRSNRV
VH:
(SEQ ID NO: 247)
EVQLVESGGGLVRPGGSLRLSCAASGFTLSTYSMNWVRQAPGKCLEWVS
TFSRSSSHIYYADSVKGRFTISRDNAKNSLYLQMNSLRAEDTAVYYCAR
EQWNLLYFDYWGQGTLVTVSS
VL:
(SEQ ID NO: 248)
NLMLTQPHSVSESPGKTVTISCTRSSGSIASNYVQWYQQRPGSSPTTVI
YEDNQRPSGVPDRFSGSIDSSSNSASLTISGLKTEDEADYYCQSYDRSN
RVFGCGTKLTVL
14C10A
HCDR1:
(SEQ ID NO: 249)
SRYSMN
HCDR2:
(SEQ ID NO: 250)
SISSGSRYIYYRDSVRG
HCDR3:
(SEQ ID NO: 251)
EPRNHFDY
LCDR1:
(SEQ ID NO: 252)
TRSSGSIASNYVQ
LCDR2:
(SEQ ID NO: 253)
EDNQRPS
LCDR3:
(SEQ ID NO: 254)
QSYDSSIRV
VH:
(SEQ ID NO: 255)
EVQLVESGGGLVKPGGSLKLSCAASGFTFSRYSMNWVRQASGKCLEWVS
SISSGSRYIYYRDSVRGRFTISRDNAKNSLYLQMNSLRAEDTALYYCAR
EPRNHFDYWGQGTLVTVSS
VL:
(SEQ ID NO: 256)
NFMLIQPHSVSESPGKTVTISCTRSSGSIASNYVQWYQHHPGSSPTTVI
YEDNQRPSGVPDRFSGSIDSSSNSASLTISGLKTEDEADYYCQSYDSSI
RVFGCGTKLTVL
VL-Linker-VH scFv:
(SEQ ID NO: 313)
NFMLIQPHSVSESPGKTVTISCTRSSGSIASNYVQWYQHHPGSSPTTVI
YEDNQRPSGVPDRFSGSIDSSSNSASLTISGLKTEDEADYYCQSYDSSI
RVFGCGTKLTVLGGSEGKSSGSGSESKSTGGSEVQLVESGGGLVKPGGS
LKLSCAASGFTFSRYSMNWVRQASGKCLEWVSSISSGSRYIYYRDSVRG
RFTISRDNAKNSLYLQMNSLRAEDTALYYCAREPRNHFDYWGQGTLVTV
SS
scFv-Fc:
(SEQ ID NO: 314)
NFMLIQPHSVSESPGKTVTISCTRSSGSIASNYVQWYQHHPGSSPTTVI
YEDNQRPSGVPDRFSGSIDSSSNSASLTISGLKTEDEADYYCQSYDSSI
RVFGCGTKLTVLGGSEGKSSGSGSESKSTGGSEVQLVESGGGLVKPGGS
LKLSCAASGFTFSRYSMNWVRQASGKCLEWVSSISSGSRYIYYRDSVRG
RFTISRDNAKNSLYLQMNSLRAEDTALYYCAREPRNHFDYWGQGTLVTV
SSEPKSSDKTHTCPPCPAPEAAGGPSVFLFPPKPKDTLMISRTPEVTCV
VVDVSHEDPEVKFNWYVDGVEVHNAKTKPREEQYNSTYRVVSVLTVLHQ
DWLNGKEYKCKVSNKALPAPIEKTISKAKGQPREPQVYTLPPCRDELTK
NQVSLWCLVKGFYPSDIAVEWESNGQPENNYKTTPPVLDSDGSFFLYSK
LTVDKSRWQQGNVFSCSVMHEALHNHYTQKSLSLSPGK
04D18A
HCDR1:
(SEQ ID NO: 257)
RYSMN
HCDR2:
(SEQ ID NO: 258)
TISSSSSYRYYADSVKG
HCDR3:
(SEQ ID NO: 259)
AGGNTHDAFEI
LCDR1:
(SEQ ID NO: 260)
TRSSGSIARNYVQ
LCDR2:
(SEQ ID NO: 261)
EDNKRPS
LCDR3:
(SEQ ID NO: 262)
QSYDNSNRV
VH:
(SEQ ID NO: 263)
EVQLVESGGGLVKPGGSLRLSCEASGFTFSRYSMNWVRQAPGKCLGWVS
TISSSSSYRYYADSVKGRFTISRDNAKNSLYLQMNSLRAEDTAVYYCAR
AGGNTHDAFEIWGQGTMVTVSS
VL:
(SEQ ID NO: 264)
NFMLTQPHSVSASPGKTVTISCTRSSGSIARNYVQWYQQRPGSSPTTVI
FEDNKRPSGVPDRFSGSIDSSSNSASLTISGLKTEDEADYYCQSYDNSN
RVFGCGTKLTVL
02J12A
HCDR1:
(SEQ ID NO: 265)
SYTMN
HCDR2:
(SEQ ID NO: 266)
SITRSSRYIYYADSVKG
HCDR3:
(SEQ ID NO: 267)
AGAATHDAFDI
LCDR1:
(SEQ ID NO: 268)
TRSSGSIASNYVQ
LCDR2:
(SEQ ID NO: 269)
EDNQRPS
LCDR3:
(SEQ ID NO: 270)
QSYDGNNRV
VH:
(SEQ ID NO: 271)
EVQLVESGGGLVKPGGSLRLSCAASGFTFSSYTMNWVRQAPGKCLEWVS
SITRSSRYIYYADSVKGRFIISRDNAKNSLYLQMNSLRAEDTAVYYCAR
AGAATHDAFDIWAQGTMVTVSS
VL:
(SEQ ID NO: 272)
NFMLTQPHSVSESPGKTVTISCTRSSGSIASNYVQWYQQRPGSSPATVI
YEDNQRPSGVPDRFSGSIDSSSNSASLTISGLKTEDEADYYCQSYDGNN
RVFGCGTKLTVL
07N22A
HCDR1:
(SEQ ID NO: 273)
IYSMN
HCDR2:
(SEQ ID NO: 274)
SISSSSSYIHYADSVKG
HCDR3:
(SEQ ID NO: 275)
DRRERLLAY
LCDR1:
(SEQ ID NO: 276)
SGDALPKKYAY
LCDR2:
(SEQ ID NO: 277)
EDSKRPS
LCDR3:
(SEQ ID NO: 278)
YSTDSRGNLWV
VH:
(SEQ ID NO: 279)
EVQLVESGGGLVKPGGSLRLSCAASGFTFSIYSMNWVRQAPGKCLEWVS
SISSSSSYIHYADSVKGRFTISRDNAKNSMHLQLNSLRAEDTAVYYCAR
DRRERLLAYWGQGTLVTVSS
VL:
(SEQ ID NO: 280)
SYELTQPPSVSVSPGQTARITCSGDALPKKYAYWYQQKSGQAPVLVIYE
DSKRPSGIPERFSGSSSGTMATLTISGAQVEDEADYYCYSTDSRGNLWV
FGCGTKVTVL
08M01A
HCDR1:
(SEQ ID NO: 281)
SYSMN
HCDR2:
(SEQ ID NO: 282)
TISRSSSYIYYADSVKG
HCDR3:
(SEQ ID NO: 283)
EKWNLLYFDA
LCDR1:
(SEQ ID NO: 284)
TRSSGSIASNYVQ
LCDR2:
(SEQ ID NO: 285)
EDNQRPS
LCDR3:
(SEQ ID NO: 286)
QSYDSSNRV
VH:
(SEQ ID NO: 287)
EVQLVESGGGLVKPGGSLRLSCAASGFTVSSYSMNWVRQAPGKCLEWVS
TISRSSSYIYYADSVKGRFTISRDNAKNSLYLQMNSLRAEDTAVYFCAR
EKWNLLYFDAWGQGTLVTVSS
VL:
(SEQ ID NO: 288)
NFMLTQPHSVSESPGKTVTISCTRSSGSIASNYVQWYQQRPGSSPTTVI
YEDNQRPSGVPDRFSGSIDSSSNSASLTISGLKTEDEADYYCQSYDSSN
RVFGCGTKLTVL
10B09A
HCDR1:
(SEQ ID NO: 289)
SYSMN
HCDR2:
(SEQ ID NO: 290)
SISRSSNYIYYADSVKG
HCDR3:
(SEQ ID NO: 291)
EKWNLLYFDY
LCDR1:
(SEQ ID NO: 292)
TRSSGSIATNYVQ
LCDR2:
(SEQ ID NO: 293)
EDNKRPS
LCDR3:
(SEQ ID NO: 294)
QSYDSSNRV
VH:
(SEQ ID NO: 295)
EVQLVESGGGLVKPGGSLRLSCAASGFTLSSYSMNWVRQAPGKCLEWVS
SISRSSNYIYYADSVKGRFTISRDNAKNSLYLQMNSLRAEDTAVYYCAR
EKWNLLYFDYWGQGTLVTVS
VL:
(SEQ ID NO: 296)
NLMLTQPHSVSGSPGKTVTISCTRSSGSIATNYVQWYQQRPGSSPTTVI
YEDNKRPSGVPDRFSGSIDSSSNSASLTISGLKTEDEADYYCQSYDSSN
RVFGCGTKLTVL
14E03A
HCDR1:
(SEQ ID NO: 297)
RYWMT
HCDR2:
(SEQ ID NO: 298)
NIKKDGSENYYVDSVKG
HCDR3:
(SEQ ID NO: 299)
VRVIYDWLDP
LCDR1:
(SEQ ID NO: 300)
SGDKLGNKYAA
LCDR2:
(SEQ ID NO: 301)
QDSKRPS
LCDR3:
(SEQ ID NO: 302)
QAWDNNIVV
VH:
(SEQ ID NO: 303)
EVQVVESGGGLVQPGGSLRLSCAASGFTFSRYWMTWVRQAPGKCLEWVA
NIKKDGSENYYVDSVKGRFTISRDNAKDSLYLQMNSLRAEDTAVYYCVR
VIYDWLDPWGQGTLVTVSS
VL:
(SEQ ID NO: 304)
SYELTQPPSVSVSPGQTASITCSGDKLGNKYAAWYQQKPGQSPVLVIYQ
DSKRPSGIPERFSGSNSGNTATLTISGTQAMDEADYYCQAWDNNIVVFG
CGTKLTVL
14O18A
HCDR1:
(SEQ ID NO: 305)
RYSMN
HCDR2:
(SEQ ID NO: 306)
SISMSSRYIYYVDSVKG
HCDR3:
(SEQ ID NO: 307)
ERGPGPGMDV
LCDR1:
(SEQ ID NO: 308)
TRSSGSIASNYVQ
LCDR2:
(SEQ ID NO: 309)
EDNQRPS
LCDR3:
(SEQ ID NO: 310)
QSYDSSNRV
VH:
(SEQ ID NO: 311)
EVQLVESGGGLVKFGGSLRLSCAASGFTFSRYSMNWVRQAPGKCLDWVS
SISMSSRYIYYVDSVKGRFTISRDNAKNSLYLQMNSLRADDTAVYYCAR
ERGPGPGMDVWGQGTTVTVSS
VL:
(SEQ ID NO: 312)
NFMLTQPHSVSESPGKTVTISCTRSSGSIASNYVQWYQQRPGSSPTTVI
YEDNQRPSGVPDRFSGSIDSSSNSASLTISGLKTEDEADYYCQSYDSSN
RVFGCGTKLTVL
ANTI-CLDN6 Binding Domain of Ab897
HCDR1:
(SEQ ID NO: 315)
NYGVH
HCDR2:
(SEQ ID NO: 316)
VIWAAGRTNYNSALMS
HCDR3:
(SEQ ID NO: 317)
DRYDYGYFDV
LCDR1:
(SEQ ID NO: 318)
SASSSVSYMY
LCDR2:
(SEQ ID NO: 319)
RTSSLAS
LCDR3:
(SEQ ID NO: 320)
QQYHSYPLT
VH:
(SEQ ID NO: 321)
QVQLQESGPGLVKPSETLSITCTVSGFSLINYGVHWIRQPPGKGLEWLG
VIWAAGRTNYNSALMSRLYISVDTSKNQVSLKLSSVTAADTAVYYCARD
RYDYGYFDVWGQGTMVTVSS
VL:
(SEQ ID NO: 322)
AIVLTQSPSSLSASTGDRVTISCSASSSVSYMYWYQQKSGKAPKLWIYR
TSSLASRVPSRFSGSGSGTDYTLTISSLQSEDFATYYCQQYHSYPLTFG
GGTKVEIK
HC:
(SEQ ID NO: 323)
QVQLQESGPGLVKPSETLSITCTVSGFSLINYGVHWIRQPPGKGLEWLG
VIWAAGRTNYNSALMSRLYISVDTSKNQVSLKLSSVTAADTAVYYCARD
RYDYGYFDVWGQGTMVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVCTLPPSRDELTKNQVSLSCAVKGFYPSDIAVEWESNGQPENNY
KTTPPVLDSDGSFFLVSKLTVDKSRWQQGNVFSCSVMHEALHNRFTQKS
LSLSPGK
LC:
(SEQ ID NO: 324)
AIVLTQSPSSLSASTGDRVTISCSASSSVSYMYWYQQKSGKAPKLWIYR
TSSLASRVPSRFSGSGSGTDYTLTISSLQSEDFATYYCQQYHSYPLTFG
GGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC
scFv Linker
Linker:
(SEQ ID NO: 325)
GGSEGKSSGSGSESKSTGGS
Antigens
CD3G:
(SEQ ID NO: 326)
MEQGKGLAVLILAIIILQGTLAQSIKGNHLVKVYDYQEDGSVLLTCDAE
AKNITWFKDGKMIGFLTEDKKKWNLGSNAKDPRGMYQCKGSQNKSKPLQ
VYYRMCQNCIELNAATISGFLFAEIVSIFVLAVGVYFIAGQDGVRQSRA
SDKQTLLPNDQLYQPLKDREDDQYSHLQGNQLRRN
CD3D:
(SEQ ID NO: 327)
MEHSTFLSGLVLATLLSQVSPFKIPIEELEDRVFVNCNTSITWVEGTVG
TLLSDITRLDLGKRILDPRGIYRCNGTDIYKDKESTVQVHYRMCQSCVE
LDPATVAGIIVTDVIATLLLALGVFCFAGHETGRLSGAADTQALLRNDQ
VYQPLRDRDDAQYSHLGGNWARNK
CD3E:
(SEQ ID NO: 328)
MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVC
YPRGSKPEDANFYLYLRARVCENCMEMDVMSVATIVIVDICITGGLLLL
VYYWSKNRKAKAKPVTRGAGAGGRQRGQNKERPPPVPNPDYEPIRKGQR
DLYSGLNQRRI
CD3Z:
(SEQ ID NO: 329)
MKWKALFTAAILQAQLPITEAQSFGLLDPKLCYLLDGILFIYGVILTAL
FLRVKFSRSADAPAYQQGQNQLYNELNLGRREEYDVLDKRRGRDPEMGG
KPQRRKNPQEGLYNELQKDKMAEAYSEIGMKGERRRGKGHDGLYQGLST
ATKDTYDALHMQALPPR
CLDN6:
(SEQ ID NO: 330)
MASAGMQILGVVLTLLGWVNGLVSCALPMWKVTAFIGNSIVVAQVVWEG
LWMSCVVQSTGQMQCKVYDSLLALPQDLQAARALCVIALLVALFGLLVY
LAGAKCTTCVEEKDSKARLVLTSGIVFVISGVLTLIPVCWTAHAIIRDF
YNPLVAEAQKRELGASLYLGWAASGLLLLGGGLLCCTCPSGGSQGPSHY
MARYSTSAPAISRGPSEYPTKNYV
CD3ϵ epitope 1:
(SEQ ID NO: 331
GSEIL
CD3ϵ epitope 2:
(SEQ ID NO: 332)
CYPRGSKPE
CD3ϵ epitope 3:
(SEQ ID NO: 333)
TCPQYP
CD3ϵ epitope 4:
(SEQ ID NO: 334)
QSGYYV
CD3ϵ epitope 5:
(SEQ ID NO: 335)
DANFYLYLRA
CD3ϵ epitope:
(SEQ ID NO: 336)
RLDLGKRILDPRGIY

1. An anti-CD3 antibody or antigen-binding fragment thereof comprising:
a) a heavy chain complementarity determining region 1 (HCDR1) comprising the amino acid sequence of any one of SEQ ID NOs: 1, 9, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289, 297, and 305;
b) an HCDR2 comprising the amino acid sequence of any one of SEQ ID NOs: 2, 10, 18, 26, 34, 42, 50, 58, 66, 74, 82, 90, 98, 106, 114, 122, 130, 138, 146, 154, 162, 170, 178, 186, 194, 202, 210, 218, 226, 234, 242, 250, 258, 266, 274, 282, 290, 298, and 306;
c) an HCDR3 comprising the amino acid sequence of any one of SEQ ID NOs: 3, 11, 19, 27, 35, 43, 51, 59, 67, 75, 83, 91, 99, 107, 115, 123, 131, 139, 147, 155, 163, 171, 179, 187, 195, 203, 211, 219, 227, 235, 243, 251, 259, 267, 275, 283, 291, 299, and 307;
d) a light chain complementarity determining region 1 (LCDR1) comprising the amino acid sequence of any one of SEQ ID NOs: 4, 12, 20, 28, 36, 44, 52, 60, 68, 76, 84, 92, 100, 108, 116, 124, 132, 140, 148, 156, 164, 172, 180, 188, 196, 204, 212, 220, 228, 236, 244, 252, 260, 268, 276, 284, 292, 300, and 308;
e) an LCDR2 comprising the amino acid sequence of any one of SEQ ID NOs: 5, 13, 21, 29, 37, 45, 53, 61, 69, 77, 85, 93, 101, 109, 117, 125, 133, 141, 149, 157, 165, 173, 181, 189, 197, 205, 213, 221, 229, 237, 245, 253, 261, 269, 277, 285, 293, 301, and 309; and
f) an LCDR3 comprising the amino acid sequence of any one of SEQ ID NOs: 6, 14, 22, 30, 38, 46, 54, 62, 70, 78, 86, 94, 102, 110, 118, 126, 134, 142, 150, 158, 166, 174, 182, 190, 198, 206, 214, 222, 230, 238, 246, 254, 262, 270, 278, 286, 294, 302, and 310.

2. (canceled)

3. The anti-CD3 antibody or antigen-binding fragment thereof of claim 1, comprising an HCDR1, HCDR2, HCDR3, LCDR1, LCDR2, and LCDR3 comprising the amino acid sequences of:
a) SEQ ID NOs: 1, 2, 3, 4, 5, and 6, respectively;
b) SEQ ID NOs: 9, 10, 11, 12, 13, and 14, respectively;
c) SEQ ID NOs: 17, 18, 19, 20, 21, and 22, respectively;
d) SEQ ID NOs: 25, 26, 27, 28, 29, and 30, respectively;
e) SEQ ID NOs: 33, 34, 35, 36, 37, and 38, respectively;
f) SEQ ID NOs: 41, 42, 43, 44, 45, and 46, respectively;
g) SEQ ID NOs: 49, 50, 51, 52, 53, and 54, respectively;
h) SEQ ID NOs: 57, 58, 59, 60, 61, and 62, respectively;
i) SEQ ID NOs: 65, 66, 67, 68, 69, and 70, respectively;
j) SEQ ID NOs: 73, 74, 75, 76, 77, and 78, respectively;
k) SEQ ID NOs: 81, 82, 83, 84, 85, and 86, respectively;
l) SEQ ID NOs: 89, 90, 91, 92, 93, and 94, respectively;
m) SEQ ID NOs: 97, 98, 99, 100, 101, and 102, respectively;
n) SEQ ID NOs: 105, 106, 107, 108, 109, and 110, respectively;
o) SEQ ID NOs: 113, 114, 115, 116, 117, and 118, respectively;
p) SEQ ID NOs: 121, 122, 123, 124, 125, and 126, respectively;
q) SEQ ID NOs: 129, 130, 131, 132, 133, and 134, respectively;
r) SEQ ID NOs: 137, 138, 139, 140, 141, and 142, respectively;
s) SEQ ID NOs: 145, 146, 147, 148, 149, and 150, respectively;
t) SEQ ID NOs: 153, 154, 155, 156, 156, and 158, respectively;
u) SEQ ID NOs: 161, 162, 263, 164, 165, and 166, respectively;
v) SEQ ID NOs: 169, 170, 171, 172, 173, and 174, respectively;
w) SEQ ID NOs: 177, 178, 179, 180, 181, and 182, respectively;
x) SEQ ID NOs: 185, 186, 187, 188, 189, and 190, respectively;
y) SEQ ID NOs: 193, 194, 195, 196, 197, and 198, respectively;
z) SEQ ID NOs: 201, 202, 203, 204, 205, and 206, respectively;
aa) SEQ ID NOs: 209, 210, 211, 212, 213, and 214, respectively;
ab) SEQ ID NOs: 217, 218, 219, 220, 221, and 222, respectively;
ac) SEQ ID NOs: 225, 226, 227, 228, 229, and 230, respectively;
ad) SEQ ID NOs: 233, 234, 235, 236, 237, and 238, respectively;
ae) SEQ ID NOs: 241, 242, 243, 244, 245, and 246, respectively;
af) SEQ ID NOs: 249, 250, 251, 252, 253, and 254, respectively;
ag) SEQ ID NOs: 257, 258, 259, 260, 261, and 262, respectively;
ah) SEQ ID NOs: 265, 266, 267, 268, 269, and 270, respectively;
ai) SEQ ID NOs: 273, 274, 275, 276, 277, and 278, respectively;
aj) SEQ ID NOs: 281, 282, 283, 284, 285, and 286, respectively;
ak) SEQ ID NOs: 289, 290, 291, 292, 293, and 294, respectively;
al) SEQ ID NOs: 297, 298, 299, 300, 301, and 302, respectively; or
am) SEQ ID NOs: 305, 306, 307, 308, 309, and 310, respectively.

4. (canceled)

5. The anti-CD3 antibody or antigen-binding fragment thereof of claim 1, comprising a VH and a VL comprising the amino acid sequences of:
a) SEQ ID NOs: 7 and 8, respectively;
b) SEQ ID NOs: 15 and 16, respectively;
c) SEQ ID NOs: 23 and 24, respectively;
d) SEQ ID NOs: 31 and 32, respectively;
e) SEQ ID NOs: 39 and 40, respectively;
f) SEQ ID NOs: 47 and 48, respectively;
g) SEQ ID NOs: 55 and 56, respectively;
h) SEQ ID NOs: 63 and 64, respectively;
i) SEQ ID NOs: 71 and 72, respectively;
j) SEQ ID NOs: 79 and 80, respectively;
k) SEQ ID NOs: 87 and 88, respectively;
l) SEQ ID NOs: 95 and 96, respectively;
m) SEQ ID NOs: 103 and 104, respectively;
n) SEQ ID NOs: 111 and 112, respectively;
o) SEQ ID NOs: 119 and 120, respectively;
p) SEQ ID NOs: 127 and 128, respectively;
q) SEQ ID NOs: 135 and 136, respectively;
r) SEQ ID NOs: 143 and 144, respectively;
s) SEQ ID NOs: 151 and 152, respectively;
t) SEQ ID NOs: 159 and 160, respectively;
u) SEQ ID NOs: 167 and 168, respectively;
v) SEQ ID NOs: 175 and 176, respectively;
w) SEQ ID NOs: 183 and 184, respectively;
x) SEQ ID NOs: 191 and 192, respectively;
y) SEQ ID NOs: 199 and 200, respectively;
z) SEQ ID NOs: 207 and 208, respectively;
aa) SEQ ID NOs: 215 and 216, respectively;
ab) SEQ ID NOs: 223 and 224, respectively;
ac) SEQ ID NOs: 231 and 232, respectively;
ad) SEQ ID NOs: 239 and 240, respectively;
ae) SEQ ID NOs: 247 and 248, respectively;
af) SEQ ID NOs: 255 and 256, respectively;
ag) SEQ ID NOs: 263 and 264, respectively;
ah) SEQ ID NOs: 271 and 272, respectively;
ai) SEQ ID NOs: 279 and 280, respectively;
aj) SEQ ID NOs: 287 and 288, respectively;
ak) SEQ ID NOs: 295 and 296, respectively;
al) SEQ ID NOs: 303 and 304, respectively; or
am) SEQ ID NOs: 311 and 312, respectively.

6. The anti-CD3 antibody or antigen-binding fragment thereof of claim 1, wherein the antibody specifically binds:
an epitope of CD3ε (SEQ ID NO:328) comprising:
at least one amino acid in at least one of amino acid sequence selected from SEQ ID NOs: 331-335;
at least one amino acid sequence selected from SEQ ID NOs: 331-335; or
SEQ ID NOs: 331-335;
and an epitope of CD3δ (SEQ ID NO:327) comprising:
at least one amino acid in SEQ ID NO:336; or
SEQ ID NO:336.

7-8. (canceled)

9. The anti-CD3 antibody or antigen-binding fragment thereof of claim 1, wherein the antibody is human or humanized.

10. The anti-CD3 antibody or antigen-binding fragment thereof of claim 1, wherein the antibody comprises an Fc domain.

11. (canceled)

12. The anti-CD3 antibody or antigen-binding fragment thereof of claim 10, wherein the Fc domain is an IgG1 Fc domain.

13. The anti-CD3 antibody or antigen-binding fragment thereof of claim 12, wherein the Fc domain comprises M252Y, S254T, and T256E (YTE) substitutions, wherein numbering is according to the EU index.

14. The anti-CD3 antibody or antigen-binding fragment thereof of claim 1, wherein the antibody is a bispecific or multispecific antibody.

15. A composition comprising:
a) an isolated nucleic acid encoding the anti-CD3 antibody or antigen-binding fragment thereof of claim 1;
b) a vector comprising the isolated nucleic acid of a); or
c) a host cell comprising the vector of b).

16-31. (canceled)

32. The anti-CD3 antibody or antigen-binding fragment thereof of claim 14, wherein the bispecific or multispecific antibody comprises two Fc chains.

33. The anti-CD3 antibody or antigen-binding fragment thereof of claim 32, wherein the first Fc domain comprises T366S, L368A, and Y407V substitutions and the second Fc domain comprises a T366W substitution, wherein numbering is according to the EU index.

34. The anti-CD3 antibody or antigen-binding fragment thereof of claim 33, wherein the Fc domains further comprise an S354C and a Y349C substitution, wherein numbering is according to the EU index.

35. The anti-CD3 antibody or antigen-binding fragment thereof of claim 14, wherein the second antigen binding domain specifically binds a cell surface antigen that is expressed on a cell other than an immune effector cell.

36. The anti-CD3 antibody or antigen-binding fragment thereof of claim 35, wherein the cell surface antigen is a tumor associated antigen.

37. A composition comprising:
a) the anti-CD3 antibody or antigen-binding fragment thereof of claim 14;
b) an isolated nucleic acid encoding the bispecific or multispecific antibody;
c) a vector comprising the isolated nucleic acid of b); or
d) a host cell comprising the vector of c).

38-58. (canceled)

59. A method of treating a disease or disorder in a subject in need thereof comprising administering to the subject the antibody or antigen-binding fragment thereof of claim 1.

60. The method of claim 59, wherein the disease or disorder is cancer.

61. (canceled)

62. A method of treating a disease or disorder in a subject in need thereof comprising administering to the subject the anti-CD3 antibody or antigen-binding fragment thereof of claim 14.

63. The method of claim 62, wherein the disease or disorder is cancer.

64. (canceled)
